(19) United States
(12) Patent Application Publication
(10) Pub. No.: US 2026/0355318 A1
(43) Pub. Date: Nov. 12, 2026

(54) MOLECULAR MACHINES FOR TREATMENT OF CANCER, FUNGAL INFECTIONS, OR BACTERIAL INFECTIONS

(71) Applicants: William Marsh Rice University, Houston, TX (US); Fundació Institut d'Investigació Sanitària Illes Balears, Palma (ES)

(72) Inventors: James M. TOUR, Houston, TX (US); Ana L. SANTOS, Houston, TX (US); Jacob L. BECKHAM, Houston, TX (US); Gang LI, Houston, TX (US); Ciceron AYALA-OROZCO, Houston, TX (US); David IZHAKY, Hod Hasharon (IL); Shlomo NEDVETZKI, Hod Hasharon (IL)

(73) Assignees: William Marsh Rice University, Houston, TX (US); Fundació Institut d'Investigació Sanitària Illes Balears, Palma (ES)

(21) Appl. No.: 19/115,760

(22) PCT Filed: Sep. 28, 2023
(86) PCT No.: PCT/US2023/075445

§ 371 Date: Mar. 26, 2025

Related U.S. Application Data
(60) Provisional application No. 63/411,012, filed on Sep. 28, 2022.

(51) Int. Cl. A61K 41/00; A61K 31/136; A61K 31/382; A61K 31/496; A61P 35/00; C07C 211/61; C07D 335/12; C07D 335/20

(52) U.S. Cl. CPC A61K 41/0057; A61K 31/136; A61K 31/382; A61K 31/496; A61P 35/00; C07C 211/61; C07D 335/12; C07D 335/20

(57) ABSTRACT

The present disclosure relates to stimulus activated molecular machines comprising rotor and stator moieties and designed to treat bacterial or fungal infections, or cancer. In certain embodiments, the stimulus activated molecular machines are activated by light, including laser, which stimulates mechanical action that can be precisely controlled.

### PRIORITY CLAIM

This application claims benefit of priority to U.S. Provisional Application Ser. No. 63/411,012, filed Sep. 28, 2022, the entire contents of which are hereby incorporated by reference.

### STATEMENT REGARDING FEDERALLY SPONSORED RESEARCH

This invention was made with government support under Grant No. 1842494 awarded by the National Science Foundation. The government has certain rights in the invention.

### BACKGROUND

### I. Field

The present disclosure relates to the fields of chemistry, biology, and medicine. More particularly, it relates to molecular machines for treating or preventing diseases or disorders.

### II. Related Art

Treatment of diseases caused by microorganisms is a continuing problem. In particular, these types of diseases are often complicated by the fact that microorganisms often develop resistance to commonly used treatments. For example, antifungal resistance is common given that there are only three major classes of antifungal agents: azoles, echinocandins, and polyenes. Similarly, bacteria often develop resistance to antibiotics especially when antibiotics are not properly used. Resistance is not merely limited to microbial infections, and can occur in other conditions. In particular, cancers often become resistant to particular types of treatments.

The mechanism of molecular machines, which is involves mechanical action, is less likely to permit development of resistance. Therefore, the need for developing new therapeutics that reduce the likelihood of resistance and can be used to treat multiple different types of conditions.

This invention was funded in part by the Robert A. Welch Foundation under Welch Grant No. C-2017-20190330.

### SUMMARY

The present disclosure provides a method for the use of stimulus activated molecular machines to treat a fungal or bacterial disease or a cancer. In some embodiments the stimulus activated molecular machines are not targeted by the natural defensive arsenal of microorganisms, such as fungi, bacteria, or cancer cells. In this way, the methods disclosed herein represent an unexpected and unforeseeable approach to treating fungal infections, bacterial infections, or cancer. In some embodiments, the presently disclosed methods allow for improved control over the therapeutic compounds, more particularly the stimulus activated molecular machines, in time and/or in space, thereby mitigating detrimental side effects to human cells and providing an advantage over corresponding known methods.

In one aspect, the present disclosure provides methods of treating a disease or disorder in a patient caused by an infection of a microorganism comprising:
(A) administering to the patient in need thereof a stimulus activated molecular machine, wherein the stimulus activated molecule machine is configured to be activated by an appropriate stimulus; and
(B) exposing the patient to a stimulus sufficient to activate the molecular machine.

In another aspect, the present disclosure provides methods of treating a fungal infection in a patient comprising:
(A) administering to the patient in need thereof a stimulus activated molecular machine, wherein the stimulus activated molecule machine is configured to be activated by an appropriate stimulus; and
(B) exposing the patient to a stimulus sufficient to activate the molecular machine.

In another aspect, the present disclosure provides compositions for use in the treatment of a disease or disorder in a patient caused by an infection of a microorganism comprising a stimulus activated molecule machine is configured to be activated by an appropriate stimulus. In some embodiments, the present disclosure provides for use of a stimulus activated molecular machine in the treatment of a disease or disorder in a patient caused by an infection of a microorganism.

In another aspect, the present disclosure provides methods of inhibiting the growth of a microorganism comprising contacting the microorganism with a stimulus activated molecular machine, wherein the stimulus activated molecule machine is configured to be activated by an appropriate stimulus, and exposing the microorganism to an appropriate stimulus. In another aspect, the present disclosure provides compositions for use in inhibiting the growth of a microorganism comprising a stimulus activated molecular machine, wherein the stimulus activated molecule machine is configured to be activated by an appropriate stimulus. In another aspect, the present disclosure provides for use of a stimulus activated molecular motor for inhibiting the growth of a microorganism, wherein the stimulus activated molecule machine is configured to be activated by an appropriate stimulus.

In another aspect, the present disclosure provides methods of killing a microorganism comprising contacting the microorganism with a stimulus activated molecular machine, wherein the stimulus activated molecule machine is configured to be activated by an appropriate stimulus, and exposing the microorganism to an appropriate stimulus. In another aspect, the present disclosure provides compositions for use in killing a microorganism comprising a stimulus activated molecular machine, wherein the stimulus activated molecule machine is configured to be activated by an appropriate stimulus. In another aspect, the present disclosure provides uses of a stimulus activated molecular motor for killing a microorganism, wherein the stimulus activated molecule machine is configured to be activated by an appropriate stimulus.

In another aspect, the present disclosure provides methods of inhibiting a biofilm formation comprising contacting the biofilm with a stimulus activated molecular machine, wherein the stimulus activated molecule machine is configured to be activated by an appropriate stimulus, and exposing the biofilm to an appropriate stimulus. In another aspect, the present disclosure provides compositions for use in inhibiting a biofilm formation comprising a stimulus activated molecular machine, wherein the stimulus activated molecule machine is configured to be activated by an appropriate stimulus. In another aspect, the present disclosure provides In another aspect, the present disclosure provides uses of a stimulus activated molecular motor for inhibiting a biofilm formation, wherein the stimulus activated molecule machine is configured to be activated by an appropriate stimulus.

In another aspect, the present disclosure provides methods of eliminating a biofilm comprising contacting the biofilm with a stimulus activated molecular machine, wherein the stimulus activated molecule machine is configured to be activated by an appropriate stimulus, and exposing the biofilm to an appropriate stimulus. In another aspect, the present disclosure provides compositions for use in eliminating a biofilm comprising a stimulus activated molecular machine, wherein the stimulus activated molecule machine is configured to be activated by an appropriate stimulus. In another aspect, the present disclosure provides uses of a stimulus activated molecular motor for eliminating a biofilm, wherein the stimulus activated molecule machine is configured to be activated by an appropriate stimulus.

In another aspect, the present disclosure provides methods of inducing necrosis in a microorganism comprising contacting the microorganism with a stimulus activated molecular machine, wherein the stimulus activated molecule machine is configured to be activated by an appropriate stimulus, and exposing the microorganism to an appropriate stimulus. In another aspect, the present disclosure provides compositions for use in inducing necrosis in a microorganism comprising a stimulus activated molecular machine, wherein the stimulus activated molecule machine is configured to be activated by an appropriate stimulus. In another aspect, the present disclosure provides uses of a stimulus activated molecular motor for inducing necrosis in a microorganism, wherein the stimulus activated molecule machine is configured to be activated by an appropriate stimulus.

In another aspect, the present disclosure provides methods of causing oxidative stress in a cell comprising contacting the cell with a stimulus activated molecular machine, wherein the stimulus activated molecule machine is configured to be activated by an appropriate stimulus, and exposing the cell to an appropriate stimulus. In another aspect, the present disclosure provides compositions for use in causing oxidative stress in a cell comprising a stimulus activated molecular machine, wherein the stimulus activated molecule machine is configured to be activated by an appropriate stimulus. In another aspect, the present disclosure provides uses of a stimulus activated molecular motor in causing oxidative stress in a cell, wherein the stimulus activated molecule machine is configured to be activated by an appropriate stimulus.

In another aspect, the present disclosure provides methods of inhibiting mitochondria function in a cell comprising contacting the cell with a stimulus activated molecular machine, wherein the stimulus activated molecule machine is configured to be activated by an appropriate stimulus, and exposing the cell to an appropriate stimulus. In another aspect, the present disclosure provides compositions for use inhibiting mitochondria function in a cell comprising a stimulus activated molecular machine, wherein the stimulus activated molecule machine is configured to be activated by an appropriate stimulus. In another aspect, the present disclosure provides uses of a stimulus activated molecular motor for inhibiting mitochondria function in a cell, wherein the stimulus activated molecule machine is configured to be activated by an appropriate stimulus.

In another aspect, the present disclosure provides methods of overcoming drug resistance in a microorganism comprising contacting the microorganism with a stimulus activated molecular machine, wherein the stimulus activated molecule machine is configured to be activated by an appropriate stimulus, and exposing the microorganism to an appropriate stimulus in the presence of a drug to which the microorganism was resistant to. In another aspect, the present disclosure provides compositions for use in overcoming drug resistance in a microorganism comprising a stimulus activated molecular machine, wherein the stimulus activated molecule machine is configured to be activated by an appropriate stimulus. In another aspect, the present disclosure provides uses of a stimulus activated molecular motor for overcoming drug resistance in a microorganism, wherein the stimulus activated molecule machine is configured to be activated by an appropriate stimulus.

In some embodiments, the microorganism is a bacterium. In further embodiments, the bacterium is gram positive bacteria. In other embodiments, the bacterium is gram negative bacteria. In still other embodiments, the bacterium is a gram indeterminate bacteria.

In some embodiments, the bacterium is sensitive to one or more antibiotics. In further embodiments, the bacterium is sensitive to two or more antibiotics. In some embodiments, the antibiotic is methicillin, cefoxitin, oxacillin, gentamicin, ciprofloxacin, levofloxacin, moxifloxacin, erythromycin, clindamycin, linezolid, daptomycin, vancomycin, doxycycline, tobramycin, tetracycline, tigecycline, nitrofurantoin, rifampin, trimethoprim-sulfamethoxazole, amoxicillin-clavulanic acid, ampicillin-sulbactam, piperacillin-tazobactam, cefepime, ertapenem, imipenem, and meropenem. In some embodiments, the bacterium is a gram positive bacterium and is resistant to cefoxitin, oxacillin, gentamicin, ciprofloxacin, levofloxacin, moxifloxacin, erythromycin, clindamycin, linezolid, daptomycin, vancomycin, doxycycline, tetracycline, tigecycline, nitrofurantoin, rifampin, or trimethoprim-sulfamethoxazole. In some embodiments, the bacterium is a gram negative bacterium and is resistant to amoxicillin-clavulanic acid, ampicillin-sulbactam, piperacillin-tazobactam, cefepime, ertapenem, imipenem, meropenem, gentamicin, tobramycin, ciprofloxacin, levofloxacin, nitrofurantoin, or trimethoprim-sulfamethoxazole.

In some embodiments, the bacterium is from a hospital acquired infection. In some embodiments, the bacterium is Staphylococcus saprophyticus (S. saprophyticus), Staphylococcus aureus (S. aureus), methicillin-resistant Staphylococcus aureus (MRSA), coagulase negative staphylococcus (CNS), methicillin-resistant CNS (MRCNS), E. coli, multi-drug resistance (MDR) E. coli, MDR-Citrobacter koseri, MDR-Enterobacter cloacae complex, MDR-Morganella morganii, MDR-Klebsiella pneumonia or MDR-Acinetobacter baumannii.

In some embodiments, the method further comprises administering a second antibiotic agent.

In some embodiments, the microorganism is a fungus. In some embodiments, the fungus is a Basidiomycota fungus, such as a Cryptococcus fungus. In other embodiments, the fungus is an Ascomycota fungus. In some embodiments, the fungus is an Aspergillus, Candida, Coccidioides, Histoplasma, or Blastomyces fungus. In other embodiments, the fungus is a Mucoromycorina fungus.

In some embodiments, the method further comprises administering a second anti-fungal therapy. In further embodiments, the second anti-fungal therapy is a therapy targeting the ergosterol biosynthetic pathway. In some embodiments, the second anti-fungal therapy is Amphotericin B, fluconazole, itraconazole, posaconazole, or voriconazole. In further embodiments, the second anti-fungal therapy is voriconazole. In other embodiments, the second anti-fungal therapy is echinocandins or flucytosine.

In some embodiments, the fungus has infected the central nervous system. In other embodiments, the fungus has infected the lungs. In some embodiments, the fungus is present in its spore form.

In one aspect, the present disclosure provides methods of treating a disease or disorder in a patient caused by an infection of a fungus comprising:
(A) administering to the patient in need thereof a stimulus activated molecular machine, wherein the stimulus activated molecule machine is configured to be activated by an appropriate stimulus; and
(B) exposing the patient to a stimulus sufficient to activate the molecular machine;
wherein the stimulus activated molecular motor comprises a rotor of the formula:

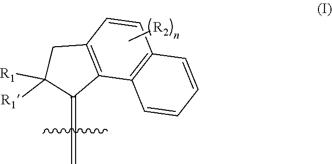

wherein:
   R₁ and R₁′ are each independently hydrogen, a C1-C12 alkyl or a C1-C12 substituted alkyl;
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁—X₁—R₂′, wherein:
      Y₁ is —O—, —S—, or —NRₐ—, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R₂′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4;
and a stator of the formula:

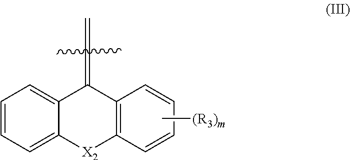

wherein:
   X₂ is a covalent bond, O, S, NR_{c}, or CR_{d}R_{d}′, wherein R_{c}, R_{d}, and R_{d}′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
   m is 0, 1, 2, 3, or 4.

In one aspect, the present disclosure provides methods of treating a disease or disorder in a patient caused by an infection of a bacteria comprising:
(A) administering to the patient in need thereof a stimulus activated molecular machine, wherein the stimulus activated molecule machine is configured to be activated by an appropriate stimulus; and
(B) exposing the patient to a stimulus sufficient to activate the molecular machine.
wherein the stimulus activated molecular motor comprises a rotor of the formula:

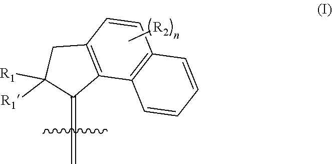

wherein:
   R₁ and R₁′ are each independently hydrogen, a C1-C12 alkyl or a C1-C12 substituted alkyl;
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁—X₁—R₂′, wherein:
      Y₁ is —O—, —S—, or —NRₐ —, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R₂′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4;
and a stator of the formula:

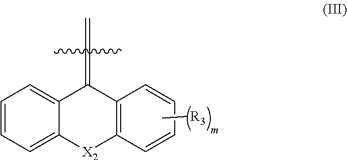

wherein:
   X₂ is a covalent bond, O, S, NR_{c}, or CR_{d}R_{d}′, wherein R_{c}, R_{d}, and R_{d}′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f} and R_{f}′ are each independently hydrogen, C1-C6 alkyl, or C1-C6 substituted alkyl, and R₃″ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
   m is 0, 1, 2, 3, or 4.

In another aspect, the present disclosure provides methods of treating cancer in a patient in need thereof comprising administering to the patient a stimulus activated molecular machine, wherein the stimulus activated molecule machine is configured to be activated by an appropriate stimulus, and exposing the patient to an appropriate stimulus. In another aspect, the present disclosure provides compositions for use in treating cancer comprising a stimulus activated molecular machine, wherein the stimulus activated molecule machine is configured to be activated by an appropriate stimulus. In another aspect, the present disclosure provides uses of a stimulus activated molecular motor in treating cancer, wherein the stimulus activated molecule machine is configured to be activated by an appropriate stimulus.

In another aspect, the present disclosure provides methods of reducing the tumor burden in a patient comprising administering to the patient a stimulus activated molecular machine, wherein the stimulus activated molecule machine is configured to be activated by an appropriate stimulus, and exposing the patient to an appropriate stimulus. In another aspect, the present disclosure provides compositions for use in reducing the tumor burden in a patient comprising a stimulus activated molecular machine, wherein the stimulus activated molecule machine is configured to be activated by an appropriate stimulus. In another aspect, the present disclosure provides uses of a stimulus activated molecular motor in reducing the tumor burden in a patient, wherein the stimulus activated molecule machine is configured to be activated by an appropriate stimulus.

In another aspect, the present disclosure provides methods of enhancing the effect of a chemotherapeutic compound in a patient comprising administering to the patient a stimulus activated molecular machine, wherein the stimulus activated molecule machine is configured to be activated by an appropriate stimulus, and exposing the patient to an appropriate stimulus after the patient has been administered the chemotherapeutic compound. In another aspect, the present disclosure provides compositions for use in enhancing the effect of a chemotherapeutic compound comprising a stimulus activated molecular machine, wherein the stimulus activated molecule machine is configured to be activated by an appropriate stimulus. In another aspect, the present disclosure provides uses of a stimulus activated molecular motor in enhancing the effect of a chemotherapeutic compound, wherein the stimulus activated molecule machine is configured to be activated by an appropriate stimulus.

In another aspect, the present disclosure provides methods of killing a cancerous cell comprising contacting the cancerous cell with a stimulus activated molecular machine, wherein the stimulus activated molecule machine is configured to be activated by an appropriate stimulus, and exposing the cancerous cell to an appropriate stimulus. In another aspect, the present disclosure provides compositions for use in killing a cancerous cell comprising a stimulus activated molecular machine, wherein the stimulus activated molecule machine is configured to be activated by an appropriate stimulus. In another aspect, the present disclosure provides uses of a stimulus activated molecular motor in killing a cancerous cell, wherein the stimulus activated molecule machine is configured to be activated by an appropriate stimulus.

In another aspect, the present disclosure provides methods of inducing necrosis in a cancerous cell comprising contacting the cancerous cell with a stimulus activated molecular machine, wherein the stimulus activated molecule machine is configured to be activated by an appropriate stimulus, and exposing the cancerous cell to an appropriate stimulus. In another aspect, the present disclosure provides compositions for use in inducing necrosis in a cancerous cell comprising a stimulus activated molecular machine, wherein the stimulus activated molecule machine is configured to be activated by an appropriate stimulus. In another aspect, the present disclosure provides uses of a stimulus activated molecular motor in inducing necrosis in a cancerous cell, wherein the stimulus activated molecule machine is configured to be activated by an appropriate stimulus.

In another aspect, the present disclosure provides methods of treating cancer in a patient in need thereof comprising administering to the patient a stimulus activated molecular machine, wherein the stimulus activated molecule machine is configured to be activated by an appropriate stimulus, and exposing the patient to an appropriate stimulus
wherein the stimulus activated molecular machine comprises a rotor of the formula:

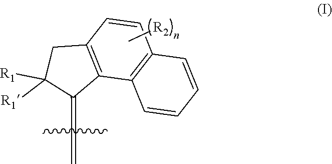

wherein:
   R₁ and R₁′ are each independently hydrogen, a C1-C12 alkyl or a C1-C12 substituted alkyl;
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁—X₁—R₂′, wherein:
      Y₁ is —O—, —S—, or —NRₐ—, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R_{f}′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4; and a stator of the formula:

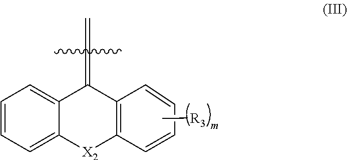

wherein:
   X₂ is a covalent bond, O, S, NR_{c}, or CR_{d}R_{d}′, wherein R_{c}, R_{d}, and R₃′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
   m is 0, 1, 2, 3, or 4;
   provided that the compound is not:

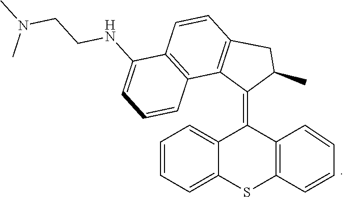

In some embodiments, the stimulus activated molecular motor comprises a rotor of the formula:

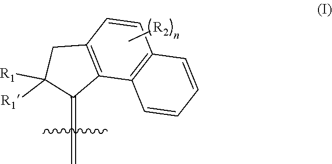

wherein:
   R₁ and R₁′ are each C1-C12 alkyl or C1-C12 substituted alkyl;
   R₁′ is hydrogen; and
   n is 0,
and a stator of the formula:

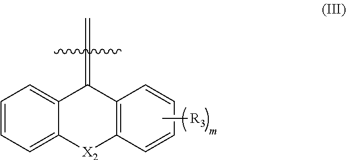

wherein:
   X₂ is S;
   R₃ is hydrogen; or
   R₃ is a group of the formula: —Y₂—X₃—R₃′, wherein:
      Y₂ is —NRₑ—, wherein: Rₑ is hydrogen, C1-C6 alkyl, or substituted C1-C6 alkyl;
      X₃ is C1-C12 alkanediyl or C1-C12 substituted alkanediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl; and
m is 1.

In some embodiments, the stimulus activated molecular machine is further defined as:

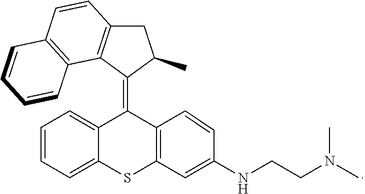

In some embodiments, the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

In some embodiments, the cancer is resistant to one or more chemotherapeutic compounds. In some embodiments, the method further comprises administering a second therapeutic agent. In further embodiments, the second therapeutic agent is a second chemotherapeutic agent, surgery, photodynamic therapy, sonodynamic therapy, radiotherapy, or immunotherapy.

In some embodiments, the stimulus activated molecular machine comprises a Feringa-type molecular machine. In some embodiments, the stimulus activated molecular machine comprises a rotor that is connected to a stator. In further embodiments, the stimulus activated molecular machine comprises a rotor that is connected to a stator through an alkenyl or alkynyl group. In some embodiments, the stimulus activated molecular machine comprises a rotor that is connected to a stator through an atropisomeric alkene.

In some embodiments, the rotor comprises one, two, three, four, or five rings. In further embodiments, the rotor comprises one, two, or three aromatic rings. In some embodiments, the rotor further comprises one, two, or three aliphatic rings. In some embodiments, the rotor comprises one, two, or three aromatic rings and one or two aliphatic rings. In some embodiments, the rotor comprises one, two, or three aliphatic or aromatic rings. In further embodiments, the rotor comprises two aromatic rings and an aliphatic ring.

In some embodiments, the rotor is further defined as:

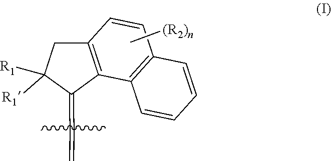

wherein:
   R₁ and R₁′ are each independently hydrogen, a C1-C12 alkyl or a C1-C12 substituted alkyl;
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁—X₁—R₂′, wherein:
      Y₁ is —O—, —S—, or —NRₐ—, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R₂′ is —NR_{b}R_{b}′Rₑ″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4.

In some embodiments, wherein the rotor is further defined as:

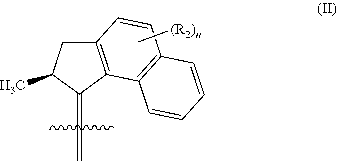

wherein:
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁—X₁—R₂′, wherein:
      Y₁ is —O—, —S—, or —NRₐ—, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R₂′ is —NR_{b}R_{b}′R_{b}, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4.

In some embodiments, R₁ is C1-C12 alkyl or substituted C1-C12 alkyl. In further embodiments, R₁ is C1-C12 alkyl. In still further embodiments, R₁ is methyl. In some embodiments, R₁′ is hydrogen. In some embodiments, R₂ is hydrogen. In other embodiments, R₂ is —Y₁-X₁-R₂′. In some embodiments, Y₁ is —NRₐ—. In further embodiments, R₃ is hydrogen.

In some embodiments, X₁ is C1-C12 alkanediyl or C1-C12 substituted alkanediyl. In further embodiments, X₁ is C1-C12 alkanediyl. In still further embodiments, X₁ is ethylene.

In some embodiments, R_{b} is C1-C6 alkyl or C1-C6 substituted alkyl. In further embodiments, R₁ is C1-C6 alkyl, such as methyl.

In some embodiments, R_{b}′ is C1-C6 alkyl or C1-C6 substituted alkyl. In some embodiments, R_{b}′ is C1-C6 alkyl, such as methyl. In some embodiments, R_{b}″ is absent. In some embodiments, R₂ is —NHCH₂CH₂N(Me)₂. In some embodiments, n is 0 or 1. In some embodiments n is 0. In other embodiments, n is 1.

In some embodiments, the molecular machine or switch comprises a stator, wherein the stator comprises one, two, three, four, or five rings. In some embodiments, the stator comprises one, two, three, four, or five aromatic rings. In some embodiments, the stator comprises one, two, or three aromatic rings. In some embodiments, the stator comprises one, two, three, four, or five aliphatic rings. In some embodiments, the stator comprises one, two, or three aliphatic rings. In some embodiments, the stator comprises two, three, or four rings. In some embodiments, the stator comprises three rings. In some embodiments, the stator comprises three rings with at least 2 aromatic rings.

In some embodiments, the stator is further defined as:

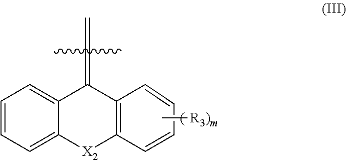

wherein:
   X₂ is a covalent bond, O, S, NR_{c}, or CR_{d}R_{d}′, wherein Rₑ, R_{d}, and R_{d}′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f} and R_{f}′ are each independently hydrogen, C1-C6 alkyl, or C1-C6 substituted alkyl, and Rr′ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
   m is 0, 1, 2, 3, or 4.

In some embodiments, the stator is further defined as:

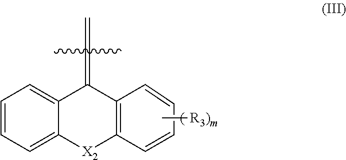

wherein:
   X₂ is a covalent bond, O, S, NR_{c}, or CRₐR_{d}′, wherein R_{c}, R_{d}, and R_{d}′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
   m is 0, 1, 2, 3 or 4.

In some embodiments, the stator is further defined as:

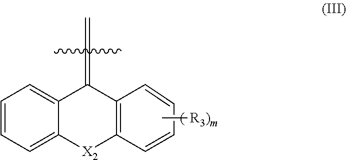

wherein:
   X₂ is a covalent bond, O, S, NRₑ, or CRₐR_{d}′, wherein R_{c}, R_{d}, and R_{d}′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
   m is 0 or 1.

In some embodiments, the stimulus activated molecular motor comprises a rotor of the formula:

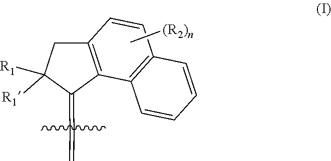

wherein:
   R₁ is C1-C12 alkyl or C1-C12 substituted alkyl;
   R₁′ is hydrogen;
   R₂ is hydrogen, C1-C12 alkoxy, or substituted C1-C12 alkoxy; or
   R₂ is a group of the formula: —Y₁ —X₁—R₂′, wherein:
      Y₁ is —NRₐ—, wherein: R₃ is hydrogen, C1-C6 alkyl, or substituted C1-C6 alkyl;
      X₁ is C1-C12 alkanediyl or C1-C12 substituted alkanediyl; and
      R₂′ is —NR_{b}R_{b}′R_{b} —, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0 or 1.
and a stator of the formula:

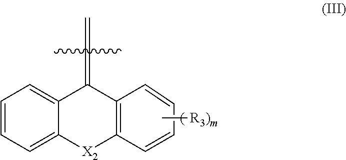

wherein:
   X₂ is a covalent bond or S;
   R₃ is hydrogen or halo; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C2-C12 alkynediyl, or C2-C12 substituted alkynediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C1-C12 heterocycloalkyl, C1-C12 substituted heterocycloalkyl, C18-C24 triarylphosphine or C18-C24 substituted triarylphosphine; and
   m is 1 or 2.

In some embodiments, the stimulus activated molecular motor comprises a rotor of the formula:

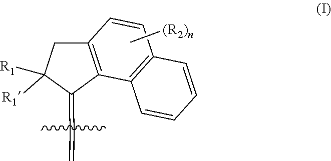

wherein:
   R₁ is C1-C12 alkyl or C1-C12 substituted alkyl;
   R₁′ is hydrogen;
   R₂ is hydrogen, C1-C12 alkoxy, or substituted C1-C12 alkoxy; or
   R₂ is a group of the formula: —Y₁—X₁—R₂′, wherein:
      Y₁ is —NRₐ —, wherein: R₃ is hydrogen, C1-C6 alkyl, or substituted C1-C6 alkyl;
      X₁ is C1-C12 alkanediyl or C1-C12 substituted alkanediyl; and
      R₂′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0 or 1.
and a stator of the formula:

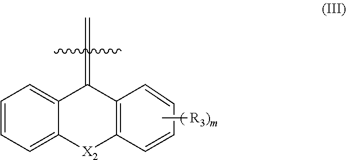

wherein:
   X₂ is a covalent bond or S;
   R₃ is hydrogen or halo; or
   R₃ is a group of the formula: —Y₂—X₃-R₃′, wherein:
      Y₂ is a covalent bond or —NRₑ—, wherein: Rₑ is hydrogen, C1-C6 alkyl, or substituted C1-C6 alkyl;
      X₃ is C1-C12 alkanediyl, C1-C12 substituted alkanediyl, C2-C12 alkynediyl, or C2-C12 substituted alkynediyl; and
      R₃′ is —NR_{f}R_{f}R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C1-C12 heterocycloalkyl, C1-C12 substituted heterocycloalkyl, C18-C24 triarylphosphine or C18-C24 substituted triarylphosphine; and
   m is 0, 1, 2, 3, or 4.

In some embodiments, the stimulus activated molecular motor comprises a rotor of the formula:

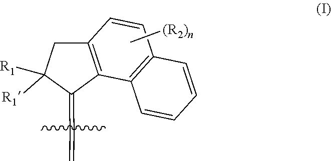

wherein:
   R₁ and R₁′ are each C1-C12 alkyl or C1-C12 substituted alkyl;
   R₁′ is hydrogen; and
   n is 0.
and a stator of the formula:

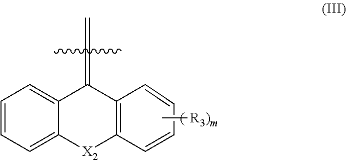

wherein:
   X₂ is S;
   R₃ is hydrogen; or
   R₃ is a group of the formula: —Y₂—X₃—R₃′, wherein:
      Y₂ is —NRₑ—, wherein: Rₑ is hydrogen, C1-C6 alkyl, or substituted C1-C6 alkyl;
      X₃ is C1-C12 alkanediyl or C1-C12 substituted alkanediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}′ is absent, hydrogen, C1-C6 alkyl, or C1-C6 substituted alkyl; and
   m is 1.

In some embodiments, R₃ is —Y₂—X₃—R₃′. In some embodiments, Y₁ is —NRₑ—. In some embodiments, Rₑ is hydrogen. In some embodiments, X₃ is C1-C12 alkanediyl or C1-C12 substituted alkanediyl. In some embodiments, X₃ is C1-C12 alkanediyl. In some embodiments, is ethylene. In some embodiments, R₃′ is —NR_{f}R_{f}′R_{f}″. In some embodiments, R_{f} is C1-C6 alkyl or C1-C6 substituted alkyl. In some embodiments, R_{f}′ is C1-C6 alkyl. In some embodiments, R_{f} is methyl. In some embodiments, R_{f}′ is C1-C6 alkyl or C1-C6 substituted alkyl. In some embodiments, R_{f}″ is C1-C6 alkyl. In some embodiments, R_{f}′ is methyl. In some embodiments, R_{f}″ is absent. In some embodiments, R₃′ is C1-C12 heterocycloalkyl or C1-C12 heterocycloalkyl. In some embodiments, wherein R₃′ is C1-C12 heterocycloalkyl. In some embodiments, wherein R₃′ is 1,4-piperazinyl. In some embodiments, R₃ is —NHCH₂CH₂N(Me)₂ or —NHCH₂CH₂N(CH₂CH₂)₂NH.

In some embodiments, wherein m is 0 or 1. In some embodiments, m is 0. In some embodiments, n is 1.

In some embodiments, X₂ is S. In some embodiments, X₂ is a covalent bond. In some embodiments, X₂ is CR_{d}R_{d}′. In some embodiments, R_{d} is C1-C6 alkyl or C1-C6 substituted alkyl. In some embodiments, R_{d} is C1-C6 alkyl. In some embodiments, R_{d} is methyl. In some embodiments, R_{d}′ is C1-C6 alkyl or C1-C6 substituted alkyl. In some embodiments, R_{d}′ is C1-C6 alkyl. R_{d}′ is methyl.

In some embodiments, the stimulus activated molecular motor is further defined as:

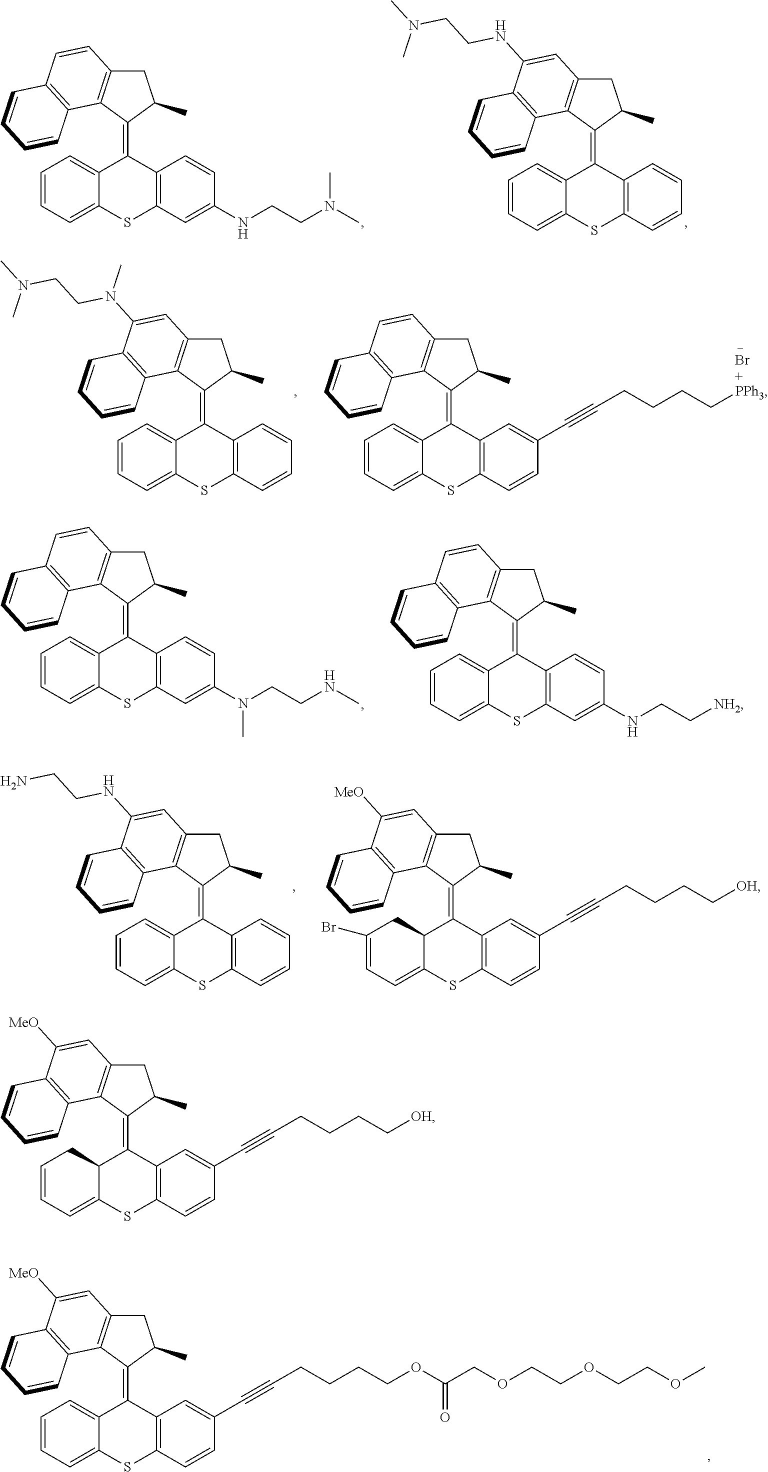

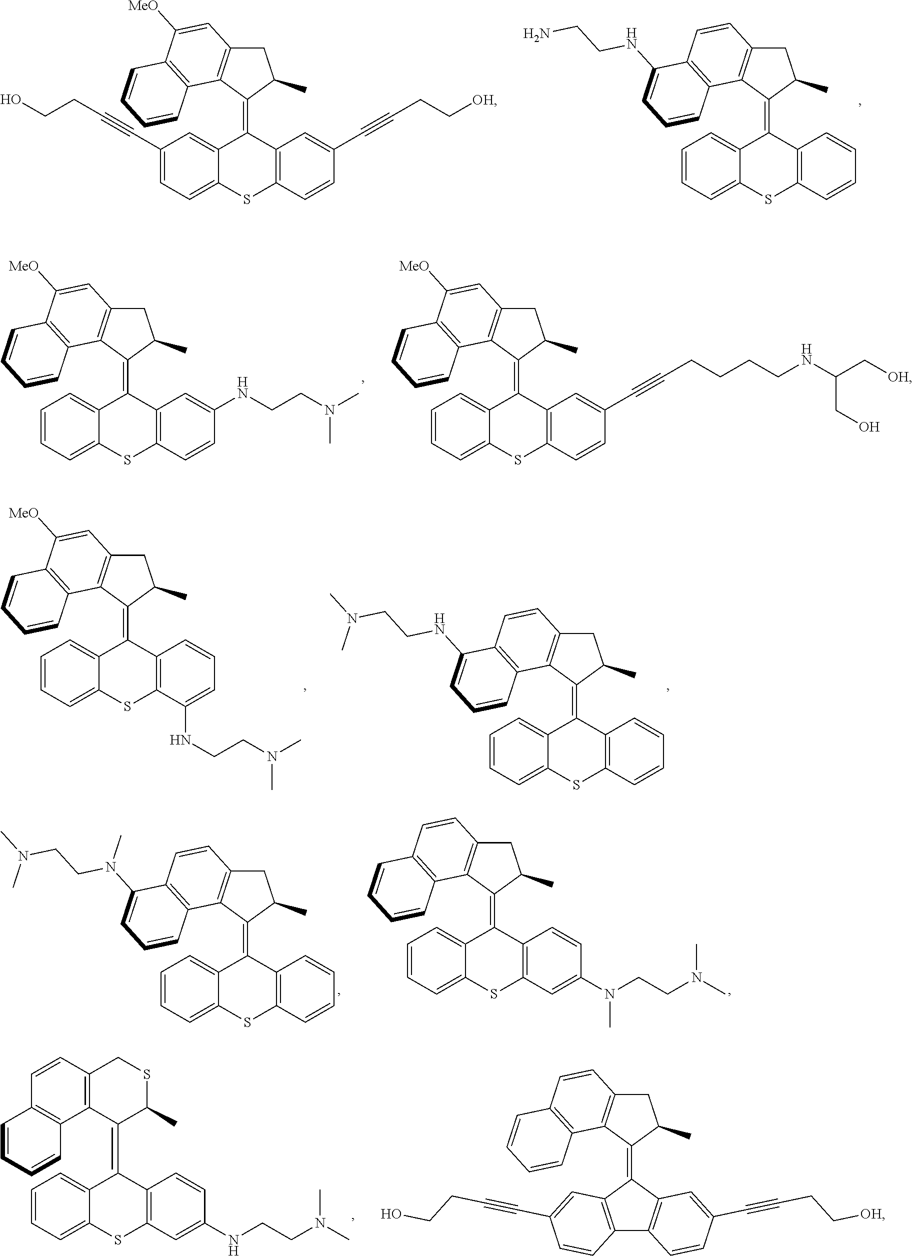

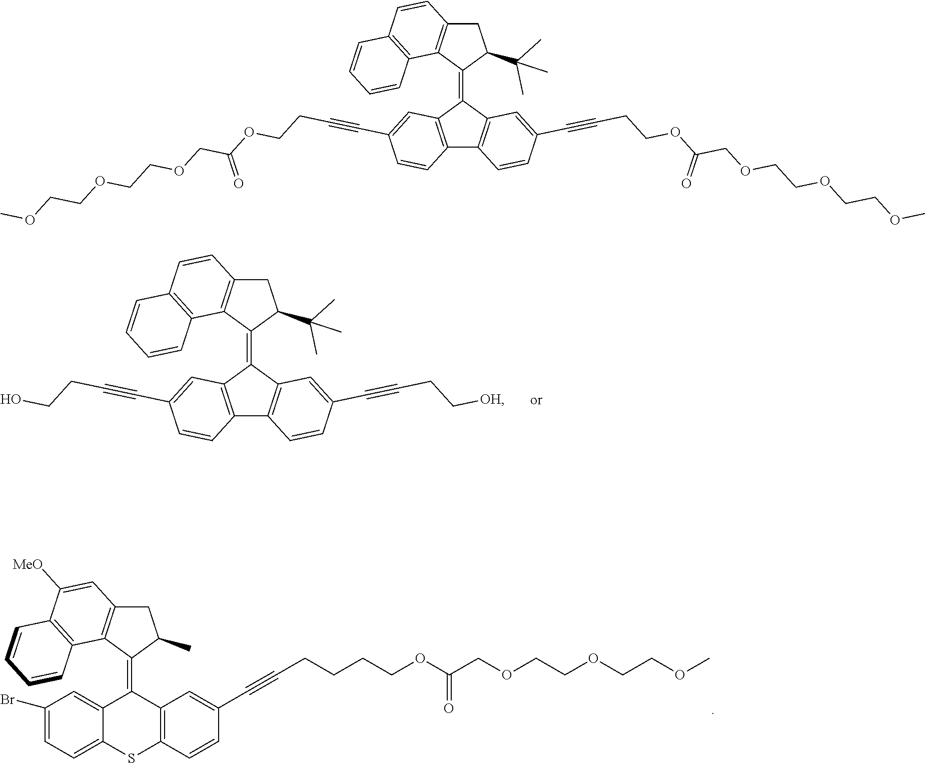

In some embodiments, the stimulus activated molecular motor is further defined as:

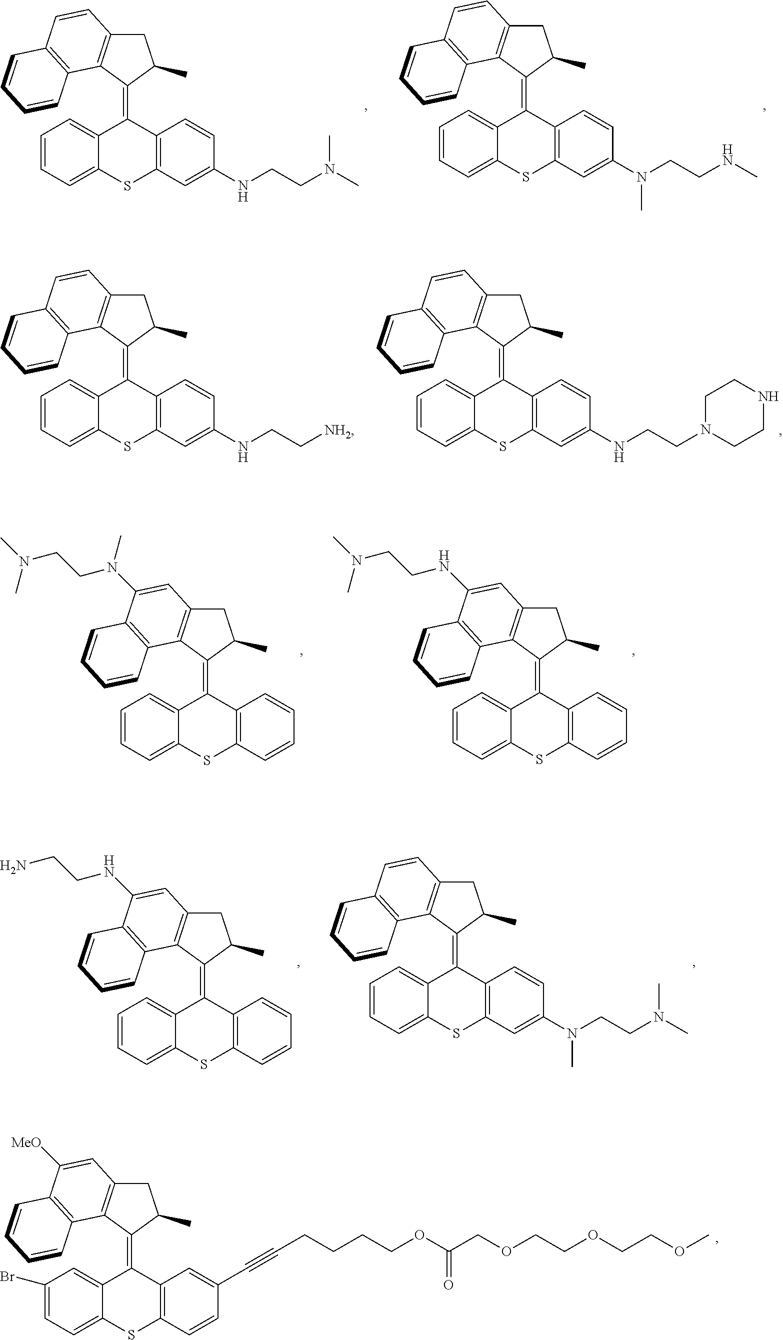

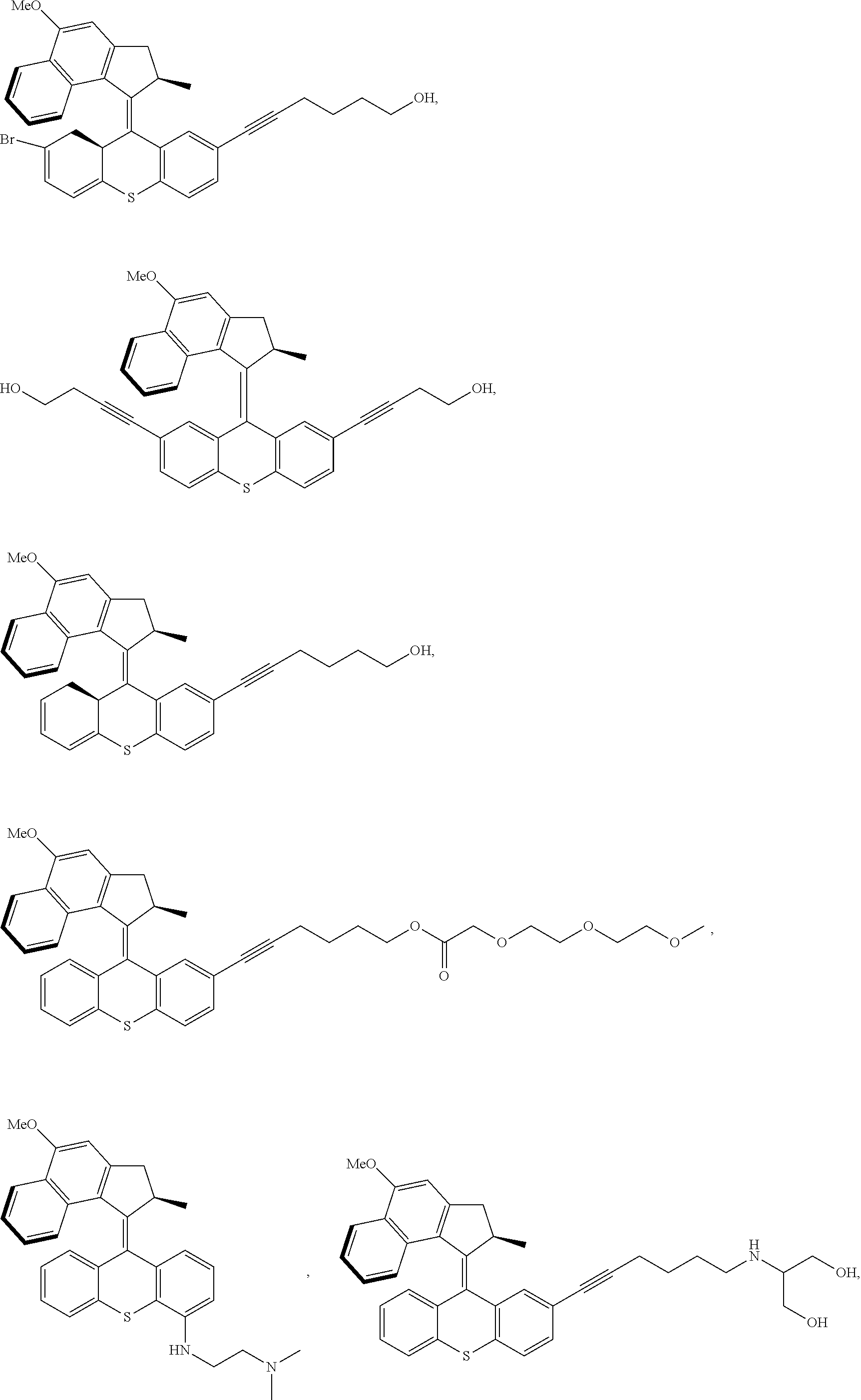

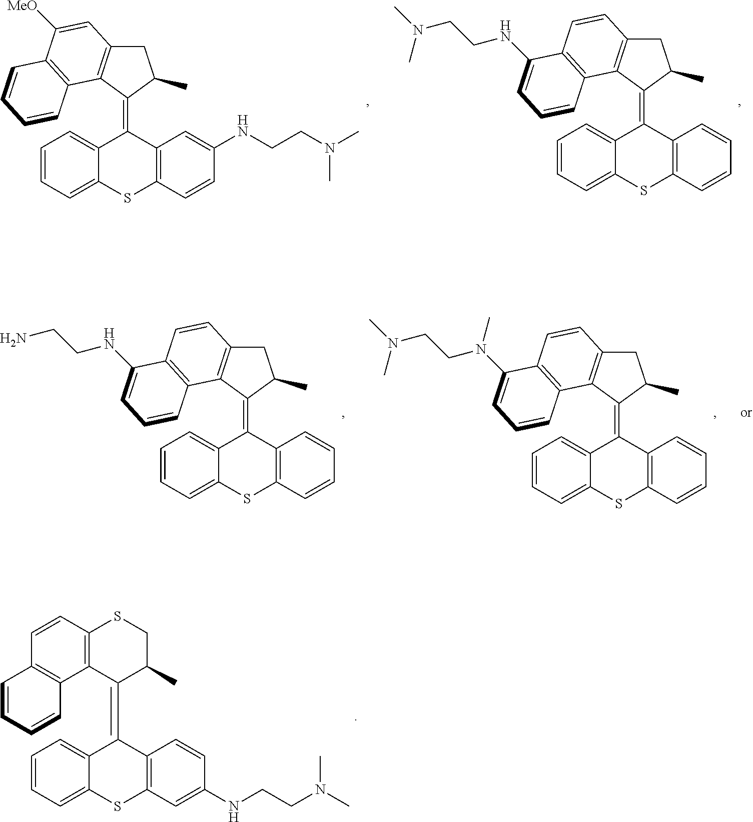

In some embodiments, the stimulus activated molecular machine is further defined as:

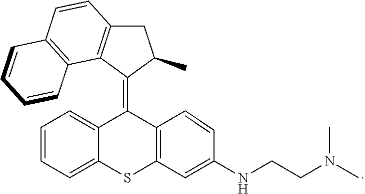

In some embodiments, the stimulus activated molecular machine is further defined as:

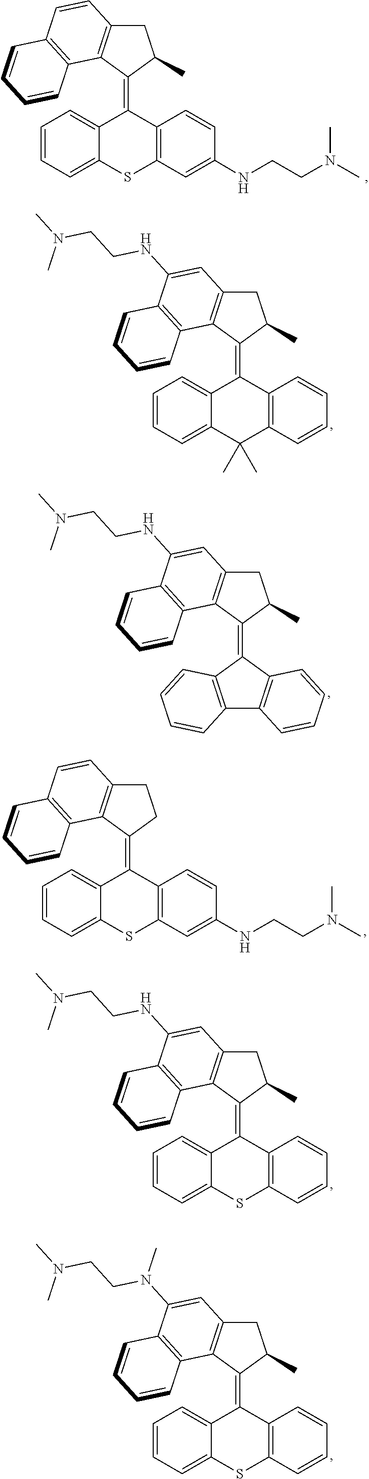

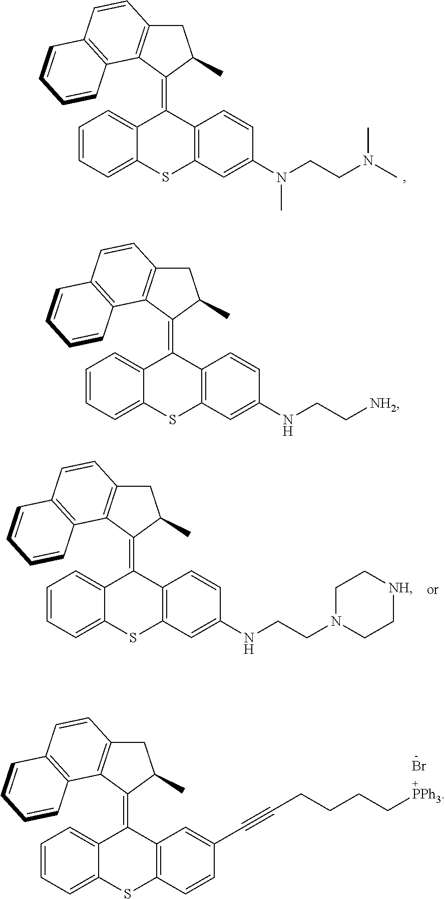

In some embodiments, the stimulus activated molecular machine is not a compound of the formula:

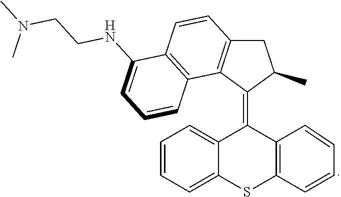

In some embodiments, the stimulus activated molecular machine rotates unidirectionally. In some embodiments, the stimulus activated molecular machine rotates bidirectionally.

In some embodiments, the rotational component of the stimulus activated molecular machine rotates at a speed greater than 1 Hz. In some embodiments, the stimulus activated molecular machine rotates at a speed greater than 10⁵ Hz. In some embodiments, the rotational component of the molecular machine or switch rotates at a speed of about 10⁶ Hz. In some embodiments, the rotational component of the molecular machine or switch rotates at a speed of about 10⁸ Hz.

In some embodiments, the stimulus activated molecular machine is activated by a stimulus. In some embodiments, the stimulus is electromagnetic radiation. In some embodiments, the electromagnetic radiation comprises gamma rays, X-rays, UV light, visible light, near infrared light, infrared light, microwaves, or radio waves. In some embodiments, the electromagnetic radiation comprises UV light, visible light, or near infrared light. In some embodiments, the electromagnetic radiation comprises visible light. In some embodiments, the electromagnetic radiation comprises a wavelength of 400 nm.

In some embodiments, the stimulus activated molecular machine is activated for a controlled time period. In some embodiments, the stimulus activated molecular machine is activated for less than 5 seconds. In some embodiments, the stimulus activated molecular machine is activated for less than 2 seconds. In some embodiments, the stimulus activated molecular machine is activated for about 250 milliseconds.

In some embodiments, the energy source is a laser. In some embodiments, the intensity of the energy source is controlled.

In some embodiments, the patient is a mammal. In some embodiments, the mammal is a human.

In another aspect, the present disclosure provides molecular machines comprising:
(A) a rotor of the formula:

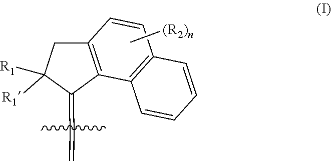

wherein:
   R₁ and R₁′ are each independently hydrogen, a C1-C12 alkyl or a C1-C12 substituted alkyl;
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁—X₁—R₂′, wherein:
      Y₁ is —O—, —S—, or —NRₐ—, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R₂′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4; and
(B) a stator of the formula:

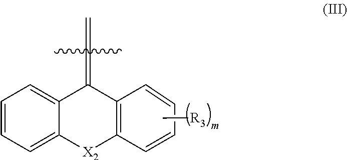

wherein:
   X₂ is a covalent bond, O, S, NR_{c}, or CR_{d}R_{d}′, wherein R_{c}, R_{d}, and R_{d}′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
   m is 0, 1, 2, 3, or 4;
provided at least one of R₂ is a group of the formula: —Y₁—X₁—R₂′ or at least one of R₃ is a group of the formula: —Y₂—X₃—R₃′; and provided that the molecular machine is not a compound of the formula:

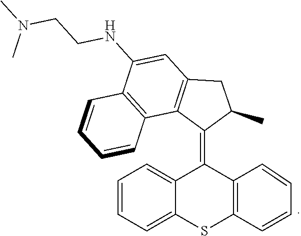

In some embodiments, the rotor is further defined as:

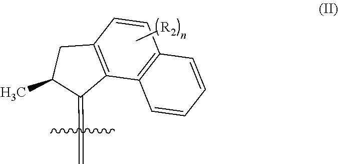

wherein:
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁—X₁—R₂′, wherein:
      Y₁ is —O—, —S—, or —NRₐ—, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R₂′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4.

In some embodiments, R₁ is C1-C12 alkyl or substituted C1-C12 alkyl. In some embodiments, R₁ is C1-C12 alkyl. In some embodiments, R₁ is methyl. In some embodiments, R₁′ is hydrogen. In some embodiments, R₂ is hydrogen. In some embodiments, R₂ is —Y₁ —X₁—R₂′. In some embodiments, Y₁ is —NRₐ—. In some embodiments, R₃ is hydrogen. In some embodiments, X₁ is C1-C12 alkanediyl or C1-C12 substituted alkanediyl. In some embodiments, X₁ is C1-C12 alkanediyl. In some embodiments, X₁ is ethylene. In some embodiments, R_{b} is C1-C6 alkyl or C1-C6 substituted alkyl. In some embodiments, R_{b} is C1-C6 alkyl. In some embodiments, R_{b} is methyl. In some embodiments, R_{b}′ is C1-C6 alkyl or C1-C6 substituted alkyl. In some embodiments, R₃′ is C1-C6 alkyl, such as methyl. In some embodiments, R_{b}″ is absent. In some embodiments, R₂ is —NHCH₂CH₂N(Me)₂. In some embodiments, n is 0. In some embodiments, n is 1.

In some embodiments, the stator is further defined as:

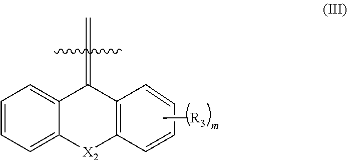

wherein:
   Y₂ is a covalent bond, —O—, —S—, or —NRₑ—, wherein: Rₑ is hydrogen, C1-C6 alkyl, or substituted C1-C6 alkyl;
   X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
   R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
m is 0 or 1.

In some embodiments, R₃ is —Y₂—X₃—R₃′. In some embodiments, Y₂ is —NRₑ—. In some embodiments, Rₑ is hydrogen.

In some embodiments, wherein X₃ is C1-C12 alkanediyl or C1-C12 substituted alkanediyl. In some embodiments, X₃ is C1-C12 alkanediyl. In some embodiments, X₃ is ethylene.

In some embodiments, R₃′ is —NR_{f}R_{f}′R_{f}″. In some embodiments, R_{f} is C1-C6 alkyl or C1-C6 substituted alkyl. In some embodiments, R_{f} is C1-C6 alkyl. In some embodiments, R_{f} is methyl. In some embodiments, R_{f}′ is C1-C6 alkyl or C1-C6 substituted alkyl. In some embodiments, R_{f}′ is C1-C6 alkyl. In some embodiments, Rr′ is methyl. In some embodiments, R_{f}″ is absent. In some embodiments, R₃′ is C1-C12 heterocycloalkyl or C1-C12 heterocycloalkyl. In some embodiments, R₃′ is C1-C12 heterocycloalkyl. In some embodiments, R₃′ is 1,4-piperazinyl. In some embodiments, R₃ is —NHCH₂CH₂N(Me)₂ or —NHCH₂CH₂N(CH₂CH₂)₂NH.

In some embodiments, wherein n is 0. In some embodiments, n is 1.

In some embodiments, X₂ is S. In some embodiments, X₂ is a covalent bond. In some embodiments, X₂ is CR_{d}R_{d}′. In some embodiments, R_{d} is C1-C6 alkyl or C1-C6 substituted alkyl. In some embodiments, R_{d} is C1-C6 alkyl. In some embodiments, R_{d} is methyl. In some embodiments, R_{d}′ is C1-C6 alkyl or C1-C6 substituted alkyl. In some embodiments, R_{d}′ is C1-C6 alkyl. In some embodiments, R_{d}′ is methyl.

In some embodiments, the stimulus activated molecular machine is further defined as:

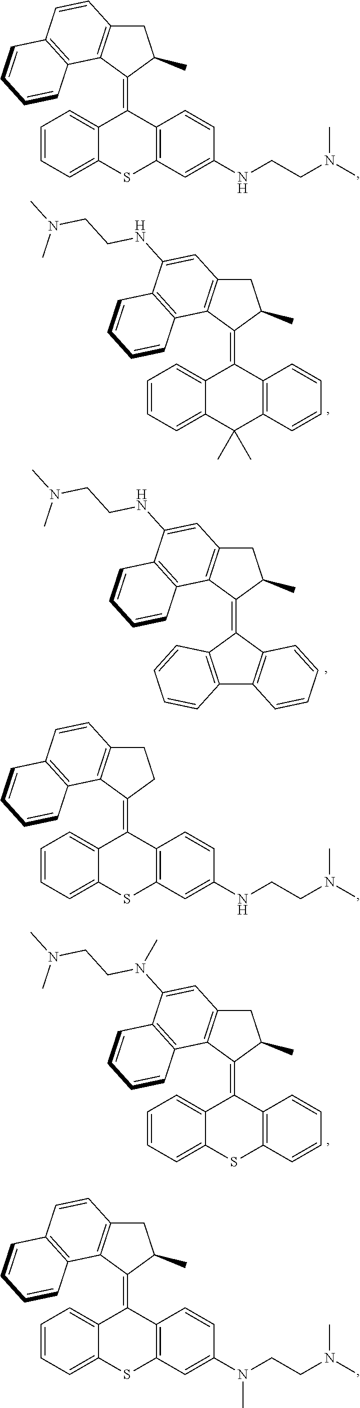

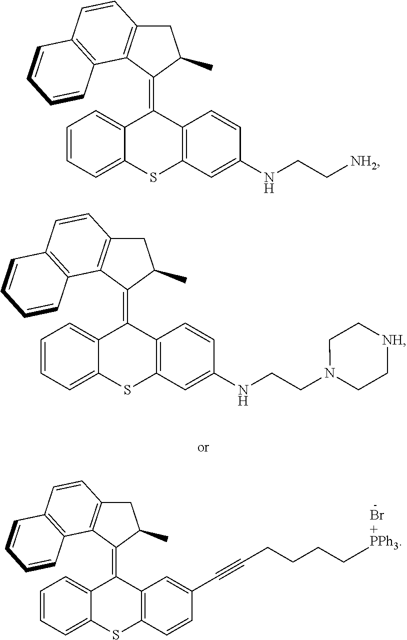

It is contemplated that any methods, compounds, or compositions described herein can be implemented with respect to any other methods, compounds, or compositions described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### EMBODIMENTS OF THE INVENTION

1. A method of treating a disease or disorder in a patient caused by an infection of a microorganism comprising:
(A) administering to the patient in need thereof a stimulus activated molecular machine, wherein the stimulus activated molecule machine is configured to be activated by an appropriate stimulus; and
(B) exposing the patient to a stimulus sufficient to activate the molecular machine.

2. A composition for use in the treatment of a disease or disorder in a patient caused by an infection of a microorganism comprising a stimulus activated molecule machine is configured to be activated by an appropriate stimulus.

3. Use of a stimulus activated molecular machine in the treatment of a disease or disorder in a patient caused by an infection of a microorganism.

4. A method of inhibiting the growth of a microorganism comprising contacting the microorganism with a stimulus activated molecular machine, wherein the stimulus activated molecule machine is configured to be activated by an appropriate stimulus, and exposing the microorganism to an appropriate stimulus.

5. A composition for use in inhibiting the growth of a microorganism comprising a stimulus activated molecular machine, wherein the stimulus activated molecule machine is configured to be activated by an appropriate stimulus.

Use of a stimulus activated molecular motor for inhibiting the growth of a microorganism, wherein the stimulus activated molecule machine is configured to be activated by an appropriate stimulus.

7. A method of killing a microorganism comprising contacting the microorganism with a stimulus activated molecular machine, wherein the stimulus activated molecule machine is configured to be activated by an appropriate stimulus, and exposing the microorganism to an appropriate stimulus.

8. A composition for use in killing a microorganism comprising a stimulus activated molecular machine, wherein the stimulus activated molecule machine is configured to be activated by an appropriate stimulus.

9. Use of a stimulus activated molecular motor for killing a microorganism, wherein the stimulus activated molecule machine is configured to be activated by an appropriate stimulus.

10. A method of inhibiting a biofilm formation comprising contacting the biofilm with a stimulus activated molecular machine, wherein the stimulus activated molecule machine is configured to be activated by an appropriate stimulus, and exposing the biofilm to an appropriate stimulus.

11. A composition for use in inhibiting a biofilm formation comprising a stimulus activated molecular machine, wherein the stimulus activated molecule machine is configured to be activated by an appropriate stimulus.

12. Use of a stimulus activated molecular motor for inhibiting a biofilm formation, wherein the stimulus activated molecule machine is configured to be activated by an appropriate stimulus.

13. A method of eliminating a biofilm comprising contacting the biofilm with a stimulus activated molecular machine, wherein the stimulus activated molecule machine is configured to be activated by an appropriate stimulus, and exposing the biofilm to an appropriate stimulus.

14. A composition for use in eliminating a biofilm comprising a stimulus activated molecular machine, wherein the stimulus activated molecule machine is configured to be activated by an appropriate stimulus.

15. Use of a stimulus activated molecular motor for eliminating a biofilm, wherein the stimulus activated molecule machine is configured to be activated by an appropriate stimulus.

16. A method of inducing necrosis in a microorganism comprising contacting the microorganism with a stimulus activated molecular machine, wherein the stimulus activated molecule machine is configured to be activated by an appropriate stimulus, and exposing the microorganism to an appropriate stimulus.

17. A composition for use in inducing necrosis in a microorganism comprising a stimulus activated molecular machine, wherein the stimulus activated molecule machine is configured to be activated by an appropriate stimulus.

18. Use of a stimulus activated molecular motor for inducing necrosis in a microorganism, wherein the stimulus activated molecule machine is configured to be activated by an appropriate stimulus.

19. A method of causing oxidative stress in a cell comprising contacting the cell with a stimulus activated molecular machine, wherein the stimulus activated molecule machine is configured to be activated by an appropriate stimulus, and exposing the cell to an appropriate stimulus.

20. A composition for use in causing oxidative stress in a cell comprising a stimulus activated molecular machine, wherein the stimulus activated molecule machine is configured to be activated by an appropriate stimulus.

21. Use of a stimulus activated molecular motor in causing oxidative stress in a cell, wherein the stimulus activated molecule machine is configured to be activated by an appropriate stimulus.

22. A method of inhibiting mitochondria function in a cell comprising contacting the cell with a stimulus activated molecular machine, wherein the stimulus activated molecule machine is configured to be activated by an appropriate stimulus, and exposing the cell to an appropriate stimulus.

23. A composition for use inhibiting mitochondria function in a cell comprising a stimulus activated molecular machine, wherein the stimulus activated molecule machine is configured to be activated by an appropriate stimulus.

24. Use of a stimulus activated molecular motor for inhibiting mitochondria function in a cell, wherein the stimulus activated molecule machine is configured to be activated by an appropriate stimulus.

25. A method of overcoming drug resistance in a microorganism comprising contacting the microorganism with a stimulus activated molecular machine, wherein the stimulus activated molecule machine is configured to be activated by an appropriate stimulus, and exposing the microorganism to an appropriate stimulus in the presence of a drug to which the microorganism was resistant to.

26. A composition for use in overcoming drug resistance in a microorganism comprising a stimulus activated molecular machine, wherein the stimulus activated molecule machine is configured to be activated by an appropriate stimulus.

27. Use of a stimulus activated molecular motor for overcoming drug resistance in a microorganism, wherein the stimulus activated molecule machine is configured to be activated by an appropriate stimulus.

28. The method, composition for use, or use according to any one of embodiments 1-27, wherein the microorganism is a bacterium.

29. The method, composition for use, or use of embodiment 28, wherein the bacterium is gram positive bacteria.

30. The method, composition for use, or use of embodiment 28, wherein the bacterium is gram negative bacteria.

31. The method, composition for use, or use of embodiment 28, wherein the bacterium is a gram indeterminate bacteria.

32. The method, composition for use, or use according to any one of embodiments 28-30, wherein the bacterium is sensitive to one or more antibiotics.

33. The method, composition for use, or use according to any one of embodiments 28-32, wherein the bacterium is sensitive to two or more antibiotics.

34. The method, composition for use, or use of embodiment 33, wherein the antibiotic is methicillin, cefoxitin, oxacillin, gentamicin, ciprofloxacin, levofloxacin, moxifloxacin, erythromycin, clindamycin, linezolid, daptomycin, vancomycin, doxycycline, tobramycin, tetracycline, tigecycline, nitrofurantoin, rifampin, trimethoprim-sulfamethoxazole, amoxicillin-clavulanic acid, ampicillin-sulbactam, piperacillin-tazobactam, cefepime, ertapenem, imipenem, and meropenem.

35. The method, composition for use, or use of embodiment 34, wherein the bacterium is a gram positive bacterium and is resistant to cefoxitin, oxacillin, gentamicin, ciprofloxacin, levofloxacin, moxifloxacin, erythromycin, clindamycin, linezolid, daptomycin, vancomycin, doxycycline, tetracycline, tigecycline, nitrofurantoin, rifampin, or trimethoprim-sulfamethoxazole.

36. The method, composition for use, or use of embodiment 34, wherein the bacterium is a gram negative bacterium and is resistant to amoxicillin-clavulanic acid, ampicillin-sulbactam, piperacillin-tazobactam, cefepime, ertapenem, imipenem, meropenem, gentamicin, tobramycin, ciprofloxacin, levofloxacin, nitrofurantoin, or trimethoprim-sulfamethoxazole.

37. The method, composition for use, or use according to any one of embodiments 28-36, wherein the bacterium is from a hospital acquired infection.

38. The method, composition for use, or use according to any one of embodiments 28-37, wherein the bacterium is Staphylococcus saprophyticus (S. saprophyticus), Staphylococcus aureus (S. aureus), methicillin-resistant Staphylococcus aureus (MRSA), coagulase negative staphylococcus (CNS), methicillin-resistant CNS (MRCNS), E. coli, multi-drug resistance (MDR) E. coli, MDR-Citrobacter koseri, MDR-Enterobacter cloacae complex, MDR-Morganella morganii, MDR-Klebsiella pneumonia or MDR-Acinetobacter baumannii.

39. The method, composition for use, or use according to any one of embodiments 28-38, wherein the method further comprises administering a second antibiotic agent.

40. The method, composition for use, or use according to any one of embodiments 1-27, wherein the microorganism is a fungus.

41. The method, composition for use, or use of embodiment 40, wherein the fungus is a Basidiomycota fungus.

42. The method, composition for use, or use of embodiment 41, wherein the fungus is a Cryptococcus fungus.

43. The method, composition for use, or use of embodiment 40, wherein the fungus is an Ascomycota fungus.

44. The method, composition for use, or use of embodiment 43, wherein the fungus is an Aspergillus, Candida, Coccidioides, Histoplasma, or Blastomyces fungus.

45. The method, composition for use, or use of embodiment 40, wherein the fungus is a Mucoromycorina fungus.

46. The method, composition for use, or use according to any one of embodiments 40-45, wherein the method further comprises administering a second anti-fungal therapy.

47. The method, composition for use, or use of embodiment 46, wherein the second anti-fungal therapy is a therapy targeting the ergosterol biosynthetic pathway.

48. The method, composition for use, or use of embodiment 47, wherein the second anti-fungal therapy is Amphotericin B, fluconazole, itraconazole, posaconazole, or voriconazole.

49. The method, composition for use, or use of embodiment 48, wherein the second anti-fungal therapy is voriconazole.

50. The method, composition for use, or use of embodiment 46, wherein the second anti-fungal therapy is echinocandins or flucytosine.

51. The method, composition for use, or use according to any one of embodiments 40-50, wherein the fungus has infected the central nervous system.

52. The method, composition for use, or use according to any one of embodiments 40-50, wherein the fungus has infected the lungs.

53. The method, composition for use, or use according to any one of embodiments 40-52, wherein the fungus is present in its spore form.

54. A method of treating cancer in a patient in need thereof comprising administering to the patient a stimulus activated molecular machine, wherein the stimulus activated molecule machine is configured to be activated by an appropriate stimulus, and exposing the patient to an appropriate stimulus.

55. A composition for use in treating cancer comprising a stimulus activated molecular machine, wherein the stimulus activated molecule machine is configured to be activated by an appropriate stimulus.

56. Use of a stimulus activated molecular motor in treating cancer, wherein the stimulus activated molecule machine is configured to be activated by an appropriate stimulus.

57. A method of reducing the tumor burden in a patient comprising administering to the patient a stimulus activated molecular machine, wherein the stimulus activated molecule machine is configured to be activated by an appropriate stimulus, and exposing the patient to an appropriate stimulus.

58. A composition for use in reducing the tumor burden in a patient comprising a stimulus activated molecular machine, wherein the stimulus activated molecule machine is configured to be activated by an appropriate stimulus.

59. Use of a stimulus activated molecular motor in reducing the tumor burden in a patient, wherein the stimulus activated molecule machine is configured to be activated by an appropriate stimulus.

60. A method of enhancing the effect of a chemotherapeutic compound in a patient comprising administering to the patient a stimulus activated molecular machine, wherein the stimulus activated molecule machine is configured to be activated by an appropriate stimulus, and exposing the patient to an appropriate stimulus after the patient has been administered the chemotherapeutic compound.

61. A composition for use in enhancing the effect of a chemotherapeutic compound comprising a stimulus activated molecular machine, wherein the stimulus activated molecule machine is configured to be activated by an appropriate stimulus.

62. Use of a stimulus activated molecular motor in enhancing the effect of a chemotherapeutic compound, wherein the stimulus activated molecule machine is configured to be activated by an appropriate stimulus.

63. A method of killing a cancerous cell comprising contacting the cancerous cell with a stimulus activated molecular machine, wherein the stimulus activated molecule machine is configured to be activated by an appropriate stimulus, and exposing the cancerous cell to an appropriate stimulus.

64. A composition for use in killing a cancerous cell comprising a stimulus activated molecular machine, wherein the stimulus activated molecule machine is configured to be activated by an appropriate stimulus.

65. Use of a stimulus activated molecular motor in killing a cancerous cell, wherein the stimulus activated molecule machine is configured to be activated by an appropriate stimulus.

66. A method of inducing necrosis in a cancerous cell comprising contacting the cancerous cell with a stimulus activated molecular machine, wherein the stimulus activated molecule machine is configured to be activated by an appropriate stimulus, and exposing the cancerous cell to an appropriate stimulus.

67. A composition for use in inducing necrosis in a cancerous cell comprising a stimulus activated molecular machine, wherein the stimulus activated molecule machine is configured to be activated by an appropriate stimulus.

68. Use of a stimulus activated molecular motor in inducing necrosis in a cancerous cell, wherein the stimulus activated molecule machine is configured to be activated by an appropriate stimulus.

69. The method, composition for use, or use according to any one of embodiments 54-68, wherein the cancer is a carcinoma, sarcoma, lymphoma, leukemia, melanoma, mesothelioma, multiple myeloma, or seminoma.

70. The method, composition for use, or use according to any one of embodiments 54-68, wherein the cancer is of the bladder, blood, bone, brain, breast, central nervous system, cervix, colon, endometrium, esophagus, gall bladder, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

71. The method, composition for use, or use according to any one of embodiments 54-70, wherein the cancer is resistant to one or more chemotherapeutic compounds.

72. The method, composition for use, or use according to any one of embodiments 54-71, wherein the method further comprises administering a second therapeutic agent.

73. The method, composition for use, or use of embodiment 72, wherein the second therapeutic agent is a second chemotherapeutic agent, surgery, photodynamic therapy, sonodynamic therapy, radiotherapy, or immunotherapy.

74. The method, composition for use, or use according to any one of embodiments 1-73, wherein the stimulus activated molecular machine comprises a Feringa-type molecular machine.

75 The method, composition for use, or use according to any one of embodiments 1-74, wherein the stimulus activated molecular machine comprises a rotor that is connected to a stator.

76. The method, composition for use, or use according to any one of embodiments 1-75, wherein the stimulus activated molecular machine comprises a rotor that is connected to a stator through an alkenyl or alkynyl group.

77. The method, composition for use, or use of embodiment 75, wherein the stimulus activated molecular machine comprises a rotor that is connected to a stator through an atropisomeric alkene.

78. The method, composition for use, or use according to any one of embodiments 1-77, wherein the rotor comprises one, two, three, four, or five rings.

79. The method, composition for use, or use according to any one of embodiments 1-78, wherein the rotor comprises one, two, or three aromatic rings.

80. The method, composition for use, or use according to any one of embodiments 1-79, where the rotor further comprises one, two, or three aliphatic rings.

81. The method, composition for use, or use according to any one of embodiments 1-80, wherein the rotor comprises one, two, or three aromatic rings and one or two aliphatic rings.

82. The method, composition for use, or use according to any one of embodiments 25-81, wherein the rotor comprises one, two, or three aliphatic or aromatic rings.

83. The method, composition for use, or use according to any one of embodiments 1-82, wherein the rotor comprises two aromatic rings and an aliphatic ring.

84. The method, composition for use, or use according to any one of embodiments 1-83, wherein the rotor is further defined as:

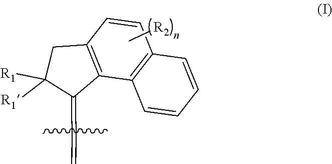

wherein:
   R₁ and R₁′ are each independently hydrogen, a C1-C12 alkyl or a C1-C12 substituted alkyl;
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁—X₁—R₂′, wherein:
      Y₁ is —O—, —S—, or —NRₐ—, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R₃′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4.

85. The method, composition for use, or use of embodiment 84, wherein the rotor is further defined as:

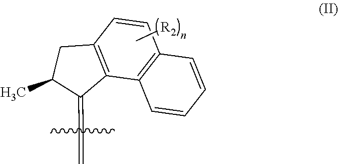

wherein:
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁—X₁—R₂′, wherein:
      Y₁ is —O—, —S—, or —NRₐ—, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R₂′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4.

86. The method, composition for use, or use of embodiment 84, wherein R₁ is C1-C12 alkyl or substituted C1-C12 alkyl.

87. The method, composition for use, or use of embodiment 86, wherein R₁ is C1-C12 alkyl.

88. The method, composition for use, or use of embodiment 86 or embodiment 87, wherein R₁ is methyl.

89. The method, composition for use, or use according to any one of embodiments 84 and 86-88, wherein R₁′ is hydrogen.

90. The method, composition for use, or use according to any one of embodiments 84-89, wherein R₂ is hydrogen.

91. The method, composition for use, or use according to any one of embodiments 84-89, wherein R₂ is —Y₁—X₁—R₂′.

92. The method, composition for use, or use of embodiment 91, wherein Y₁ is —NRₐ—.

93. The method, composition for use, or use of embodiment 92, wherein Rₐ is hydrogen.

94. The method, composition for use, or use according to any one of embodiments 91-93, wherein X₁ is C1-C12 alkanediyl or C1-C12 substituted alkanediyl.

95. The method, composition for use, or use according to any one of embodiments 91-94, wherein X₁ is C1-C12 alkanediyl.

96. The method, composition for use, or use according to any one of embodiments 91-95, wherein X₁ is ethylene.

97. The method, composition for use, or use according to any one of embodiments 91-96, wherein R_{b} is C1-C6 alkyl or C1-C6 substituted alkyl.

98. The method, composition for use, or use according to any one of embodiments 91-97, wherein R_{b} is C1-C6 alkyl.

99. The method, composition for use, or use according to any one of embodiments 91-98, wherein R_{b} is methyl.

100 The method, composition for use, or use according to any one of embodiments 91-99, wherein R_{b}′ is C1-C6 alkyl or C1-C6 substituted alkyl.

101. The method, composition for use, or use according to any one of embodiments 91-100, wherein R_{b}′ is C1-C6 alkyl.

102. The method, composition for use, or use according to any one of embodiments 91-101, wherein R_{b}′ is methyl.

103 The method, composition for use, or use according to any one of embodiments 91-102, wherein R_{b}″ is absent.

104. The method, composition for use, or use according to any one of embodiments 84-102, wherein R₂ is —NHCH₂CH₂N(Me)₂.

105. The method, composition for use, or use according to any one of embodiments 84-104, wherein n is 0.

106 The method, composition for use, or use according to any one of embodiments 84-104, wherein n is 1.

107. The method, composition for use, or use according to any one of embodiments 1-107, wherein the molecular machine or switch comprises a stator, wherein the stator comprises one, two, three, four, or five rings.

108. The method, composition for use, or use of embodiment 107, wherein the stator comprises one, two, three, four, or five aromatic rings.

109. The method, composition for use, or use of either embodiment 107 or embodiment 108, wherein the stator comprises one, two, or three aromatic rings.

110. The method, composition for use, or use of embodiment 107, wherein the stator comprises one, two, three, four, or five aliphatic rings.

111. The method, composition for use, or use of either embodiment 107 or embodiment 110, wherein the stator comprises one, two, or three aliphatic rings.

112. The method, composition for use, or use according to any one of embodiments 107-111, wherein the stator comprises two, three, or four rings.

113. The method, composition for use, or use according to any one of embodiments 107-112, wherein the stator comprises three rings.

114. The method, composition for use, or use according to any one of embodiments 107-113, wherein the stator comprises three rings with at least 2 aromatic rings.

115. The method, composition for use, or use according to any one of embodiments 107-114, wherein the stator is further defined as:

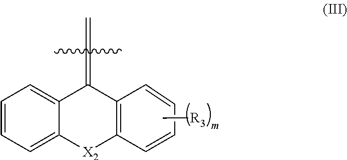

wherein:
   X₂ is a covalent bond, O, S, NR_{c}, or CR_{d}R_{d}′, wherein R_{c}, R_{d}, and R_{d}′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—Rₑ′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is —OC(O)CH₂(OCH₂CH₂)₂R₃″, wherein R₃″ is hydroxy, C1-C6 alkoxy, or C1-C6 substituted alkoxy, and a is 1, 2, 3, or 4 or R₃′ is hydroxy, C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
   m is 0, 1, 2, 3, or 4.

116. The method, composition for use, or use according to any one of embodiments 107-115, wherein the stator is further defined as:

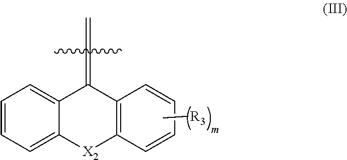

wherein:
   X₂ is a covalent bond, O, S, NR_{c}, or CR_{d}R_{d}′, wherein R_{c}, R_{d}, and R_{d}′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
m is 0, 1, 2, 3, or 4.

117. The method, composition for use, or use of embodiment 115, wherein the stator is further defined as:

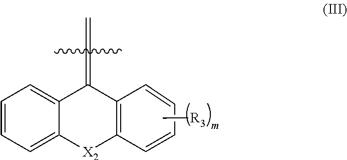

wherein:
   X₂ is a covalent bond, O, S, NR_{c}, or CR_{d}R_{d}′, wherein R_{c}, R_{d}, and R_{d}′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
   m is 0 or 1.

118. The method, composition for use, or use according to any one of embodiments 1-117, wherein the stimulus activated molecular motor comprises a rotor of the formula:

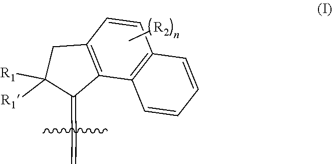

wherein:
   R₁ is C1-C12 alkyl or C1-C12 substituted alkyl; R₁′ is hydrogen;
   R₂ is hydrogen, C1-C12 alkoxy, or substituted C1-C12 alkoxy; or
   R₂ is a group of the formula: —Y₁ —X₁—R₂′, wherein:
      Y₁ is —NRₐ—, wherein: R₃ is hydrogen, C1-C6 alkyl, or substituted C1-C6 alkyl;
      X₁ is C1-C12 alkanediyl or C1-C12 substituted alkanediyl; and
      R₃′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0 or 1.
and a stator of the formula:

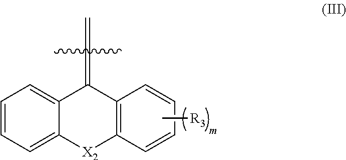

wherein:
   X₂ is a covalent bond or S;
   R₃ is hydrogen, halo; or
   R₃ is a group of the formula: —Y₂ X₃-R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C2-C12 alkynediyl, or C2-C12 substituted alkynediyl; and
      R₃′ is —NR_{f}R_{f}′R_{f}″, wherein R_{f} and R_{f}″ are each independently hydrogen, C1-C6 alkyl, or C1-C6 substituted alkyl, and R_{f}″ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C1-C12 heterocycloalkyl, C1-C12 substituted heterocycloalkyl, C18-C24 triarylphosphine or C18-C24 substituted triarylphosphine; and
   m is 1 or 2.

119. The method, composition for use, or use according to any one of embodiments 1-117, wherein the stimulus activated molecular motor comprises a rotor of the formula:

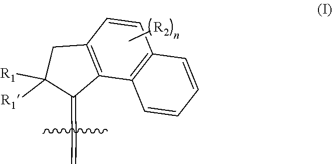

wherein:
   R₁ is C1-C12 alkyl or C1-C12 substituted alkyl;
   R₁′ is hydrogen;
   R₂ is hydrogen, C1-C12 alkoxy, or substituted C1-C12 alkoxy; or
   R₂ is a group of the formula: —Y₁—X₁—R₂′, wherein:
      Y₁ is —NRₐ—, wherein: R₃ is hydrogen, C1-C6 alkyl, or substituted C1-C6 alkyl;
      X₁ is C1-C12 alkanediyl or C1-C12 substituted alkanediyl; and
      R₃′ is —NR_{b}R_{b}′R_{b}″, wherein R_{b} and R₃′ are each independently hydrogen, C1-C6 alkyl, or C1-C6 substituted alkyl, and R_{b}″ is absent, hydrogen, C1-C6 alkyl, or C1-C6 substituted alkyl; and
   n is 0 or 1.
and a stator of the formula:

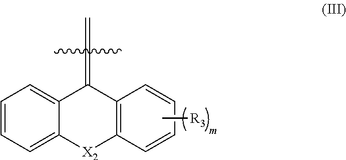

wherein:
   X₂ is a covalent bond or S;
   R₃ is hydrogen or halo; or
   R₃ is a group of the formula: —Y₂—X₃—R₃′, wherein:
      Y₂ is a covalent bond or —NRₑ—, wherein: Rₑ is hydrogen, C1-C6 alkyl, or substituted C1-C6 alkyl;
      X₃ is C1-C12 alkanediyl, C1-C12 substituted alkanediyl, C2-C12 alkynediyl, or C2-C12 substituted alkynediyl; and
      R₃′ is —NR_{f}R_{f}′R_{f}″, wherein R_{f} and R_{f}′ are each independently hydrogen, C1-C6 alkyl, or C1-C6 substituted alkyl, and Rr″ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C1-C12 heterocycloalkyl, C1-C12 substituted heterocycloalkyl, C18-C24 triarylphosphine or C18-C24 substituted triarylphosphine; and
   m is 1 or 2.

120. The method, composition for use, or use according to any one of embodiments 1-117, wherein the stimulus activated molecular motor comprises a rotor of the formula:

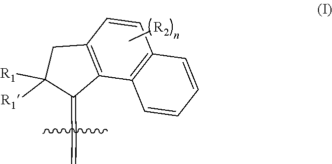

wherein:
   R₁ and R₁′ are each C1-C12 alkyl or C1-C12 substituted alkyl;
   R₁′ is hydrogen; and
   n is 0,
and a stator of the formula:

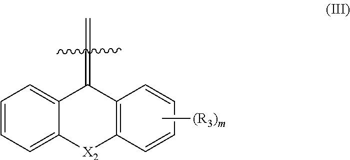

wherein:
   X₂ is S;
   R₃ is hydrogen; or
   R₃ is a group of the formula: —Y₂—X₃—R₃′, wherein:
      Y₂ is —NRₑ—, wherein: Rₑ is hydrogen, C1-C6 alkyl, or substituted C1-C6 alkyl;
      X₃ is C1-C12 alkanediyl or C1-C12 substituted alkanediyl; and
      R₃′ is —NR_{f}R_{f}R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl; and
   m is 1.

121. The method, composition for use, or use according to any one of embodiments 115-120, wherein R₃ is —Y₂—X₃—R_{3′.}

122. The method, composition for use, or use of embodiment 121, wherein Y₁ is —NRₑ—.

123. The method, composition for use, or use of embodiment 122, wherein Rₑ is hydrogen.

124. The method, composition for use, or use according to any one of embodiments 121-123, wherein X₃ is C1-C12 alkanediyl or C1-C12 substituted alkanediyl.

125. The method, composition for use, or use according to any one of embodiments 121-124, wherein X₃ is C1-C12 alkanediyl.

126. The method, composition for use, or use according to any one of embodiments 121-125, wherein X₃ is ethylene.

127 The method, composition for use, or use according to any one of embodiments 121-126, wherein R₃′ is —NR_{f}R_{f}′R_{f}″.

128. The method, composition for use, or use according to any one of embodiments 121-126, wherein R_{f} is C1-C6 alkyl or C1-C6 substituted alkyl.

129 The method, composition for use, or use according to any one of embodiments 121-128, wherein Reis C1-C6 alkyl.

130. The method, composition for use, or use according to any one of embodiments 121-129, wherein R_{f} is methyl.

131. The method, composition for use, or use according to any one of embodiments 121-130, wherein R_{f}′ is C1-C6 alkyl or C1-C6 substituted alkyl.

132. The method, composition for use, or use according to any one of embodiments 121-131, wherein R_{f}′ is C1-C6 alkyl.

133. The method, composition for use, or use according to any one of embodiments 121-132, wherein R_{f}′ is methyl.

134. The method, composition for use, or use according to any one of embodiments 121-133, wherein R_{f}′ is absent.

135. The method, composition for use, or use according to any one of embodiments 121-126, wherein R₃′ is C1-C12 heterocycloalkyl or C1-C12 heterocycloalkyl.

136. The method, composition for use, or use according to any one of embodiments 121-126 and 135, wherein R₃′ is C1-C12 heterocycloalkyl.

137. The method, composition for use, or use according to any one of embodiments 121-126, 135, and 136, wherein R₃′ is 1,4-piperazinyl.

138. The method, composition for use, or use according to any one of embodiments 115-137, wherein R₃ is —NHCH₂CH₂N(Me)₂ or —NHCH₂CH₂N (CH—CH₂)₂NH.

139. The method, composition for use, or use according to any one of embodiments 115-138, wherein n is 0.

140 The method, composition for use, or use according to any one of embodiments 115-138, wherein n is 1.

141. The method, composition for use, or use according to any one of embodiments 115-140, wherein X₂ is S.

142. The method, composition for use, or use according to any one of embodiments 115-140, wherein X₂ is a covalent bond.

143. The method, composition for use, or use according to any one of embodiments 115-140, wherein X₂ is CR_{d}R_{d}′.

144. The method, composition for use, or use of embodiment 143, wherein R_{d} is C1-C6 alkyl or C1-C6 substituted alkyl.

145. The method, composition for use, or use of either embodiment 143 or embodiment 144, wherein R_{d} is C1-C6 alkyl.

146. The method, composition for use, or use according to any one of embodiments 143-145, wherein R_{d} is methyl.

147. The method, composition for use, or use according to any one of embodiments 143-146, wherein R₃′ is C1-C6 alkyl or C1-C6 substituted alkyl.

148. The method, composition for use, or use according to any one of embodiments 143-147, wherein R₃′ is C1-C6 alkyl.

149. The method, composition for use, or use according to any one of embodiments 143-148, wherein R₃′ is methyl.

150. The method, composition for use, or use according to any one of embodiments 1-149, wherein the stimulus activated molecular motor is further defined as:

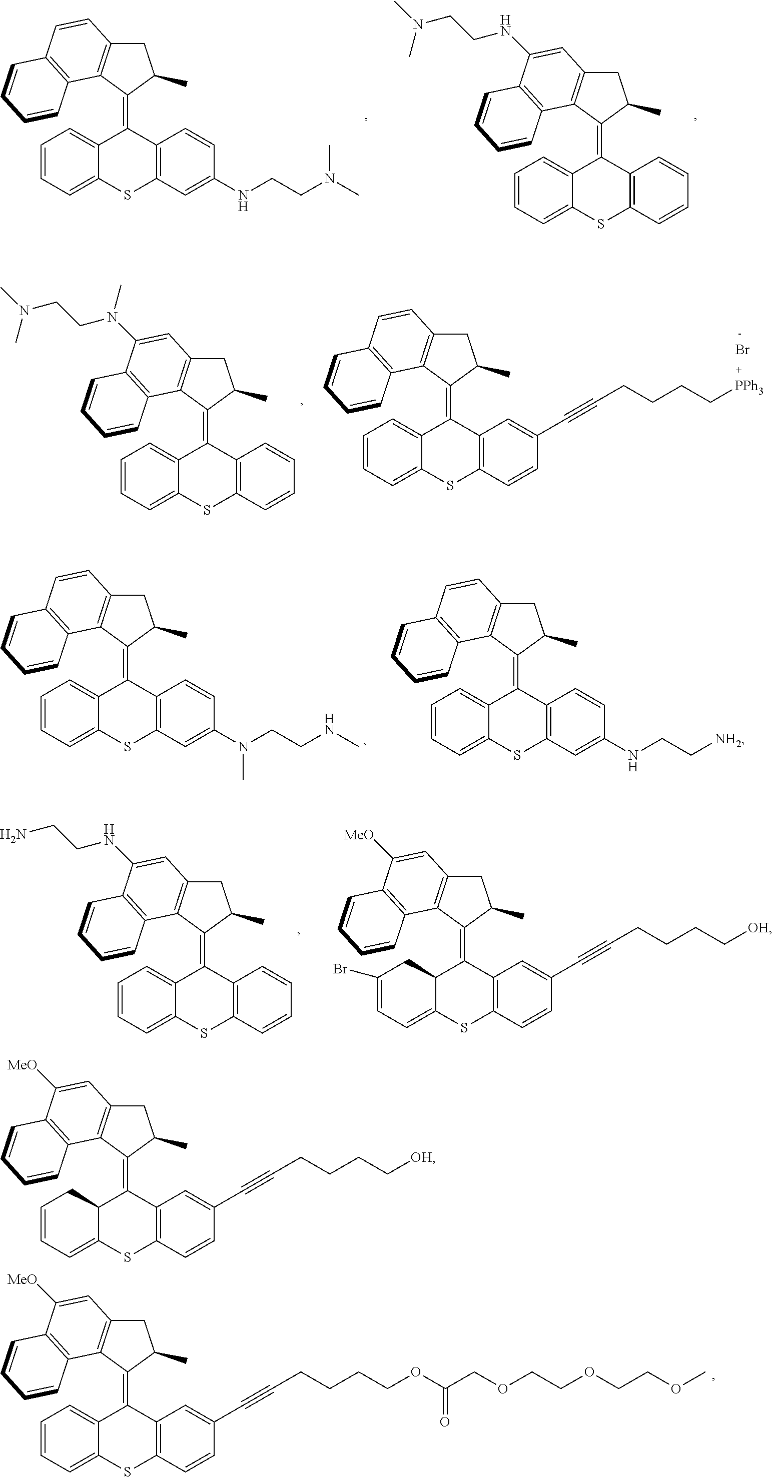

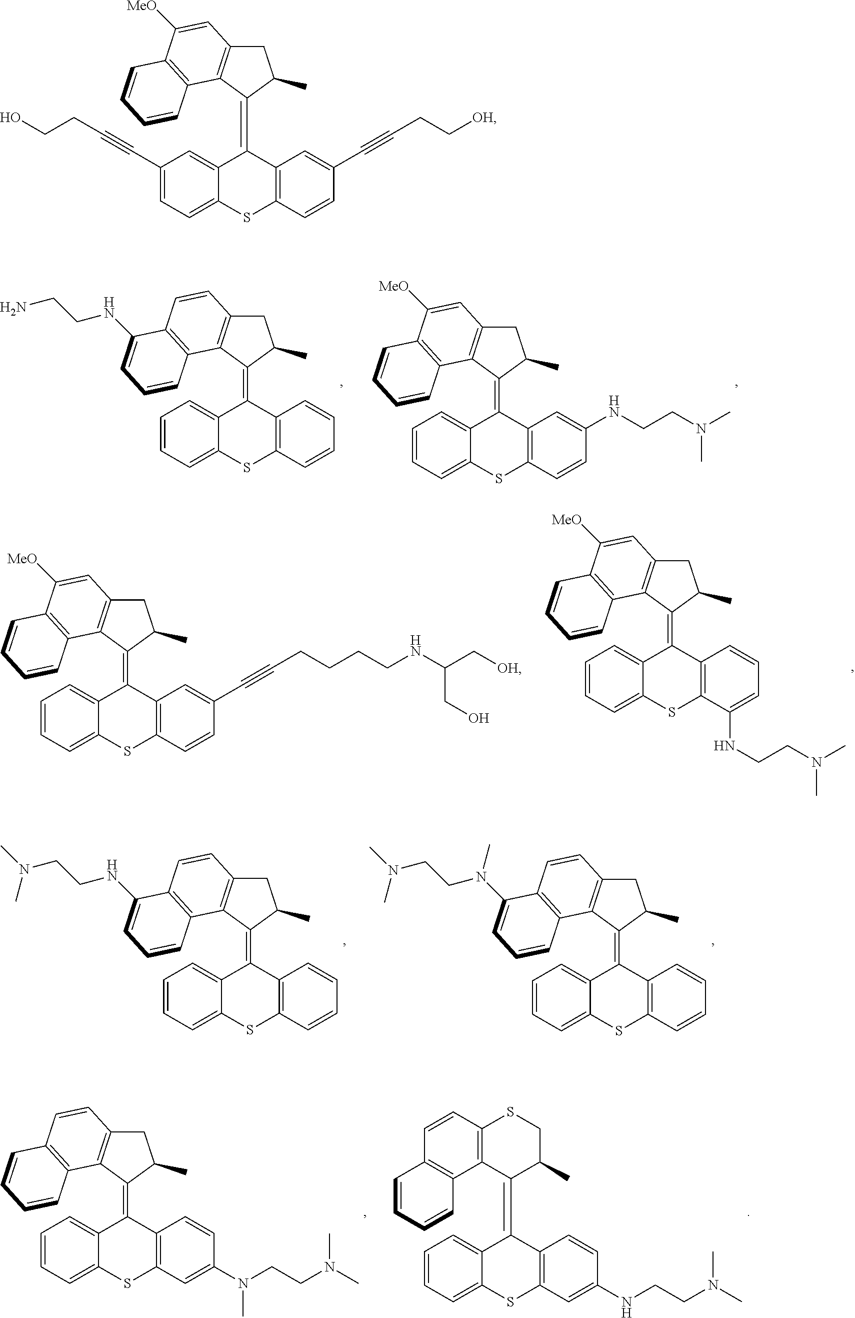

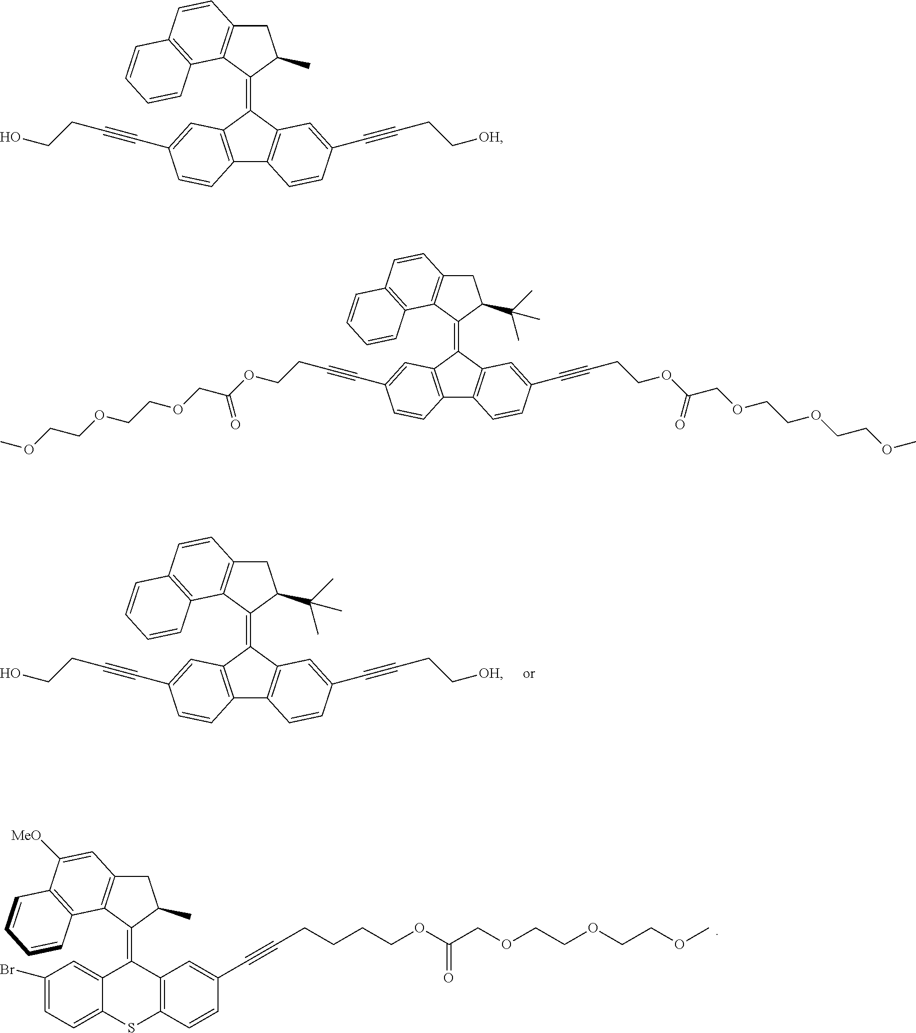

151. The method, composition for use, or use according to any one of embodiments 1-149, wherein the stimulus activated molecular motor is further defined as:

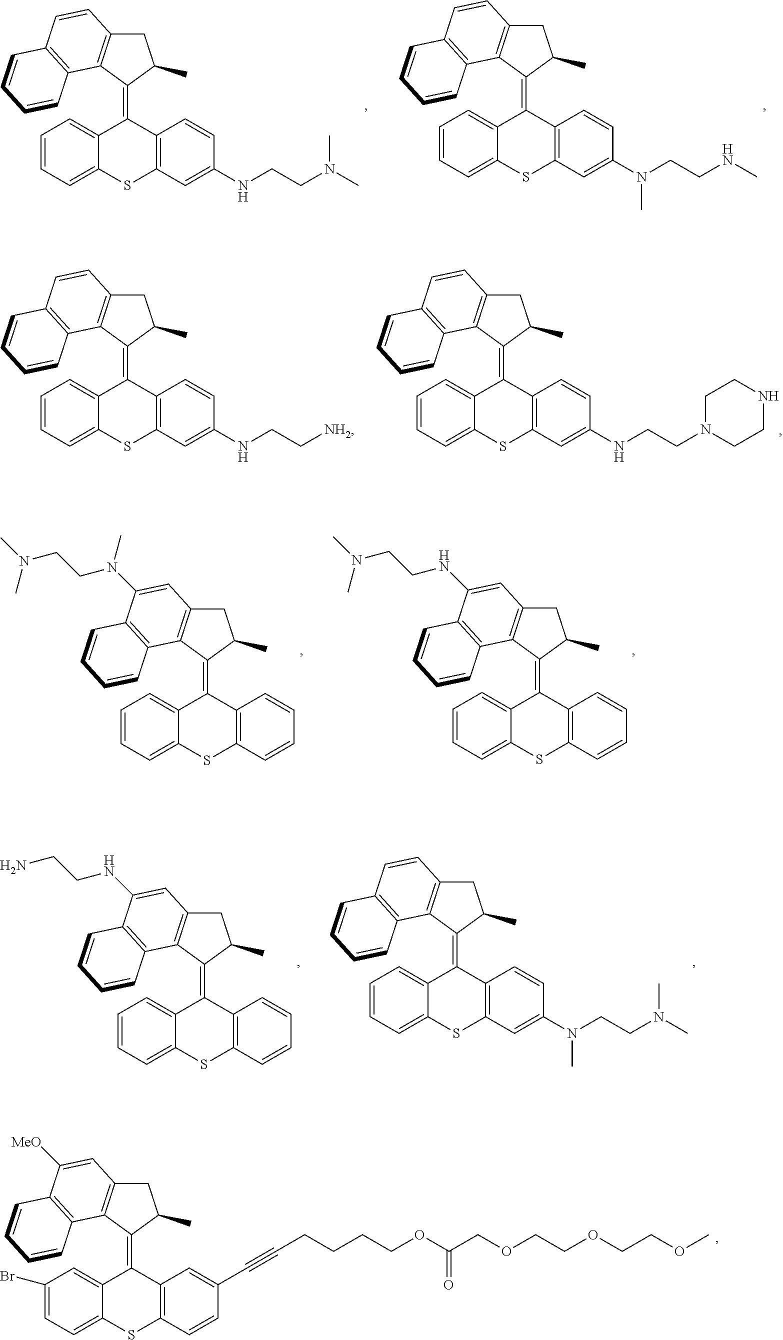

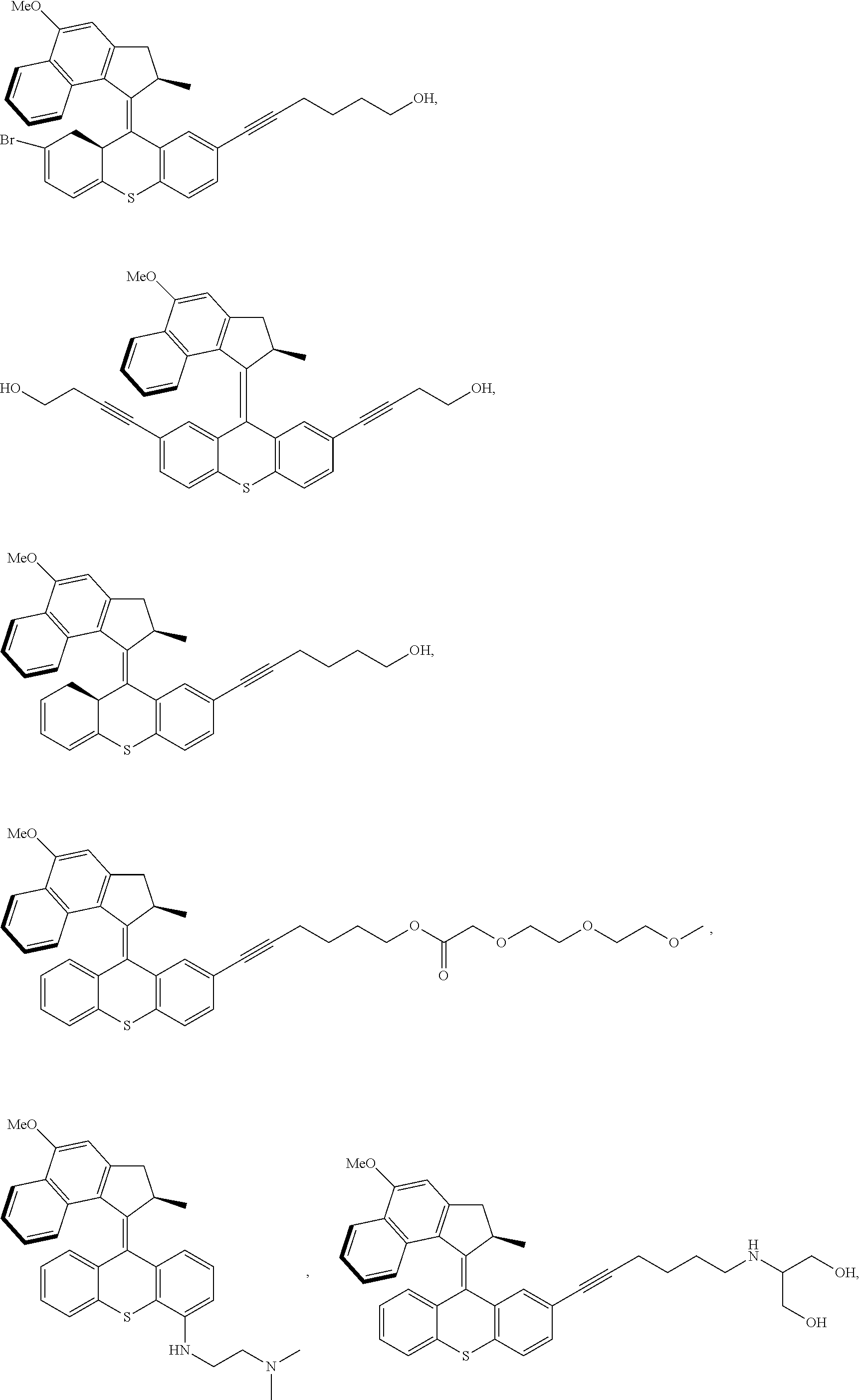

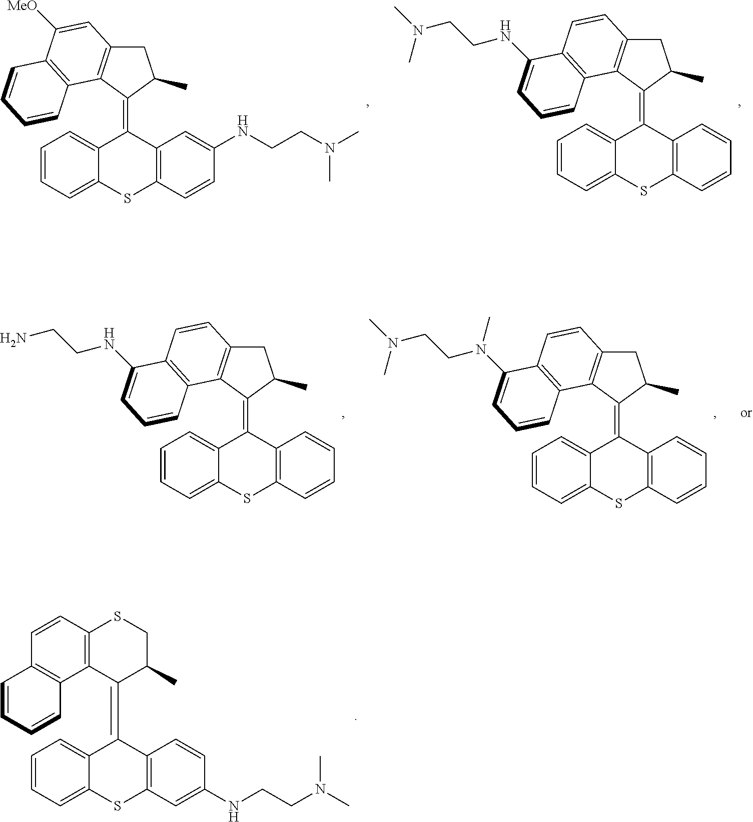

152. The method, composition for use, or use according to any one of claims 1-149, wherein the stimulus activated molecular machine is further defined as:

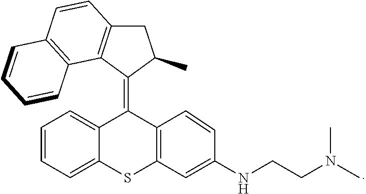

153. The method, composition for use, or use according to any one of embodiments 1-149, wherein the stimulus activated molecular machine is further defined as:

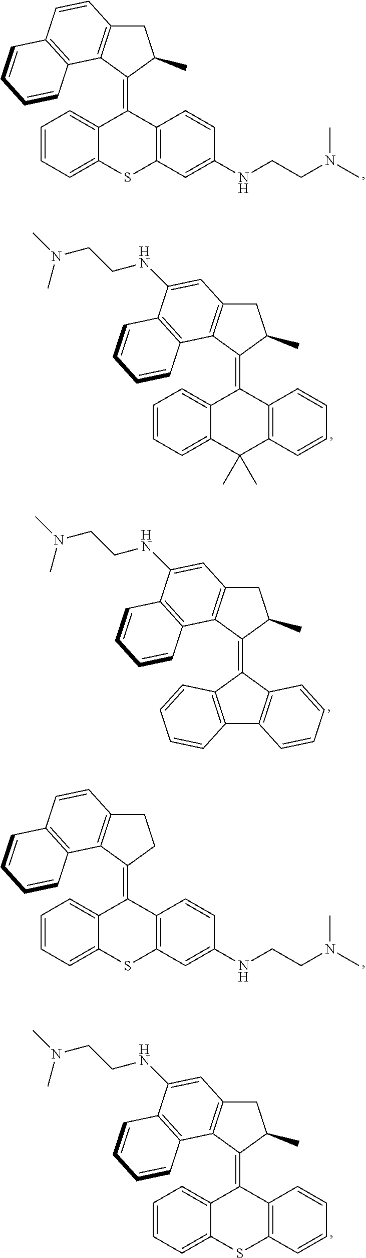

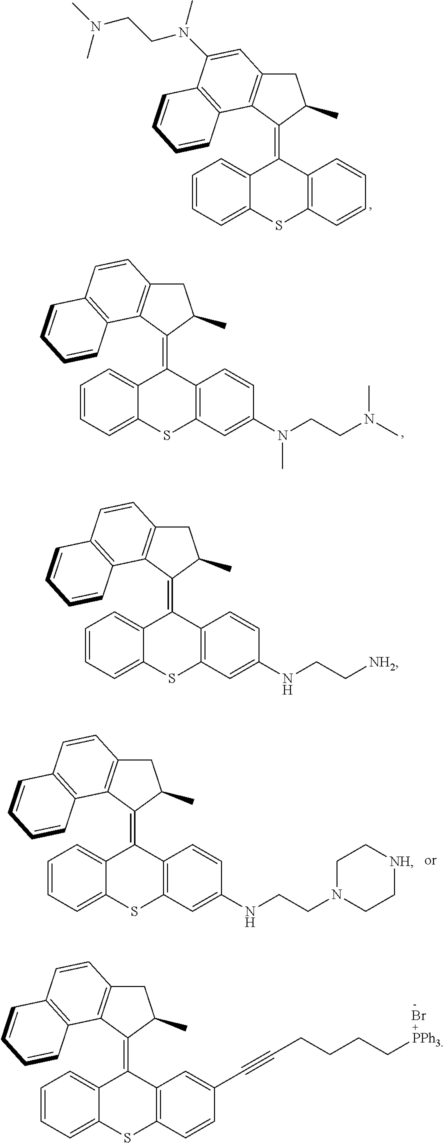

154. The method, composition for use, or use according to any one of embodiments 1~153, wherein the stimulus activated molecular machine is not a compound of the formula:

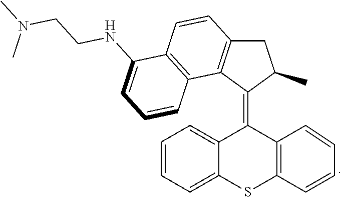

155. The method, composition for use, or use according to any one of embodiments 1-154, wherein the stimulus activated molecular machine rotates unidirectionally.

156. The method, composition for use, or use according to any one of embodiments 1-153, wherein the stimulus activated molecular machine rotates bidirectionally.

157. The method, composition for use, or use according to any one of embodiments 1-156, wherein the rotational component of the stimulus activated molecular machine rotates at a speed greater than 1 Hz.

158. The method, composition for use, or use of embodiment 157, wherein the rotational component of the stimulus activated molecular machine rotates at a speed greater than 105 Hz.

159. The method, composition for use, or use of embodiment 158, wherein the rotational component of the molecular machine or switch rotates at a speed of about 106 Hz.

160. The method, composition for use, or use of embodiment 159, wherein the rotational component of the molecular machine or switch rotates at a speed of about 108 Hz.

161. The method, composition for use, or use according to any one of embodiments 1-160, wherein the stimulus activated molecular machine is activated by a stimulus.

162. The method, composition for use, or use of embodiment 161, wherein the stimulus is electromagnetic radiation.

163. The method, composition for use, or use of either embodiment 161 or embodiment 162, wherein the electromagnetic radiation comprises gamma rays, X-rays, UV light, visible light, near infrared light, infrared light, microwaves, or radio waves.

164. The method, composition for use, or use of embodiment 163, wherein the electromagnetic radiation comprises UV light, visible light, or near infrared light.

165. The method, composition for use, or use of embodiment 164, wherein the electromagnetic radiation comprises visible light.

166. The method, composition for use, or use of embodiment 165, wherein the electromagnetic radiation comprises a wavelength of 400 nm.

167. The method, composition for use, or use according to any one of embodiments 1-166, wherein the stimulus activated molecular machine is activated for a controlled time period.

168. The method, composition for use, or use of embodiment 167, wherein the stimulus activated molecular machine is activated for less than 5 seconds.

169. The method, composition for use, or use of embodiment 168, wherein the stimulus activated molecular machine is activated for less than 2 seconds.

170. The method, composition for use, or use of embodiment 169, wherein the stimulus activated molecular machine is activated for about 250 milliseconds.

171. The method, composition for use, or use according to any one of embodiments 1-170, wherein the energy source is a laser.

172. The method, composition for use, or use according to any one of embodiments 1-171, wherein the intensity of the energy source is controlled.

173. The method, composition for use, or use according to any one of embodiments 1-172, wherein the patient is a mammal.

174. The method, composition for use, or use of embodiment 173, wherein the mammal is a human.

175. A molecular machine comprising:
(A) a rotor of the formula:

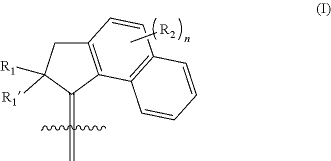

wherein:
   R₁ and R₁′ are each independently hydrogen, a C1-C12 alkyl or a C1-C12 substituted alkyl;
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁ —X₁—R₂′, wherein:
      Y₁ is —O—, —S—, or —NRₐ—, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R₂′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4; and
(B) a stator of the formula:

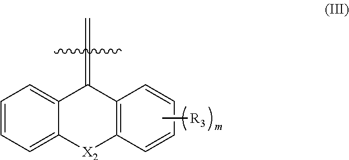

wherein:
   X₂ is a covalent bond, O, S, NR_{c}, or CRₐR_{d}′, wherein R_{c}, R_{d}, and R_{d}′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
   m is 0, 1, 2, 3, or 4;
provided at least one of R₂ is a group of the formula: —Y₁—X₁—R_{f}′ or at least one of R₃ is a group of the formula: —Y₂—X₃—R₃′ provided that the molecular machine is not a compound of the formula:

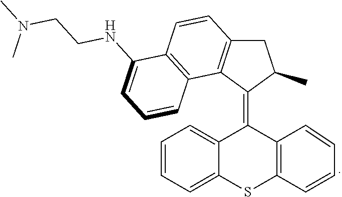

176. The molecular machine of embodiment 175, wherein the rotor is further defined as:

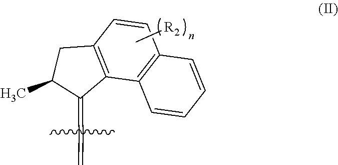

wherein:
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁ —X₁—R₂′, wherein:
      Y₁ is —O—, —S—, or —NRₐ—, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R₂′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4.

177. The molecular machine of embodiment 175, wherein R₁ is C1-C12 alkyl or substituted C1-C12 alkyl.

178. The molecular machine of embodiment 177, wherein R₁ is C1-C12 alkyl.

179. The molecular machine of embodiment 177 or claim 178, wherein R₁ is methyl.

180 The molecular machine according to any one of embodiments 175 and 177-179, wherein R₁′ is hydrogen.

181. The molecular machine according to any one of embodiments 175-180, wherein R₂ is hydrogen.

182 The molecular machine according to any one of embodiments 175-180, wherein R₂ is —Y₁ —X₁—Rₐ—.

183. The molecular machine of embodiment 182, wherein Y₁ is —NRₐ—.

184. The molecular machine of embodiment 183, wherein Rₐ is hydrogen.

185. The molecular machine according to any one of embodiments 182-184, wherein X₁ is C1-C12 alkanediyl or C1-C12 substituted alkanediyl.

186. The molecular machine according to any one of embodiments 182-185, wherein X₁ is C1-C12 alkanediyl.

187. The molecular machine according to any one of embodiments 182-186, wherein X₁ is ethylene.

188. The molecular machine according to any one of embodiments 182-187, wherein R_{b} is C1-C6 alkyl or C1-C6 substituted alkyl.

189. The molecular machine according to any one of embodiments 182-188, wherein R_{b} is C1-C6 alkyl.

190. The molecular machine according to any one of embodiments 182-189, wherein R_{b} is methyl.

191. The molecular machine according to any one of embodiments 182-190, wherein R_{b}′ is C1-C6 alkyl or C1-C6 substituted alkyl.

192. The molecular machine according to any one of embodiments 182-191, wherein R₃′ is C1-C6 alkyl.

193. The molecular machine according to any one of embodiments 182-192, wherein R_{b}′ is methyl.

194. The molecular machine according to any one of embodiments 182-193, wherein R_{b}″ is absent.

195. The molecular machine according to any one of embodiments 175-194, wherein R₂ is —NHCH₂CH₂N(Me) 2.

196. The molecular machine according to any one of embodiments 175-195, wherein n is 0.

197. The molecular machine according to any one of embodiments 175-195, wherein n is 1.

198. The molecular machine according to any one of embodiments 175-197, wherein the stator is further defined as:

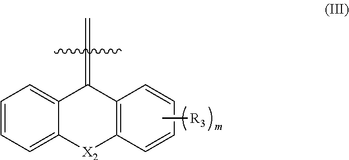

wherein:
   Y₂ is a covalent bond, —O—, —S—, or —NRₑ—, wherein: Rₑ is hydrogen, C1-C6 alkyl, or substituted C1-C6 alkyl;
   X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
   R₃′ is —NR (R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
m is 0 or 1.

199. The molecular machine according to any one of embodiments 175-198, wherein R₃ is —Y₂—X₃—R₃′.

200. The molecular machine according to any one of embodiments 175-199, wherein Y₁ is —NRₑ—.

201. The molecular machine according to any one of embodiments 175-200, wherein Rₑ is hydrogen.

202. The molecular machine according to any one of embodiments 175-201, wherein X₃ is C1-C12 alkanediyl or C1-C12 substituted alkanediyl.

203 The molecular machine according to any one of embodiments 175-202, wherein X₃ is C1-C12 alkanediyl.

204. The molecular machine according to any one of embodiments 175-203, wherein X₃ is ethylene.

205. The molecular machine according to any one of embodiments 175-204, wherein R₃′ is —NR_{f}R_{f}′R_{f}″.

206. The molecular machine according to any one of embodiments 175-205, wherein Rr is C1-C6 alkyl or C1-C6 substituted alkyl.

207. The molecular machine according to any one of embodiments 175-206, wherein R_{f} is C1-C6 alkyl.

208. The molecular machine according to any one of embodiments 175-207, wherein R_{f} is methyl.

209. The molecular machine according to any one of embodiments 175-208, wherein R_{f}′ is C1-C6 alkyl or C1-C6 substituted alkyl.

210. The molecular machine according to any one of embodiments 175-209, wherein R_{f}′ is C1-C6 alkyl.

211. The molecular machine according to any one of embodiments 175-210, wherein R_{f}′ is methyl.

212. The molecular machine according to any one of embodiments 175-211, wherein R_{f}″ is absent.

213. The molecular machine according to any one of embodiments 175-204, wherein R₃′ is C1-C12 heterocycloalkyl or C1-C12 heterocycloalkyl.

214. The molecular machine according to any one of embodiments 175-204 and 213, wherein R₃′ is C1-C12 heterocycloalkyl.

215. The molecular machine according to any one of embodiments 175-204, 213, and 214, wherein R₃′ is 1,4-piperazinyl.

216. The molecular machine according to any one of embodiments 175-215, wherein R₃ is —NHCH₂CH₂N(Me) 2 or —NHCH₂CH₂N (CH₂CH₂) 2NH.

217. The molecular machine according to any one of embodiments 175-216, wherein n is 0.

218. The molecular machine according to any one of embodiments 175-216, wherein n is 1.

219. The molecular machine according to any one of embodiments 175-218, wherein X₂ is S.

220. The molecular machine according to any one of embodiments 175-218, wherein X₂ is a covalent bond.

221. The molecular machine according to any one of embodiments 175-218, wherein X₂ is CR_{d}R_{d}′.

222. The molecular machine of embodiment 221, wherein R_{d} is C1-C6 alkyl or C1-C6 substituted alkyl.

223. The molecular machine of either embodiment 221 or embodiment 222, wherein R₃ is C1-C6 alkyl.

224. The molecular machine according to any one of claims 221-223, wherein R₃ is methyl.

225. The molecular machine according to any one of embodiments 221-224, wherein R₃′ is C1-C6 alkyl or C1-C6 substituted alkyl.

226. The molecular machine according to any one of embodiments 221-225, wherein R₃′ is C1-C6 alkyl.

2.27. The molecular machine according to any one of embodiments 221-226, wherein R₃′ is methyl.

228. The molecular machine according to any one of embodiments 175-227, wherein the stimulus activated molecular machine is further defined as:

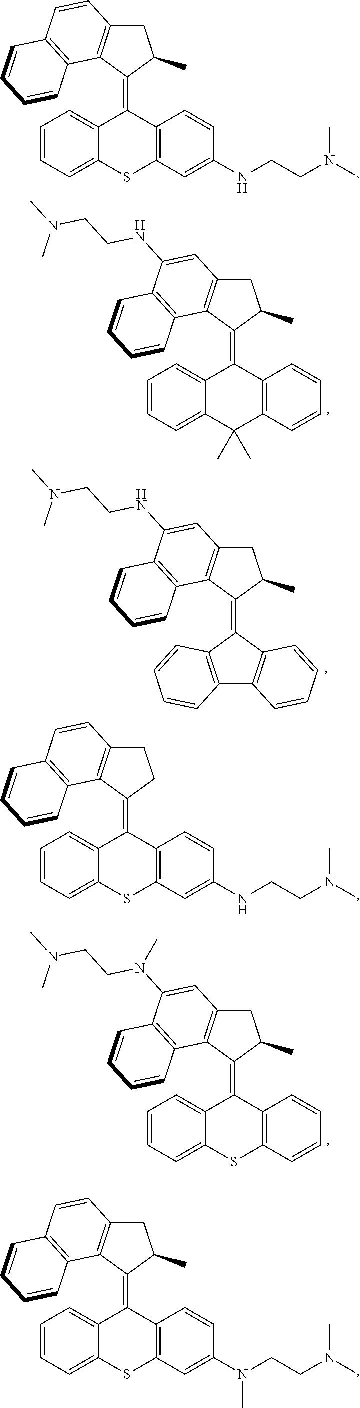

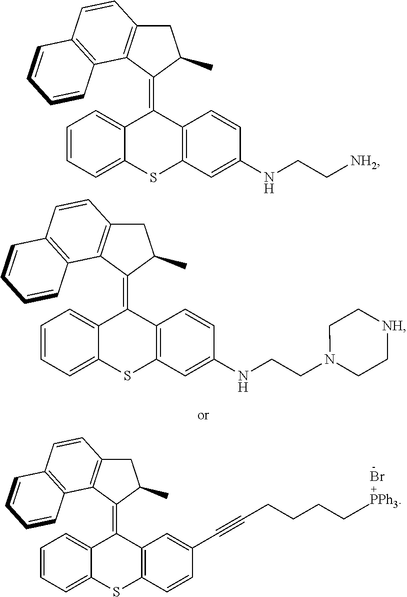

229. A method of treating a fungal infection in a patient comprising:
(A) administering to the patient in need thereof a stimulus activated molecular machine, wherein the stimulus activated molecule machine is configured to be activated by an appropriate stimulus; and
(B) exposing the patient to a stimulus sufficient to activate the molecular machine.

230. A method of treating cancer in a patient in need thereof comprising administering to the patient a stimulus activated molecular machine, wherein the stimulus activated molecule machine is configured to be activated by an appropriate stimulus, and exposing the patient to an appropriate stimulus wherein the stimulus activated molecular machine comprises a rotor of the formula:

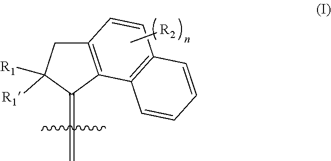

wherein:
   R₁ and R₁′ are each independently hydrogen, a C1-C12 alkyl or a C1-C12 substituted alkyl;
   R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₂ is a group of the formula: —Y₁ —X₁—R₂′, wherein:
      Y₁ is —O—, —S—, or —NRₐ—, wherein: R₃ is hydrogen, C1-C6 alkyl, or substituted C1-C6 alkyl;
      X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
      R₂′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
   n is 0, 1, 2, 3, or 4; and
a stator of the formula:

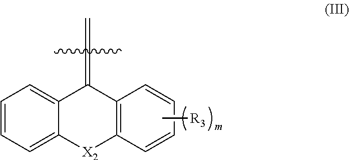

wherein:
   X₂ is a covalent bond, O, S, NR_{c}, or CR_{d}R_{d}′, wherein R_{c}, R_{d}, and R_{d}′ are each independently hydrogen, C1-C6 alkyl, or substituted C1-C6 alkyl;
   R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
   R₃ is a group of the formula: —Y₂—X₃—R₃′, wherein:
      Y₂ is a covalent bond, —O—, —S—, or —NRₑ—, wherein: Rₑ is hydrogen, C1-C6 alkyl, or substituted C1-C6 alkyl;
      X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}′ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
   m is 0, 1, 2, 3, or 4;
   provided that the compound is not:

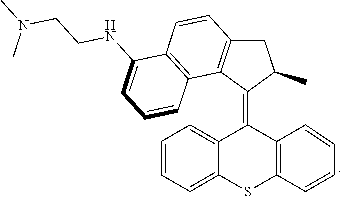

231. The method of embodiment 230, wherein the stimulus activated molecular motor comprises a rotor of the formula:

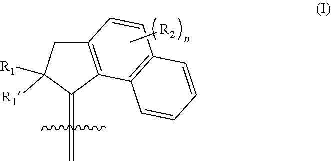

wherein:
   R₁ and R₁′ are each C1-C12 alkyl or C1-C12 substituted alkyl;
   R₁′ is hydrogen; and n is 0.
and a stator of the formula:

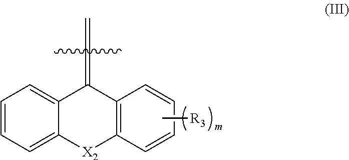

wherein:
   X₂ is S;
   R₃ is hydrogen; or
   R₃ is a group of the formula: —Y₂—X₃—R₃′, wherein:
      Y₂ is —NRₑ—, wherein: Rₑ is hydrogen, C1-C6 alkyl, or substituted C1-C6 alkyl;
      X₃ is C1-C12 alkanediyl or C1-C12 substituted alkanediyl; and
      R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl; and
   m is 1.

232. The method according to any one of embodiments 1-231, wherein the stimulus activated molecular motor is not a compound of the formula:

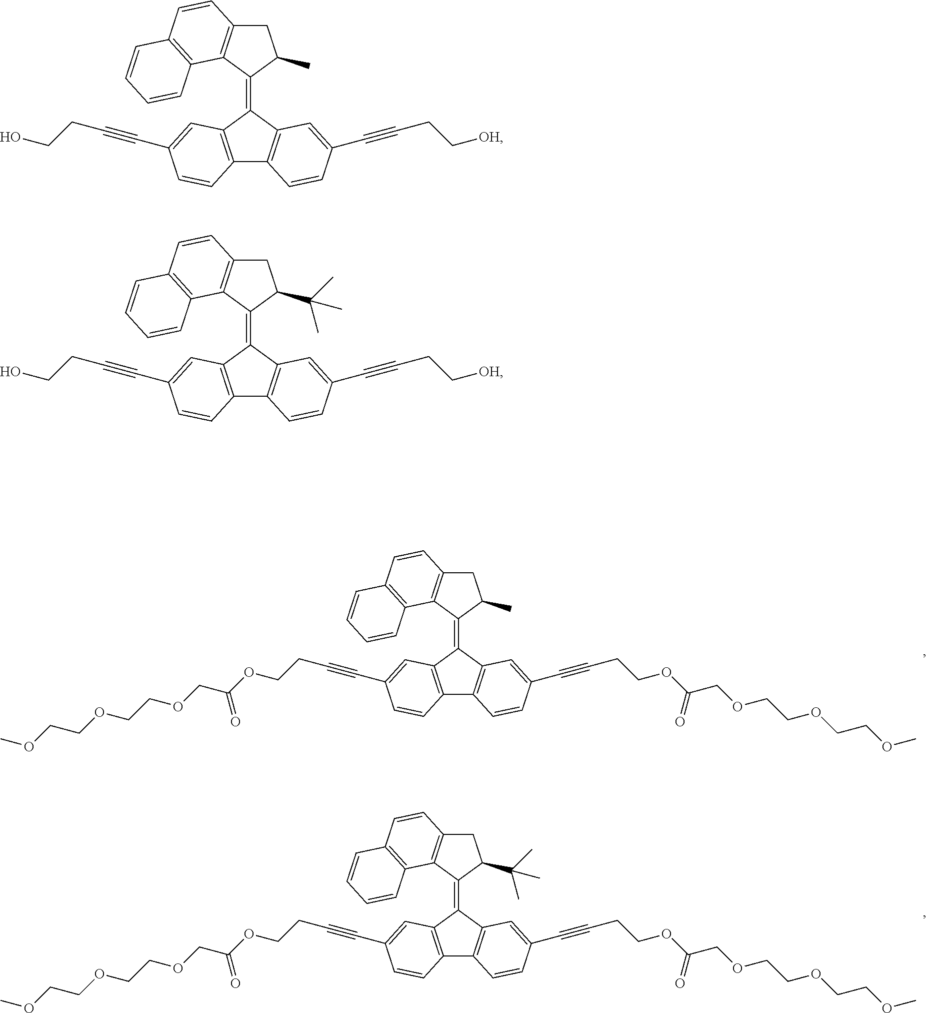

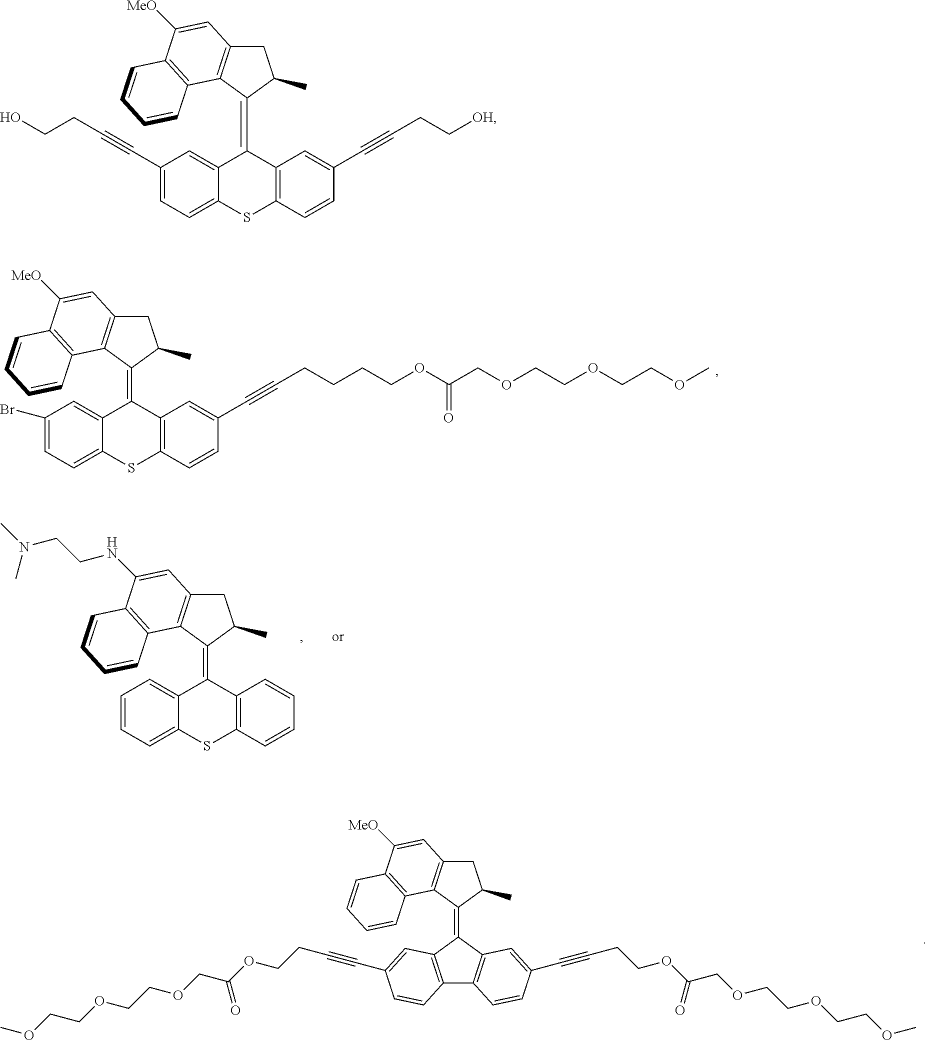

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1I: MMs show antifungal activity against planktonic cells and established biofilms. (FIG. 1A) Exemplary structure of an MM. MMs consist of a stator and a rotor that is light-activated. After light activation, the rotor portion of the molecule undergoes successive cycles of unidirectional rotation around the central carbon-carbon double bond, resulting in a fast (~3 MHz) or slow (~0.1 Hz) drill-like motion, depending on the molecular design. (FIG. 1B) Minimum inhibitory concentration (MIC, μM) of the different MMs investigated for antifungal activity in C. albicans in the presence of 405-nm light (87.6 J cm⁻²). The chemical structures of all compounds tested in Example 1 are shown in Table 1. (FIG. 1C)Chemical structures of the most potent antifungal MMs identified by the inventors, their MIC, and minimal fungicidal concentration (MFC) in different fungal strains. Results are shown as the average of at least three biological replicas. Concentration is expressed in μM. (FIG. 1D) Time-kill curves of different fungal strains treated with visible-light-activated MMs (2×MIC) or 1% DMSO in the presence of 405-nm light at 292 mW cm⁻² or control antifungal amphotericin B (AMB, 4×MIC). (FIG. 1E)Concentration-dependent killing of C. albicans by different MMs in the presence of 405-nm light (87.6 J cm⁻²). (FIG. 1F) Light dose-dependent killing of C. albicans by different MMs at 2×MIC. Killing was assessed as the reduction in colony forming units (CFU) expressed as the logarithm of base 10 of the ratio between the CFU at each time point (N) and the CFU at time zero (NO). The results are expressed as the average of at least three replicates±the standard error of the mean. The dashed line denotes the limit of detection of the method. (FIG. 1G) Reduction of C. albicans biofilm viability by amphotericin B (AMB), 1% DMSO or different MMs (2×, 4×MIC) in the presence of 405-nm light (5 min at 292 mW cm⁻²). (FIG. 1H) Reduction of C. albicans biofilm biomass by amphotericin B (AMB), 1% DMSO or different MMs (2×, 4×MIC) in the presence of 405-nm light (5 min at 292 mW cm⁻²). The results are the average of at least three independent replicates #the standard error of the mean. Asterisks denote the significance of the differences in pairwise comparisons with 1% DMSO controls performed in GraphPad Prism. * p<0.05, ** p<0.01, *** p<0.001, **** p<0.0001. (FIG. 1I) Development of resistance to conventional antifungals (caspofungin, CAS, fluconazole, FLC, or amphotericin B, AMB) or different visible-light-activated MMs in C. albicans, assessed as the MIC fold change over 20 cycles of repeated treatment. Note that curves for amphotericin B (AMB), MM 1, MM 5, MM 6, and MM 7 are superimposed. Unless otherwise indicated, the results for MMs and DMSO are always reported in the presence of light.

FIG. 2: Time-kill curves of different fungal strains treated with 2×MIC of different MMs in the absence of light. The results are the average of at least three independent biological replicates #the standard error of the mean.

FIG. 3: Evaluation of the development of resistance to MMs in C. albicans. For the stepwise resistance assessment by serial passage (left), C. albicans cell suspensions were treated with increasing concentrations (0.3125-160 μM) of different MMs (8 mM stock in DMSO) and then irradiated with 405 μm light (87.6 J cm⁻²). The irradiated cell suspensions were then inoculated in MOPS-buffered RPMI 1640 (pH 7.0, Sigma, MO, USA), and the tubes were incubated at 30 ° C for 48 h. The minimum inhibitory concentration (MIC) was identified as the concentration of antifungal or MMs that resulted in no visible growth after incubation (Rayens et al., 2022). Cells able to grow at 0.5×MIC of each MM were collected by centrifugation (5,000×g, 5 min), resuspended and re-challenged with a range of MM concentrations and irradiated with 405 nm light (87.6 J cm⁻²). The procedure was repeated for a total of 20 consecutive cycles. The isolation of MM-resistant mutants was also attempted using a single-step strategy (right) (Fisher et al., 2018), whereby high-density (~10⁹ c.f.u. ml 1) cell suspensions of C. albicans cells were treated with 4×MIC of the various MMs and irradiated with 405 nm light (87.6 J cm⁻²). Irradiated cells were then inoculated in YPD and incubated at 30 °C. However, MM-resistant colonies could not be recovered even after 14 days of incubation. Created with Biorender.com.

FIGS. 4A-4K provide evidence that MMs bind fungal mitochondrial phospholipids. (FIG. 4A) Representative temporal profile of propidium iodide (PI) fluorescence in C. albicans treated with MM 1 (0.5-2×MIC) or 1% DMSO and irradiated with 405-nm light (87.6 J cm⁻²). Lines are the average of at least three biological replicates, and the shaded area is the standard error of the mean. (FIG. 4B) PI uptake in C. albicans treated with different MMs (0.5-2×MIC) or 1% DMSO in the presence of 405-nm light (87.6 J cm⁻²). PI uptake was calculated as the area under the curve (AUC) of the temporal profiles of PI fluorescence, as shown in (FIG. 4A). The results are the average of at least three independent replicates #the standard deviation. (FIG. 4C) Representative histogram of calcein AM fluorescence in C. albicans cells treated with 1% DMSO or MM 1 (0.5-2×MIC) and irradiated with 405-nm light (87.6 J cm⁻²), assessed by flow cytometry. (FIG. 4D) Decrease in calcein AM fluorescence in C. albicans treated with 1% DMSO or different MMs (0.5-2×MIC) and irradiated with 405-nm light (87.6 J cm⁻²). The results are expressed as the arithmetic mean the standard deviation of fluorescence obtained by flow cytometry. (FIG. 4E) Extracellular ATP levels in C. albicans treated with increasing concentrations of different MMs (0.5-2×MIC) or 1% DMSO and irradiated with 405-nm light (87.6 J cm⁻²). The results are the average of at least three independent replicates±the standard deviation. (FIG. 4F) DPH fluorescence of C. albicans cells treated with 1% DMSO or different MMs (0.5-2×MIC) and irradiated with 405-nm light (87.6 J cm⁻²). Amphotericin B (AMB) was used as a control. (FIG. 4G) Effect of exogenous ergosterol, plasma membrane phospholipids (phosphatidylethanolamine, PE, and phosphatidylcholine, PC) or mitochondrial phospholipids (phosphatidylglycerol, PG, and cardiolipin, CL) on the sensitivity of C. albicans to MMs, evaluated as the MIC, in the presence of 405-nm light (87.6 J cm⁻²). Symbols denote the average of three replicas. Asterisks denote the significance of the differences in pairwise comparisons between the MIC in the absence and in the presence of increasing concentrations of different exogenous phospholipids. (FIG. 4H) SEM images of C. albicans treated with 1% DMSO or 0.5×MIC of visible-light-activated MM 1. (FIG. 41) TEM images of C. albicans treated with 1% DMSO or 0.5×MIC of visible-light-activated MM 1. Arrowheads indicate enlarged mitochondria in MM-treated samples compared with normal mitochondria in DMSO-treated samples (arrows). The bar indicates the scale. Unless otherwise indicated, the results for MMs and DMSO are always reported in the presence of light. (FIG. 4J)Confocal microscopy images of C. albicans treated with MM 1 (8 μM) and then labeled with the fluorescent mitochondrial dye MitoTracker™ Green (10 nM) and the fluorescent plasma membrane dye FM™ 4-64 (40 nM). The image identified as “combined” is a merger of the natural fluorescence of MM 1, Mito Tracker™ Green, and FM™ 4-64. The bar indicates the scale. (FIG. 4K) Box-and-whisker plot of the percentage overlap of fluorescence from MitoTracker™ Green or FM™ 4-64 with the natural fluorescence from MM 1. Light was omitted in colocalization experiments. Results are shown as the average of five independent cells±the standard deviation. Asterisks denote the significance of the differences in pairwise comparisons with 1% DMSO controls performed in GraphPad prism. * p<0.05, ** p<0.01, *** p<0.001, **** p<0.0001.

FIG. 5: Effect of increasing concentrations of glucose-6-phosphate, used as a representative of the negatively charged polysaccharides of the fungal cell wall, on the MIC of different MMs in C. albicans determined by competition binding experiments. Further details on the experimental procedure are provided in Example 1. Note that the lines from MM 5 and MM 6 are superimposed. The results are the average of at least three independent biological replicates.

FIG. 6: MICs of different visible-light-activated MMs in C. albicans grown in the presence and absence of sorbitol (0.8 M). Sorbitol protects cells from drugs that target the fungal cell wall, resulting in an increase in the MIC compared with untreated samples (Perfect, 2017). Experimental details on the determination of the MM MIC can be found in Example 1 below. The results are the average of at least three independent biological replicates.

FIGS. 7A-7I provide evidence that visible-light-activated MMs trigger mitochondrial dysfunction and oxidative stress. (FIG. 7A) Mitochondrial dehydrogenase activity in C. albicans treated with 1% DMSO or different MMs (0.5-2×MIC) in the presence of 405-nm light (87.6 J cm⁻²). (FIG. 7B) Intracellular ATP levels in C. albicans treated with 1% DMSO or different MMs (0.5-2×MIC) and 405-nm light (87.6 J cm⁻²). (FIG. 7C) Mitochondrial ROS levels detected by spectrofluorimetry using the MitoROS™ 580 probe in C. albicans treated with 1% DMSO or different MMs (0.5-2×MIC) and 405-nm light (87.6 J cm⁻²). (FIG. 7D) Mitochondrial ROS levels detected by confocal microscopy using the MitoROS™ 580 probe in C. albicans treated with MM 1 (1×MIC) before and after light activation under the microscope. The bar indicates the scale. (FIG. 7E) Temporal profile of MitoROS™ 580 fluorescence detected by confocal microscopy, shown as the average fluorescence intensity (line) and standard error of the mean (shaded area). (FIG. 7F) SOD activity normalized to the protein content in C. albicans treated with 1% DMSO or different MMs (0.5-2×MIC) and 405-nm light (87.6 J cm⁻²). (FIG. 7G) Lipid peroxidation assessed from malondialdehyde levels (MDA) normalized by protein content in C. albicans treated with 1% DMSO or different MMs (0.5-2×MIC) and 405-nm light (87.6 J cm⁻²). (FIG. 7H) Representative shifts in the fluorescence of JC-1 in C. albicans treated with 1% DMSO or MM 1 (0.5-2×MIC) and 405-nm light (87.6 J cm⁻²) detected by flow cytometry denoting MM-induced depolarization of the mitochondrial membrane. (FIG. 7I)Changes in the percentage of depolarized cells in C. albicans treated with 1% DMSO or different MMs (0.5-2×MIC) and 405-nm light (87.6 J cm⁻²) detected with JC-1 by flow cytometry. All results are shown as the average of at least three independent replicates #the standard deviation. Asterisks denote the significance of the differences in pairwise comparisons with 1% DMSO controls performed in GraphPad Prism. * p<0.05, ** p<0.01, *** p<0.001, **** p<0.0001. Unless otherwise stated, the results for MMs and DMSO are always reported in the presence of light.

FIG. 8: Survival curves of de-energized versus exponential, fully energized cells of C. albicans treated with visible-light-activated MM 1 (2×MIC). Energy depletion of C. albicans was achieved by resuspending cells in de-energization buffer (1 μM antimycin A, 5 mM 2-deoxy-D-glucose, 50 mM HEPES buffer, pH 7.0) for 3 h, as described for the determination of efflux pump activity in Example 1. Survival curves were generated according to the procedure described in Example 1 for time-kill assays. The dashed line indicates the detection limit of the method. The results are expressed as the average of at least three independent replicates±the standard error of the mean.

FIG. 9: Effect of pre-treatment with drugs targeting different individual components of the electron transport chain (see table inset) on the killing of C. albicans by light-activated MM 1 (2×MIC). Survival curves were generated according to the procedure described in Example 1 for time-kill assays. The dashed line indicates the detection limit of the method. The results are expressed as the average of at least three independent replicates±the standard error of the mean.

FIG. 10: Effect of pre-treatment with the uncoupling agents carbonyl cyanide 4-(trifluoromethoxy)phenylhydrazone (FCCP) and carbonyl cyanide 3-chlorophenylhydrazone (CCCP) on the killing of C. albicans by light-activated MM 1 (2×MIC). Survival curves were generated according to the procedure described in Example 1 for time-kill assays. The dashed line indicates the detection limit of the method. The results are expressed as the average of at least three independent replicates #the standard error of the mean.

FIG. 11 shows the effect of growth with a fermentable carbon source (glucose) or a non-fermentable carbon source (glycerol) on the killing of C. albicans by light-activated MM 1 (2×MIC). Survival curves were generated according to the procedure described in Example 1 for time-kill assays. The dashed line indicates the detection limit of the method. The results are expressed as the average of at least three independent replicates±the standard error of the mean.

FIGS. 12A-12C show the effect of growth in the presence of different reactive oxygen species (ROS) scavengers and the iron scavenger 2,2′-dipyridyl (DP) on MM 1-induced killing of C. albicans. (FIG. 12A) Inactivation profiles of C. albicans grown in the presence of different scavengers by visible-light-activated MM 1 (2×MIC). Survival curves were generated according to the procedure described in Example 1 for time-kill assays. The dashed line indicates the detection limit of the method. The results are expressed as the average of at least three independent replicates±the standard error of the mean. (FIG. 12B) Growth curves of C. albicans in the presence and absence of the iron scavenger DP, determined by monitoring the absorbance at 630 nm over time in a microplate reader. The results are shown as the average (line) and standard error of the mean (shaded area). (FIG. 12C) Time profiles of mitochondrial ROS levels in C. albicans grown with and without DP after treatment with visible-light-activated MM 1 (2×MIC), detected with the MitoROS™ 580 fluorescent probe, according to the experimental procedure described in Example 1. The results are given as the average (line) and standard error of the mean (shaded area). AA: ascorbic acid. DP: 2,2′-dipyridyl. NAC: N-acetyl-cysteine. TU: thiourea.

FIGS. 13A-13B show the effect of the mitochondrial superoxide scavenger MitoTEMPO (Farmakiotis and Kontoyiannis, 2017) on MM 1-induced killing of C. albicans. (FIG. 13A) Mitochondrial ROS levels detected with the MitoROS™ 580 fluorescent probe according to the experimental procedure described in Example 1 in untreated C. albicans cells or cells pre-treated with MitoTEMPO (1.5 μM, MedChem Express, Princeton, NJ, USA), which were then challenged with increasing concentrations of visible-light-activated MM 1. Asterisks denote the significance of differences in pairwise comparisons performed in GraphPad Prism (San Diego, CA, USA). * p<0.05, ** p<0.01, *** p<0.001, **** p<0.0001. (FIG. 13B) Inactivation profiles of C. albicans treated with increasing concentrations of MitoTEMPO by visible-light-activated MM 1 (2×MIC). Survival curves were generated according to the procedure described in Example 1 for time-kill assays. The dashed line indicates the detection limit of the method. The results are expressed as the average of at least three independent replicates±the standard error of the mean.

FIGS. 14A-14H provide evidence that visible-light-activated MMs elicit intracellular calcium overload. (FIG. 14A) Representative histograms of Callbryte™ 520 AM fluorescence used to detect cytosolic calcium levels in C. albicans treated with increasing concentrations of MM 1 or 1% DMSO in the presence of 405-nm light (87.6 J cm⁻²) by flow cytometry. (FIG. 14B)Cytosolic calcium levels detected with Callbryte™ 520 AM by spectrofluorimetry in C. albicans treated with increasing concentrations of different MMs (0.5-2×MIC) or 1% DMSO in the presence of 405-nm light (87.6 J cm⁻²). (FIG. 14C) Mitochondrial calcium levels detected with Rhod-2 AM by spectrofluorimetry in C. albicans treated with increasing concentrations of different MMs (0.5-2×MIC) or 1% DMSO in the presence of 405-nm light (87.6 J cm⁻²). (FIG. 14D) Mitochondrial calcium levels detected with Rhod-2 AM by confocal microscopy in C. albicans treated with MM 1 (1×MIC) before and after light activation. (FIG. 14E) Temporal profile of Rhod-2 AM fluorescence detected by confocal microscopy, shown as the average fluorescence intensity (line) and standard error of the mean (shaded area). (FIG. 14F) Effect of different concentrations (0.25-1.25 mM) of the intracellular calcium chelator BAPTA-AM on the killing of C. albicans by MM 1 (2× MIC). Killing was assessed as the reduction in colony forming units (CFU), expressed as the logarithm of base 10 of the ratio between the CFU at each time point (N) and the CFU at time zero (NO). The results are expressed as the average of at least three replicates #the standard error of the mean. The dashed line denotes the limit of detection of the method. (FIG. 14G) Cytosolic calcium levels detected by spectrofluorimetry with Callbryte™ 520 AM in C. albicans amended with 1.25 mM BAPTA-AM and then treated with increasing concentrations of MM 1 or 1% DMSO in the presence of 405-nm light (87.6 J cm⁻²). (FIG. 14H)Cytosolic calcium levels detected with Rhod-2 AM by spectrofluorimetry in C. albicans amended with 1.25 mM BAPTA-AM and then treated with increasing concentrations of MM 1 or 1% DMSO in the presence of 405-nm light (87.6 J cm⁻²). The results are the average of at least three independent replicates±the standard deviation. Asterisks denote the significance of the differences in pairwise comparisons with 1% DMSO controls performed in GraphPad Prism. * p<0.05, ** p<0.01, *** p<0.001, **** p<0.0001. unless otherwise stated, the results for MMs and DMSO are always reported in the presence of light.

FIGS. 15A-15E provide evidence that visible-light-activated MMs cause mitochondrial swelling, release of mitochondrial cytochrome c, and necrosis. (FIG. 15A) Representative histograms of MitoTracker™ Green fluorescence in C. albicans treated with 1% DMSO or MM 1 (0.5-2×MIC) and 405-nm light (87.6 J cm⁻²) detected by flow cytometry. (FIG. 15B) Altered mitochondrial mass/volume determined from changes in MitoTracker™ Green fluorescence detected by flow cytometry in C. albicans treated with 1% DMSO or different MMs (0.5-2×MIC) and 405-nm light (87.6 J cm⁻²). (FIG. 15C) Mitochondrial cytochrome c levels in C. albicans treated with 1% DMSO or different MMs (2×MIC) and 405-nm light (87.6 J cm⁻²). (FIG. 15D) Representative changes in the percentage of PI-positive/negative and Annexin V-positive/negative cells in C. albicans treated with 1% DMSO or MM 1 (0.5-2×MIC) and 405-nm light (87.6 J cm⁻²) detected by flow cytometry. (FIG. 15E) Percentage of PI-positive and Annexin V-positive cells in C. albicans treated with different MMs (0.5-2×MIC) or 1% DMSO and 405-nm light (87.6 J cm⁻²) detected by flow cytometry. The results are the average of at least three independent replicates±the standard deviation. Unless otherwise indicated, the results for MMs and DMSO are always reported in the presence of light. Asterisks denote the significance of the differences in pairwise comparisons with 1% DMSO controls performed in GraphPad prism. * p<0.05, ** p<0.01, *** p<0.001, **** p<0.0001.

FIGS. 16A-16I provide evidence that visible-light-activated MMs synergize with conventional antifungals in vitro, in vivo, and ex vivo. (FIG. 16A) Representative checkerboard patterns showing the interaction between visible-light-activated MM 1 and various conventional antifungal drugs in C. albicans and the respective fractional inhibitory concentration indices (FICI) for the interaction between MM 1 and each antifungal. The results are shown as a heatmap, with the white color denoting no growth (0%) and the blue color denoting growth (100%). Results are the average of three independent replicates. Growth was assessed as the absorbance at 630 nm. 5-FC: 5-Fluorocytosine. AMB: Amphotericin B. FLC: Fluconazole. VRC: Voriconazole. CAS: Caspofungin. CPX: Ciclopirox. (FIG. 16B) Decrease in intracellular rhodamine 6G fluorescence, used to assess energy-dependent efflux pump activity, in C. albicans treated with increasing concentrations of different MMs (0.5-2×MIC) or 1% DMSO in the presence of 405-nm light (87.6 J cm⁻²). The lines represent the average of at least three independent replicates, and the shaded area represents the standard error. Unless otherwise noted, the results for MMs and DMSO are always reported in the presence of light. (FIG. 16C) Effect of increasing concentrations of different MMs plus 405-nm light (87.6 J cm⁻²) on the viability of a mammalian cell line (HEK293T). The dashed line indicates the IC₅₀, i.e., the concentration of MM that results in a 50% reduction in cell viability. Results are the average of three independent replicates. (FIG. 16D) Therapeutic index (TI) calculated as the ratio between the MIC for each MM in C. albicans and A. fumigatus and their respective IC₅₀ values. (FIG. 16E) Workflow used to study the anti-infective activity of MMs in vivo. Created in Biorender.com. (FIG. 16F) Survival curves of worms infected with C. albicans or A. fumigatus subjected to monotherapy with visible-light-activated MM 1 (1×MIC plus 405-nm light at 87.6 J cm⁻²), conventional antifungal agents (1×MIC) or combination therapy with visible-light-activated MM 1 (1×MIC plus 405-nm light at 87.6 J cm⁻²) followed by treatment with conventional antifungals (1×MIC). Data represent pooled results from three independent biological replicates, each containing eight individuals (n=24). (FIG. 16G) Fungal load of worms (n=4) infected with C. albicans or A. fumigatus subjected to monotherapy with visible-light-activated MM 1 (1×MIC plus 405-nm light at 87.6 J cm⁻²), conventional antifungal agents (1×MIC), or combination therapy with visible-light-activated MM 1 (1×MIC plus 405-nm light at 87.6 J cm⁻²) followed by treatment with conventional antifungal agents (1×MIC) 48 h after infection. (FIG. 16H) Workflow used to study the anti-infective activity of MMs ex vivo. Created in Biorender.com. (FIG. 161) Fungal load of porcine nail samples (n=9) infected with T. rubrum and subjected to five consecutive rounds of monotherapy with visible-light-activated MM 1 plus 405-nm light at 87.6 J cm-2, different topical formulations of the conventional antifungal ciclopirox (“Lotion” and “Lacquer”) or combination therapy with visible-light-activated MM 1 plus 405-nm light at 87.6 J cm⁻² followed by treatment with a conventional antifungal agent. Asterisks denote the significance of the differences in pairwise comparisons performed in GraphPad prism. * p<0.05, ** p<0.01, *** p<0.001, **** p<0.0001. unless otherwise stated, the results for MMs and DMSO are always reported in the presence of light.

FIG. 17 shows the effect of increasing doses of 405 nm light on the viability of mammalian HEK293T cells. Viability was assessed from ATP levels detected using the CellTiter-Glo® Luminescent Cell Viability Assay. Results are expressed as the average of three biological replicas±standard error of the mean. Asterisks denote the significance of differences in pairwise comparisons between the viability in unirradiated cells and cells irradiated with different doses of 405 nm light. * p<0.05, ** p<0.01, *** p<0.001, **** p <0.0001. Statistical analysis were performed in GraphPad Prism (San Diego, CA, USA).

FIGS. 18A-18B show the effects of visible light activated MMs on the biofilms of Saccharomyces cerevisiae. (FIG. 18A) Reduction of S. cerevisiae biofilm viability by amphotericin B (AMB), 1% DMSO or different MMs (2×, 4×MIC) in the presence of 405-om light (5 min at 292 mW cm⁻²). (b) Reduction of S. cerevisiae biofilm biomass by amphotericin B (AMB), 1% DMSO or different MMs (2×, 4×MIC) in the presence of 405-nm light (5 min at 292 mW cm⁻²). The results are the average of at least three independent replicates±the standard deviation. Asterisks denote the significance of the differences in pairwise comparisons with 1% DMSO controls performed in GraphPad Prism. * p<0.05, ** p<0.01, *** p<0.001, **** p<0.0001.

FIGS. 19A-19F provide evidence that the antifungal activity of MMs against C. albicans requires light activation of the fast rotation rates of the motors. (FIG. 19A) Time-kill curves of C. albicans treated with 1% DMSO, a slow MM (10 AM) or MM 1 (2×MIC) in the presence of 405 nm light (87.6 J cm⁻²), or MM 1 (2×MIC) in the absence of light activation. (FIG. 19B) Mitochondrial dehydrogenase activity in C. albicans treated with 1% DMSO, a slow MM (10 μM) or MM 1 (2×MIC) in the presence of 405 nm light (87.6 J cm⁻²) or MM 1 (2×MIC) in the absence of light activation. (FIG. 19C) Intracellular ATP levels in C. albicans treated with 1% DMSO, a slow MM (10 μM) or MM 1 (2×MIC) in the presence of 405 nm light (87.6 J cm⁻²) or MM 1 (2×MIC) in the absence of light activation. (FIG. 19D) Temporal profiles of PI fluorescence in C. albicans treated with 1% DMSO, a slow MM (10 AM) or MM 1 (2×MIC) in the presence of 405 nm light (87.6 J cm⁻²) or MM 1 (2×MIC) in the absence of light activation. The lines are the average of at least three biological replicates, and the shaded area is the error. (FIG. 19E) Temporal profiles of the MitoROS™ 580 probe used to detect mitochondrial ROS in C. albicans treated with 1% DMSO, a slow MM (10 AM) or MM 1 (2×MIC) in the presence of 405 nm light (87.6 J cm⁻²) or MM 1 (2×MIC) in the absence of light activation. (FIG. 19F) Temporal profiles of Rhod 2-AM used to quantify mitochondrial calcium levels by spectrofluorimetry in C. albicans treated with 1% DMSO, a slow MM (10 μM) or MM 1 (2×MIC) in the presence of 405 nm light (87.6 J cm⁻²) or MM 1 (2×MIC) in the absence of light activation. Asterisks denote the significance of differences in pairwise comparisons performed in GraphPad Prism (San Diego, CA, USA). * p<0.05, ** p<0.01, *** p<0.001, **** p<0.0001. Further experimental details are provided in the text.

FIG. 20 shows a schematic representation of the mechanisms of action of antifungal MMs. MMs bind cardiolipin and phosphatidylglycerol in the inner mitochondrial membrane, destabilizing the electron transport chain. This leads to increased electron leakage and superoxide radical formation, causing oxidative stress. Consequently, ATP synthesis and mitochondrial membrane potential are reduced. ATP-dependent calcium transporters in the plasma membrane and intracellular organelles stop functioning, leading to increased cytosolic calcium levels, which activate calcium-dependent degradative enzymes. Increased water influx ensues, leading to swelling of organelles, which eventually burst, releasing even more degradative enzymes and intramitochondrial contents to the cytoplasm. Eventually, the integrity of the plasma membrane is compromised, and intracellular contents leak out of the cell. Created in Biorender.com.

FIGS. 21A-21F provide evidence for the mechanism of action of visible-light-activated MMs in Saccharomyces cerevisiae. (FIG. 21A) Representative temporal profile of PI fluorescence after treatment of S. cerevisiae with increasing concentrations of MM 1 or 1% DMSO and irradiation with 405 nm light (87.6 J cm⁻²). The lines are the average of at least three biological replicates, and the shaded area is the error. (FIG. 21B) Extracellular ATP levels in S. cerevisiae treated with increasing concentrations of MM 1 (0.5-2×MIC) or 1% DMSO and irradiated with 405 nm light (87.6 J cm⁻²). The results are expressed as the average of at least three independent replicates #the standard error of the mean. (FIG. 21C) Intracellular ATP levels in S. cerevisiae treated with increasing concentrations of MM 1 (0.5-2×MIC) or 1% DMSO in the presence of 405 nm light (87.6 J cm⁻²). Asterisks denote the significance of differences in pairwise comparisons performed in GraphPad Prism (San Diego, CA, USA). * p<0.05, ** p<0.01, *** p<0.001, **** p<0.0001. (FIG. 21D) Temporal profiles of MitoROS™ 580 fluorescence measured by spectrofluorimetry in S. cerevisiae treated with increasing concentrations of MM 1 (0.5-2×MIC) or 1% DMSO in the presence of 405 nm light (87.6 J cm⁻²). Temporal profiles of Rhod-2 AM (FIG. 21E) and Calbryte™ 520 AM (FIG. 21F) fluorescence obtained by spectrofluorimetry to quantify mitochondrial and cytosolic calcium levels, respectively, in S. cerevisiae treated with increasing concentrations of MM 1 (0.5-2×MIC) or 1% DMSO in the presence of 405 nm light (87.6 J cm⁻²). Lines are the average of at least three biological replicates, and the shaded area is the error.

FIG. 22 shows the effect of pre-treatment of C. albicans with the calcineurin inhibitor cyclosporin A (80 μM) on susceptibility to killing by visible-light-activated MM 1 (2×MIC). Survival curves were generated according to the procedure described in the main text for time-kill assays. The dashed line indicates the detection limit of the method. The results are expressed as the average of at least three independent replicates±the standard error of the mean.

FIG. 23 shows representative checkerboard patterns showing the interaction between visible-light-activated MM 1 and various conventional antifungal drugs in S. cerevisiae and the respective fractional inhibitory concentration indices (FICI) for the interactions. The results are shown as a heatmap, with the white color denoting no growth (0%) and the blue color denoting growth (100%). Results are the average of three independent replicates. Growth was assessed as the absorbance at 630 nm. AMB: Amphotericin B. VRC: Voriconazole. CAS: Caspofungin.

FIG. 24 shows the UV-Vis spectra of antibacterial MM. Spectra of MM solutions in DMSO (final concentration of 27 μM) were acquired in a 1-cm quartz cuvette using a Shimadzu UV-2450 spectrophotometer.

FIG. 25A-25G illustrates the use of MMs as antibacterials, as provided in the present disclosure. (FIG. 25A) General structure of an MM. (FIG. 25B) Rotation cycle of an MM. Photoisomerization of the MMs (1->2) generates the metastable conformer, 2. Following the thermal helix inversion step (2->3), during which the methyl group moves from the pseudo-equatorial to pseudo-axial position and the naphthalene moiety in the rotor moves behind the stator, a second stable conformer, 3, is generated. A subsequent photoisomerization step (3->4) and corresponding thermal helix inversion (4->1) generate the full 360° rotation cycle. (FIG. 25C) Schematic representation of an MM drilling through the cell membrane as would occur following light activation. (FIG. 25D) Overview of the workflow used in this study and the different MMs examined at each step. MOA, mechanism of action. (FIG. 25E) MIC value of different MMs in E. coli BW25113. Arrows next to the bars denote that the MIC value was higher than the maximal concentration (40 μM) tested. Bars represent the results from at least three biological replicas. (FIG. 25F)Chemical structure of the antibacterial MMs identified in this study. Functional groups highlighted in red, and blue were introduced to tune the activation wavelength of the motor and increase water solubility, respectively. MW, molecular weight. (FIG. 25G) Schematic representation of the different positioning of MM 1 and MM 2 in the bacterial membrane based on results from molecular dynamics simulations.

FIG. 26 is a schematic depiction of the protocol used to determine the MIC of MMs. The appropriate volume of an MM stock at 8 mM necessary to achieve a concentration ranging from 0.31 to 40 UM was pipetted into a 2 mL microcentrifuge tube to which 1 mL of cell suspension (OD600 ≈0.05) was added. Following a 30 min incubation in the dark, cells were transferred to one well of a 24-well plate positioned in the center of the light beam (405 nm LED Light, Prizmatix, UHP—F-5-405) placed at a distance of 15 cm, corresponding to an intensity of 146 mW cm⁻², and irradiated one at a time for 5 min, for a total light dose of 43.8 J cm⁻². after which irradiated aliquots were collected and inoculated into 1 mL MHB in a 2 mL microcentrifuge tube. Samples were incubated overnight at 37 ° C without agitation. The following day, cultures were inspected for growth, and the MIC was identified as the lowest MM concentration resulting in no visible growth.

FIG. 27 demonstrates that slow rotating MMs do not display antibacterial activity against E. coli. E. coli cells were treated with 8 AM of different slow MM (chemical structure depicted in Table 7) and irradiated with 146 mW cm⁻² of 405 nm light. Cells were then collected, and spot plated as described for fast MMs in the Methods of Example 2. Results are expressed as the logarithm of the ratio between the cell number (CFU per mL) at every time point and the cell number at time zero. The dashed line denotes the limit of detection of the method. Results are the mean of at least 3 independent biological replicas.

FIG. 28 shows the free energy barriers for the rate-limiting thermal helix inversion step of the rotation cycle of the motor. This step brings the metastable state to the ground state, used as proxies of the rotation rate of the MM (Klok et al., 2008). Depicted on top is the chemical structure of a representative MM. The core represents the basic MM without any additional functional group, used as the starting point for DFT calculations and as a reference to assess the impact of different functional groups on rotation rate. The impact of the different functional groups on the rotation rate of the MM was assessed by calculating the ideal unimolecular reaction rate using the Eyring equation (Eyring, 1935). The directionality of the rotation of MMs was assessed from the irreversibility rate, defined as the forward rate over the barrier, divided by the reverse rate over the barrier. For each MM, the electronic energy was calculated using two different approximations to the electronic energy of the system, depicted as “M06-2X” and “TPSSTPSS”, which were then averaged (“TMiX71”) (10, 75, 76). Further details are provided below in Example 2.

FIGS. 29A-29B are molecular dynamics simulations that provide insights into the antibacterial activity of different molecular machines. (FIG. 29A) Histograms of the distribution of angles between the MM axle and XY plane of the membrane for MM 1 and MM 2. An angle of 0° corresponds to the axle being parallel to the membrane plane, while an angle of 90° corresponds to it being perpendicular to the membrane plane (both directions along the Z-axis are treated identically). (FIG. 29B) Histograms of the distributions of distances between geometric centers of axles of MM and membrane center. Z-axis only. Details are provided in Example 2.

FIG. 30 shows potential of mean force (PMF) curves obtained from umbrella sampling simulations. The curves show how the free energy of the system changes as MMs are being pulled out of the membrane. Z is the distance between the center of mass of the MM and the center of the bilayer membrane along the Z-axis (axis perpendicular to the membrane). KT is a unit of energy, where k is a Boltzmann constant and T is absolute temperature. Shaded regions show standard deviations obtained using bootstrapping. Details are provided in Example 2.

FIG. 31 provides the concentration and light-dose dependent time-kill curves of MMs in different bacterial strains. Time-dependent reduction in colony-forming units (expressed as the logarithm of the ratio between the cell number at every time point and the cell number at time zero) of different bacterial strains treated with varying concentrations of different MMs at different light intensities or in the absence of light. Curves may not be clearly distinguishable because they are superimposed. The dashed line denotes the limit of detection of the method. All results are shown as the mean of at least 3 biological replicas±standard error of the mean.

FIG. 32 shows the effect of light dose on the antibacterial action of increasing concentrations of MMs against different bacterial strains. Light dose-dependent reduction in colony-forming units (expressed as the logarithm of the ratio between the cell number at every time point and the cell number at time zero) of different bacterial strains treated with different concentrations of different MMs. The dashed line denotes the limit of detection of the method. All results are shown as the mean of at least 3 biological replicas±standard error of the mean.

FIGS. 33A-33C demonstrate MMs are fast-acting, broad-spectrum antibacterials. (FIG. 33A) Time-dependent reduction in the abundance of different exponentially growing bacterial strains in the presence of 1% DMSO or 2×MIC of each MM and 146 mW cm⁻² of 405 nm light, or 2× and 4×the MIC of conventional antibiotics. The dotted line denotes the limit of detection of the method. Results are the means of at least 3 biological replicas #standard error of the mean. (FIG. 33B) MIC value of MM 1, MM 5, MM 6 in different Gram-negative and Gram-positive strains, including MRSA. Bars represent the results from at least 3 biological replicas. Unless otherwise noted, results for MM and DMSO are always reported in the presence of light. (FIG. 33C) Box and whiskers plot (median values with min/max range) of the MIC values of MM 1, MM 5, and MM 6 among the Gram-negative and Gram-positive strains examined in this study. * P<0.05; ns, not significant.

FIG. 34 provides evidence for the susceptibility (assessed as the MIC) of different E. coli single-gene efflux knockouts to different MM. Gene efflux knockouts are listed in Table 11. The MIC value was determined as described in Example 2. Results are the mean of at least three biological replicas.

FIGS. 35A-35F provides evidence that MMs eliminate persisters and biofilms without detectable resistance. (FIG. 35A) Time-dependent reduction in the abundance of persister cells of different bacterial strains in the presence of 1% DMSO or 1×MIC of each MM and 405-nm light at 146 mW cm⁻² or 2× and 4×the MIC of conventional antibiotics. The dotted line denotes the limit of detection of the method. Reduction in (FIG. 35B) total bacterial cell number assessed using acridine orange, (FIG. 35C) metabolically active cells assessed from ATP levels, (FIG. 35D) total protein assessed using fluorescein isothiocyanate (FITC) fluorescence, and (FIG. 35E) total biomass assessed using crystal violet in established biofilms of P. aeruginosa and S. aureus, following irradiation (146 mW cm⁻² of 405-nm light) for different time periods in the presence of 1% DMSO or 2×MIC of MMs or in the presence of 2×MIC of conventional antibiotics. Results are shown as the mean of at least three biological replicas #standard error of the mean. (FIG. 35F) MIC fold change relative to the original MIC following repeated exposure to MMs and control antibiotics. Results are shown as the average of at least three biological replicas. Unless otherwise noted, results for MMs and DMSO are always reported in the presence of light. * P<0.05; ** P<0.01.

FIG. 36 shows the antibiofilm activity of different concentrations of MMs. Reduction in biofilm biomass of P. aeruginosa and S. aureus following 15 min of irradiation (146 mW cm⁻², 405 nm light) in the presence of 2×MIC or 4×MIC of MM 1, MM 5, and MM 6. Biofilm biomass was determined using the crystal violet method. Unless otherwise noted, results for MM and DMSO are always reported in the presence of light. Each bar represents the mean±standard error of the mean of at least 3 biological replicas. Pairwise comparisons were performed in GraphPad Prism 8 using a Kruskal-Wallis test. n.s.=not significant.

FIGS. 37A-37D provide evidence that MM- and DMSO-treated cells display distinct transcriptomic profiles. (FIG. 37A) RNA-seq workflow created with Biorender.com. (FIG. 37B) Venn diagram of the transcriptomic profiles of MM- and DMSO-treated samples. (FIG. 37C) Heatmap representation of z scores for gene transcripts displaying an adjusted P<0.01 and the highest fold change in abundance in MM- and DMSO-treated samples. (FIG. 37D) Volcano plot of statistically significant (P<0.05) differentially expressed genes identified from the RNA-seq libraries. Results are the average of three biological replicas.

FIG. 38 shows the susceptibility of different single-gene knockouts of E. coli to MM 1. Single deletions of the different genes whose transcripts showed the highest changes in expression in MM-treated samples listed in Table 16 were investigated for their susceptibility (assessed as the MIC) to MM 1. The MIC value was determined as described below in Example 2. Results are the mean of at least three biological replicas; wild-type=WT.

FIGS. 39A-39F provides data regarding the mechanisms of action of visible light-activated MMs. (FIG. 39A) Uptake of NPN by the E. coli outer membrane following treatment with 1% DMSO or different concentrations of MMs. AU, arbitrary units. (FIG. 39B) Time progression of PI fluorescence following treatment of E. coli with different concentrations of MMs or 1% DMSO. (FIG. 39C) Extracellular ATP levels following treatment of E. coli with 1% DMSO or different concentrations of MMs. (FIG. 39D) Fluorescence of the membrane potential probe 3,3-dipropylthiadicarbocyanine iodide [DiSC3 (5)] following treatment of E. coli with 1% DMSO or different concentrations of MMs. All results are shown as the means of at least three biological replicas #standard error of the mean. (FIG. 39E) Transmission electron microscopy (TEM) images of E. coli treated with 1% DMSO or 0.5×MIC of MMs. (FIG. 39F) Scanning electron microscopy (SEM) images of E. coli treated with 1% DMSO or 0.5×MIC of MM 1. Unless otherwise noted, results for MMs and DMSO are always reported in the presence of light. * P<0.05; ** P<0.01; *** P<0.001; **** P<0.0001.

FIG. 40 demonstrates that visible light-activated MMs damage the cell membrane of S. aureus. Time progression of propidium iodide fluorescence following treatment of S. aureus with different concentrations of MMs or 1% DMSO in the presence and absence of light. Further details on the methodology are provided in Example 2. All results are shown as the mean of at least 3 biological replicas±standard error of the mean.

FIG. 41 provides evidence that visible light-activated MM cause depolarization of the membrane of S. aureus. Fluorescence of the membrane potential probe DiSC3 (5) following treatment of S. aureus with 1% DMSO or different concentrations of visible light-activated MMs in the presence and absence of light. Further details on the methodology are provided in Example 2. All results are shown as the mean of at least 3 biological replicas #standard error of the mean. Asterisks denote the significance of the difference between MM and DMSO-treated samples using a Kruskal-Wallis test in GraphPad Prism. * p<0.05, ** p<0.01, *** p <0.001, **** p<0.0001.

FIGS. 42A-42G provide evidence that MMs sensitize bacteria to conventional antibiotics. (FIG. 42A) MIC values of different antibiotics in E. coli with or without pretreatment with light-activated MMs. (FIG. 42B) FIC index for the interaction between MMs and different antibiotics in E. coli. (FIG. 42C) Workflow used to investigate the ability of MMs to potentiate antibiotic activity created with Biorender.com. (FIG. 42D) Reduction in cell numbers following treatment of E. coli with 1% DMSO, 0.5×MIC of different MMs, 4×MIC of different antibiotics alone or in combination, or upon challenging 0.5×MIC MM-treated cells with 4×MIC of antibiotics. (FIG. 42E) Time-dependent increase in tetracycline fluorescence in E. coli following pretreatment of cells with 1% DMSO or MMs. (FIG. 42F) Representative checkerboards depicting the interaction between visible light-activated MMs and vancomycin in P. aeruginosa. A slow MM (ARV-3-202) was used as a control. Results are shown as a heatmap with the white color denoting no growth (0%) and the blue color denoting growth. Growth was assessed as optical density at 600 nm (OD600). (FIG. 42G) Time-kill curves of P. aeruginosa treated with 0.25×MIC of the different visible light-activated MMs and subsequently challenged with vancomycin. Vancomycin-only and MM-only treated samples, as well as DMSO controls, were also examined. AMP, ampicillin; CIPRO, ciprofloxacin; GENTA, gentamicin; NOVO, novobiocin. All experiments were conducted at least in triplicate. Where appropriate, results are shown as the mean of at least three biological replicas±standard error of the mean. Unless otherwise noted, results for MMs and DMSO are always reported in the presence of light.

FIG. 43 shows the light dose-dependent reduction in viability of HEK cells treated with different concentrations of different MMs. All results are shown as the mean of at least 3 biological replicas±standard error of the mean.

FIGS. 44A-44B provide evidence that MMs mitigate mortality in vivo. (FIG. 44A) Workflow used to assess the in vivo antibacterial effects of MMs created with Biorender.com. (FIG. 44B) Percent survival of G. mellonella infected with A. baumannii or S. aureus and treated with 1×MIC of different MMs, 1% DMSO in the presence or absence of 405-nm light, or the antibiotics polymyxin or tobramycin. Data represent the pooled results from three independent biological replicas, each containing 16 individuals. Unless otherwise noted, results for MMs and DMSO are always reported in the presence of light.

FIGS. 45A-45J show that ROS and oxidative stress do not play a significant role in the antibacterial mode of action of MMs. ROS levels in E. coli treated with 1% DMSO or 1×MIC of different MMs in the presence and absence of light, as detected using the fluorescent probes DCFH-DA (FIG. 45A) and APF (FIG. 45B) as previously described (Santos et al., 2013; Brudzynski and Lannigan, 2012) in a microplate format and by flow cytometry (FIG. 45C, FIG. 45D). Percentage of DCFH-DA-(FIG. 45E) and APF-(FIG. 45F) positive cells, as detected using flow cytometry. UV-VIS spectra of the singlet oxygen trap DPBF in the presence of the fast antibacterial motor MM 1 (FIG. 45G) or the inert slow motor ARV 3-202 (FIG. 45H). (FIG. 451) Rate of decrease of the absorption of DPBF at 410 nm, indicative of singlet oxygen generation, in the presence of MM 1 or its slow analog ARV 3-202. (FIG. 45J) Protein carbonyl levels normalized by the protein content in E. coli treated with 1% DMSO or 1×MIC of MM 1 in the presence of light, determined as previously described (Belenky et al., 2015). Flow cytometry data (at least 10 000 cells) was acquired in a spectral analyzer (Sony SA3800) and analyzed using FlowJo software. Results are shown as the mean of at least 3 biological replicas±standard error of the mean. Comparisons between samples were performed in GraphPad Prism 8 using a Kruskal-Wallis test for comparison between three or more samples or an unpaired t-test for comparisons between two samples. * p<0.05. not significant=n.s.

FIG. 46 provides evidence that growth in the presence of antioxidants does not protect against MM-induced killing in E. coli. Reduction in bacterial numbers following irradiation of cell suspensions of E. coli treated with 2×MIC of MM 1 in the presence or absence of the ROS scavengers N-acetyl-L-cysteine (NAC, 1 mM) and thiourea (TU, 100 mM) (Rowe-Magnus et al., 2019). All results are shown as the mean of at least 3 biological replicas±standard error of the mean.

FIG. 47 shows the temperature variation profiles during irradiation of fast and slow MMs. Temperature (°C) during irradiation of samples treated with 40 μM of MM 1 or 40 μM of the slow analog ARV 3-202 (Table 7) was assessed using a temperature probe (Model SC-TT-K-30-36-PP; Omega Engineering, Inc.). Results are the average of three independent experimental measurements. Error bars are the standard deviation. Asterisks denote the significance of difference (ANOVA with Dunnett's multiple comparisons test) between the temperature at a certain time point and the temperature at time 0. * p<0.05, ** p<0.01.

FIG. 48 provides evidence that irradiation does not cause detectable photodegradation of MMs. Aromatic region of ¹H NMR (600 MHz) spectra of MM 1 (300 mM in DMSO-d6) before and after irradiation for 5 min with 146 mW cm⁻² of 405 nm light.

FIG. 49 provides evidence that pre-irradiation does not lead to loss of MM antibacterial activity. Influence of MM pre-irradiation on the inactivation of E. coli. Top: (1) To test the influence of pre-irradiating MMs on their antibacterial effects, MM 1 was added to a Petri dish containing PBS (final concentration of 8 μM) and irradiated for 25 min at 146 mW cm⁻² with 405 nm light. Cells were then added to an OD of ≈0.02, incubated in the dark for 30 min and then irradiated for up to 10 min at 146 mW cm⁻² with 405 nm light; (2) Independently, MM 1 was added to a Petri dish containing PBS (final concentration of 8 mM), cells were then added to an OD600 of ≈0.02, incubated in the dark for 30 min and then irradiated for up to 10 min at 146 mW cm⁻² with 405 nm light. Bottom: Time-dependent reduction in colony-forming units (expressed as the logarithm of the ratio between the cell number at every time point and the cell number at time zero) of cells treated with pre-irradiated MM 1 or non-irradiated MM 1.

FIG. 50 shows the chemical structure of the molecular machine (M96) used in the experiments described in Example 3.

FIGS. 51A-51E provide evidence of the in vitro therapeutic efficacy of M96 in mouse melanoma B16-F10 cells. (FIG. 51A) Representative images of clonogenic assay. In a clonogenic assay, each surviving cell should form a colony under standard cell culture conditions. The surviving cells were stained with crystal violet. DMSO=0.1% DMSO in the media, M96=8 μM in the media, and Light=illumination with 405 nm blue light at 300 mW/cm² for 5 min. (FIG. 51B)Calibration of the light using clonogenic assay to measure the therapeutic effect. D=0.1% DMSO, L=irradiation with 405 nm blue LED light and M96=8 μM. The concentration of M96 was maintained constant at 8 AM but the irradiation time and light intensity (100 mW/cm2, 150 mW/cm2, and 300 mW/cm⁻²) were varied. (FIG. 51C) Therapeutic effect of M96 with all parameters fixed: M96=8 AM and light dose=5 min of irradiation with 405 nm light at 300 mW/cm2, and experimental triplicates. (FIG. 51D) Calibration of the concentration of M96. The light dose was maintained constant at 200 mW/cm² for 5 min (405 nm light). The method of analysis is based on the propidium iodide (PI) staining of nuclear DNA in membrane permeabilized death cells. The PI staining (fluorescence) was quantified using the cell counter COUNTESS III FL. (FIG. 51E) Therapeutic efficacy of M96 measured by flow cytometry. DMSO=0.1% DMSO, M96=8 AM of M96, Light=irradiation for 5 min of 405 nm light at 300 mW/cm2. In all the cases, DMSO was used to solubilize and store M96 at 8 mM stock solution and was used at 1:1000 dilution in the cells to achieve 0.1% DMSO and 8 μM M96. 0.1% DMSO was therefore used as a control. * p<0.05, ** p<0.01, *** p<0.001 Statistical significance p<0.05.

FIGS. 52A-52D provide data for the in vitro IC₅₀ of molecular machine M96 at constant light intensity. (FIG. 52A) The IC₅₀ of M96 under 405 nm light illumination at 200 mW/cm² for 5 min (IC₅₀~3 μM) in mouse melanoma B16-F10 cells. (FIG. 52B) The IC₅₀ of M96 under 405 nm light illumination at 150 mW/cm² for 5 min (IC₅₀ ~2 μM). (FIG. 52C) The IC₅₀ of M96 under 405 nm light illumination at 200 mW/cm² for 5 min (IC₅₀~2 μM). (FIG. 52D) The IC₅₀ of M96 under 405 nm light illumination at 200 mW/cm² for 5 min in various human skin conditions.

FIGS. 53A-53B provide the flow cytometry analysis of the therapeutic efficacy of molecular machine M96 in mouse melanoma B16-F10 cells. (FIG. 53A) Analysis of the PI positive (dead) cells by flow cytometry. The PI enters into the cells upon disruption of the integrity of the cellular membrane and stains the cellular DNA. D=0.1% DMSO, M=8 μM of M96, L=irradiation for 5 min of 405 nm light at 300 mW/cm² A time course analysis is conducted to show that the PI staining of B16-F10 cells is immediate upon treatment with 8 AM M96 and illumination with 405 nm light at 300 mW/cm² for 5 min. One subpopulation of PI positive cells (at relatively low PI fluorescence intensity ~3×10³) are permeabilized to PI (death or in the process of dying) but staining continues over time until they reach ~10⁵ PI fluorescence intensity. The control “death cells” consisted of B16-F10 cells that were treated by heating at 95 ° C for 10 min and PI added for staining. (FIG. 53B) Quantification of the PI positive cells in B16-F10 cells upon treatment with 8 μM M96 and illumination with 405 nm light at 300 mW/cm² for 5 min. The quantification is conducted by flow cytometry analysis at 2 h after the treatment.

FIGS. 54A-54B show the in vivo therapeutic efficacy of molecular machine M96 in subcutaneous tumors of B16-F10 in C57BL/6J mice with light at 300 mW/cm2. (FIG. 54A) Tumor growth inhibition by the treatment with 50 μL intratumoral injection of 8 μM M96, 30 min incubation, and irradiation with 300 mW/cm² of 405 nm light for 5 min. (FIG. 54B) Representative pictures of the mice with tumors under the various treatments. DMSO=0.1% DMSO solution in PBS. M96=8 μM solution of M96 in PBS. Light=irradiation with 405 nm light at 300 mW/cm² for 5 min. The DMSO is used to solubilize and prepare a stock solution of 8 mM M96 and stored at −20 ° C The 8 mM stock solution of M96 in DMSO is diluted 1:1000 in PBS and the final solution contains 8 μM M96 and 0.1% DMSO in PBS buffer. 0.1% DMSO is used as control. * p<0.05, ** p<0.01, *** p<0.001 Statistical significance p<0.05, ns=non-significance.

FIGS. 55A-SSC show the in vivo therapeutic efficacy of molecular machine M96 in subcutaneous tumors of B16-F10 in C57BL/6J mice with light at 200 mW/cm2. (FIG. 55A) Tumor growth inhibition by the treatment with 20 μL intratumoral injection of 8 AM M96, 30 min incubation, and irradiation with 200 mW/cm² of 405 nm light for 5 min (one treatment per day for 4 days). (FIG. 55B) Survival curve of mice with tumors upon the treatment. The threshold value for mice euthanasia was a tumor size of 2000 m³. (FIG. 55C) Representative pictures of the mice with tumors under the various treatments. DMSO=0.1% DMSO solution in PBS. M96=8 μM solution of M96 in PBS. Light=irradiation with 405 nm light at 200 mW/cm² for 5 min. The DMSO is used to solubilize and prepare a stock solution of 8 mM M96 and stored at −20 °C. The 8 mM stock solution of M96 in DMSO is diluted 1:1000 in PBS and the final solution contains 8 μM M96 and 0.1% DMSO in PBS buffer. 0.1% DMSO is used as control. * p<0.05, ** p<0.01, *** p<0.001 Statistical significance p<0.05, ns=non-significance. The statistical analysis t-test compared M96 group versus M96+Light group.

FIGS. 56A-56C are a comparison of in vivo therapeutic efficacy experiments in subcutaneous tumors of B16-F10 in C57BL/6J mice. (FIG. 56A) Tumor growth inhibition by the treatment with 20 AL intratumoral injection of 8 μM M96, 30 min incubation, and irradiation with 200 mW/cm² of 405 nm light for 5 min (once a day for 4 days). DMSO=0.1% DMSO solution in PBS. M96=8 UM solution of M96 in PBS. Light=irradiation with 405 nm light at 200 mW/cm² for 5 min. The DMSO is used to solubilize and prepare a stock solution of 8 mM M96 and stored at −20 °C. The 8 mM stock solution of M96 in DMSO is diluted 1:1000 in PBS and the final solution contains 8 μM M96 and 0.1% DMSO in PBS buffer. 0.1% DMSO is used as control. (FIG. 56B) Tumor growth inhibition by the treatment with 20 μL intratumoral injection of 20 μM M96, 30 min incubation, and irradiation with 250 mW/cm2 of 405 nm light for 5 min (once a day for 4 days). DMSO=0.25% DMSO solution in PBS. M96=20 μM solution of M96 in PBS. Light=irradiation with 405 nm light at 250 mW/cm2 for 5 min. (FIG. 56C) Tumor growth inhibition by the treatment with 20 AL intratumoral injection of 400 μM M96, 30 min incubation, and irradiation with 250 mW/cm² of 405 nm light for 5 min (once a day for 4 days). DMSO=5% DMSO solution in PBS. M96=400 μM solution of M96 in PBS. Light=irradiation with 405 nm light at 250 mW/cm² for 5 min. * p<0.05, ** p<0.01, *** p<0.001 Statistical significance p<0.05, ns=non-significance. The statistical analysis t-test was applied to compare M96 groups versus M96+Light groups.

FIGS. 57A-57C demonstrate the effect of combination light-activated-molecular machine M96 therapy and anti-PD1 immunotherapy. (FIG. 57A) Schematics of the dose regime of molecular machine therapy and anti-PD1 immunotherapy combination in B16-F10 subcutaneous tumors. For molecular machine therapy the molecular machine M96 was applied by intratumoral injection of 30 μL M96 at 8 μM in PBS solution, 30 min incubation, then light treatment with 405 nm LED at 300 mW/cm² for 5 min. Each immunotherapy treatment consisted of an intraperitoneal injection of 100 AL antibody (anti-PD1 or isotype IgG) solution in PBS at the concentration of 2 μg/μL (Injection of 200 μg of antibody per mouse). (FIG. 57B) Tumor sizes over the time in the different treatment groups. “M96 only” consisted in the intratumoral injection of 30 μL M96 at 8 AM in PBS solution without light treatment at day 8. Isotype control consisted in the intraperitoneal injection of IgG (200 μg per mouse) in the dose regime shown in FIG. 57A. Anti-PD1 consisted in the intraperitoneal injection of anti-PD1 (200 μg per mouse) in the dose regime shown in A. M96+Light consisted of intratumoral injection of 30 μL M96 at 8 AM in PBS solution, 30 min incubation, then light treatment with 405 nm LED at 300 mW/cm² for 5 min at day 8. M96+Light+anti-PD1 consisted in the combination of M96+Light treatment and anti-PD1 treatment. 20 (FIG. 57C) Survival of mice over time in the different treatment groups.

FIG. 58 provides the structures for which DFT computations were performed. For motors with asymmetric stators, “A” indicates the functionalized side of the stator. “B” denotes the conformation of the MM following a 180°-degree rotation of the motor. In the case of 3 and 4, the molecules are symmetrical thus conformation “A” and “B” are the same.

FIG. 59 is an illustration of the system used in MD simulations created using VMD. The lipid bilayer membrane (POPE and POPG) is shown in blue. Water molecules are shown in red, sodium and chlorine ions are shown as purple and green spheres, respectively. A representative MM molecule (in red) is shown embedded inside the membrane. Dimensions of the system: 5×5×14 nm.

### DETAILED DESCRIPTION

The present disclosure features stimulus-activated molecular machines that cross lipid bilayers and methods for treating bacterial or fungal infections, or for treating cancer, using such molecular machines. Each of these embodiments will be described below in more detail.

### A. Definitions

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Throughout this application, the term “about” is used to indicate that a value includes the inherent variation of error for the device, the method being employed to determine the value, or the variation that exists among the study subjects or patients.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,”

“Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiary butylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Subject,” as used herein, refers to the recipient of the implantable construct described herein. The subject may include a human and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development (e.g., a male or female of any age group, e.g., a pediatric subject (e.g., infant, child, adolescent) or adult subject (e.g., young adult, middle-aged adult, or senior adult). A non-human animal may be a transgenic animal.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease. In some embodiments, “treatment,” “treat,” and “treating” require that signs or symptoms of the disease or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### B. Chemical Definitions

When used in the context of a chemical group: “hydrogen” means-H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

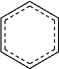

covers, for example,

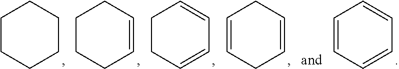

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

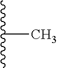

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

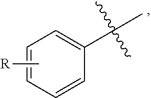

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

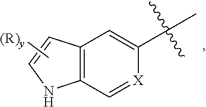

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(c≤8)}”, “alkynyl_{(c≤3)}”, and “heterocycloalkyl (ccs)” is two, the minimum number of carbon atoms in the group “cycloalkyl_{(c≤8)}” is three, and the minimum number of carbon atoms in the groups “aryl_{(c≤8)}” and “arenediyl_{(c≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C₁₋₄-alkyl”, “alkyl_{(c1-4)}”, and “alkyl_{(c≤4)}” are all synonymous. Except as noted below, every carbon atom is counted to determine whether the group or compound falls with the specified number of carbon atoms. For example, the group dihexylamino is an example of a dialkylamino_{(c12)} group; however, it is not an example of a dialkylamino (cc) group. Likewise, phenylethyl is an example of an aralkyl_{(c=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

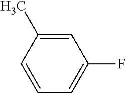

is also taken to refer to

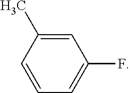

Aromatic compounds may also be depicted using a circle to represent the delocalized nature of the electrons in the fully conjugated cyclic a system, two non-limiting examples of which are shown below:

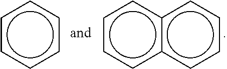

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃(Et), —CH₂CH₂CH₃ (n-Pr or propyl), —CH(CH₃)₂ (i-Pr, ⁱPr or isopropyl), —CH₂CH₂CH₂CH₃ (n-Bu), —CH₂(CH₃)CH₂CH₃ (sec-butyl), —CH₂CH (CH₃)₂ (isobutyl), —C(CH₃)₃ (tert-butyl, (-butyl, t-Bu or ^{t}Bu), and —CH₂C(CH₃)₃ are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂-(methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl.

Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. If more than one ring is present, the rings may be fused, bridged, or spirocyclic. Non-limiting examples include: —CH(CH₂)2 (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂(vinyl), —CH═CHCH3, —CH═CHCH2CH₃, —CH₂CH—CH₂(allyl), —CH₂CH═CHCH3, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups-CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R. is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

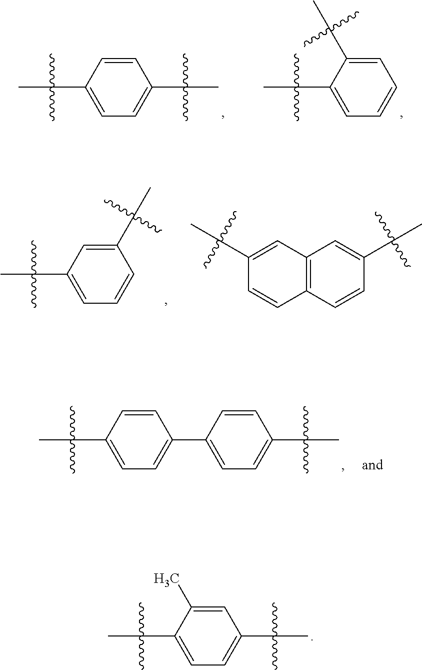

An “arene” refers to the class of compounds having the formula H—R, wherein R is aryl as that term is defined above. Benzene and toluene are non-limiting examples of arenes.

The term “aralkyl” refers to the monovalent group —alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heteroaralkyl” refers to the monovalent group-alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings may be fused, bridged, or spirocyclic. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, tetrahydropyridinyl, pyranyl, oxiranyl, and oxetanyl. The term “N heterocycloalkyl” refers to a heterocycloalkyl group with a nitrogen atom as the point of attachment. N pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O) R has been replaced with a sulfur atom, —C(S)R. The term “aldehyde” corresponds to an alkyl group, as defined above, attached to a —CHO group.

The term “alkoxy” refers to the group-OR, in which R is an alkyl, as that term is defined above. Non-limiting examples include: —OCH₃ (methoxy), —OCH CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group-SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group-NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH: , —NHC(O)CH₃, —S(O)₂OH, or —S(O)₂NH₂. For example, the following groups are non-limiting examples of substituted alkyl groups: —CH₂OH, —CHCl, —CF₃, —CH₂CN, —CH₂C(O) OH, —CH₂C(O) OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂(carbamoyl), and —CON(CH₃)₂, are non-limiting examples of substituted acyl groups. The groups —NHC(O)OCH₃ and —NHC(O)NHCH₃ are non-limiting examples of substituted amido groups.

An “isomer” of a first compound is a separate compound in which each molecule contains the same constituent atoms as the first compound, but where the configuration of those atoms in three dimensions differs.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### C. Compounds of the Present Invention

The compounds of the present invention (also referred to as “compounds of the present disclosure”) are shown, for example, above, in the summary of the invention section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the compounds of the present invention may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present invention have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present invention function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present invention exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### D. Infections

In some aspects of the present disclosure, the stimulus activated molecular machines as described herein may be used to treat a microbial infection (an infection of a microorganism). Some non-limiting examples of mircoorganisms which may be treated with the compounds herein include bacteria, viruses, parasites, and fungi.

### 1. Fungal Infections

Fungi are plentiful, with about 1.5 million different species on earth. Only about 300 of these are known to cause disease. Fungal diseases are called mycoses and those affecting humans can be divided into four groups based on the level of penetration into the body tissues. Superficial mycoses are caused by fungi that grow on the surface of the skin or hair. Cutaneous mycoses or dermatomycoses include such infections as athlete's foot and ringworm, where growth occurs only in the superficial layers of skin, nails, or hair. Subcutaneous mycoses penetrate below the skin to involve the subcutaneous, connective, and bone tissue. Systemic or deep mycoses are able to infect internal organs and become widely disseminated throughout the body. This type is often fatal.

Some of the more common diseases include Aspergillosis, Blastomycosis, Candidiasis, Coccidioidomycosis, C. neoformans infection, C. gattii infection, fungal eye infection, Histoplasmosis, Mucormycosis, Pneumocystis pneumonia, Ringworm and Sportotrichosis. Candidemia infections occur can be predicted at around 300,000 worldwide per year—with a mortality of 30-55%. Invasive aspergillosis can occur in different patients groups-so around 10% of new leukaemic cases will go on to develop invasive aspergillosis—so 30,000 per year. Of stem cell transplants-54,000 are carried out in USA, UK, Europe and Japan annually, of which 5,400 will need treatment for aspergillus infection. In chronic obstructive pulmonary disease −1.2% of these will need antifungals for aspergillosis −216,000 per year. Over 50% of invasive aspergillosis patients will die from their infection—even with treatment. In AIDS patients 1 million contract cryptococcal meningitis resulting in 600,000 deaths −70% of which are in sub-saharan Africa. Less fatal infections but which affect large numbers of people worldwide include cutaneous fungal infections, nail infections and athletes foot—affects some 1.5 billion people—or 25% of the world's population. Tinea capitis—or hair infection—which is common in young children is predicted to affect some 200 million worldwide.

Fungi that may be treated in accordance with the present disclosure include, e.g., Candida spp. including C. albicans, C. tropicalis, C. kerr, C. krusei and C. galbrata; Aspergillus spp. including A. fumigatus and A. flavus; Cryptococcus neofornans; Blastomyces spp. including Blastomyces dermatitidis; Pneumocystis carinii; Coccidioides immitis; Basidiobolus ranarum; Conidiobolus spp.; Histoplasma capsulatum; Rhizopus spp. including R. oryzae and R. microsporus; Cunninghamella spp.; Zygomycetes such as Rhizomucor spp. (R. oryzae, R. microspores); Paracoccidioides brasiliensis; Pseudallescheria boydii; Rhinosporidium seeberi; and Sporothrix schenckii.

### 2. Bacterial Infections

In some aspects, the present disclosure provides stimulus activated molecular machines described herein that may be used to treat a bacterial infection. While humans contain numerous different bacteria on and inside their bodies, an imbalance in bacterial levels or the introduction of pathogenic bacteria can cause a symptomatic bacterial infection. Pathogenic bacteria cause a variety of different diseases including but not limited to numerous foodbome illness, typhoid fever, tuberculosis, pneumonia, syphilis, and leprosy.

Additionally, different bacteria have a wide range of interactions with the body and those interactions can modulate the ability of the bacteria to cause an infection. For example, bacteria can be conditionally pathogenic such that they only cause an infection under specific conditions. For example, Staphylococcus and Streptococcus bacteria exist in the normal human bacterial biome, but these bacteria when they are allowed to colonize other parts of the body causing a skin infection, pneumonia, or sepsis. Other bacteria are known as opportunistic pathogens and only cause diseases in a patient with a weakened immune system or another disease or disorder.

Bacteria can also be intracellular pathogens which can grow and reproduce within the cells of the host organism. Such bacteria can be divided into two major categories as either obligate intracellular parasites or facultative intracellular parasites. Obligate intracellular parasites require the host cell in order to reproduce and include such bacteria as but are not limited to Chlamydophila, Rickettsia, and Ehrlichia which are known to cause pneumonia, urinary tract infections, typhus, and Rocky Mountain spotted fever. Facultative intracellular parasites can reproduce either intracellular or extracellular. Some non-limiting examples of facultative intracellular parasites include Salmonella, Listeria, Legionella, Mycobacterium, and Brucella which are known to cause food poisoning, typhoid fever, sepsis, meningitis, Legionnaire's disease, tuberculosis, leprosy, and brucellosis.

The stimulus activated molecular machines described herein may be used in the treatment of bacterial infections, including those caused by Staphylococcus aureus. S. aureus is a major human pathogen, causing a wide variety of illnesses ranging from mild skin and soft tissue infections and food poisoning to life-threatening illnesses such as deep post-surgical infections, septicaemia, endocarditis, necrotizing pneumonia, and toxic shock syndrome. These organisms have a remarkable ability to accumulate additional antibiotic resistance determinants, resulting in the formation of multiply-drug-resistant strains.

Methicillin, being the first semi-synthetic penicillin to be developed, was introduced in 1959 to overcome the problem of penicillin-resistant S. aureus due to β-lactamase (penicillinase) production (Livermore, 2000). However, methicillin-resistant S. aureus (MRSA) strains were identified soon after the introduction of methicillin (Barber, 1961; Jevons, 1961). The methods described herein may be used in the treatment of MRSA bacterial strains.

Additionally, the stimulus activated molecular machines described herein may be used to treat a Streptococcus pneumoniae infection. Streptococcus pneumoniae is a gram-positive, alpha-hemolytic, bile soluble aerotolerant anaerobe and a member of the genus Streptococcus. A significant human pathogenic bacterium, S. pneumoniae was recognized as a major cause of pneumonia in the late 19th century and is the subject of many humoral immunity studies.

Despite the name, the organism causes many types of pneumococcal infection other than pneumonia, including acute sinusitis, otitis media, meningitis, bacteremia, sepsis, osteomyelitis, septic arthritis, endocarditis, peritonitis, pericarditis, cellulitis, and brain abscess. S. pneumoniae is the most common cause of bacterial meningitis in adults and children, and is one of the top two isolates found in ear infection, otitis media. Pneumococcal pneumonia is more common in the very young and the very old.

S. pneumoniae can be differentiated from S. viridans, some of which are also alpha hemolytic, using an optochin test, as S. pneumoniae is optochin sensitive. S. pneumoniae can also be distinguished based on its sensitivity to lysis by bile. The encapsulated, gram-positive coccoid bacteria have a distinctive morphology on gram stain, the so-called, “lancet shape.” It has a polysaccharide capsule that acts as a virulence factor for the organism; more than 90 different serotypes are known, and these types differ in virulence, prevalence, and extent of drug resistance.

S. pneumoniae is part of the normal upper respiratory tract flora but as with many natural flora, it can become pathogenic under the right conditions (e.g., if the immune system of the host is suppressed). Invasins such as Pneumolysin, an anti-phagocytic capsule, various adhesins and immunogenic cell wall components are all major virulence factors.

Finally, bacterial infections could be targeted to a specific location in or on the body. For example, bacteria could be harmless if only exposed to the specific organs, but when it comes in contact with a specific organ or tissue, the bacteria can begin replicating and cause a bacterial infection.

### Gram-Positive Bacteria

In some aspects of the present disclosure, the stimulus activated molecular machines described herein may be used to treat a bacterial infection by a gram-positive bacteria. Gram-positive bacteria contain a thick peptidoglycan layer within the cell wall which prevents the bacteria from releasing the stain when dyed with crystal violet. Without being bound by theory, the gram-positive bacteria are often more susceptible to antibiotics. Generally, gram-positive bacteria, in addition to the thick peptidoglycan layer, also comprise a lipid monolayer and contain teichoic acids which react with lipids to form lipoteichoic acids that can act as a chelating agent. Additionally, in gram-positive bacteria, the peptidoglycan layer is outer surface of the bacteria. Many gram-positive bacteria have been known to cause disease including, but are not limited to, Streptococcus, Straphylococcus, Corynebacterium, Enterococcus, Listeria, Bacillus, Clostridium, Rathybacter, Leifsonia, and Clavibacter.

### Gram-Negative Bacteria

In some aspects of the present disclosure, the stimulus activated molecular machines described herein may be used to treat a bacterial infection by a gram-negative bacteria. Gram-negative bacteria do not retain the crystal violet stain after washing with alcohol. Gram-negative bacteria, on the other hand, have a thin peptidoglycan layer with an outer membrane of lipopolysaccharides and phospholipids as well as a space between the peptidoglycan and the outer cell membrane called the periplasmic space. Gram-negative bacterial generally do not have teichoic acids or lipoteichoic acids in their outer coating. Generally, gram-negative bacteria also release some endotoxin and contain prions which act as molecular transport units for specific compounds. Most bacteria are gram-negative. Some non-limiting examples of gram-negative bacteria include Bordetella, Borrelia, Burcelia, Campylobacteria, Escherichia, Francisella, Haemophilus, Helicobacter, Legionella, Leptospira, Neisseria, Pseudomonas, Rickettsia, Salmonella, Shigella, Treponema, Vibrio, and Yersinia.

### Gram-Indeterminate Bacteria

In some aspects of the present disclosure, the stimulus activated molecular machines described herein may be used to treat a bacterial infection by a gram-indeterminate bacteria. Gram-indeterminate bacteria do not full stain or partially stain when exposed to crystal violet. Without being bound by theory, a gram-indeterminate bacteria may exhibit some of the properties of the gram-positive and gram-negative bacteria. A non-limiting example of a gram-indeterminate bacteria include Mycobacterium tuberculosis or Mycobacterium leprae.

### E. Cancer and Hyperproliferative Diseases

While hyperproliferative diseases can be associated with any disease which causes a cell to begin to reproduce uncontrollably, the prototypical example is cancer. One of the key elements of cancer is that the cell's normal apoptotic cycle is interrupted and thus agents that interrupt the growth of the cells are important as therapeutic agents for treating these diseases. In some embodiments, the cell membrane that may be disrupted is a human cell, such as a cancer cell. In some embodiments, the compounds of the disclosure may disrupt a human cell, such as an adipose cell. The methods described in the present disclosure contemplate the disruption of either or both a healthy cell or a cancerous cell. In this disclosure, the cell membrane disrupting compounds described herein may be used to lead to decreased cell counts and as such can potentially be used to treat a variety of types of cancer lines. In some embodiments, the cell membrane disrupting compounds described herein are contemplated to open the cell membrane. In further embodiments, the cell membrane disrupting compounds described herein thus allow at least a second therapeutic agent to enter the cell. In some aspects, it is anticipated that the cell membrane disrupting compounds described herein may be used to treat virtually any malignancy.

Cancer cells that may be treated with the compounds of the present disclosure include but are not limited to cells from the skin, bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma, juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### F. Molecular Machines

An important aspect of biomedical therapy is the effective delivery of various molecules such as drugs and genetic information into cells. In order to be effective, such delivery methods must facilitate the passage of the molecules across the lipid bilayer of cell membranes. Thus, several physical techniques have been used to open lipid bilayers of cellular membranes. Such techniques use physical energies such as electric fields, magnetic fields, temperature, ultrasound, and light. These techniques have been used to intentionally introduce foreign materials into cells, release molecular species from cells, or to induce necrosis,

The methods of the present disclosure involve the use of stimuli-responsive molecular machines. The molecular machines used in the methods disclosed herein are examples of compounds that, in response to a stimulus, undergo a sequential conformational change, which generates a drill-like motion that can propel the molecule through lipid bilayers (García-López et al., 2017; Feringa, 2007, Klok, 2008). In some embodiments, the molecular machine rotates unidirectionally. In some embodiments, the molecular machine rotates bidirectionally. In some embodiments, the rotational component of the molecular machine rotates at a speed greater than 1 Hz. In some embodiments, the rotational component of the molecular machine rotates at a speed greater than 10 Hz. In some embodiments, the rotational component of the molecular machine rotates at a speed greater than 10³ Hz. In some embodiments, the rotational component of the molecular machine rotates at a speed of about 10⁴ Hz, 10⁵ Hz, 10⁶ Hz, 10⁷ Hz. 10⁸ Hz, 10⁹ Hz, or 10¹⁰ Hz, or any range derivable therein. In some embodiments, the rotational component of the molecular machine rotates at a speed of about 10⁵ Hz, 10⁶ Hz, 10⁷ Hz, 10⁸ Hz, or any range derivable therein. In some embodiments, the rotational component of the molecular machine rotates at a speed of about 10⁵ Hz. In some embodiments, the rotational component of the molecular machine rotates at a speed of about 10⁶ Hz. In some embodiments, the rotational component of the molecular machine rotates at a speed of about 10⁸ Hz.

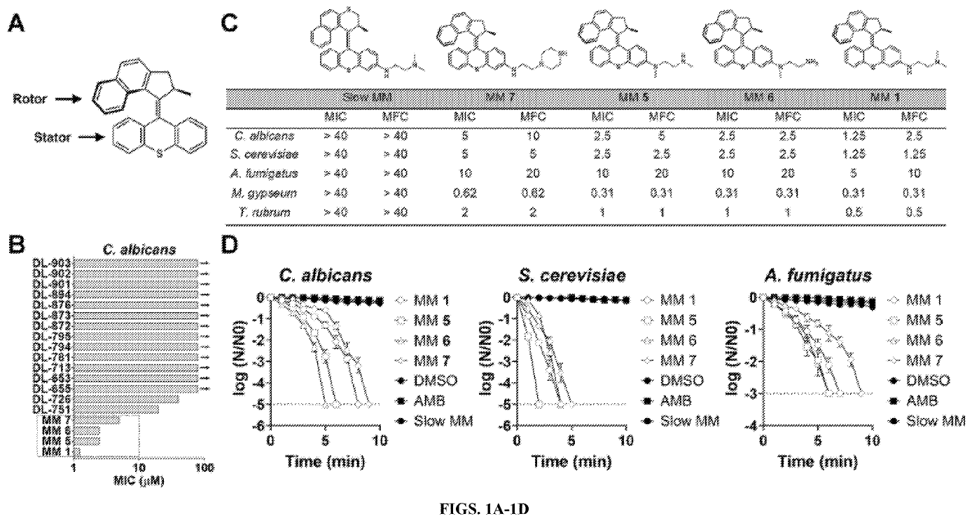

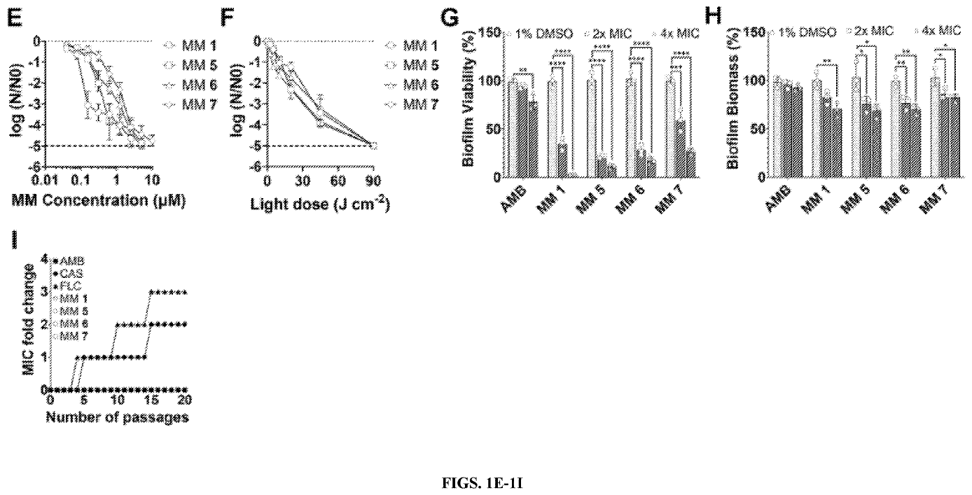

In some embodiments, molecular machines (MMs) of the present disclosure consist of a stator and a stimulus-activated rotor (FIG. 1A). In some embodiments, the molecular machine comprises a rotor that is connected to a stator by an alkenyl group. In other embodiments, the molecular machine comprises a rotor connected to a stator by an alkynyl group. In some embodiments, the molecular machine comprises a rotor connected to a stator by an atropisomeric alkene. In some embodiments, the moving components (that is, the rotor) of the present disclosure can include one or more conjugated systems. In some embodiments, the rotor comprises a plurality of rings. In some embodiments, the rotor comprises 1, 2, 3, 4, 5, 6, 7, 8, 9 or 10 aliphatic or aromatic rings. In some embodiments, the rotor comprises 1, 2, 3, 4, or 5 aliphatic or aromatic rings. In some embodiments, the rotor comprises 1, 2, or 3 aliphatic or aromatic rings. In some embodiments, the rotor comprises at least one ring that is aromatic. In some embodiments, the rotor comprises 1, 2, 3, 4, or 5 aromatic rings. In some embodiments, the rotor comprises 1 aromatic ring. In some embodiments, the rotor comprises 2 aromatic rings. In some embodiments, the rotor comprises 3 aromatic rings. In some embodiments, the rotor comprises at least one aliphatic ring. In some embodiments, the rotor comprises 1, 2, 3, 4, 5, 6, 7, 8, 9, or 10 aliphatic rings. In some embodiments, the rotor comprises 1, 2, 3, 4, or 5 aliphatic rings. In some embodiments, the rotor comprises 1 aliphatic ring. In some embodiments, the rotor comprises 2 aliphatic rings. In some embodiments, the rotor comprises 3 aliphatic rings. In some embodiments, the rotor comprises both aromatic and aliphatic rings. In some embodiments, the rotor comprises any combination of 1, 2, 3, 4, or 5 aromatic rings and 1, 2, 3, 4, or 5 aliphatic rings. In some embodiments, the rotor comprises any combination of 1, 2, or 3 aromatic rings and 1, 2, or 3 aliphatic rings. In some embodiments, the rotor comprises 1, 2, or 3 aromatic rings and 1 or 2 aliphatic rings.

In some embodiments, the stimulus that generates the drill-like motion is electromagnetic radiation. In some embodiments of the present disclosure, the electromagnetic radiation that stimulates the presently disclosed molecular machines or is used in the presently disclosed methods to stimulate molecular machines comprises gamma rays, X-rays, UV light, visible light, near infrared light, infrared light, microwaves, or radio waves, or any combination thereof. In some embodiments, the electromagnetic radiation comprises UV light, visible light, or near infrared light, or a combination thereof. In some embodiments, the electromagnetic radiation comprises visible light. In some embodiments of the present disclosure, the electromagnetic radiation that stimulates the presently disclosed molecular machines or is used in the presently disclosed methods to stimulate molecular machines has a wavelength of between about 10⁻⁹ nm to about 100 km. In some embodiments, the electromagnetic radiation used in the presently disclosed methods has a wavelength of between 100 nm and 5000 nm. In some embodiments, the wavelength of electromagnetic radiation used in the presently disclosed methods is about 100 nm, about 200 nm, about 300 nm, about 400 nm, about 500 nm, about 600 nm, about 700 nm, about 800 nm, 900 nm, about 1000 nm, about 1100 nm, about 1200 nm, about 1300 nm, about 1400 nm, about 1500 nm, about 1600 nm, about 1700 nm, about 1900 nm, about 2000 nm, about 2100 nm, about 2200 nm, about 2300 nm, about 2400 nm, about 2500 nm, or any range derivable therein. In some embodiments, the wavelength of electromagnetic radiation used in the presently disclosed methods is about 100 nm, about 200 nm, about 300 nm, about 400 nm, about 500 nm, about 600 nm, about 700 nm, about 800 nm, 900 nm, about 1000 nm, about 1100 nm, about 1200 nm, about 1300 nm, about 1400 nm, about 1500 nm, about 1600 nm, about 1700 nm, about 1900 nm, about 2000 nm, or any range derivable therein. In some embodiments, the wavelength of electromagnetic radiation used in the presently disclosed methods is about 300 nm, about 400 nm, about 500 nm, about 600 nm, about 700 nm, about 800 nm, or any range derivable therein. In some embodiments, the wavelength of electromagnetic radiation used in the presently disclosed methods is about 300 nm, about 400 nm, or about 500 nm. In some embodiments, the wavelength of electromagnetic radiation used in the presently disclosed methods is about 400 nm. In some embodiments, the electromagnetic radiation is delivered by a laser.

In some embodiments, the activation of the stimulus activated molecular machines according to the presently disclosed methods occurs for a defined or controlled time period. In some embodiments, the controlled time period is less than about 10 seconds, such as less than 9 seconds, less than 8 seconds, less than 7 seconds, less than 6 seconds, less than 5 seconds, less than 4 seconds, less than 3 seconds, less than 2 seconds, less than 1 second, or any range derivable therein. In some embodiments, the controlled time period is less than 5 seconds, less than 4 seconds, less than 3 seconds, less than 2 seconds, or less than 1 second. In some embodiments, the molecular machine is activated for less than 5 seconds. In some embodiments, the molecular machine is activated for less than 2 seconds. In some embodiments, the molecular machine is activated for about 500 milliseconds, about 400 milliseconds, about milliseconds, about 200 milliseconds, about 100 milliseconds, or any range derivable therein. In some embodiments, the molecular machines are activated for between about 200 milliseconds and about 300 milliseconds. In some embodiments, the molecular machines are activated for between about 250 milliseconds.

The stimulus-responsive molecular machines disclosed herein and the presently disclosed methods of use thereof are particularly valuable due to the mechanical mechanism of action. Therapeutic effects that can be achieved by mechanical rather than traditional chemical means, as are shown for MMs disclosed herein or as used according to the presently disclosed methods, facilitate a reduction in the selective pressure created by high therapeutic doses, which in turn has the benefit of retarding or mitigating the emergence of resistance to therapies. Among the stimuli that can activate MMs, light is particularly appealing due to its non-chemical, non-invasive nature, and ease of control. More specifically, activation by light facilitates precise localization and temporal control of therapeutic action.

### H. Pharmaceutical Formulations and Routes of and Administration

The present disclosure features methods comprising a stimulus activated molecular machine. In some embodiments, the stimulus activated molecular machine is administered in an effective amount. In some embodiments, the effective amount is a therapeutically effective amount. In some embodiments, the methods comprise contacting a microorganism, a biofilm, or a cell with a stimulus activated molecular machine. In some embodiments, the methods comprise contacting a microorganism or a cell with an amount of stimulus activated molecular machine that is sufficient to effect a desired change. In some embodiments, the methods comprise inhibiting the growth of a microorganism or cell. In some embodiments, the methods comprise killing a microorganism or cell. In some embodiments, the methods comprise inducing necrosis in a microorganism or cell. In some embodiments, the methods comprise causing oxidative stress or inhibiting mitochondrial function in a cell. In some embodiments, the methods comprise overcoming drug resistance in a microorganism or cell. In some embodiments, the methods comprise contacting a biofilm with an amount of stimulus activated molecular machine that is sufficient to effect a desired change. In some embodiments, the methods comprise contacting the biofilm with an amount of stimulus activated molecular machine that is sufficient to inhibit the formation of a biofilm or to eliminate a biofilm.

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a stimulus activated molecular machine disclosed herein formulated with one or more excipients and/or drug carriers appropriate to the indicated route of administration. In some embodiments, the stimulus activated molecular machines disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the stimulus activated molecular machines may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the stimulus activated molecular machines disclosed herein may be coated in a material to protect the compound from the action of acids and other natural conditions which may inactivate the compound. To administer the active compound by other than parenteral administration, it may be necessary to coat the stimulus activated molecular machine with, or co-administer the stimulus activated molecular machine with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The stimulus activated molecular machines disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The stimulus activated molecular machines or pharmaceutical formulations or compositions thereof disclosed herein can be administered orally, for example, with an inert diluent or an assimilable edible carrier. The stimulus activated molecular machines and other ingredients may also be enclosed in a hard or soft-shell gelatin capsule, compressed into tablets, or incorporated directly into the patient's diet. For oral therapeutic administration, the stimulus activated molecular machines disclosed herein may be incorporated with excipients and used in the form of ingestible tablets, buccal tablets, troches, capsules, elixirs, suspensions, syrups, wafers, and the like. The percentage of the stimulus activated molecular machines in the compositions and preparations may, of course, be varied. The amount of the stimulus activated molecular machines in such pharmaceutical formulations is such that a suitable dosage will be obtained.

The stimulus activated molecular machines or pharmaceutical formulations or compositions thereof may also be administered topically to the skin, eye, ear, or mucosal membranes. Administration of the stimulus activated molecular machines topically may include formulations of the compounds as a topical solution, lotion, cream, ointment, gel, foam, transdermal patch, or tincture. When the stimulus activated molecular machine is formulated for topical administration, the stimulus activated molecular machines may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the stimulus activated molecular machines to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the stimulus activated molecular machines may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of stimulus activated molecular machines calculated to produce the desired therapeutic effect in association with the required pharmaceutical carrier. In some embodiments, the specification for the dosage unit forms of the invention are dictated by and directly dependent on (a) the unique characteristics of the stimulus activated molecular machines and the particular therapeutic effect to be achieved, and (b) the limitations inherent in the art of compounding such a stimulus activated molecular machine for the treatment of a selected condition in a patient. In some embodiments, compounds of the present disclosure are administered at a therapeutically effective dosage sufficient to treat a condition associated with a condition in a patient. For example, the efficacy of a stimulus activated molecular machine can be evaluated in an animal model system that may be predictive of efficacy in treating the disease in a human or another animal.

In some embodiments, the effective dose range for the stimulus activated molecular machines disclosed herein can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED(mg/kg)=Animal dose(mg/kg)×(Animal Km/Human Km)

Use of the Km factors in conversion results in HED values based on body surface area (BSA) rather than only on body mass. Km values for humans and various animals are well known. For example, the Km for an average 60 kg human (with a BSA of 1.6 m²) is 37, whereas a 20 kg child (BSA 0.8 m²) would have a Km of 25. Km for some relevant animal models are also well known, including: mice Km of 3 (given a weight of 0.02 kg and BSA of 0.007); hamster Km of 5 (given a weight of 0.08 kg and BSA of 0.02); rat Km of 6 (given a weight of 0.15 kg and BSA of 0.025) and monkey Km of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a stimulus activated molecular machine of the present disclosure or composition comprising a stimulus activated molecular machine of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount of stimulus activated molecular machine typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the stimulus activated molecular machine in the pharmaceutical formulation is from about 0.1% and 100% (w/w). In some embodiments, the amount of active compound is from about 2 to about 75 weight percent. In further embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### Combination Therapy

In many clinical situations, it is advisable to use a combination of distinct therapies. Thus, it is envisioned that, in addition to the therapies described above, one would also wish to provide to the patient more “traditional” pharmaceutical anti-fungal therapies. Examples of standard therapies are described above. Combinations may be achieved by administering a single composition or pharmacological formulation that includes both agents, or with two distinct compositions or formulations, at the same time, wherein one composition includes the agents of the present disclosure and the other includes the standard therapy. Alternatively, standard therapy may precede or follow the present agent treatment by intervals ranging from minutes to weeks to months. In embodiments where the treatments are applied separately, one would generally ensure that a significant period of time did not expire between the time of each delivery, such that the agents would still be able to exert an advantageously combined effect on the subject. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the agent of the present disclosure, or the standard therapy will be desired. Various combinations may be employed, where the present disclosure compound is “A” and the standard therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated as well. In an embodiment, the presently disclosed methods comprise administration of an additional pharmaceutical agent, the additional pharmaceutical agent is an anti-fungal agent, e.g., one or more of an agent that selectively reduces or eliminates fungal pathogens from a patient or host with minimized toxicity to the host. In an embodiment, the additional anti-fungal agent is a polyene antifungal drug (e.g., interacts with sterols in the cell membrane to form channels through which small molecules leak from the inside of the fungal cell to the outside). In some embodiments, the additional anti-fungal agent is Amphotericin B, fluconazole, itraconazole, posaconazole, or voriconazole. In some embodiments, the additional anti-fungal agent is an azole, an allylamine or a morpholine, or an antimetabolite. In some embodiments, the additional anti-fungal agent is echinocandins or flucytosine. It is contemplated that other anti-fungal compounds may be used in combination with the present compounds.

In some embodiments, the additional pharmaceutical agent is an antibiotic. In some embodiments, the presently disclosed methods comprise administration of a second therapeutic agent. In some embodiments, the second therapeutic agent is a second chemotherapeutic agent, surgery, photodynamic therapy, sonodynamic therapy, radiotherapy, or immunotherapy.

### I. Kits

The technology disclosed herein includes kits for treating diseases or disorders. A “kit” refers to a combination of physical elements. For example, a kit may include, for example, one or more components, such as dispensing apparatus, an instruction sheet, and other elements useful to practice the technology described herein. These physical elements can be arranged in any way suitable for carrying out the disclosure.

The components of the kits may be packaged either. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present disclosure also will typically include a means for containing the stimulus activated molecular motor and any other reagent containers or instruments in close confinement for commercial sale. Such containers may include injection or blow molded plastic containers into which the desired vials are retained.

A kit will also include instructions for employing the kit components as well the use of any other reagent not included in the kit. Instructions may include variations that can be implemented. It is contemplated that such reagents are embodiments of kits of the disclosure. Such kits, however, are not limited to the particular items identified above.

### J. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Visible-Light-Activated Molecular Machines Kill Fungi by Necrosis Following Mitochondrial Dysfunction and Calcium Overload

Molecular machines (MMs) (FIG. 1A) are examples of stimuli-responsive compounds that, in response to light, undergo a conformational change, generating a drill-like motion that disrupts lipid bilayers (García-López et al., 2017). These stimuli-responsive systems are particularly promising because they enable attack using a mechanical mechanism at the molecular scale. MMs can be spatially and temporally activated by light, allowing precise localization and temporal control of, for example, antimicrobial action. Additionally, the presently disclosed methods may facilitate a reduction in the selective pressure created by high antimicrobial doses due to the mechanical mechanism of action of the MMs described herein, which in turn may retard or mitigate the emergence of therapeutic resistance.

The details that follow describe the use of stimulus activated molecular machines to rapidly kill planktonic and biofilm fungi without resistance development via a new mechanism of action in which molecular machines bind fungal mitochondrial phospholipids, eliciting mitochondrial dysfunction, calcium overload, and necrosis following light activation. At sublethal concentrations, stimulus activated molecular machines also potentiated the effects of conventional antifungals, at least in part by impairing efflux pump function. Finally, the presently disclosed stimulus activated molecular machines synergized with conventional antifungals in vivo, reducing mortality and fungal burden associated with systemic fungal infections, and ex vivo, outperforming monotherapy with conventional antifungals in reducing the fungal load in an onychomycosis porcine model. Details of these benefits are provided in the sections that follow.

MMs Kill Planktonic and Biofilm Fungi without Resistance Development

Nineteen fast, unidirectionally rotating (~3 MHz) visible-light-activated MMs (Table 1) (Santos et al., 2022) and a slow motor control (10⁻⁶ Hz) were examined for antifungal activity against a strain of the human pathogen Candida albicans isolated from a skin lesion (ATCC 18804). Since substituted piperazines are known improve molecule lipophilicity to increase antimicrobial activity (Ozdemir et al., 2018), a piperazine-modified molecular machine (MM 7) was also investigated.

TABLE 1
Chemical structures of the visible-light-activated MMs investigated in Example 1

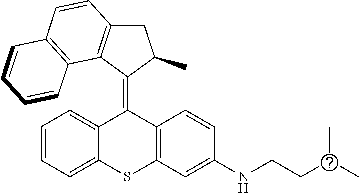

MM 1 (DL -4-654)
MW = 462.8550
3 MHz

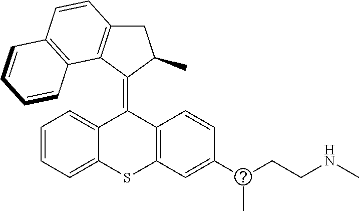

MM 5 (DL-5-877)
MW = 462.6550
3 MHz

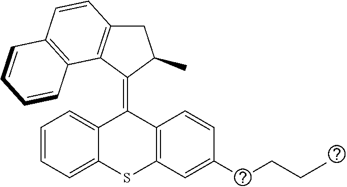

MM 6 (DL-5-878)
MW = 434.5010
3 MHz

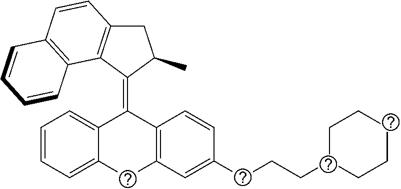

MM 7 (GL-B2)
MW = 503.2395
3 MHz

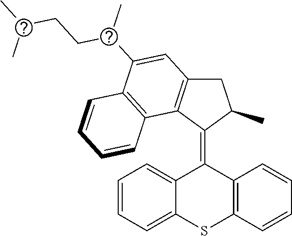

DL-4-751
MW = 476.882
3 MHz

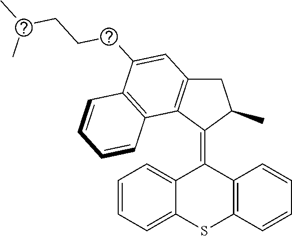

DL-4-726
MW = 4 [symbol] 2. [symbol] 850
3 MHz

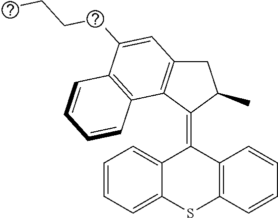

DL-5-873
MW = 434.8010
3 MHz

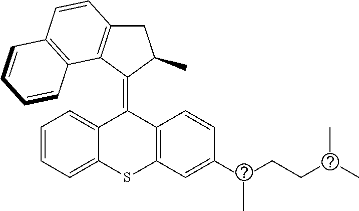

DL-5-876
MW = 476.6820
3 MHz

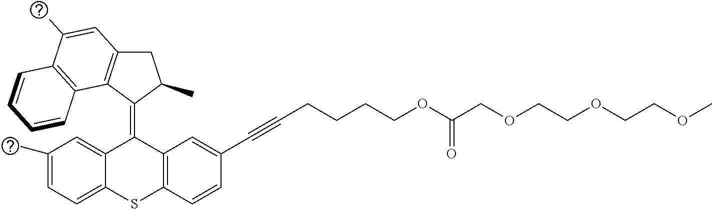

DL-4-655
MW = 662.8410
3 MHz

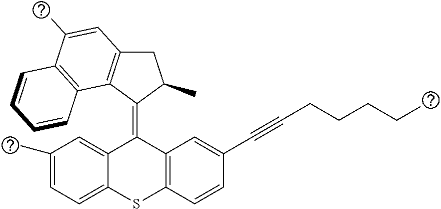

DL-4-653
MW = 485.6 [symbol] 50
3 MHz

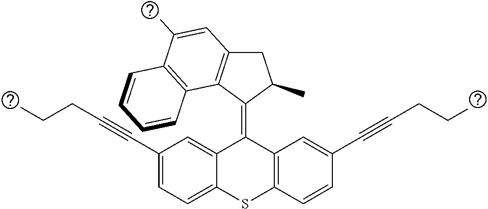

DL-4-733
MW = [symbol] 42.6930
3 MHz

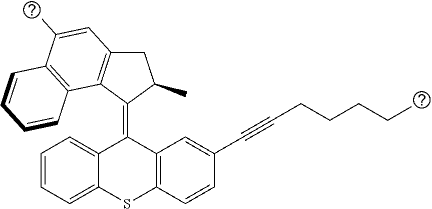

DL-4-781
MW = 504.5880
3 MHz

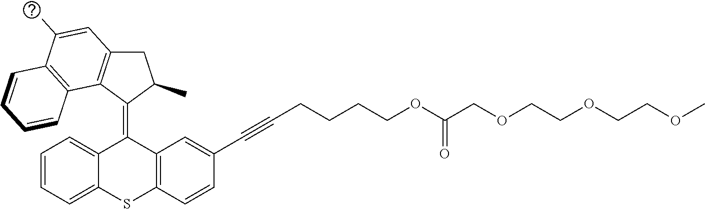

DL-4-799
MW = [symbol]
3 MHz

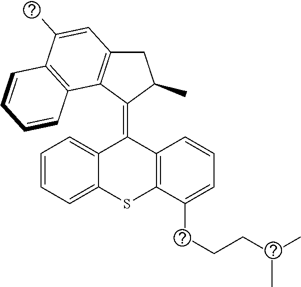

DL-5-894
MW = 492.8810
3 MHz

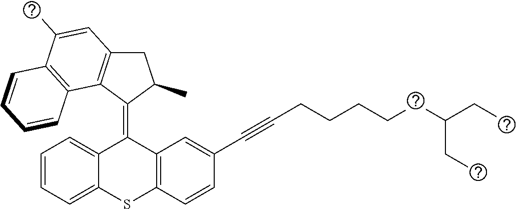

DL-4-794
MW = 579.2224
3 MHz

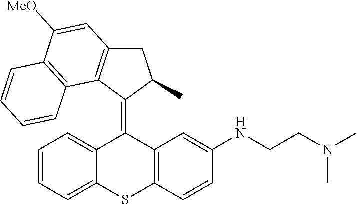 [SMILES: COc1cc2c(c3ccccc13)/C(=C1\c3ccccc3Sc3ccc(NCCN(C)C)cc31)[C@H](C)C2]

DL-5-872
MW = [symbol]
3 MHz

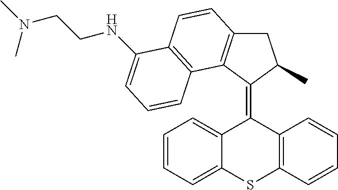 [SMILES: C[C@@H]1Cc2ccc3c(NCCN(C)C)cccc3c2C1=C1c2ccccc2Sc2ccccc21]

DL-5-901
MW = [symbol]
3 MHz

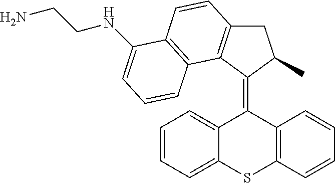 [SMILES: C[C@@H]1Cc2ccc3c(NCCN)cccc3c2C1=C1c2ccccc2Sc2ccccc21]

DL -5-903
MW = 434. [symbol] 010
3 MHz

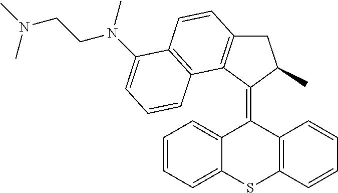 [SMILES: C[C@@H]1Cc2ccc3c(N(C)CCN(C)C)cccc3c2C1=C1c2ccccc2Sc2ccccc21]

DL-5-902
MW = 476.6820
3 MHz

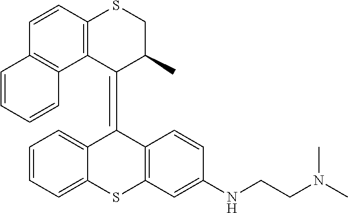 [SMILES: C[C@@H]1CSc2ccc3ccccc3c2/C1=C1\c2ccccc2Sc2cc(NCCN(C)C)ccc21]

ARV-3-202
MW = 494.7150
10 [symbol] MHz
Chemical structures of the visible-light-activated MMs investigated in this study, their corresponding molecular weights, and their estimated rotation rates after light activation calculated based on the rotation rates of known motors with similar core skeletons (Klok et al., 2008). A slow MM (ARV-3-262) was used to assess the importance of fast rotational speeds for the antifungal activity of MMs. Detailed information on the synthesis and characterization of the piperazine-modified motor MM 7 can be found in the sections that follow. For the remaining molecules, detailed information on synthesis and characterization can be found elsewhere (Santos et al., 2022). Chemical structures were created in Chemdraw Ultra 6.0 (CambridgeSoft, Cambridge, MA, USA). [symbol]
indicates data missing or illegible when filed

C. albicans cell suspensions were incubated with increasing concentrations of MMs and irradiated with 405-nm light at 292 mW cm⁻² for 5 min (87.6 J cm⁻²). The minimum inhibitory concentration (MIC) was defined as the MM concentration resulting in no visible fungal growth after irradiation with 87.6 J cm⁻² of 405-nm light.

The MICs of the different MMs for C. albicans varied from 1.25-80 μM (FIG. 1B). The inhibitory effects of the most potent MMs (MM 1, MM 5, MM 6, MM 7), displaying MIC values≤5 μM, were further investigated in the yeast Saccharomyces cerevisiae and the molds Aspergillus fumigatus, Microsporum gypseum, and Trichophyton rubrum. S. cerevisiae showed a susceptibility profile similar to that of C. albicans, with MIC values of 1.25-5 μM. Among molds, A. fumigatus had the highest mean MIC values (5-10 μM), whereas M. gypseum and T. rubrum were more sensitive to visible-light-activated MMs, with MIC values of 0.31-2.5 μM (FIG. 1C).

The minimum fungicidal concentration (MFC), i.e., the lowest MM concentration that killed ≥99.9% of the original inoculum (Guinea et al., 2008) was similar to or, at most, twice the MIC(FIG. 1C), demonstrating that MMs are indeed fungicidal and not just fungistatic.

The antifungal potential of the four most potent MMs was further investigated in time-kill experiments by treating fungal strains with MMs (2×MIC) or 1% DMSO, followed by irradiation with 405-nm light at 292 mW cm⁻² for up to 10 min. A slow (~10⁻⁶ Hz) MM control (FIG. 1C), structurally homologous to MM 1 (~3 MHz), was used to assess the importance of rotation speed for MM fungicidal activity. Amphotericin B (AMB, 4×MIC, Table 2) was used as a control antifungal.

TABLE 2
Minimum inhibitory concentration (MIC, μg ml⁻¹) of various antifungal agents

| | C. albicans | A. fumigatus | S. cerevisiae | T. rubrum |
|---|---|---|---|---|
| 5-Fluorocytosine (5-FC) | 1 | — | — | — |
| Amphotericin B (AMB) | 1 | 2 | 0.5 | — |
| Fluconazole (FLC) | 0.5 | — | — | — |
| Voriconazole (VRC) | 0.03 | 0.5 | 0.125 | — |
| Caspofungin (CAS) | 0.25 | — | 0.5 | — |
| Ciclopirox (CPX) | 1 | — | — | 0.5 |

Minimum inhibitory concentration (MIC, μg ml⁻¹) of various antifungal agents in different fungal strains used in this study determined using the broth microdilution method according to the CLSI guidelines for yeasts (CLSI M27-A2) (CLSI, 2017) and molds (CLSI M38-A2) (CLSI, 2008). Further details are provided in elsewhere in Example 1. The results are the average of at least three independent biological replicates.

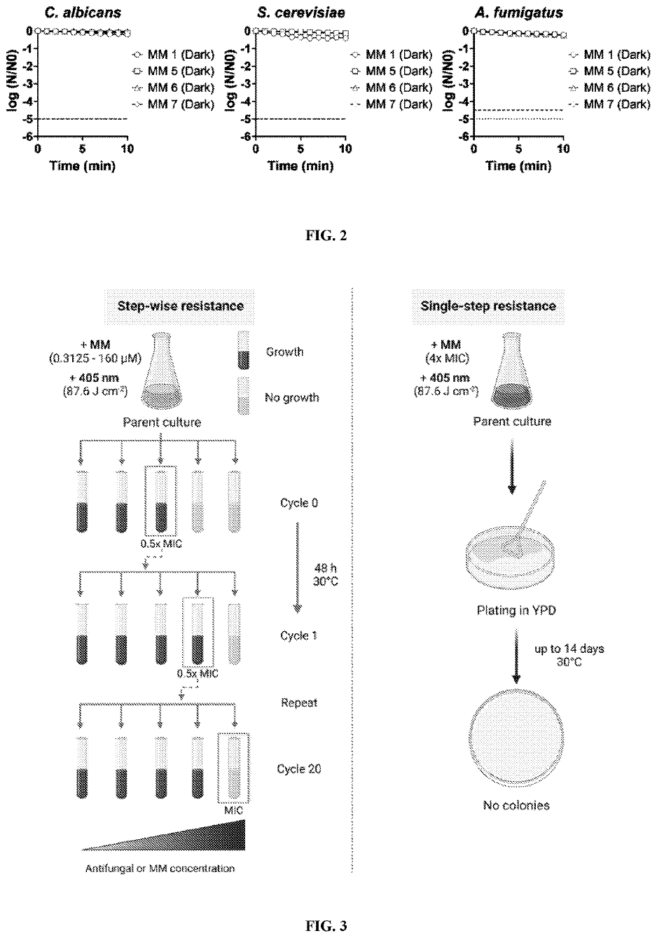

MM treatment reduced C. albicans cell numbers to the limit of detection in 5 min (MM 6) to 9 min (MM 7) (FIG. 1D). In S. cerevisiae, population eradication was achieved in 2 min (MM 5) to 5 min (MM 7) (FIG. 1D). A. fumigatus cell number reduction to the limit of detection occurred from 6 min (MM 5) to 9 min (MM 7) (FIG. 1D). Non-irradiated MMs and slow MMs had no significant effect on cell number (FIG. 1D; FIG. 2), demonstrating the importance of light-induced fast rotation rates for the fungicidal activity of MMs. Treatment with AMB resulted only in a non-significant reduction in cell numbers (FIG. 1D). Under the same irradiation conditions, killing of C. albicans by MMs varied in a concentration-dependent manner (FIG. 1E), with increasing MM concentrations enhancing killing. At the same MM concentration, killing could also be remotely controlled by adjusting the light dose, with higher light doses leading to enhanced killing (FIG. 1F).

The antibiofilm potential of the most effective visible-light-activated MMs (2×, 4× MIC plus 87.6 J cm⁻² of 405-nm light) against mature C. albicans biofilms was evaluated in a 96-well plate format using the XTT assay (Nett et al., 2011) and crystal violet assay (Martins et al., 2010) to assess effects on viability and biomass, respectively, against the control antifungal AMB (2×, 4×MIC). Compared with DMSO controls, visible-light-activated MMs reduced biofilm viability by up to 96% (MM 1, p<0.0001), whereas AMB reduced biofilm viability by only 20% (p<0.01) (FIG. 1G). Relative to DMSO controls, visible-light-activated MMs reduced biofilm biomass by up to 35% (MM 5, p<0.05), whereas AMB treatment achieved only a non-significant 6% reduction (FIG. 1H).

Resistance development to visible-light activated MMs was assessed by serial passage experiments. C. albicans cells surviving 0.5×MIC of MM plus light (405 nm at 87.6 J cm⁻²) were subjected to 20 cycles of repeated MM treatment. Unlike caspofungin (CAS) and fluconazole (FLC), repeated MM treatment did not increase the MM MIC(FIG. 1I). Furthermore, antifungal-resistant mutants did not exhibit cross-resistance to MMs (Table 3). A single-step strategy to isolate MM-resistant mutants was attempted, but no resistant colonies were recovered (FIG. 3).

TABLE 3
Susceptibility (assessed as the MIC in μM)
of antifungal-resistant (^{R}) C. albicans to MMs

| Passage | FLC^{R} | | | | CAS^{R} | | | |
|---|---|---|---|---|---|---|---|---|
| number | MM 1 | MM 5 | MM 6 | MM 7 | MM 1 | MM 5 | MM 6 | MM 7 |
| 1 | 1.25 | 2.5 | 2.5 | 5 | 1.25 | 2.5 | 2.5 | 5 |
| 2 | 1.25 | 2.5 | 2.5 | 5 | 1.25 | 2.5 | 2.5 | 5 |
| 3 | 1.25 | 2.5 | 2.5 | 5 | 1.25 | 2.5 | 2.5 | 5 |
| 4 | 1.25 | 2.5 | 2.5 | 5 | 1.25 | 2.5 | 2.5 | 5 |
| 5 | 1.25 | 2.5 | 2.5 | 5 | 1.25 | 2.5 | 2.5 | 5 |
| 6 | 1.25 | 2.5 | 2.5 | 5 | 1.25 | 2.5 | 2.5 | 5 |
| 7 | 1.25 | 2.5 | 2.5 | 5 | 1.25 | 2.5 | 2.5 | 5 |
| 8 | 1.25 | 2.5 | 2.5 | 5 | 1.25 | 2.5 | 2.5 | 5 |
| 9 | 1.25 | 2.5 | 2.5 | 5 | 1.25 | 2.5 | 2.5 | 5 |
| 10 | 1.25 | 2.5 | 2.5 | 5 | 1.25 | 2.5 | 2.5 | 5 |
| 11 | 1.25 | 2.5 | 2.5 | 5 | 1.25 | 2.5 | 2.5 | 5 |
| 12 | 1.25 | 2.5 | 2.5 | 5 | 1.25 | 2.5 | 2.5 | 5 |
| 13 | 1.25 | 2.5 | 2.5 | 5 | 1.25 | 2.5 | 2.5 | 5 |
| 14 | 1.25 | 2.5 | 2.5 | 5 | 1.25 | 2.5 | 2.5 | 5 |
| 15 | 1.25 | 2.5 | 2.5 | 5 | 1.25 | 2.5 | 2.5 | 5 |
| 16 | 1.25 | 2.5 | 2.5 | 5 | 1.25 | 2.5 | 2.5 | 5 |
| 17 | 1.25 | 2.5 | 2.5 | 5 | 1.25 | 2.5 | 2.5 | 5 |
| 18 | 1.25 | 2.5 | 2.5 | 5 | 1.25 | 2.5 | 2.5 | 5 |
| 19 | 1.25 | 2.5 | 2.5 | 5 | 1.25 | 2.5 | 2.5 | 5 |
| 20 | 1.25 | 2.5 | 2.5 | 5 | 1.25 | 2.5 | 2.5 | 5 |

During serial passage experiments used to assess antifungal resistance, cells that grew at 0.5 × MIC for each antifungal were collected and stored at −80 °C. These cells were then re-grown, amended with a range of MM concentrations and irradiated with 405 nm light (87.6 J cm⁻²). Subsequently, the irradiated cells were inoculated into MOPS-buffered RPMI 1640 media and grown at 30 °C for 48 h. Further experimental details can be found elsewhere in Example 1. The tubes were then inspected for growth to determine the MIC. The results are the mean of at least three biological replicates. FLC: Fluconazole. CAS: Caspofungin.

### MMs Potentiate the Activity of Conventional Antifungals

The mechanisms of action of MMs were investigated using the human pathogen C. albicans under the same irradiation conditions (405-nm light at 87.6 J cm⁻²) and varying MM concentrations (0.5×, 1×, or 2×MIC) (FIG. 1C). Comparison with 1% DMSO-treated samples irradiated under similar conditions allowed discrimination between MM-induced effects and those caused by irradiation alone.

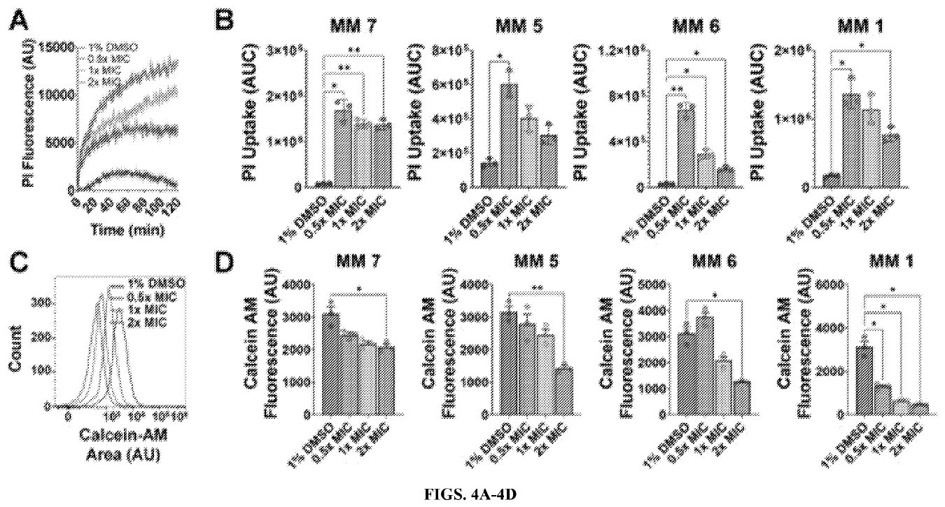

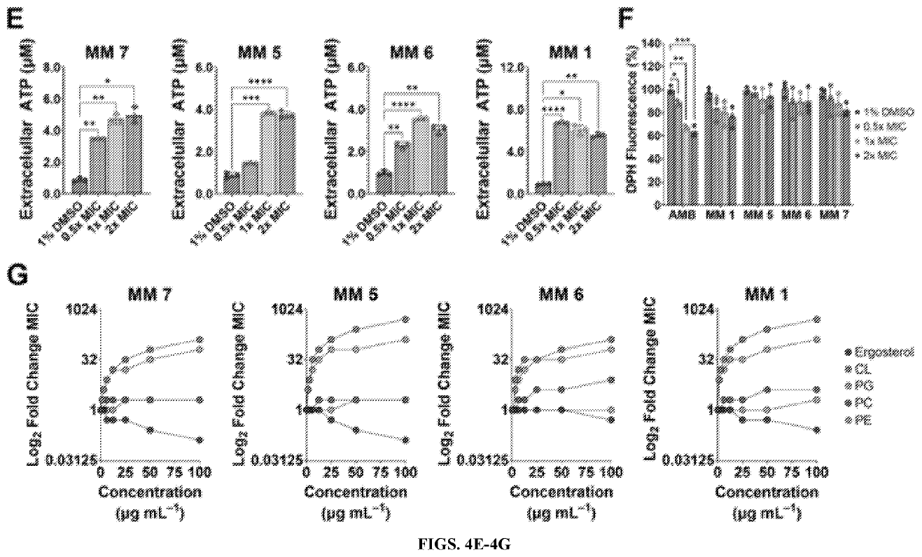

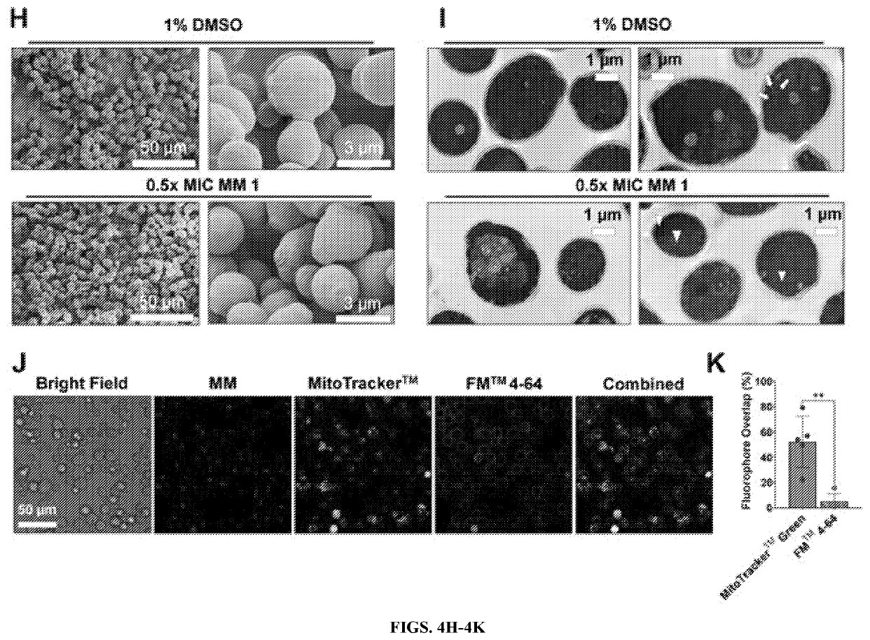

The fluorescence of the nucleic acid-binding dye propidium iodide (PI) was used to determine the effects of MMs on plasma membrane integrity. Treatment with visible-light-activated MMs resulted in increased PI uptake (FIG. 4A), particularly at 0.5×MIC(p<0.05) (FIG. 4B), indicating MM-induced plasma membrane permeabilization. Impaired plasma membrane integrity was also evidenced by decreased intracellular calcein fluorescence (FIG. 4C) in cells treated with increasing MM concentrations (FIG. 4D). Additionally, MM treatment significantly increased the extracellular ATP concentration (p<0.05) (FIG. 4E), reflecting intracellular content leakage.

To investigate whether MMs act directly on the fungal plasma membrane, the fluorescence of 1,6-diphenyl-hexa-1,3,5-triene (DPH) (Kim et al., 2009), which has a high affinity for membrane phospholipids, was monitored. In contrast to AMB, which binds plasma membrane ergosterol and reduces DPH fluorescence (FIG. 4F, p<0.05), treatment with MM had no effect on DPH fluorescence (FIG. 4F), indicating that MMs do not bind plasma membrane phospholipids of C. albicans.

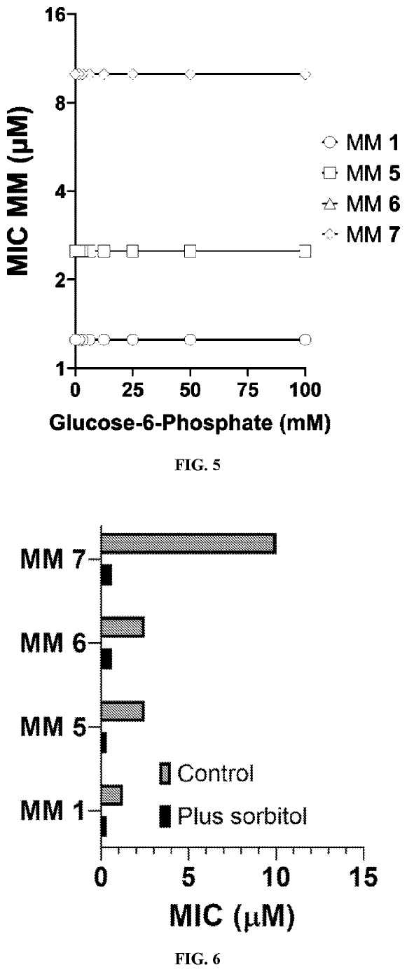

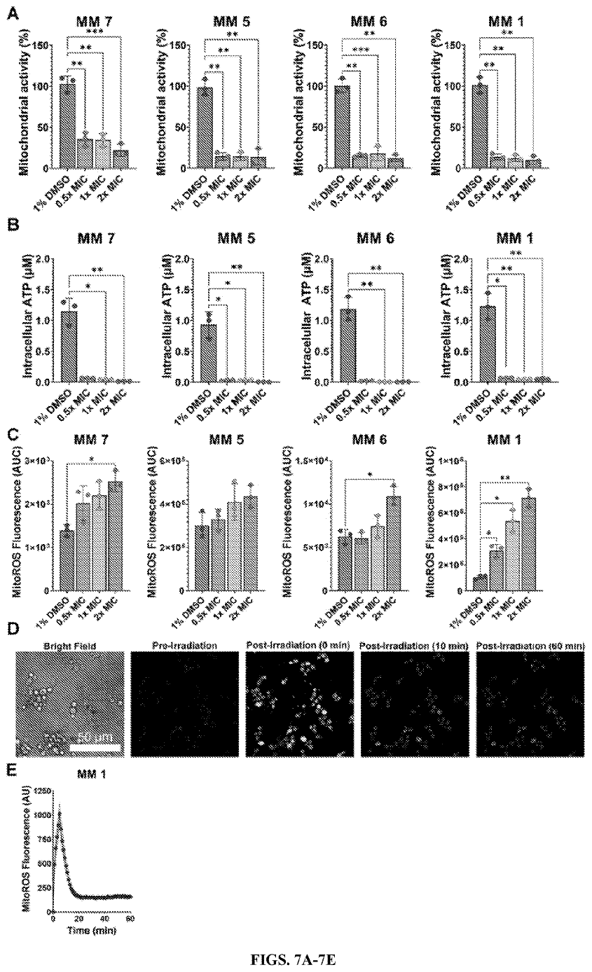

Binding of MM to the fungal plasma membrane was further investigated in competition binding assays with exogenous ergosterol, the main fungal sterol, or phosphatidylethanolamine and phosphatidylcholine, the main phospholipids of the fungal plasma membrane. Treatment with increasing concentrations of ergosterol resulted in a reduction in MM MIC, whereas phosphatidylethanolamine and phosphatidylcholine either had no significant effect or caused only a small increase in MM MIC(FIG. 4G), confirming that MMs do not bind the plasma membrane sterols or phospholipids of C. albicans. Similarly, exogenous glucose-6-phosphate, representing negatively charged fungal cell wall polysaccharides, did not affect MM MIC(FIG. 5), and sorbitol did not offer protection against MM-induced growth arrest (FIG. 6), indicating that the fungal cell wall is also not targeted by MMs.

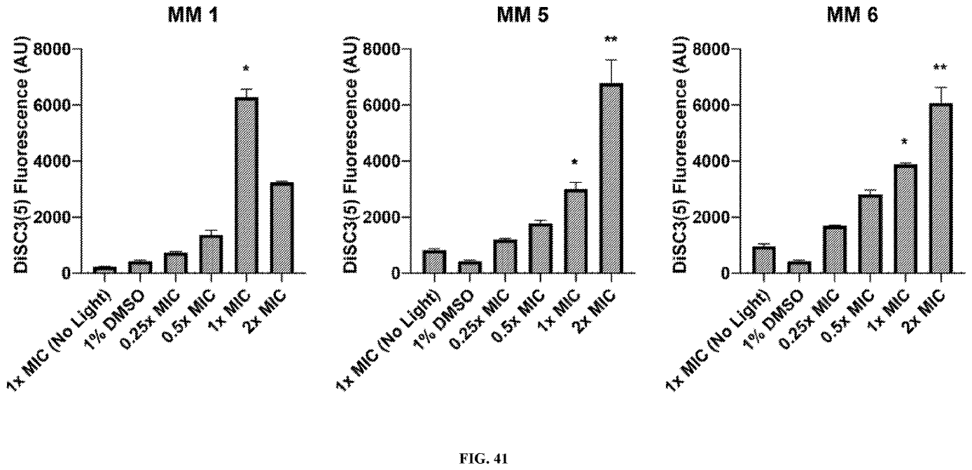

Scanning electron microscopy confirmed that MM treatment did not alter the cell surface of C. albicans (FIG. 4H). Conversely, transmission electron microscopy (TEM) revealed extensive intracellular structural damage in MM-treated C. albicans, characterized by the loss of most subcellular membrane systems (FIG. 41). Competition binding experiments with the negatively charged mitochondrial phospholipids cardiolipin and phosphatidylglycerol revealed a substantial increase in MM MIC(up to 512-fold) (FIG. 4G), suggesting that MMs bind these phospholipids.

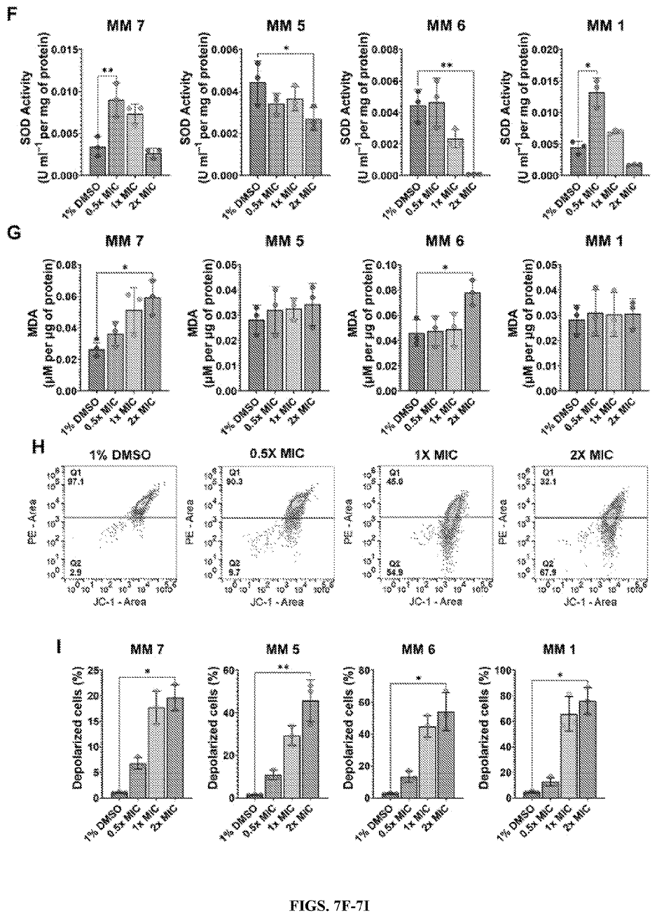

To investigate whether MMs target mitochondria, the cellular distribution of MM 1 (the most potent MM) in C. albicans was examined by confocal microscopy, which revealed that MM 1 was internalized within cells (FIG. 4J). Image analysis confirmed an average areal colocalization of MM 1 and the mitochondrial dye MitoTracker™ Green fluorescence of 52.5%, whereas that of MM 1 with the plasma membrane dye FM™ 4-64 was 5.2% (FIG. 4K, p<0.01). Investigating the effects of visible-light-activated MMs on mitochondrial function revealed a 67-92% reduction (P<0.01) in mitochondrial dehydrogenase activity in MM-treated cells compared with DMSO controls (FIG. 7A). Intracellular ATP levels were also significantly decreased (p<0.05) following MM treatment, from ~1 μM in untreated samples to ~0.005 μM in 2×MIC-treated samples (FIG. 7B). Based on these results, the effects of MM-induced mechanical disruption on intracellular processes were investigated.

A significant (p<0.05) and concentration-dependent increase in mitochondrial reactive oxygen species (ROS) levels (up to 7-fold) was observed in MM-treated samples using the mitochondrial superoxide-sensitive probe MitoROS™ 580 (FIG. 7C). Confocal microscopy revealed a sharp increase in ROS levels in irradiated MM 1-treated samples (FIG. 7D), which rapidly returned to preexposure levels after irradiation cessation (FIG. 7E), possibly reflecting mitochondrial tolerance to sublethal superoxide levels. Accordingly, cells treated with 0.5×MIC MM 7 and MM 1 displayed increased superoxide dismutase activity (FIG. 7F, p<0.05). However, the mitochondrial antioxidant capacity was eventually exhausted, resulting in oxidative damage to biomolecules, as evidenced by increased levels of the lipid peroxidation product malonaldehyde in cells treated with 2×MIC MM 6 and MM 7 (FIG. 7G). MM treatment also decreased mitochondrial membrane potential (FIG. 7H), as measured by the shift in 5,5′,6,6′-tetrachloro-1,1′,3,3′-tetraethylbenzimidazolocarbocyanine iodide (JC-1) fluorescence, in a concentration-dependent manner, with up to 75% of cells depolarized after MM treatment (FIG. 7I, p<0.05).

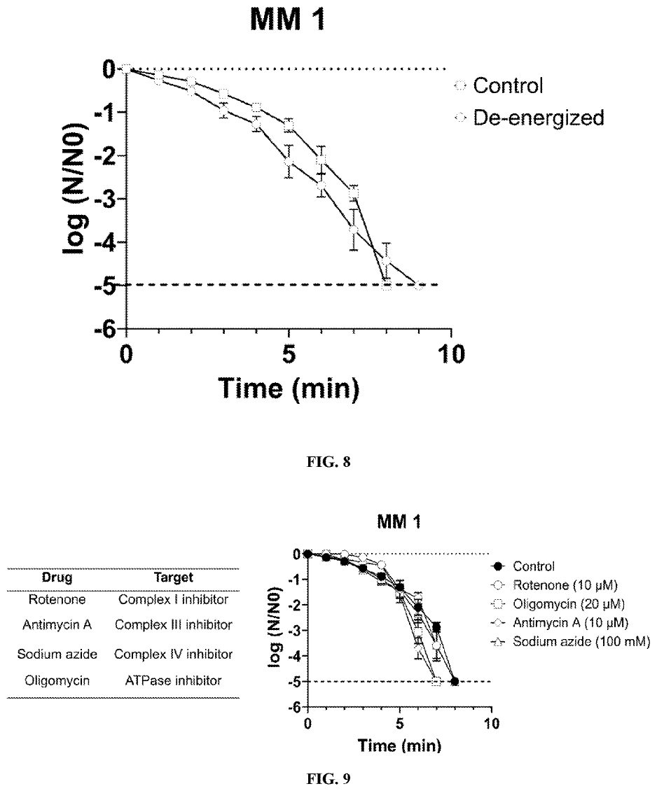

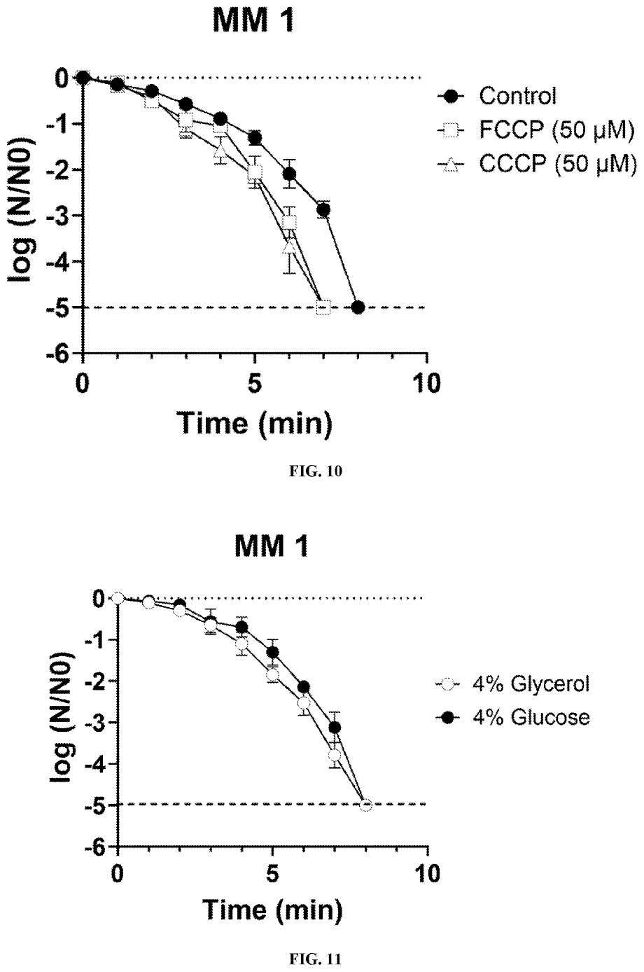

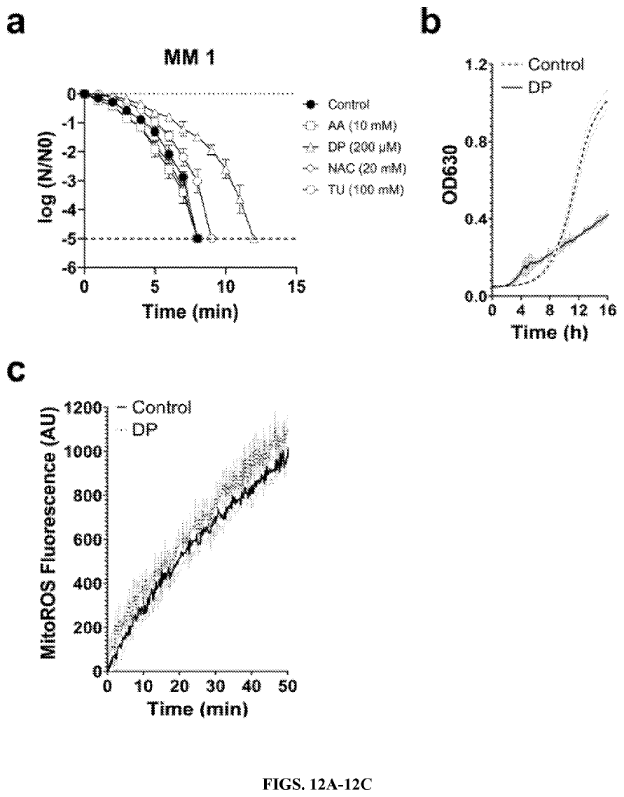

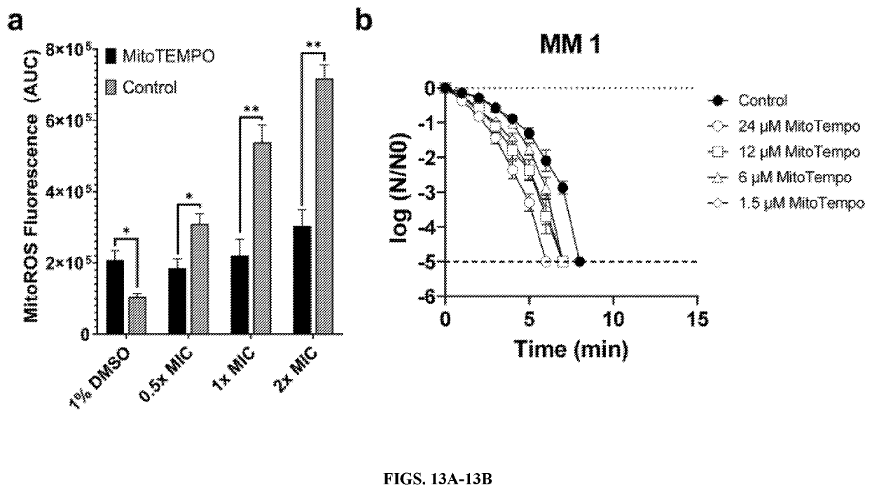

Without being bound by theory, these results identify bioenergetic deficit and oxidative stress, resulting in mitochondrial membrane depolarization, as important contributors to the antifungal mechanism of action of visible-light-activated MMs. However, cells depleted of ATP by chemically induced de-energization (FIG. 8) or electron transport chain inhibition (FIG. 9) were as susceptible to MM-induced killing as energized cells, demonstrating, without being bound by theory, that energy depletion alone cannot explain the MM killing mechanism. Likewise, cells pre-depolarized with carbonyl cyanide 3-chlorophenylhydrazone or carbonyl cyanide 4-(trifluoromethoxy)phenylhydrazone could still be killed by visible-light-activated MMs (FIG. 10). Moreover, fermentative growth did not protect against MM-mediated death (FIG. 11), unlike antifungals that induce mitochondrial dysfunction by collapsing the mitochondrial membrane potential (Shibata et al., 2012; Lanteri et al., 2004). These results indicate, without being bound by theory, that mitochondrial membrane depolarization alone also cannot explain MM-induced death. Additionally, the mitigation of MM-induced killing by the iron scavenger 2,2′-dipyridyl (FIG. 12A) could be ascribed to its effect on the growth rate (FIG. 12B) because it did not impact MM-induced ROS generation (FIG. 12C). Conversely, the mitochondrial superoxide scavenger MitoTEMPO reduced ROS generation (FIG. 13A) but did not affect survival following MM treatment (FIG. 13B).

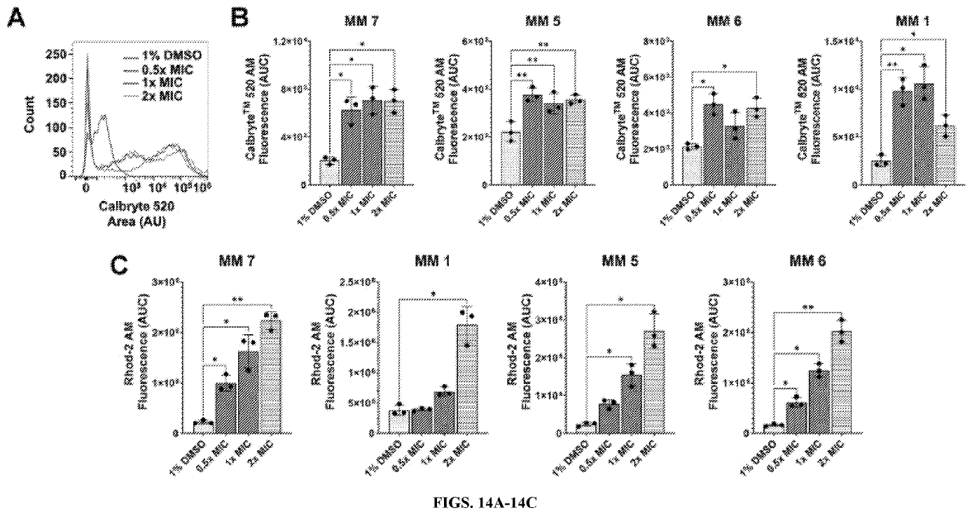

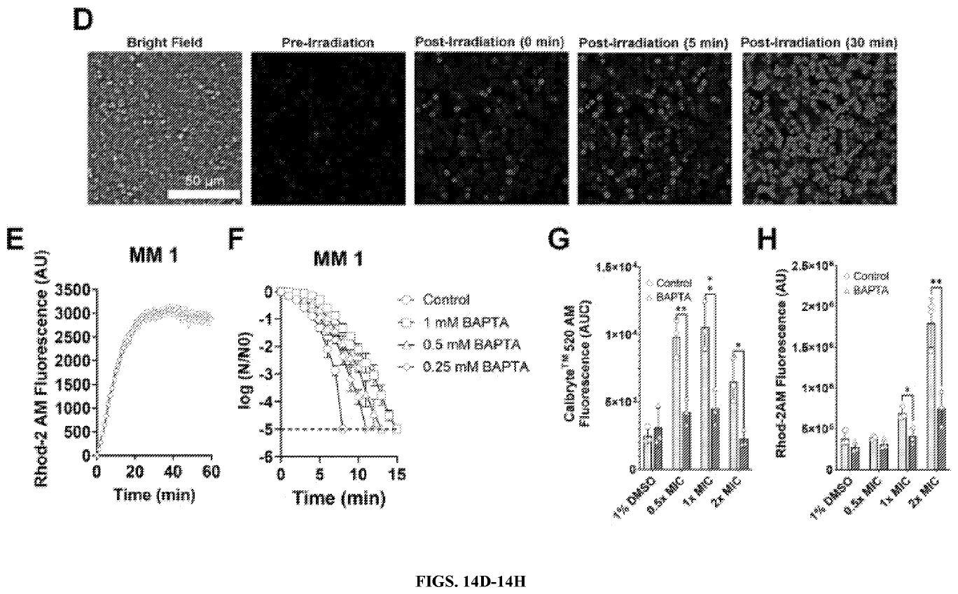

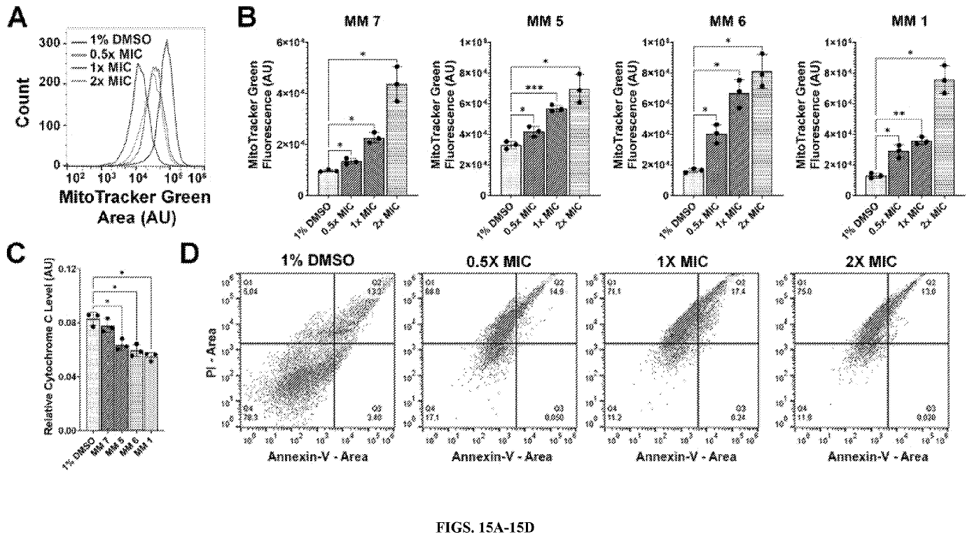

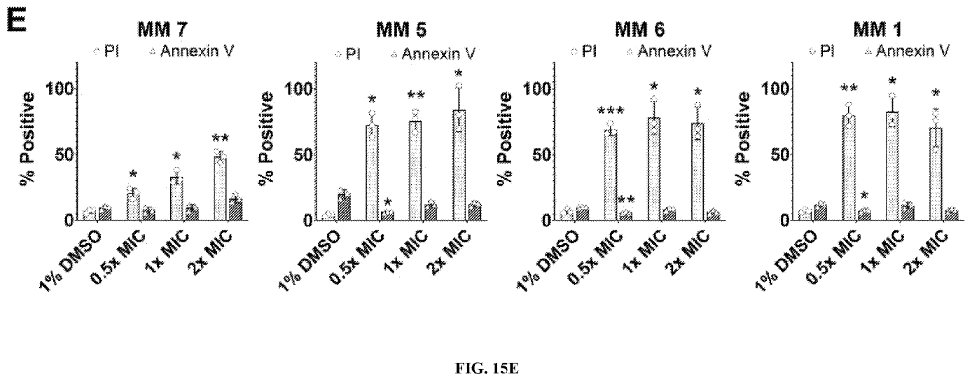

In addition to their roles in energy production and ROS generation, mitochondria are crucial for calcium homeostasis and apoptosis (McBride et al., 2006). Therefore, it was investigated whether these processes could also contribute to the MM mechanism of action. MM-treated cells showed increased cytosolic calcium levels detected with the Calbryte™ 520 AM fluorescent probe (FIG. 14A) of up to 4-fold (p<0.05) (FIG. 14B). Mitochondrial calcium levels detected using the fluorescent probe Rhod-2 AM showed an even greater increase (up to 12-fold, p<0.05) in MM-treated cells (FIG. 14C), which was also evident by live-cell calcium imaging using confocal microscopy (FIG. 14D, FIG. 14E). Mitigation of MM-induced cell death (FIG. 14F) and the MM-induced increases in cytosolic (FIG. 14G) and mitochondrial calcium (FIG. 14H) by the calcium chelator BAPTA-AM confirmed the importance of calcium homeostasis in the antifungal mechanism of action of MMs.

MM-treated cells showed increased MitoTracker™ Green fluorescence (FIG. 15A), particularly at 2×MIC(FIG. 15B, p<0.05), denoting increased mitochondrial mass/volume. This finding maybe due, without being bound by theory, to water influx into mitochondria following calcium overload, consistent with the substantial increase in mitochondrial size in MM-treated cells compared with DMSO controls detected by TEM (FIG. 4I). Additionally, significant reductions in mitochondrial cytochrome c levels (p<0.05) were observed in cells treated with 2×MIC of MMs 1, 5, and 6 (FIG. 15C), suggesting, without being bound by theory, mitochondrial outer membrane rupture and intramitochondrial content leakage.

An Annexin V-based assay was used to investigate whether the previously described MM-induced physiological changes lead to cell death by apoptosis or necrosis (Van Genderen et al., 2006). C. albicans protoplasts treated with MM (0.5-2×MIC) or 1% DMSO and irradiated with 405-nm light (87.6 J cm⁻²) were labeled with Annexin V and PI and analyzed by flow cytometry (FIG. 15D). The results confirmed that MM treatment induced cell death by necrosis, as evidenced by a significant increase in the percentage of PI-positive protoplasts by up to 80% (p<0.01), but only a non-significant change in the percentage of Annexin V-positive protoplasts (FIG. 15E).

### MMs Potentiate the Activity of Conventional Antifungals

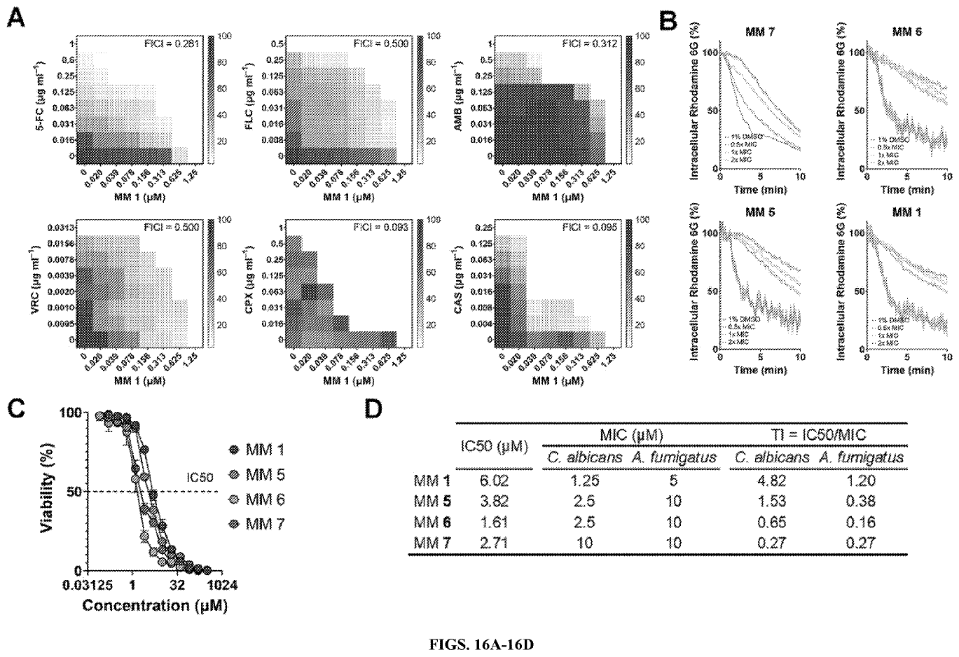

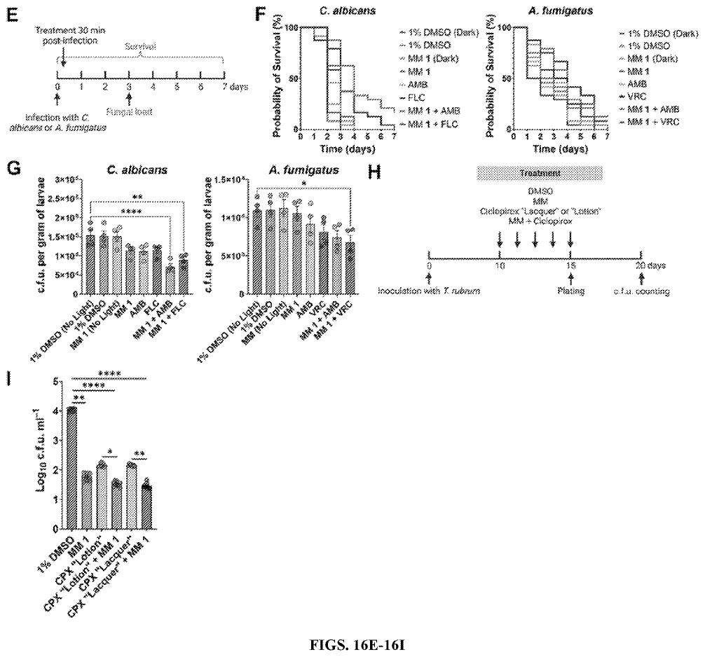

A modified checkerboard assay was used to study the interaction of visible-light-activated MMs with conventional antifungals in C. albicans. Cells were treated with increasing concentrations of MMs (up to 1×MIC), irradiated with 405-nm light (87.6 J cm⁻²), and then challenged with increasing concentrations of different antifungals (up to 1×MIC, Table 2). The type of interaction between MMs and conventional antifungals was assessed by calculating the fractional inhibitory concentration index (FICI), with a FICI of ≤0.5, 0.5<x≤4, or >4, denoting synergistic, additive, or antagonistic interactions, respectively (Odds, 2003). MM 1 synergized with all antifungals tested (FIG. 16A), with FICIs ranging from 0.093 (MM 1-ciclopirox) to 0.500 (MM 1-fluconazole and MM 1-voriconazole).

Rhodamine 6G efflux was used to assess whether the potentiation of conventional antifungals by MMs was due to impaired activity of energy-dependent efflux pumps. DMSO controls effluxed 75-85% of the accumulated rhodamine 6G, whereas MM-treated cells effluxed only 31-68% (FIG. 16B), denoting the interference of MMs with the activity of efflux pumps.

### MMs Potentiate Conventional Antifungals In Vivo and Ex Vivo

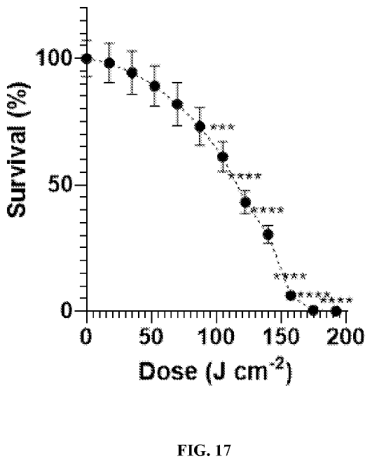

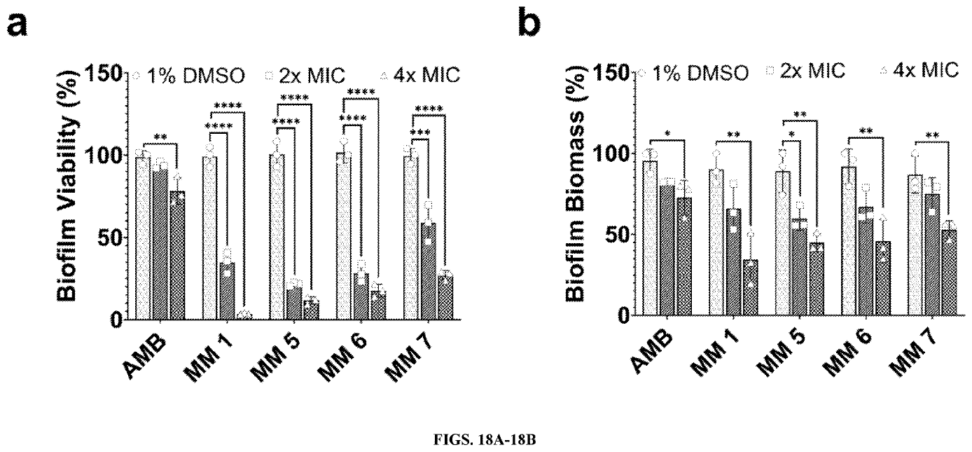

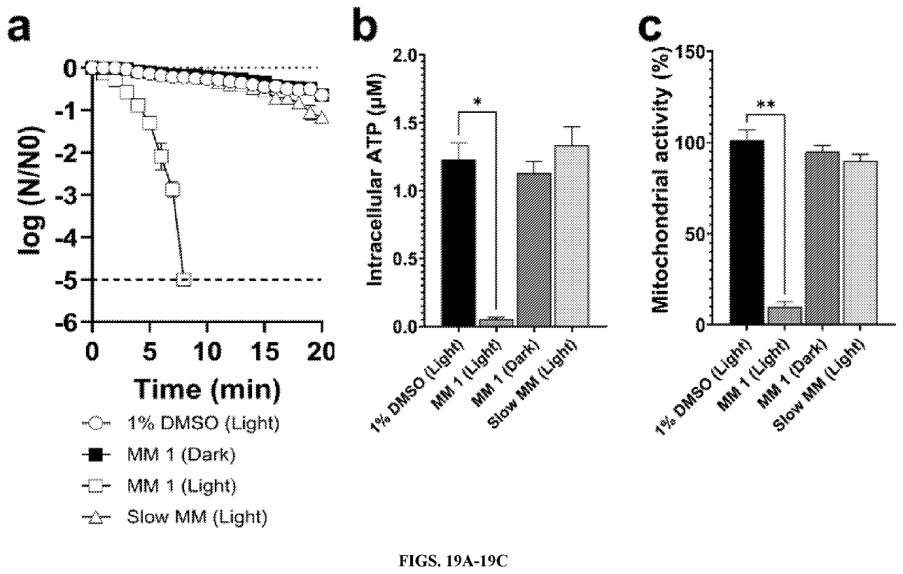

The toxicity of visible-light-activated MMs to mammalian cells was investigated in human embryonic kidney cells (HEK293T) treated with increasing MM concentrations and 87.6 J cm⁻² of 405-nm light. Vehicle-treated controls exposed to this light dose showed only a non-significant reduction in cell viability (FIG. 17). The MM concentration that reduced viability by 50% (IC₅₀), calculated from dose-response curves (FIG. 16C), ranged from 1.61-6.02 UM (FIG. 16D). The IC₅₀ and MIC were used to calculate the therapeutic index. With a therapeutic index ≥1 (FIG. 16D), MM 1 was used for in vivo and ex vivo studies.

The in vivo antibacterial activity of MM 1 was evaluated in a Galleria mellonella model of systemic infection with C. albicans or A. fumigatus. Infected worms were treated with 1% DMSO or MM 1 (1×MIC) with or without light or with conventional antifungals (1×MIC), namely, the polyene AMB and the azole fluconazole (FLC, C. albicans) or voriconazole (VRC, A. fumigatus). The effect of dual therapy combining light-activated MM 1 (1×MIC) and conventional antifungals (AMB or azole, 1×MIC) was also evaluated. Worm survival was monitored for 7 days, and fungal burden was assessed in a larval subset 48 h post-infection (FIG. 16E).

All C. albicans-infected worms treated with DMSO, MM, single antifungals, or MM plus fluconazole died within 3 days (FIG. 16F). However, MM 1+AMB significantly improved survival compared with individual treatments (p<0.0001), with ~17% of worms surviving to day 7 (FIG. 16F; Table 4). A significant reduction (p<0.01) in fungal burden was also observed in worms subjected to combination therapy compared with DMSO controls (FIG. 16G).

TABLE 4
Statistical comparison of survival curves of worms infected with C. albicans

| | 1% DMSO | | MM 1 | | | | MM 1 + | MM 1 + |
|---|---|---|---|---|---|---|---|---|
| | (Dark) | 1% DMSO | (No Light) | MM 1 | Amphotericin B | Azole | Amphotericin B | Fluconazole |
| 1% DMSO (Dark) | — | ns | ns | 0.0022 | 0.0232 | 0.0002 | <0.0001 | 0.0009 |
| 1% DMSO | — | — | 0.0324 | 0.001 | 0.0123 | <0.0001 | <0.0001 | 0.0032 |
| MM 1 (Dark) | — | — | — | 0.002 | 0.0198 | 0.0002 | <0.0001 | 0.0118 |
| MM 1 | — | — | — | — | ns | 0.0152 | 0.0001 | ns |
| Amphotericin B | — | — | — | — | — | 0.0114 | 0.0001 | 0.0440 |
| Fluconazole | — | — | — | — | — | — | 0.0002 | ns |
| MM 1 + Amphotericin B | — | — | — | — | — | — | — | 0.0122 |

Statistical comparison of survival curves of worms infected with C. albicans and treated with DMSO, MM 1, antifungal or a combination of MM 1 and antifungal using the log-rank (Mantel-Cox) test in GraphPad Prism 8.0 (San Diego, CA, USA). ns: non-significant.

TABLE 5
Statistical comparison of survival curves of worms infected with A. fumigatus

| | 1% DMSO | | MM 1 | | | | MM 1 + | MM 1 + |
|---|---|---|---|---|---|---|---|---|
| | (Dark) | 1% DMSO | (Dark) | MM 1 | Amphotericin B | Voriconazole | Amphotericin B | Voriconazole |
| 1% DMSO (Dark) | — | ns | ns | ns | ns | ns | 0.0096 | 0.006 |
| 1% DMSO | — | — | ns | ns | ns | ns | 0.0407 | 0.0278 |
| MM 1 (Dark) | — | — | — | ns | ns | ns | 0.0135 | 0.0067 |
| MM 1 | — | — | — | — | ns | ns | ns | ns |
| Amphotericin B | — | — | — | — | — | ns | ns | ns |
| Voriconazole | — | — | — | — | — | — | ns | ns |
| MM 1 + Amphotericin B | — | — | — | — | — | — | — | ns |

Statistical comparison of survival curves of worms infected with A. fumigatus and treated with DMSO, MM 1, antifungal or a combination of MM 1 and antifungal using the log-rank (Mantel-Cox) test in GraphPad Prism 8.0 (San Diego, CA, USA). ns: non-significant.

In A. fumigatus-infected worms, dual therapy (MM 1 plus antifungal) also improved survival compared with untreated samples (FIG. 16F). Moreover, MM 1+VRC significantly reduced (p<0.05) worm fungal burden compared with DMSO controls (FIG. 16G). However, statistically significant differences in the survival of worms subjected to dual therapy versus MM or antifungal alone were not detected (Table 5).

The ability of MMs to reduce fungal burden in mammals was investigated using an ex vivo onychomycosis porcine model infected with a strain of T. rubrum (ATCC 10218) isolated from a human onychomycosis case. T. rubrum-infected porcine nails were treated with 1% DMSO or MM 1 alone (0.77% (w/v) in DMSO) plus 405-nm light (87.6 J cm⁻²) or two formulations of the topical synthetic hydroxypyridone ciclopirox: a 0.77% “lotion” and an 8% “lacquer.” The effect of dual therapy (MM 1 plus ciclopirox) was also evaluated. Fungal load was assessed 5 days post-treatment (FIG. 16H). Compared with DMSO controls, MM 1 alone significantly reduced fungal burden by ~2 log₁₀ (FIG. 161). Dual therapy (MM 1+ciclopirox) performed significantly better than ciclopirox alone (p<0.001) but did not outperform MM 1 alone (FIG. 161; Table 6).

TABLE 6
Statistical comparison of fungal load in porcine nails infected with T. rubrum

| | Mean | | | Adjusted |
|---|---|---|---|---|
| Treatments compared | rank diff. | Significant? | Summary | P Value |
| 1% DMSO vs CPX “Lotion” | 13.33 | No | ns | >0.9999 |
| 1% DMSO vs CPX “Lacquer” | 13.67 | No | ns | 0.9794 |
| 1% DMSO vs MM 1 | 28.00 | Yes | ** | 0.0024 |
| 1% DMSO vs CPX “Lotion” + MM 1 | 37.72 | Yes | **** | <0.0001 |
| 1% DMSO vs CPX “Lacquer” + MM 1 | 42.28 | Yes | **** | <0.0001 |
| CPX “Lotion” vs CPX “Lacquer” | 0.3333 | No | ns | >0.9999 |
| CPX “Lotion” vs MM 1 | 14.67 | No | ns | 0.7188 |
| CPX “Lotion” vs CPX “Lotion” + MM 1 | 24.39 | Yes | * | 0.0151 |
| CPX “Lotion” vs CPX “Lacquer” + MM 1 | 28.94 | Yes | ** | 0.0014 |
| CPX “Lacquer” vs MM 1 | 14.33 | No | ns | 0.7983 |
| CPX “Lacquer” vs CPX “Lotion” + MM 1 | 24.06 | Yes | * | 0.0177 |
| CPX “Lacquer” vs CPX “Lacquer” + MM 1 | 28.61 | Yes | ** | 0.0017 |
| MM vs CPX “Lotion” + MM 1 | 9.722 | No | ns | >0.9999 |
| MM vs CPX “Lacquer” + MM 1 | 14.28 | No | ns | 0.8122 |
| CPX “Lotion” + MM vs CPX “Lacquer” + MM 1 | 4.556 | No | ns | >0.9999 |

Statistical comparison of fungal load in porcine nails infected with T. rubrum and treated with DMSO plus 405 nm light at 87.6 J cm⁻², visible-light-activated MM 1 plus 405 nm light at 87.6 J cm⁻², topical antifungal ciclopirox in two formulations (“lacquer” and “ lotion”), or a combination of visible-light-activated MM 1 plus 405 nm light at 87.6 J cm⁻² and ciclopirox using a Kruskal-Wallis test with Dunn's multiple comparison test in GraphPad Prism 8.0 (San Diego, CA, USA). Further experimental details are provided elsewhere in Example 1.
CPX: ciclopirox.
ns: non-significant.

### DISCUSSION

Herein is reported the use of synthetic 405-nm-visible-light-activated MMs to kill unicellular and multicellular planktonic fungi (FIG. 1C, FIG. 1D). At up to 2×MIC, killing was observed to depend entirely on light activation of the fast rotation rates of MMs (FIG. 1D; FIG. 2) and the presently disclosed methods may involve remotely controlled by adjusting the light dose, with higher light doses enhancing antifungal activity (FIG. 1F). In contrast to conventional antifungals, MM MIC remained stable over 20 cycles of repeated treatment (FIG. 11), suggesting, without being bound by theory, that resistance to MMs is not easily achieved.

In addition to planktonic cells, stimulus-activated MMs were also able, according to methods disclosed herein, to rapidly eliminate established biofilms of C. albicans, reducing both biofilm viability (FIG. 1G) and biomass (FIG. 1H) within minutes of light activation more efficiently than AMB for the same treatment time. Similar results were observed following treatment of biofilms of S. cerevisiae with light-activated MMs in accordance with presently disclosed methods (FIG. 18). Members of the Candida genus are the most common fungal species associated with biofilm infections of medical devices (Tsui et al., 2016), and biofilm formation is an important process associated with C. albicans virulence (Mayer et al., 2013). Bacteria in a biofilm can also detach from biological or artificial surfaces, enter the bloodstream, and migrate to other parts of the body through the process of hematogenous dissemination, leading to candidemia and septicemia. Fungal biofilms are highly resistant to antifungal drugs and host immune defenses, making the treatment of biofilm-associated infections particularly challenging (Tsui et al., 2016). The observed reduction in biofilm biomass and viability after treatment with MMs suggests, without being bound by theory, that the MMS as used according to the presently disclosed methods are not only capable of physically destroying the extracellular polymeric matrix of the biofilm, but also killing fungal cells within the biofilm.

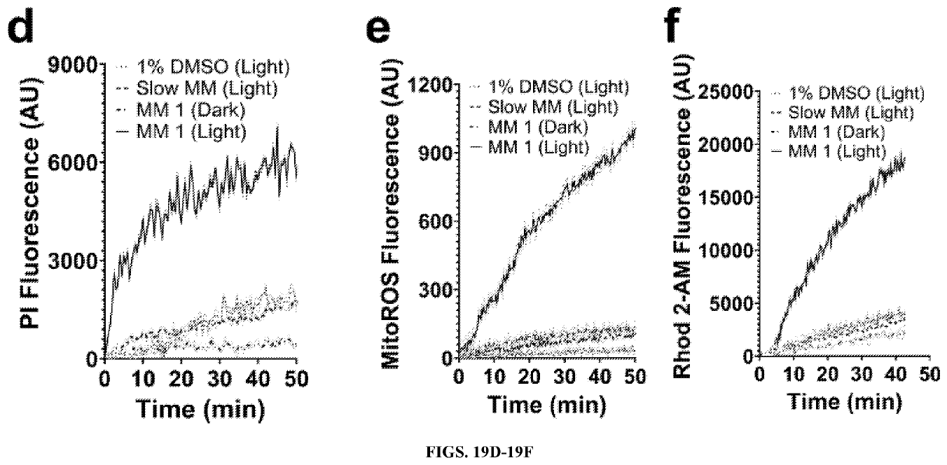

Mechanism of action studies in C. albicans showed that MMs bind the negatively charged mitochondrial phospholipids cardiolipin and phosphatidylglycerol (FIG. 4G), and confocal microscopy confirmed substantial (52.5%) colocalization of MMs with mitochondria (FIG. 4J, FIG. 4K), identifying mitochondria as the main cellular targets of MMs in fungi. Since light was omitted during colocalization experiments, binding of MMs to mitochondrial phospholipids occurs in the dark, possibly, without being bound by theory, through supramolecular interactions between the positively charged MM amine groups after protonation at biological pH (FIG. 1C) and the negatively charged phosphate groups of cardiolipin and phosphatidylglycerol. However, binding of MMs to mitochondria alone is not overtly detrimental (FIG. 19), and light was required to activate the rapid rotation of MMs bound to mitochondrial phospholipids to trigger antifungal activity.

The identification of cardiolipin and phosphatidylglycerol as MM targets reconciles findings known in the art and previous observations on the broad spectrum of biological activity of MMs, ranging from bacteria (Santos et al., 2022) to mammalian cells (García-López et al., 2017), as these phospholipids are common crucial components of all these organisms. Phosphatidylglycerol and cardiolipin are major components of the bacterial membrane but are mainly found in the mitochondrial membranes of eukaryotes, consistent with their endosymbiotic origin (Sagan, 1967). The distinct locations of these phospholipids in different organisms explain, with out being bound by theory, why MMs cause substantial damage to bacterial membranes (Santos et al., 2022) but produce predominantly intracellular effects in C. albicans.

By stabilizing the electron transport chain, cardiolipin is critical for mitochondrial function, and yeasts deficient in cardiolipin show impaired mitochondrial bioenergetics (Joshi et al., 2009). Therefore, binding of MMs to mitochondrial phospholipids and their subsequent activation by light could affect normal mitochondrial processes, as shown by decreased mitochondrial activity (FIG. 7A), intracellular ATP (FIG. 7B), and mitochondrial membrane potential (FIG. 7H, FIG. 71), as well as increased mitochondrial superoxide radical formation (FIGS. 7C-7E) in MM-treated cells.

In addition to their role in energy and ROS generation, in higher eukaryotes, mitochondria also modulate cellular calcium homeostasis due to their proximity to the endoplasmic reticulum, the main calcium reservoir (Giorgi et al., 2018). In yeast, the vacuole is the primary cellular calcium storage organelle, and the role of mitochondria in calcium homeostasis is unclear because there is no mitochondrial calcium uniporter or calcium-sensitive dehydrogenases (Pittman, 2011). However, calcium enters yeast mitochondria when cytosolic calcium levels increase (Carraro and Bernardi, 2016), and free fatty acids from mitochondrial phospholipid degradation have been shown to activate vigorous mitochondrial Ca²⁺: 2H⁺ antiporter activity (Bradshaw et al., 2001). The observations that MM treatment significantly increased intracellular calcium levels (FIG. 14B-14E) and that calcium chelation attenuated MM-induced killing (FIG. 14F) by lessening the MM-induced intracellular calcium increase (FIG. 14G, 14H) provide compelling evidence, without being bound by theory, that calcium overload is involved in the antifungal mechanism of action of MMs.

Elevated intracellular calcium levels in MM-treated cells can be attributed, without being bound by theory, to intracellular ATP depletion (FIG. 7B) resulting from mitochondrial dysfunction. Since intracellular calcium homeostasis depends on ATPases in the plasma membrane, vacuole, and other organelles (Martínez-Muñoz and Kane, 2008), ATP depletion leads to uncontrolled calcium uptake from the extracellular medium and its release from intracellular stores. This is followed by water influx leading to swelling of the cell and organelles, including mitochondria (FIG. 15B), which eventually burst and release the intramitochondrial contents into the cytoplasm, as indicated by a significant decrease in mitochondrial cytochrome C concentration in MM-treated cells.

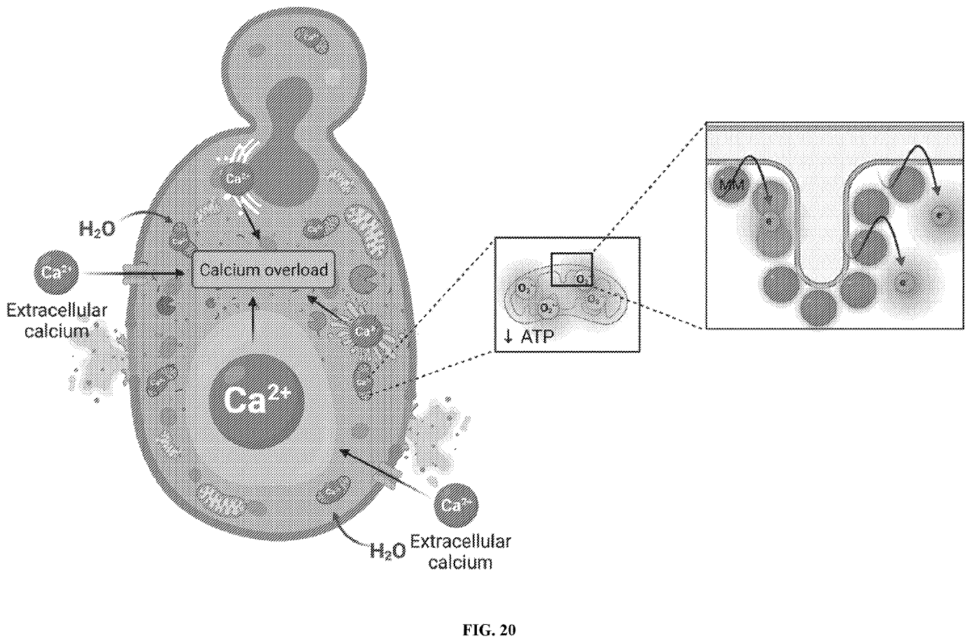

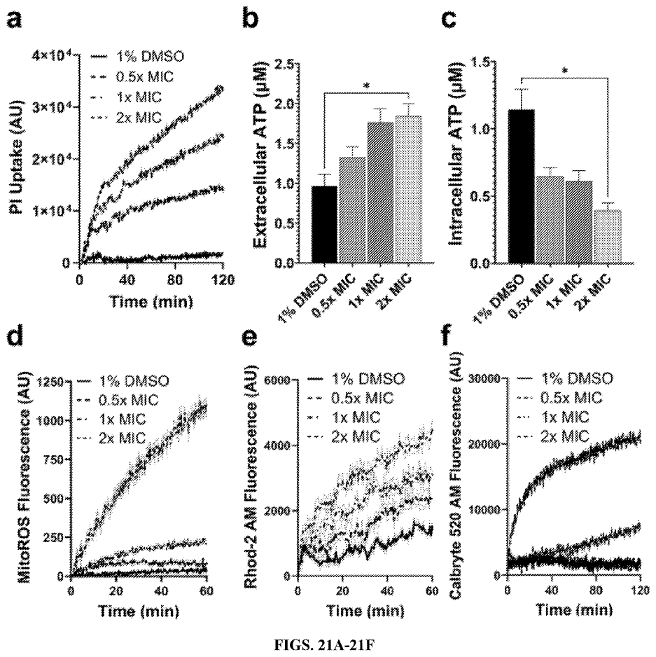

Damage to the plasma membrane, intracellular ATP depletion, leakage of cell contents, and swelling of mitochondria are common features of necrotic death (Eisenberg et al., 2010). The necrotic nature of MM killing was confirmed by the significant increase in the percentage of necrotic but not apoptotic cells after MM treatment (FIG. 15D, FIG. 15E). Overall, MM-induced fungal cell death via necrosis results from, without being bound by theory, the cumulative effects of oxidative stress and bioenergetic deficit triggered by light activation of MMs bound to mitochondrial phospholipids, leading to calcium overload and osmotic shock (FIG. 20), Because these processes occurred in C. albicans and S. cerevisiae (FIG. 21), the proposed antifungal mechanism of action of MMs appears to be conserved in yeast.

Unlike most conventional antifungals, which act on a single target in the cell, the involvement of widespread mitochondrial dysfunction and calcium overload in the mechanism of action of antifungal MMs may explain the inability to detect the development of resistance to MM treatment, as this damage cannot in principle be mitigated by one or a few concurrent mutations. Since MMs bind cardiolipin and phosphatidylglycerol and yeasts lacking both phospholipids are severely impaired or not viable (Gohil et al., 2005), simultaneous mutations in both phospholipids that could prevent MM binding and lead to resistance are unlikely.

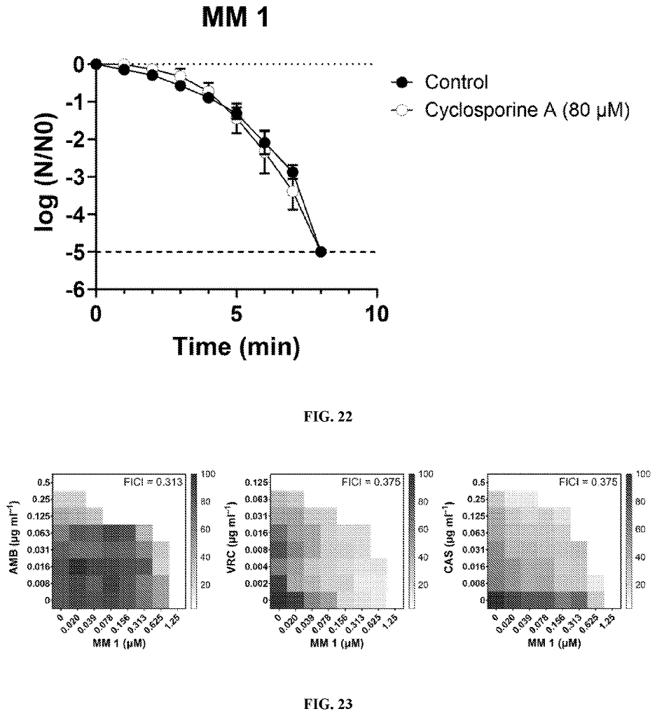

Importantly, the calcium dysfunction triggered by MMs is distinct from that involved in azole resistance (Liu et al., 2015). This is evidenced by the opposite role of calcium chelation and calcineurin in the action of azoles (Liu et al., 2015; Li et al., 2020; Juvvadi et al., 2017) compared with that of MMs (FIG. 14F; FIG. 22), which explains the lack of cross-resistance between MMs and azoles (Table 3).

In addition to their direct antifungal activity, visible-light-activated MMs synergized with conventional antifungals in C. albicans (FIG. 16A) and in S. cerevisiae (FIG. 23). This may be due, without being bound by theory, to the orthogonal targeting of different cellular processes by MMs and conventional antifungals (Jia et al., 2009). Photoinactivation of catalase by blue light (Dong et al., 2022) may also, without being bound by theory, sensitize cells to the deleterious effects of MMs. Moreover, the fluorescence of rhodamine 6G, a substrate of some of the energy-dependent efflux pumps whose overexpression has been associated with azole resistance (Parkinson et al., 1995; Clark et al., 1996), showed a significant decrease in MM-treated cells (FIG. 16B). These results suggest, without being bound by theory, that MMs also enhance the effect of conventional antifungal drugs by impairing the activity of energy-dependent efflux pumps. Enhanced efflux is an important mechanism by which microorganisms attenuate the effect of antimicrobials by reducing the amount of drug that accumulates in the cell (Cannon et al., 2009). Accordingly, inhibition of efflux pumps has been found to enhance the activity of antifungal drugs by increasing their intracellular levels (Iyer et al., 2020). The observed impairment of the activity of energy-dependent efflux pumps by MMs can be attributed, without being bound by theory, to the MM-induced decrease in intracellular ATP content (FIG. 7B), which is consistent with the previously reported increase in azole susceptibility of cells deprived of energy (Sun et al., 2013).

In vivo studies on the antifungal efficacy of MMs were performed on G. mellonella. G. mellonella is a simple invertebrate that has been used extensively as a model system for studying the in vivo efficacy of antifungal agents against Candida albicans (Li et al., 2013) and A. fumigatus (Slater et al., 2011). G. mellonella does not have adaptive immunity, but its innate immune system has similarities to that of vertebrates in terms of function and anatomy (Smith and Casadevall, 2021). Importantly, pathogenicity in mice and G. mellonella models of infections is correlated (Slater et al., 2011; Brennan et al., 2002), suggesting that findings from studies with G. mellonella are translatable to vertebrates.

Dual therapy of C. albicans- or A. fumigatus-infected worms with light-activated MMs and conventional antifungals improved survival (FIG. 16F) and reduced fungal burden (FIG. 16G) compared with vehicle-treated controls. In C. albicans, combination therapy with AMB and MM significantly improved survival compared with treatment with AMB or MM alone, suggesting a synergistic interaction between these antimicrobial modalities in vivo. Similarly, MM 1 potentiated the activity of the commonly prescribed antifungal agent ciclopirox (Gupta et al., 2018) in an ex vivo onychomycosis porcine model (FIG. 161).

Most conventional antifungal agents, such as AMB, exhibit severe toxicity leading to undesirable side effects (Stewart and Paterson, 2021). A therapeutic approach combining sublethal MMs to sensitize cells to conventional antifungals could mitigate the side effects of existing antifungal therapies. Moreover, the observation that MMs not only kill fungal cells directly but can also enhance the effect of conventional antifungal drugs by targeting a distinct process in the cell (i.e., intracellular calcium homeostasis) and/or preventing their efflux identifies MMs as dual mode-of-action antifungals that could provide a much-needed new therapeutic option to combat pan-resistant fungal strains such as C. auris (Kuehn, 2020), for which there are currently limited treatment options. MMs with improved safety profiles that specifically target fungal mitochondria can be developed by exploiting differences in the chemical composition of fungal and mammalian mitochondrial phospholipids (Schlame et al., 1993) and/or by modifying MMs with peptide addends that target mitochondrial proteins found in fungi but not in mammals, such as the fungal-type II NADH dehydrogenases (Melo et al., 2004).

### EXPERIMENTAL

### (i) Synthetic Chemistry

### Synthesis of MM 7

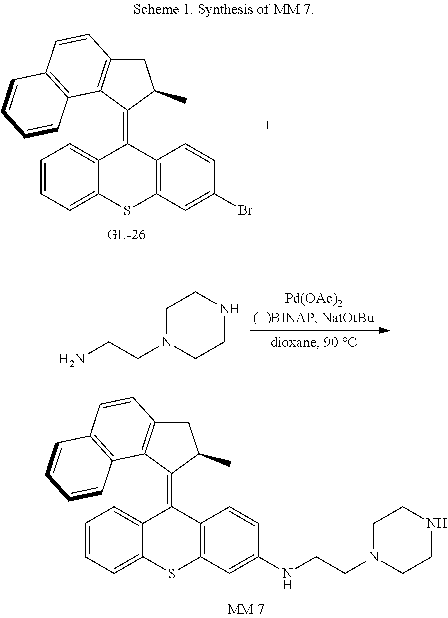

All glassware was oven-dried overnight prior to use. Reagent grade dichloromethane (DCM, CH₂Cl₂) was distilled from calcium hydride (CaH₂) under an N₂ atmosphere. All reactions were carried out under an N₂ atmosphere unless otherwise noted. All other chemicals were purchased from commercial suppliers and used without further purification.

GL-26 was synthesized according to previous literature (Saywell et al., 2016). (±) 2,2′-Bis (diphenylphosphino)-1,1′-binaphthyl (BINAP) (15 mg, 0.024 mmol) and palladium (II) acetate (1.8 mg, 0.008 mmol) were mixed in dry dioxane (4.5 ml) in an 8 ml vial. The solution was stirred for 20 min at room temperature. Afterward, NaO Bu (96 mg, 1 mmol), bromo-substituted motor GL-26 (91 mg, 0.2 mmol) and 2-(piperazin-1-yl) ethan-1-amine (129 mg, 1 mmol) were added. The mixture was stirred at 90 ° C overnight. Subsequently, the reaction mixture was cooled to room temperature and treated with H₂O (10 ml) and DCM (10 ml). The organic phase was separated with a separation funnel. The aqueous phase was extracted with DCM (3×10 ml), and the organic phases were combined and washed with H₂O (2×10 ml). After the volatiles were removed by rotary evaporation, the crude product was purified by column chromatography (silica gel, MeOH: DCM=10:90) to obtain MM 7 (9-(2-methyl-2,3-dihydro-1H-cyclopenta[a]naphthalen-1-ylidene)-N-(2-(piperazin-1-yl)ethyl)-9H-thioxanthen-3-amine) as a yellow solid product (yield: 90%, Z: E=5:5). 1H NMR (500 MHz, CD₂Cl₂) δ 7.82 (dd, J: =7.97, J2=1.43 Hz, 1H), 7.73-7.69 (m, 4H), 7.63-7.55 (m, 4H), 7.45 (dd, J1=8.12, J2=2.20 Hz, 2H), 7.36-7.32 (m, 1H), 7.24-7.13 (m, 3H), 7.01-6.98 (m, 1H), 6.89-6.84 (m, 4H), 6.79-6.76 (m, 1H), 6.70-6.68 (m, 1H), 6.65-6.63 (m, 1H), 6.62-6.58 (m, 1H), 6.50 (d, J=8.27 Hz, 1H), 5.92 (dd, J₁=8.33, J2=2.38 Hz, 1H), 4.29 (m, 2H), 3.65 (m, 2H), 3.20 (m, 2H), 3.08 (m, 2H), 2.93 (t, 4H), 2.91 (t. 4H), 2.67-2.56 (m, 10H), 0.76 (dd, J₁=22.10, J2=6.74 Hz, 6H). ¹³C NMR (125 MHz, CD₂Cl₂) δ: 147.3, 146.7, 145.7, 145.6, 144.4, 144.1, 141.1, 138.7, 136.4, 136.3, 135.7, 135.5, 135.3, 135.2, 132.9, 132.9, 129.3, 129.0, 128.9, 128.8, 128.6, 128.5, 128.2, 128.1, 127.6, 127.5, 127.3, 127.2, 126.7, 126.5, 126.1, 126.1, 125.9, 125.8, 125.6, 124.4, 124.3, 123.8, 123.8, 123.7, 111.6, 111.6, 110.5, 110.5, 56.8, 56.7, 53.9, 53.8, 53.7, 53.6, 53.4, 53.3, 53.1, 52.9, 45.4, 45.4, 40.0, 39.8, 39.6, 37.8, 37.8, 19.0, 18.9. HRMS (ESI) for C₃₃H₃₄N₃S [M+H]: 504.2473. Found: 504.2466. FTIR (KBr, cm⁻¹): 3048, 2952, 2837, 1599, 1581, 1495, 1455, 1435, 1397, 1308, 1263, 1226, 1156, 1136, 1098, 1049, 1030, 946, 809, 783, 735, 713, 648.

Information on the synthesis and characterization of the other MMs investigated in this study can be found elsewhere (Santos et al., 2022).

### (i) Strains and Reagents

Five fungal strains were used in this study: the yeast Saccharomyces cerevisiae (ATCC 13007), the yeast-like fungus Candida albicans (ATCC 18804), and the molds Aspergillus fumigatus (ATCC 1022), Microsporum gypseum (ATCC 10215), and Trichophyton rubrum (ATCC 10218). All fungi were obtained from ATCC(Manassas, VA, USA).

Unless otherwise noted, all chemicals were purchased from MedChem Express (Princeton, NJ, USA), Caymanchem (Ann Arbor, MI, USA), or Millipore-Sigma (St. Louis, MO, USA) and prepared in 100% DMSO or an appropriate solvent, per the distributor's instructions.

(iii) Antifungal Susceptibility Testing

Cell suspensions for susceptibility testing (MMs and conventional antifungals) were prepared per the Clinical & Laboratory Standards Institute (CLSI) guidelines (CLSI, 2017; CLSI, 2008). Before testing, yeasts (C. albicans and S. cerevisiae) were sub-cultured in Sabouraud Dextrose Agar-Emmons Modification (SDAE) plates and grown for 24 h at 30 °C. Five independent colonies from 24-h-old plates were collected and diluted to ~10⁴ colony forming units (CFU) per mL in sterile saline (CLSI, 2017). Molds (A. fumigatus and the dermatophytes T. rubrum and M. gypseum) were sub-cultured on SDAE medium and incubated for 7 days at 28 °C. Conidia were recovered by covering the plates with sterile distilled water and scraping the colonies. The suspensions were filtered (8-μm pore size) and diluted in saline to ~10⁴ CFU mL⁻¹ (Santos and Hamdan, 2005).

For MM MIC determination, increasing concentrations (0.3125-160 μM) of different MMs (8 mM stock in DMSO) were added to the cell suspensions. After a 30-min incubation in the dark, cell suspensions were transferred to small, sterilized glass beakers, which were then placed in a water bath. Each sample was irradiated with 405-nm light at 292 mW cm⁻² for 5 min, corresponding to a light dose of 87.6 J cm⁻², determined using an S415C thermal power sensor (Thorlabs, Newton, MA, USA). During irradiation, the cell suspensions were agitated with a small metal stirrer. A thermocouple probe (model SC-TT-K-30-36-PP; Omega Engineering, Inc., Stanford, CT, USA) was used to monitor the temperature during irradiation. Irradiated cell suspensions were inoculated in 3-(N-morpholino) propanesulfonic acid (MOPS)-buffered Roswell Park Memorial Institute Medium (RPMI) 1640 (pH 7.0). Tubes were incubated at 30 ° C for 48 h (yeasts) and 28 ° C for 7 days (molds). The antifungal or MM concentration resulting in no visible growth was defined as the minimum inhibitory concentration (MIC) (CLSI, 2017; CLSI, 2008). Similarly prepared cell suspensions were used to determine the MIC of conventional antifungals.

Aliquots (100 μL) of MIC tubes without visible fungal growth were plated on SDAE medium. Plates were incubated at 30 ° C for 48 h with confirmation after 72 h (yeasts) and for 7 days at 28 ° C with confirmation after 14 days (molds). The lowest concentration that killed ≥99.9% of the original inoculum was defined as the minimum fungicidal concentration (MFC) (Guinea et al., 2008).

### (iv) Time-Kill Assays

For yeasts, five independent colonies were collected from 24-h SDAE plates, inoculated into yeast peptone with 2% dextrose (YPD), and grown for 24 h at 30 °C. Cells were then sub-cultured in fresh medium and grown for ~9 h. Afterward, the cells were centrifuged (5,000 ×g, 5 min), washed, and resuspended in phosphate-buffered saline (PBS) to ~10⁶ CFU mL⁻¹. For A. fumigatus, conidia suspensions (~10⁴ CFU mL⁻¹) were prepared in PBS as previously described.

Cell/conidia suspensions were treated with 1% DMSO or MMs (2×MIC) and, after a 30-min dark incubation, irradiated (405-nm light at 292 mW cm⁻²) as previously described. Similarly processed samples treated with a slow MM (10 μM, corresponding to the maximum MM MIC detected across all fungal strains) served as a control for the effects of MM rotation speed on antifungal activity. Amphotericin B (AMB, 4×MIC) controls were prepared likewise, but light was omitted. Aliquots were collected in 1-min increments for up to 10 min, serially diluted in PBS, and plated on SDAE medium. Plates were incubated at 30 ° C for 48 h with confirmation after 72 h (yeasts) or at 28 ° C for 7 days with confirmation after 14 days (A. fumigatus), after which the CFU number was determined. The results were expressed as the logarithm of base 10 of the ratio between the CFU at each time point and the CFU at time 0. The detection limit of the method was ~1 log₁₀ CFU mL⁻¹.

(v) Biofilm viability and biomass

The antibiofilm activity of MMs was investigated using 96-well microtiter plates with flat-bottom wells as a closed static biofilm reactor. This setup is reliable, inexpensive, easy to use and obtain, and requires no additional equipment (Pierce et al., 2010). Two parameters were used to evaluate antibiofilm activity: biofilm biomass and biofilm viability. Biofilm biomass was determined using the crystal violet method (Martins et al, 2010), a simple, inexpensive, and readily accessible method for determining biofilm biomass. However, because crystal violet binds both live and dead cells as well as extracellular polymeric substances, it cannot be used alone to reliably assess antibiofilm activity. To overcome this limitation, the XTT assay was used to evaluate biofilm viability (Nett et al., 2011). This assay is based on the reduction of the tetrazolium salt XTT to formazan by dehydrogenases in the mitochondrial electron transport chain of living cells. The resulting formazan can be easily detected by measuring the absorbance at 490 nm, which is proportional to the number of living cells, providing a reliable quantitative measurement of metabolically active cells in biofilms (Taff et al., 2012).

C. albicans biofilms were established in 96-well flat-bottom polystyrene plates (Corning-Costar Corp., Corning, NY, USA) by diluting 24-h cultures in fresh MOPS-buffered RPMI 1640. After 48 h at 30 ° C, mature biofilms were washed with PBS and treated with AMB (2× or 4×MIC), 1% DMSO, or different MMs (2× or 4×MIC). DMSO- and MM-treated samples were then irradiated in situ with 405-nm light (87.6 J cm⁻²).

Biofilm viability was determined using an XTT cell viability assay kit (Biotium, Hayward, CA, USA) per the manufacturer's instructions. Absorbance (490 nm) and background (640 nm) were read in a microplate reader (BioTek Instruments Inc., Winooski, VT, USA). Normalized absorbance values were obtained by subtracting the background from the signal.

Biofilm biomass was determined by the crystal violet method, as previously described (Martins et al., 2010). The absorbance of the supernatant at 550 nm was determined in a microplate reader (BioTek Instruments Inc., Winooski, VT, USA). Untreated sample values minus background were defined as 100% and used to calculate biofilm viability and biomass reduction after treatment.

### (vi) Development of Resistance to Visible-Light-Activated MMs

A modified version of the broth microdilution serial passage method was used to assess the development of resistance to visible-light-activated MMs in C. albicans (Kapoor et al., 2019). C. albicans cell suspensions were prepared and irradiated as previously described for the determination of MM MIC. Cells were then inoculated into buffered RPMI 1640 and incubated at 30 ° C for 48 h. Cells able to grow at 0.5×MIC of MM were centrifuged (5,000×g, 5 min), resuspended, rechallenged with different MM concentrations, and irradiated with 405-nm light (87.6 J cm⁻²). The procedure was repeated for 20 consecutive cycles. The antifungals AMB, CAS, and FLC were processed similarly, except that light was omitted, and used as controls.

(vii) Plasma Membrane Permeability

The effects of MMs on plasma membrane permeability were determined by monitoring PI uptake (Ma et al., 2020) and calcein leakage (Edgerton et al., 1998).

For PI uptake, C. albicans cells were grown as described for time-kill experiments, centrifuged (5,000 ×g, 5 min), washed, and resuspended in 5 mM glucose and 5 mM 4-(2-hydroxyethyl)-1-piperazineethanesulfonic acid (HEPES) buffer (pH 7.2). Cell suspensions (~10⁶ CFU mL-1) were treated with 1% DMSO or visible-light-activated MMs (0.5-2×MIC) and then irradiated with 405-nm light (87.6 J cm⁻²). After irradiation, PI (10 AM final concentration) was added to the cells. PI-labeled cells were transferred to a black 96-well plate, and PI fluorescence (excitation: 535 nm, emission: 617 nm) over time was monitored in a microplate reader (BioTek Instruments Inc., Winooski, VT, USA).

For calcein leakage assays, C. albicans cells (~10⁶ CFU mL-1), grown as described for time-kill experiments, were centrifuged (5,000×g, 5 min), washed, and resuspended in assay buffer (20 mM MOPS sodium salt, 1 mM CoCl₂, 90 mM NaCl, pH 7.5) containing 0.8 mM calcein-AM. After a 2-h incubation at 30 ° C, calcein-loaded cells were diluted (~105 CFU mL⁻¹) in assay buffer, treated with MMs (0.5-2×MIC) or 1% DMSO and irradiated with 405-nm light (87.6 J cm⁻²). Afterward, the cells were centrifuged (5,000×g, 5 min) and resuspended in assay buffer. At least 10,000 cells were then analyzed in a Sony SA3800 spectral analyzer (Sony Biotechnology, CA, USA).

### (ix) Intracellular and Extracellular ATP

C. albicans cell suspensions (~10⁶ CFU mL⁻¹) were treated with 1% DMSO or MMs (0.5-2×MIC) and irradiated with 405-nm light (87.6 J cm⁻²), as described above. Following centrifugation (5,000×g, 5 min), extracellular and intracellular ATP was extracted from the supernatant and pellet, respectively, as previously described (Koshlukova et al., 1999).

ATP concentrations were measured using the CellTiter-Glo Luminescent Cell Viability Assay (Promega, Madison, WI, USA) per the manufacturer's instructions. The luminescent signal was measured using a microplate reader (BioTek Instruments Inc., Winooski, VT, USA) and converted to ATP concentration by linear regression of a standard ATP curve prepared using adenosine 5′-triphosphate disodium salt trihydrate. ATP levels were normalized to the protein concentration determined using the Pierce Assay (Pierce™ BCA Protein Assay Kit, Thermo Fisher Scientific, MA, USA).

### (x) Plasma Membrane Fluidity

The effects of MMs on C. albicans membrane dynamics were evaluated using DPH fluorescence (Kim et al., 2009). C. albicans cell suspensions (~10⁶ CFU mL⁻¹) were prepared, treated with 1% DMSO or MMs (0.5-2×MIC), and then irradiated with 405-nm light (87.6 J cm⁻²). AMB-treated cells were used as controls. Samples were fixed with 0.37% formaldehyde and labeled with 0.6 mM DPH, as previously described (Kim et al., 2009). DPH fluorescence (excitation: 350 nm, emission: 420 nm) was measured in a microplate reader (BioTek Instruments Inc., Winooski, VT, USA). DPH fluorescence of untreated samples minus background was defined as 100% and used to calculate changes in treated samples.

(xi)Competition Assays with Exogenous Ergosterol and Phospholipids

Competition assays with exogenous ergosterol and phospholipids were performed as previously described (de Castro Spadari et al., 2018) with modifications. C. albicans cell suspensions (~10⁶ CFU mL⁻¹) were prepared as described for time-kill assays to which increasing concentrations (up to 100 μg mL⁻¹) of exogenous ergosterol or the phospholipids phosphatidylcholine, phosphatidylethanolamine, phosphatidylglycerol or cardiolipin (Avanti Polar Lipids, AL, USA) were added. Increasing concentrations of MM were then added to each ergosterol- and phospholipid-treated sample. After a 30-min dark incubation, the samples were irradiated with 405-nm light (87.6 J cm⁻²) as previously described. Buffered RPMI 1640 medium was then added to the irradiated samples. After incubation at 30 ° C for 48 h, samples were examined for growth to determine the MM MIC.

### (xi) Electron Microscopy

C. albicans cell suspensions (~10⁶ CFU mL⁻¹) were prepared in PBS (1×) as described for time-kill assays, treated with 1% DMSO or 0.5×MIC MM 1, and then irradiated with 87.6 J cm-2 405-nm light. Irradiated cells were fixed with Karnovsky's fixative, postfixed with 1% osmium, and dehydrated with a series of ethanol washes. For TEM, specimens were embedded in epoxy resin (PolyBed 812; Polysciences, Inc., Warrington, PA, USA) after being dehydrated in a series of washes with a graded concentration of 50-100% ethanol. A Leica EM UC7 ultramicrotome (Leica Microsystems, Wetzlar, Germany) was used to cut ultrathin sections (65 nm), which were then poststained with uranyl acetate and lead citrate. Samples were observed using a JEOL JEM2100 TEM (Hitachi Corporation, Japan) operating at an accelerating voltage of 80 kV. For SEM, after dehydration with ethanol, samples were dried with a Leica EM CPD300 (Leica Microsystems, Wetzlar, Germany) at the critical point, sputter-coated with 10 nm gold, and imaged with an FEI Apreo SEM (FEI Apreo, ThermoFisher Scientific, Waltham, MA, USA) using a secondary electron detector.

(xiii)Colocalization Analysis

Colocalization analysis of MMs was performed as previously described (Benhamou et al., 2018; Vida and Emr, 1995) with modifications. A single isolated colony was picked from 24-h SDAE plates, diluted in liquid YPD, and grown at 30 ° C for 24 h. Cells were then re-diluted in fresh YPD medium and grown statically in Ibidi u-dishes (Ibidi GmbH, Munich, Germany) for 24 h at 30 ° C The cells were washed, and then YPD medium containing 8 μM MM 1 and 10 nM MitoTracker™ Green (Thermo Fisher Scientific, MA, USA) was added. After a 30-min dark incubation at 30 ° C, the solution was replaced with fresh medium containing 40 nM FM™ 4-64 (Thermo Fisher Scientific, MA, USA). Cells were immediately imaged in a Nikon Al—RSI confocal system mounted on a Nikon Ti-E widefield fluorescence microscope (Nikon Corporation, NY, USA). Cells were imaged directly on the Ibidi imaging dish using a 60×water immersion objective (numerical aperture of 1.27, 0.17 mm working distance). Colocalization was calculated in the Fiji version of ImageJ using the Colocalization Threshold tool and the Coloc-2 plugin.

(xiv) Mitochondrial Activity

The effect of visible-light-activated MMs on mitochondrial activity was assessed using XTT, which is metabolically reduced by mitochondrial dehydrogenases (Wu et al., 2009).

C. albicans cell suspensions (~10⁶ CFU mL⁻¹), prepared as described for time-kill experiments, were treated with 1% DMSO or MMs (0.5-2×MIC) and irradiated with 405-nm light (87.6 J cm⁻²). Irradiated cells were mixed with 25 μL of activated XTT working solution (Biotium, Hayward, CA, USA) in a 96-well plate. After 4 h at 30 ° C, the absorbance (490 nm) and background (640 nm) were measured in a microplate reader (BioTek Instruments Inc., Winooski, VT, USA). The absorbance of untreated samples minus background was defined as 100% and used to calculate the reduction in mitochondrial activity.

### (xv) Mitochondrial ROS

C. albicans cell suspensions (~10⁶ CFU mL⁻¹) prepared as described above were treated with 1% DMSO or MMs (0.5-2×MIC) and then irradiated with 405-nm light (87.6 J cm⁻²). Afterward, the cells were centrifuged (5,000×g, 5 min), washed, and resuspended in PBS (~10⁶ cells mL⁻¹). Mitochondrial ROS were quantified using the fluorescent superoxide radical-sensitive probe MitoROS™ 580 (AAT Bioquest, CA, USA) per the distributor's instructions. The fluorescence of MitoROS™ 580 (excitation: 510 nm, emission: 580 nm) over time was monitored in a microplate reader (BioTek Instruments Inc., Winooski, VT, USA).

Mitochondrial ROS generation was also monitored by confocal microscopy. Cells were prepared as previously described for colocalization analysis and then mixed with an equal volume of 2×MitoROS™ 580 working solution in Hank's Balanced Salt Solution with 20 mM HEPES (HHBS) buffer containing 1.25 AM MM 1. After a 30-min dark incubation, the solution was removed and replaced with fresh HHBS buffer. Cells were immediately imaged under a Nikon Al confocal microscope (Nikon Corporation, NY, USA) as previously described. MM light activation was performed in situ with a SOLA LED using a DAPI excitation filter (395/25 nm, 166 mW cm⁻²) for 5 min. Fluorescence intensities were extracted from microscopy images using FIJI's built-in algorithms.

(xvi) Superoxide Dismutase (SOD) Activity and Lipid Peroxidation

C. albicans cell suspensions (~10⁶ CFU mL⁻¹) were prepared as described above, challenged with 1% DMSO or MMs (0.5-2×MIC), and then irradiated with 405-nm light (87.6 J cm⁻²), after which the cells were centrifuged (5,000 ×g, 5 min). Superoxide dismutase (SOD) activity was determined using a Superoxide Dismutase Assay Kit (Caymanchem, MI, USA) per the distributor's instructions. Lipid peroxidation was determined using a TBARS assay kit (TCA method) (Caymanchem, MI, USA) per the distributor's instructions. SOD activity and MDA levels were normalized by protein content determined by the Pierce assay (Pierce™ BCA Protein Assay Kit, Thermo Fisher Scientific, MA, USA).

(xvii) Mitochondrial Membrane Potential

Changes in mitochondrial membrane potential were determined by monitoring the fluorescence shift of the ratiometric mitochondrial membrane potential probe JC-1 (Pina-Vaz et al., 2001). C. albicans cell suspensions (~10⁶ CFU mL⁻¹) were treated with DMSO or MMs (0.5-2×MIC), irradiated with 405-nm light (87.6 J cm⁻²), and then labeled with 5 μM JC-1 (ABP Biosciences, MD, USA) per the distributor's instructions. At least 10,000 cells per sample were then analyzed in a SA3800 Spectral Analyzer (Sony Biotechnology, CA, USA).

(xviii) Intracellular Calcium Levels

Calcium levels were measured using the fluorescent probes Calbryte™ 520 AM (AAT Bioquest, CA, USA) and Rhod-2 AM (AAT Bioquest, CA, USA) to determine cytosolic and mitochondrial calcium levels, respectively (Lee and Lee, 2014; Tian et al., 2017). C. albicans cell suspensions (~10⁶ CFU mL⁻¹) were prepared in HHBS containing 0.04% Pluronic® F-127 (AAT Bioquest, CA, USA) and labeled with Rhod-2 AM or Calbryte™ 520 AM (4 μM final concentration). After a 30-min dark incubation at 30 ° C, 1% DMSO or MMs (0.5-2×MIC) was added. Following an additional 30-min incubation, the cells were centrifuged (5,000×g, 5 min), resuspended in HHBS, and irradiated with 405-nm light (87.6 J cm⁻²). Afterward, the cells were centrifuged (5,000×g, 3 min) and resuspended in HHBS. The fluorescence of Calbryte™ 520 AM (excitation=490 nm, emission=525 nm) and Rhod-2 AM (excitation=540 nm, emission=590 nm) over time was monitored in a microplate reader (BioTek Instruments Inc., Winooski, VT, USA) or by flow cytometry in a SA3800 Spectral Analyzer (Sony Biotechnology, CA, USA).

Calcium levels were also monitored by live-cell calcium imaging using confocal microscopy. Cells were grown as described for colocalization experiments. The growth medium was then replaced with fresh HHBS buffer containing Rhod-2 AM (4 AM final concentration), to which MM 1 (1.25 μM) was added. After a 30-min dark incubation, the solution was replaced with fresh HHBS. Cells were immediately imaged using a Nikon Al confocal microscope (Nikon Corporation, NY, USA) directly on the Ibidi imaging dish with a 60×water immersion objective. MM light activation was performed in situ with a SOLA LED using a DAPI excitation filter (395/25 nm, 166 mW cm⁻²). Light was delivered through the microscope objective for 5 min, after which fluorescence was monitored for 60 additional minutes. Fluorescence intensities were extracted from microscopy images using FUJI's built-in algorithms.

(xix) Influence of BAPTA-AM on MM-Induced Killing and Intracellular Calcium Levels

C. albicans cells were grown as described above and resuspended in HHBS (~10⁶ CFU mL⁻¹). The cation chelator 1,2-bis(2-aminophenoxy) ethane-N,N,N′,N′-tetraacetic acid acetoxymethyl ester (BAPTA-AM) was then added (0.25-1 mM, final concentration) (Li et al., 2020). Unamended cells were used as controls. After a 30-min dark incubation at 30 ° C, the cells were centrifuged (5,000×g, 5 min), washed, and resuspended in HHBS. MM 1 (2× MIC) was then added. After 30 min, the cells were irradiated and processed as described for the time-kill experiments.

Intracellular calcium levels in untreated cells or cells treated with BAPTA-AM (1 mM) and then treated with 1% DMSO or different concentrations of MMs (0.5-2×MIC) plus 405-nm light (87.6 J cm⁻²) were determined using the probes Calbryte™ 520 AM and Rhod-2 AM, as described above.

### (xx) Mitochondrial Mass/Volume

Mitochondrial mass/volume was estimated using MitoTracker™ Green fluorescence (Puleston, 2015). C. albicans cell suspensions (~10⁶ CFU mL⁻¹) were treated with DMSO or MMs (0.5-2×MIC) and then irradiated with 405-nm light (87.6 J cm⁻²). The cells were then stained with Mito Tracker™ Green (200 nM) for 30 min at 30 ° C and washed three times with PBS. At least 10,000 cells per sample were analyzed in a SA3800 Spectral Analyzer (Sony Biotechnology, CA, USA).

(xxi)Cytochrome c Release

C. albicans cell suspensions (~10⁶ CFU mL⁻¹) were treated with DMSO or MMs (0.5-2×MIC) and irradiated with 405-nm light (87.6 J cm⁻²). Cells were harvested for protoplast preparation by digestion with zymolyase 20 T (20 mg mL-1, US Biological Life Sciences, MA, USA) in 0.1 M potassium phosphate buffer (pH 6.0) containing 1 M sorbitol for 1 h at 30 °C. Mitochondrial cytochrome c was extracted and reduced with ascorbic acid (0.5 mg mL⁻¹) as previously described (Yun and Lee, 2016). The absorbance at 550 nm was determined on a Beckman Coulter DU 800 spectrophotometer (Fullerton, CA, USA). Cytochrome c levels were normalized to the protein content determined using the Pierce assay (Pierce™ BCA Protein Assay Kit, Thermo Fisher Scientific, MA, USA).

(xxi) Detection of Necrosis and Apoptosis

The occurrence of necrosis and apoptosis was investigated using an Annexin V-FITC/PI assay (Van Genderen et al., 2006). C. albicans cells were grown as described for time-kill experiments, washed in sorbitol buffer (0.5 mM MgCl₂, 35 mM potassium phosphate, pH 6.8, containing 1.2 M sorbitol), and resuspended in the same buffer containing zymolyase 20 T (20 mg mL-1, US Biological Life Sciences, MA, USA). After 1 h of digestion at 30 ° C, protoplasts were centrifuged, washed, and resuspended in binding buffer (140 mM NaCl, 10 mM HEPES, 2.5 mM CaCl₂, 1.2 M sorbitol, pH 7.4). Protoplasts were treated with 1% DMSO or MMs (0.5-2×MIC) and then irradiated with 405-nm light (87.6 J cm⁻²). The protoplasts were immediately labeled using an Annexin V-FITC/PI Apoptosis Kit (Abnova, Taiwan) per the distributors' instructions. At least 10,000 cells per sample were analyzed in a SA3800 spectral analyzer (Sony Biotechnology, CA, USA).

(xxiii) Interaction Between Visible-Light-Activated MMs and Conventional Antifungals

The interaction of MMs with conventional antifungal agents in C. albicans was investigated by determining the MIC of different antifungals alone and after treatment with visible-light-activated MMs using a modified broth microdilution checkerboard assay (Cantón et al., 2005) in an 8×8-well configuration. C. albicans cell suspensions were prepared as described for MIC determination and treated with increasing concentrations (up to 1×MIC) of MMs. Following irradiation (87.6 J cm⁻² of 405-nm light), cells were collected and distributed along the x-axis of a 96-well plate. Increasing concentrations (up to 1×MIC) of different antifungal drugs (Table 2) in geometric twofold increments in buffered RPMI 1640 medium were added along the plate's y-axis. After 48 h at 30 ° C, the absorbance at 630 nm was measured in a microplate reader (BioTek Instruments Inc., Winooski, VT, USA). The fractional inhibitory concentration index (FICI) was determined as the sum of the MIC of the MM and the antifungal drug when used in combination divided by their MIC when used alone. An FICI index of ≤0.5, 0.5<x≤4, or >4 indicates synergistic, additive, and antagonistic interactions, respectively (Odds, 2003).

(xxiv) Efflux Activity

Efflux pump activity was evaluated by measuring the energy-dependent efflux of the fluorescent dye rhodamine 6G (Maesaki et al., 1999). C. albicans cells were grown overnight (~16 h) in YPD at 30 ° C, rediluted in fresh YPD, and grown for an additional 3 h at 30 °C. The cells were then centrifuged, washed with 50 mM HEPES buffer (pH 7.0), and resuspended in de-energization buffer containing 1 μM antimycin A and 5 mM 2-deoxy-D-glucose in 50 mM HEPES buffer (pH 7.0). After 3 h at 30 ° C, the cells were centrifuged, washed, and resuspended in cold 50 mM HEPES buffer (pH 7.0). The cells were then incubated with rhodamine 6G (10 AM final concentration) for 2 h at 30 ° C Afterward, the cells were centrifuged (1,000 ×g, 5 min), washed, and resuspended in cold HEPES buffer. Cells were then treated with 1% DMSO or MMs (0.5-2×MIC) and irradiated with 405-nm light (87.6 J cm⁻²). Irradiated cells were collected and incubated in prewarmed HEPES buffer containing 1 mM glucose for 1 h at 30 ° C to reactivate the cells. Afterward, the cells were centrifuged (1,000 ×g, 5 min), resuspended in HEPES buffer, and transferred to a 96-well plate. Rhodamine 6G fluorescence (excitation: 485 nm, emission: 535 nm) over time was measured in a microplate reader (BioTek Instruments Inc., Winooski, VT, USA). Rhodamine 6G-free cells served as unstained controls. Untreated sample fluorescence minus background was defined as 100% and used to normalize the remaining data points.

(xxx) Toxicity Profiling and Therapeutic Index Calculation

The biocompatibility of MMs with primary HEK293T cells was assessed using the CellTiter-Glo® Luminescent Cell Viability Assay (Promega, WI, USA) per the manufacturer's instructions by treating cells with increasing concentrations of different MMs plus 405-nm light (87.6 J cm⁻²). Dose-response curves were used to determine the MM concentrations that reduced cell viability by 50% (IC₅₀). The therapeutic index was calculated as the ratio between the IC₅₀ and the MIC.

(xxvi) In Vivo Antifungal Activity of MMs

The in vivo antifungal activity of MMs was assessed in G. mellonella (Li et al., 2013; (Slater et al., 2011). G. mellonella were acquired from a commercial supplier (rainbowmealworms.net) in their final instar larval stage. Worms of similar size (~0.3 g), responsive to touch, and displaying no signs of melanization were selected. C. albicans (~105 CFU mL⁻¹) and A. fumigatus conidia (~104 conidia mL⁻¹) suspensions were prepared in PBS as previously described. The fungal inoculum (5 μL) was injected into the last left proleg of the worms with a Hamilton syringe. Thirty minutes after infection, MM and/or antifungal agents (1×MIC, Table 2) diluted in sterile water were injected similarly to the right proleg. The following treatment groups (eight individuals each, from three independent batches) were established: (1) 1% DMSO with and without light, (2) monotherapy with MM 1 alone (1×MIC) with and without light, (3) monotherapy with conventional antifungals (1×MIC) amphotericin B (AMB) or azole (fluconazole, FLC, in the case of C. albicans and voriconazole, VRC, in the case of A. fumigatus), or (4) combination therapy with visible-light-activated MM 1 (1×MIC) followed by treatment with conventional antifungal (1×MIC). After 30 min, worms in the irradiated treatment groups were transferred to 24-well plates (Corning-Costar Corp., Corning, NY, USA) and irradiated with 405-nm light (87.6 J cm⁻²). Worms were incubated in sterile Petri dishes at 30 ° C in the dark. Live and dead worms were scored each day for 7 days. Melanized or unresponsive worms were considered dead.

Fungal load was assessed in a separate group of similarly treated worms 48 h after infection. Only healthy larvae (four worms per treatment group) with no melanization spots were used. After weight determination, worms were killed by freezing and homogenized using a tissue grinder (Fisherbrand, Fisher Scientific, Pittsburgh, PA, USA). For C. albicans-infected worms, after homogenization in sterile PBS, serial dilutions were plated on YPD agar containing antibiotics (100 μg mL⁻¹ ampicillin, 100 μg mL⁻¹ streptomycin, and 45 μg mL⁻¹ kanamycin) (Li et al., 2013). For A. fumigatus-infected worms, after homogenization in sterile PBS containing gentamicin (25 μg mL⁻¹) and chloramphenicol (400 μg mL⁻¹), serial dilutions were plated on potato dextrose agar (PDA) (Sheppard et al., 2006). After 48 h at 30 ° C (C. albicans) or 7 days at 28 ° C (A. fumigatus), colonies were counted to determine CFU per mg of larvae.

Work on G. mellonella was reviewed and approved by the Office of Sponsored Projects and Research Compliance (SPARC) at Rice University.

(xxvii) Ex Vivo Model of Onychomycosis

For microconidia preparation, T. rubrum was inoculated on potato dextrose agar containing 0.025% Sabouraud dextrose broth (SDB) and 1.0% penicillin-streptomycin. After a 10-day incubation at 28 ° C, the plates were flooded with PBS, which was then aspirated and filtered through a sterilized cotton gauze to recover microconidia (Ma et al., 2022).

An ex vivo onychomycosis model was established as previously described (Quatrin et al., 2020) with modifications. Pig hooves with exposed toenails were processed into ~1 cm²-sized individual toenail samples with a band saw, washed with 70% ethyl alcohol and sterilized water, and inoculated with a microconidia suspension of T. rubrum (~10⁷ conidia mL⁻¹) for 3 h. Samples were placed in a Petri dish containing moist sterilized paper and incubated at 28 ° C for 10 days. Fungal growth was confirmed by sample resuspension in PBS and plating on PDA containing 0.025% SDB and 1% penicillin-streptomycin. Infected samples were then treated with (1) 1% DMSO plus light, (2) monotherapy with MM 1 alone (0.77% in DMSO) plus light, (3) monotherapy with conventional antifungal (three drops (Quatrin et al., 2020) of Ciclopirox Topical Suspension USP, 0.77% “Lotion”, Leading Pharma, LLC, NY, USA, or Ciclopirox Topical Solution, 8% “Lacquer”, Perrigo New York Inc., NY, USA), or (4) combination therapy with MM 1 plus light and conventional antifungal. Each treatment group consisted of three samples. After 30 min, samples in the irradiated treatment groups were transferred to 24-well plates (Corning-Costar Corp., Corning, NY, USA) and irradiated with 405-nm light (87.6 J cm⁻²). Treatment was repeated every 24 h for 5 days. Afterward, the samples were transferred to tubes containing PBS plus 1% penicillin-streptomycin, vortexed, and sonicated (Quatrin et al., 2020). Triplicate aliquots of this suspension were inoculated on PDA plates containing 1% penicillin-streptomycin. After a 10-day incubation at 28 ° C, CFU numbers were determined. Untreated samples served as positive controls.

(xxviii) Statistical Analysis

Unless otherwise noted, all experiments were performed at least in triplicate. The arithmetic mean and the standard deviation or the standard error of the mean across biological and technical replicates were used as measures of mean and spread. No data points were excluded as outliers. When appropriate, data were normalized to a 0-100% range. All data processing and statistical analyses were performed using GraphPad Prism 8.0 (San Diego, CA, USA). Depending on the sample size, the normality of the data was assessed using an Anderson-Darling normality test, a D'Agostino-Pearson omnibus normality test, a Shapiro-Wilk normality test, or a Kolmogorov-Smirnov normality test with the Dallal-Wilkinson-Lilliefors test for P values. Comparisons between two groups were performed with a t-test for parametric data or a Mann-Whitney U test for nonparametric data. Comparisons between multiple groups were performed using ANOVA or a Kruskal-Wallis test with Dunn's multiple comparisons test. A Mantel-Cox test was used to determine statistical significance in G. mellonella survival experiments. Unless otherwise stated, all figures were generated in GraphPad Prism 8.0 (San Diego, CA, USA). Flow cytometry data were initially analyzed and visualized in FlowJo software (version 9, Tree Star Inc., Ashland, OR, USA) and exported to GraphPad for statistical analysis. A value of p<0.05 was considered statistically significant. Asterisks are used where appropriate to indicate the significance of differences. * p<0.05, ** p<0.01, *** p<0.001, **** p<0.0001. Confocal microscopy images were processed and analyzed using the appropriate plugins in Fiji/ImageJ (National Institutes of Health, MD, USA).

### Example 2—Use of MMs for Killing Bacteria

Described below are six visible light (405 nm) activated molecular machines (MMs) that kill Gram-negative and Gram-positive bacteria, including methicillin-resistant S. aureus (MRSA), in as little as 2 min of light activation without detectable resistance. This novel antimicrobial therapy is effective not only against exponentially growing planktonic cells but also resistant phenotypes, such as biofilms and persister cells. Using electron microscopy, RNAseq, and spectrophoto- and spectrofluorimetric methods, the mode of action of MMs was found to involve mechanical disruption of the membrane, leakage of intracellular material, and loss of membrane potential. Additionally, sublethal treatment with MM potentiated the action of conventional antibiotics as a result of MM-induced membrane permeabilization and enhanced antibiotic access to intracellular targets. Finally, at therapeutic levels, MMs mitigated mortality associated with infection by different bacterial strains (A. baumannii and S. aureus) in a burn wound infection model.

### MMs are Fast-Acting Broad-Spectrum Antibacterials

TABLE 7
Chemical structure of visible light-activated MM screened in Example 2

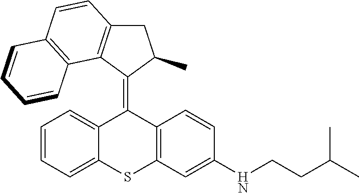

[symbol]

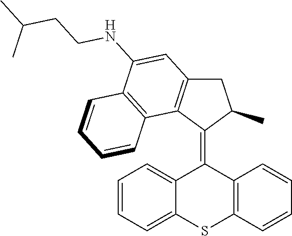

[symbol]

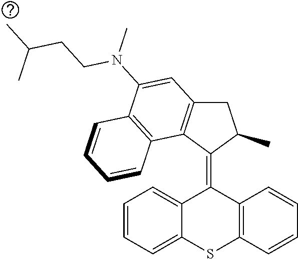

[symbol]

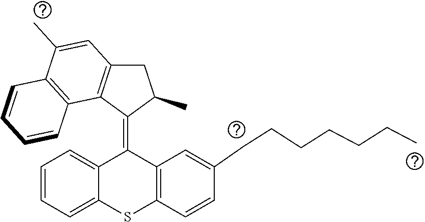

[symbol]

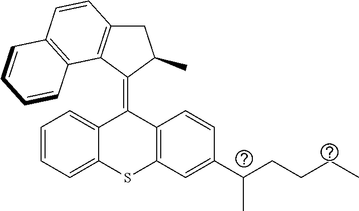

[symbol]

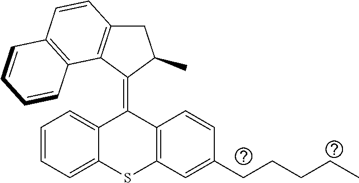

[symbol]

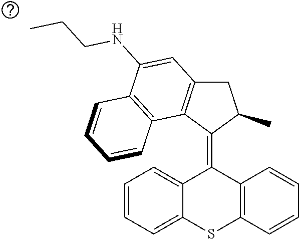

[symbol]

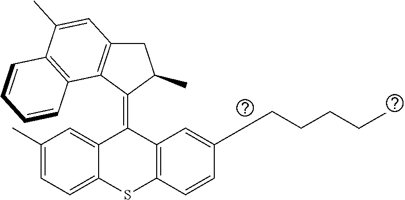

[symbol]

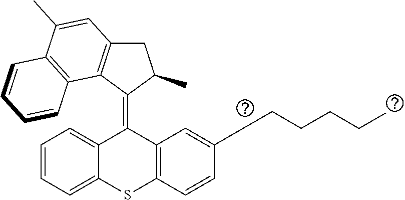

[symbol]

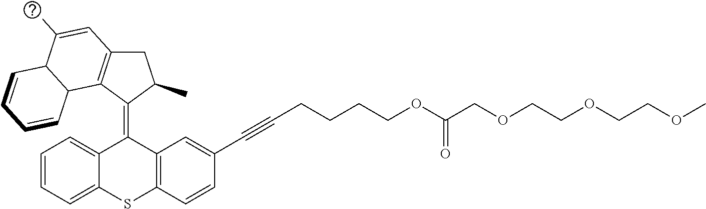

[symbol]

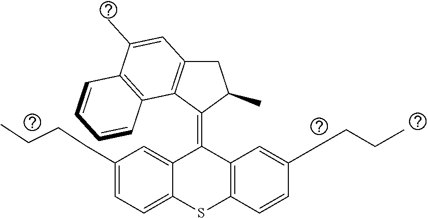

[symbol]

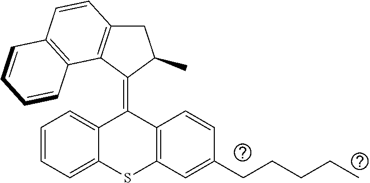

[symbol]

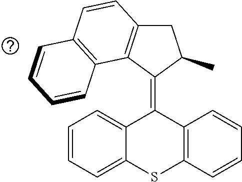

[symbol]

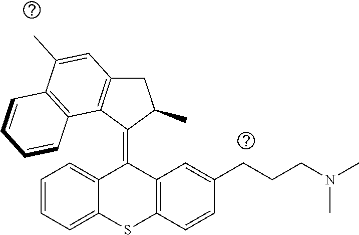

[symbol]

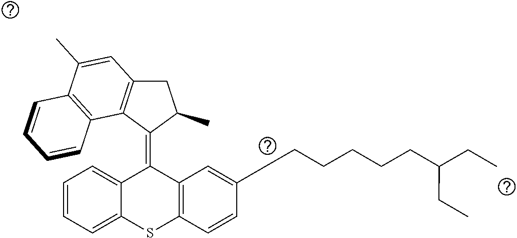

[symbol]

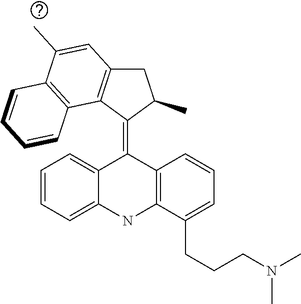

[symbol]

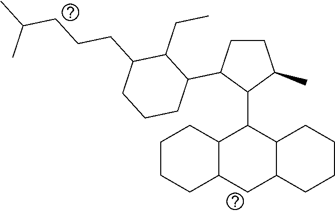 [SMILES: CCC1C(CCCC(C)C)CCCC1C1CC[C@@H](C)C1C1C2CCCCC2CC2CCCCC21]

[symbol]

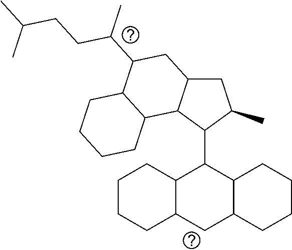 [SMILES: CC(C)CCC(C)C1CC2C[C@@H](C)C(C3C4CCCCC4CC4CCCCC43)C2C2CCCCC12]

[symbol]

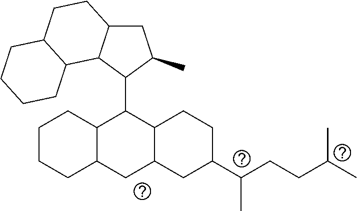 [SMILES: CC(C)CCC(C)C1CCC2C(C1)CC1CCCCC1C2C1C2C(CCC3CCCCC32)C[C@H]1C]

[symbol]

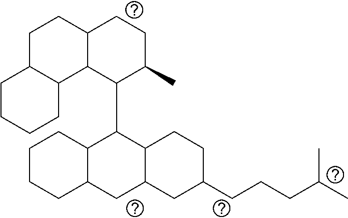 [SMILES: CC(C)CCCC1CCC2C(C1)CC1CCCCC1C2C1C2C(CCC3CCCCC32)CC[C@H]1C]

[symbol]

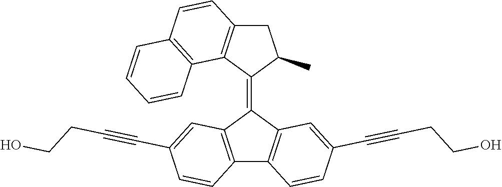 [SMILES: C[C@@H]1Cc2ccc3ccccc3c2C1=C1c2cc(C#CCCO)ccc2-c2ccc(C#CCCO)cc21]

[symbol]

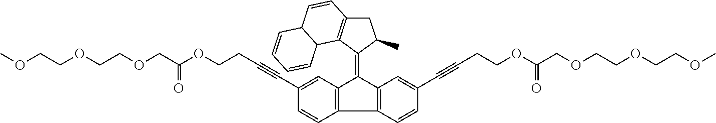

[symbol]

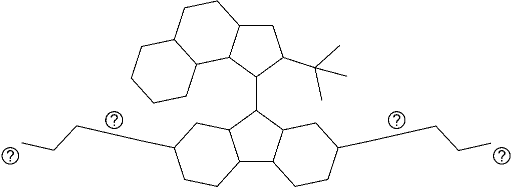 [SMILES: CCCCCC1CCC2C3CCC(CCCCC)CC3C(C3C4C(CCC5CCCCC54)CC3C(C)(C)C)C2C1]

[symbol]

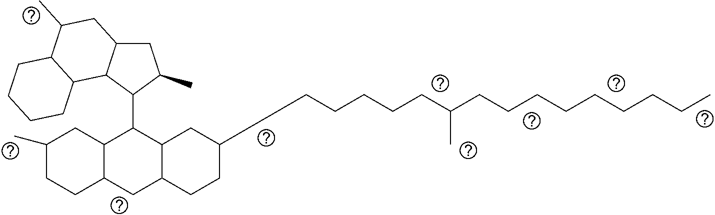 [SMILES: CCCCCCCCCC(C)CCCCCCCC1CCC2CC3CCC(C)CC3C(C3C4C(CC(C)C5CCCCC54)C[C@H]3C)C2C1]

[symbol]

Chemical structure of visible light-activated MM screened in this study, their corresponding molecular weight and estimated rotation rates following light activation based on the rotation rates of known motors containing the same core skeletons, Functional groups highlighted in red, and blue were introduced to tune the activation wavelength of the motor and increase water solubility, respectively. Slow MMs (106 Hz), chemically and spectrally analogous to fast MMs, were used as controls to test the hypothesis that mechanical action, rather than ROS generation, heat absorption, and/or other photosensitizing processes, are responsible for the antibacterial effects of MM. Detailed synthesis and characterization information is provided in the sections that follow. Chemical structures were generated in Chemdraw Ultra 6.0. [symbol]
indicates data missing or illegible when filed

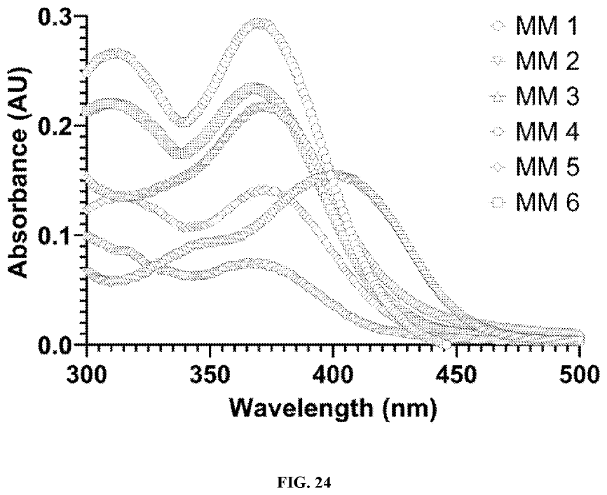

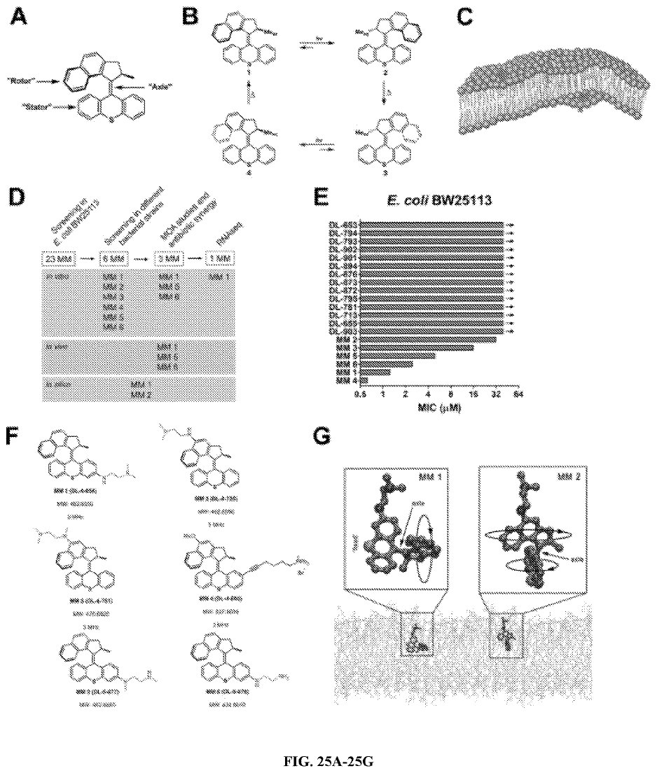

A library of 19 different visible light-activated (405 nm) MMs displaying fast rotation rates (≈3 MHz) was synthesized (Table 7). Visible light activation (FIG. 24) was achieved by introducing an amine or alkoxy electron-donating substituent into the conjugated core of the MM. The molecules were further modified with different amines in either the stator or the rotor portion (FIG. 25) of the molecule to promote the association between the protonated amines of the MMs and the negatively charged bacterial membrane.

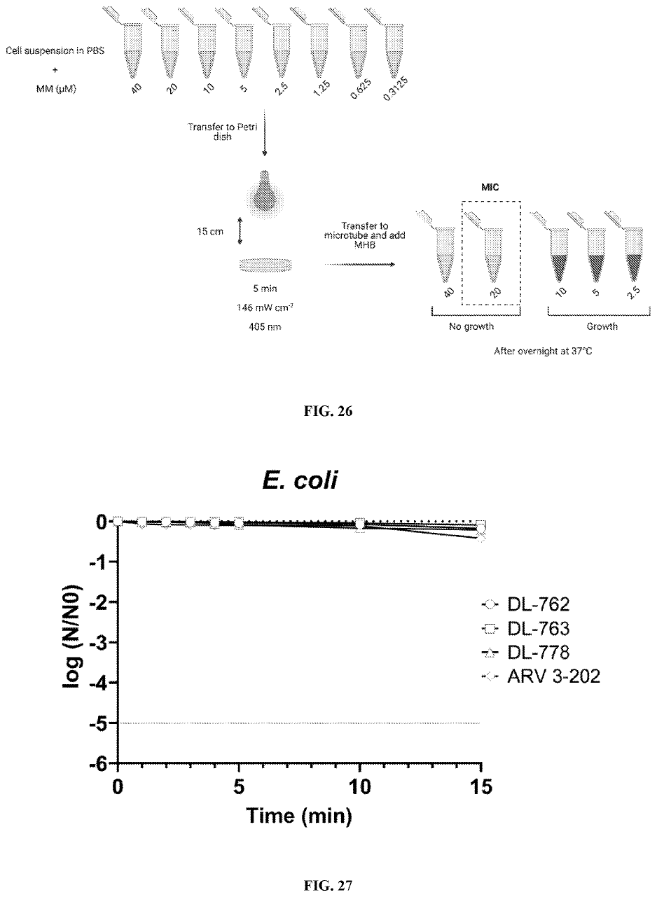

This MM library was originally screened for antibacterial activity in Escherichia coli BW25113 (FIG. 25D). E. coli cell suspensions were incubated with a range of concentrations (0.3125-40 μM) of the different MMs (8 mM stock in DMSO) and then irradiated for 5 min with 405 nm light at 146 mW cm⁻² (43.8 J cm⁻²). DMSO-only controls were included in every experiment to exclude possible effects of the vehicle. Irradiated cell suspensions were collected and inoculated into cation-adjusted Mueller-Hinton broth (MHB). Following overnight incubation (37 ° C), samples were inspected for growth. The minimal inhibitory concentration (MIC) of light-activated MM was defined as the concentration of MM resulting in no visible bacterial growth following irradiation with 43.8 J cm⁻² of 405 nm light (FIG. 26).

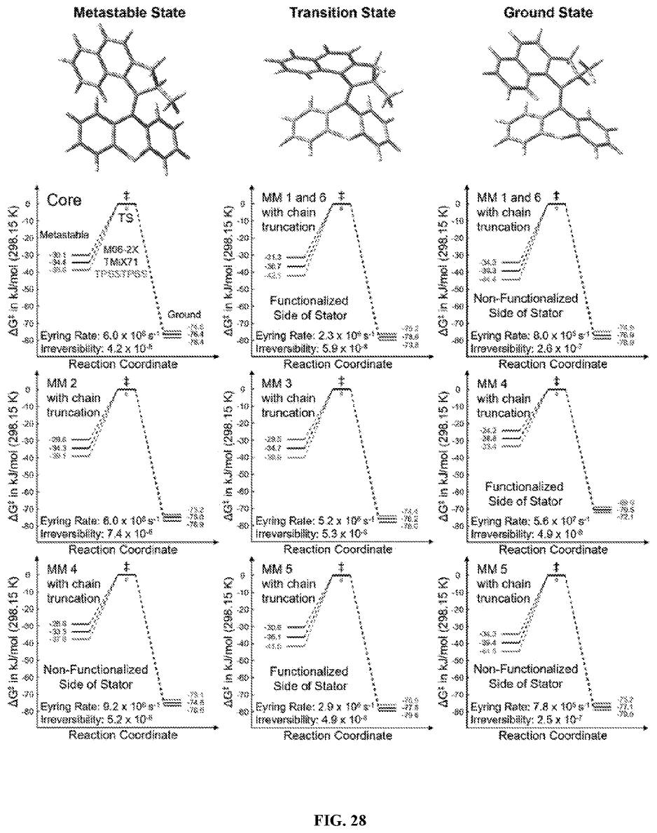

The MIC of the different MMs in E. coli is shown in FIG. 25E. Six fast-rotating MMs (MM 1 through MM 6) (FIG. 25F) displaying MIC values within the range of concentrations tested were identified. MM 4, characterized by the presence of a triphenylphosphonium (TPP+) group, displayed the lowest MIC in E. coli (0.625 μM), closely followed by MM 1 (1.25 μM). MM 2, characterized by the presence of a tertiary amine on the side chain of the rotor portion of the molecule, displayed the highest MIC(32 AM). Slow rotating MM controls (≈10⁻³ Hz) (Table 7) did not exhibit antibacterial activity (FIG. 27), denoting the importance of fast rotation rates for the antibacterial properties of MM. However, substantial differences in rotation rates of the different antibacterial MMs were not detected (FIG. 28), suggesting that small variations in the rotation rate of fast MMs cannot explain differences in their antibacterial activity.

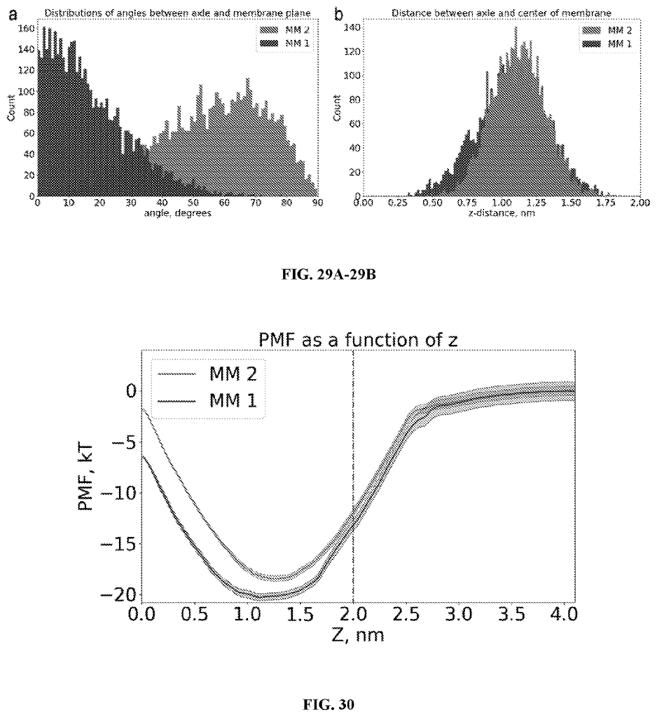

Molecular dynamics (MD) simulations revealed substantial differences in the distributions of angles between the MM axle and the plane of the membrane of the most potent (MM 1) and the least potent (MM 2) antibacterial MM (FIG. 29). In the case of MM 1, the axle of the molecule is close to parallel to the membrane (average angle of ≈15°), while for MM 2 the axle is more perpendicular to the membrane (average angle of ≈60°) (FIG. 29A). These observations indicate that MM 1 and MM 2 adopt distinct equilibrium configurations when bound to the lipid bilayer membrane (FIG. 25G), as a result of the different placement of the positively charged tertiary amine group in MM 1 and MM 2. Equilibrium simulations also revealed that the axle of MM 1 can penetrate about 2 A deeper into the membrane than that of MM 2, denoted by the slight left shift of the histogram of the distributions of distances between geometric centers of axles of the MM and membrane center of MM 1 relatively to MM 2 (FIG. 29B). This is also supported by the finding that the potential of mean force (PMF) curve for MM 1 is lower and slightly to the left of the PMF curve of MM 2 (FIG. 30), suggesting that it is more favorable for MM 1 to be located deeper inside the membrane. These differences in orientation and positioning of the two molecules within the membrane, and subsequent differences in the way they rotate following light activation, might influence the extent of the membrane deformation they exert and thus account for their distinct antibacterial activities.

TABLE 8
Bacterial Strains and Cell Lines Used in the Testing of the Compounds Described Herein

| Strains | Source | Identifier | Gram | Media | Temperature |
|---|---|---|---|---|---|
| Bacteria | | | | | |
| Bacillus cereus | ATCC | ATCC ® 13061 ™ | Positive | Tryptic Soy Broth/Agar | 30 °C |
| Frankland and Frankland | | | | | |
| Bacillus megaterium de Bary | ATCC | ATCC ® 14581 ™ | Positive | Nutrient Broth/Agar | 30 °C |
| Bacillus subtilis subsp. subtilis | ATCC | ATCC ® 6051 ™ | Positive | Nutrient Broth/Agar | 30 °C |
| (Ehrenberg) Cohn | | | | | |
| Enterococcus faecalis | ATCC | ATCC ® 29212 ™ | Positive | Brain Heart Infusion | 37 °C |
| (Andrewes and Horder) Schleifer | | | | Broth/Agar | |
| and Kilpper-Balz | | | | | |
| Enterococcus faecium | ATCC | ATCC ® 19434 ™ | Positive | Brain Heart Infusion | 37 °C |
| (Orla-Jensen) Schleifer and Kilpper-Balz | | | | Broth/Agar | |
| Staphylococcus aureus supsp. aureus | ATCC | ATCC ® 700698 | Positive | Brain Heart Infusion | 37 °C |
| Rosenbach | | | | Broth/Agar | |
| Staphylococcus epidermidis | ATCC | ATCC ® 12228 ™ | Positive | Nutrient Broth/Agar | 37 °C |
| (Winslow and Winslow) Evans | | | | | |
| Methicillin-resistant Staphylococcus aureus | ATCC | ATCC ® BAA-1680 | Positive | Tryptic Soy Broth/Agar | 37 °C |
| Stenotrophomonas maltophilia | ATCC | ATCC ® 13637 ™ | Negative | Nutrient Broth/Agar | 30 °C |
| (Hugh) Palleroni and Bradbury | | | | | |
| Acinetobacter baumannii | ATCC | ATCC ® BAA-1605 ™ | Negative | Tryptic Soy Broth/Agar | 37 °C |
| Burkholderia cepacia | ATCC | ATCC ® 25416 ™ | Negative | Nutrient Broth/Agar | 30 °C |
| (Patteroni and Holmes) Yabuuchi et al. | | | | | |
| Enterobacter cloacae subsp. cloasae | ATCC | ATCC ®13047 ™ | Negative | Nutrient Broth/Agar | 30 °C |
| (Jordan) Hormaeche and Edwards [symbol] | | | | | |
| Escherichia coli | Geroge Bennett, BRC, | BW25113 | Negative | Nutrient Broth/Agar | 37 °C |
| | Rice University | | | | |
| Pseudomonas aeruginosa | DSMZ | DSM 22644 (PAO1) | Negative | Tryptic Soy Broth/Agar | 30 °C |
| Single E. Coli gene knockouts | | | | | |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | Negative | Tryptic Soy Broth/Agar | 37 °C |
| Δ [symbol] | CGSC | JW [symbol] | | | |

| Mammalian Cell Lines | Source | Identifier | | Media | Temperature |
|---|---|---|---|---|---|
| Human Embryonic Kidney | ATCC | ATCC ® | NA | Eagle's Minimum Essential Medium | 37 °C |
| cells HEK-293 | | CRL-1573 ™ | | (EMEM) (ATCC ® 30-2023 ™) | |
| Primary normal human | ATCC | ATCC ® | NA | Fibroblast Basal Medium | 37 °C |
| dermal fibroblasts (NHDFs) | | PCS-201-012 ™ | | (ATCC ® PCS-201-030 ™) | |

[symbol]

indicates data missing or illegible when filed

The bacteriostatic potential of the identified MM (FIG. 25F) was further investigated in additional Gram-negative and Gram-positive bacterial strains (Table 8, Table 9).

TABLE 9
MIC of six MMs in different bacterial strains.

| | A. baumannii | E. coli | P. aeruginosa | S. aureus |
|---|---|---|---|---|
| MM 1 | 2.5 | 1.25 | 0.625 | 0.156 |
| MM 2 | 20 | 32 | 5 | 1.25 |
| MM 3 | 20 | 16 | 5 | 1.25 |
| MM 4 | 2.5 | 1 | 0.625 | 0.078 |
| MM 5 | 2.5 | 5 | 0.625 | 0.078 |
| MM 6 | 2.5 | 2.5 | 0.625 | 0.078 |

The MIC value was determined as described in a section that follows. Results are the mean of at least three biological replicas.

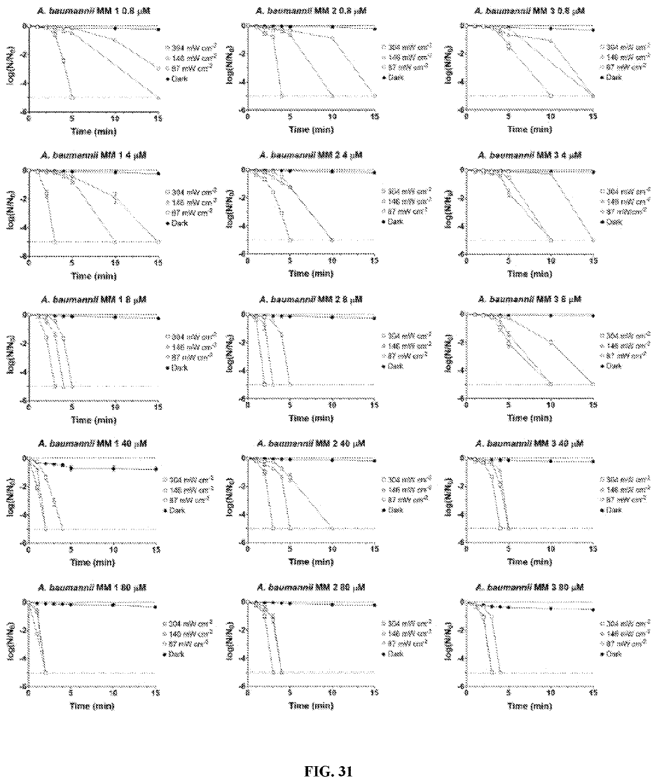

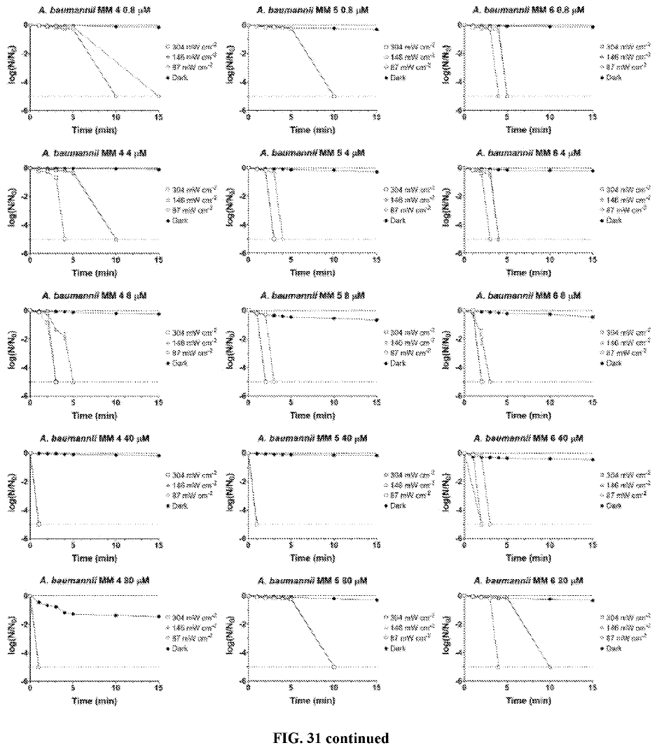

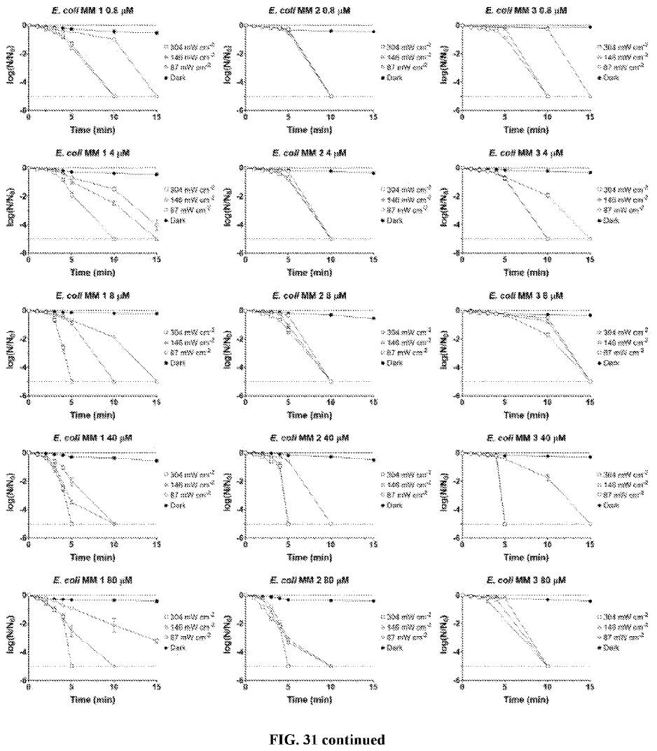

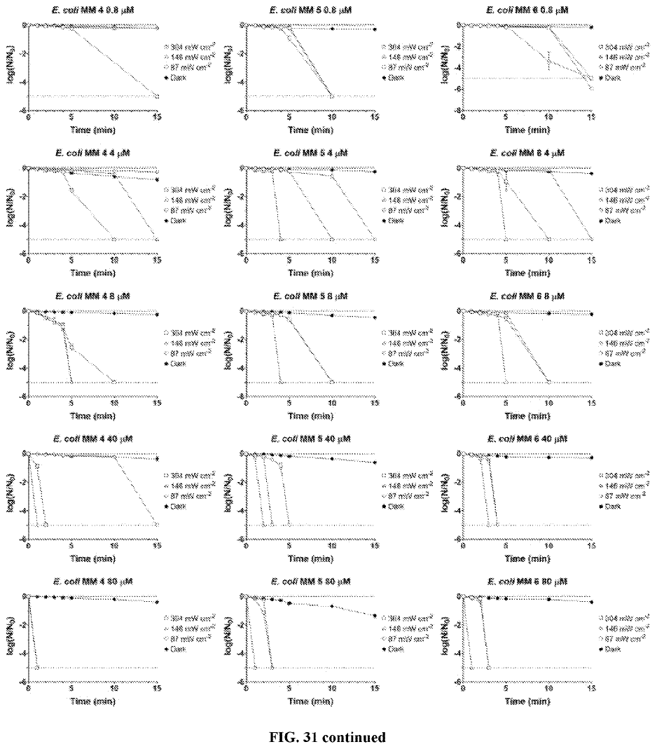

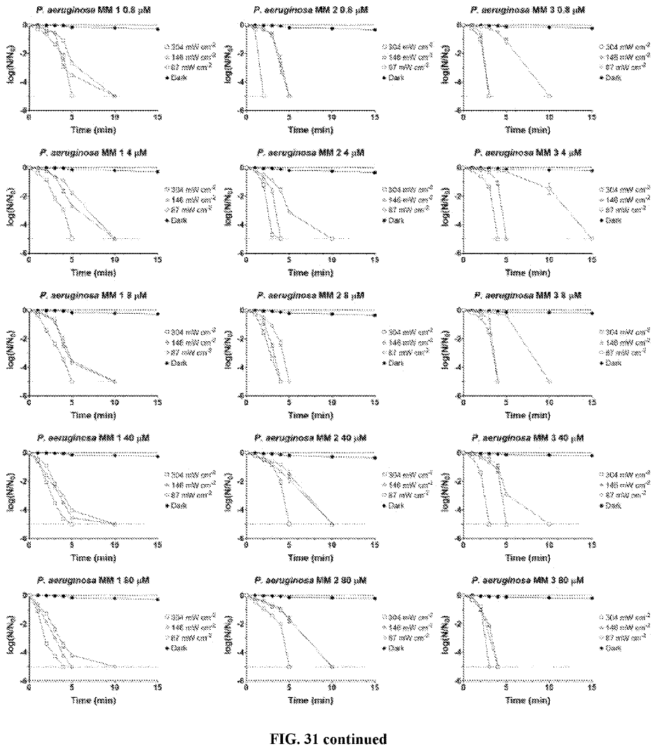

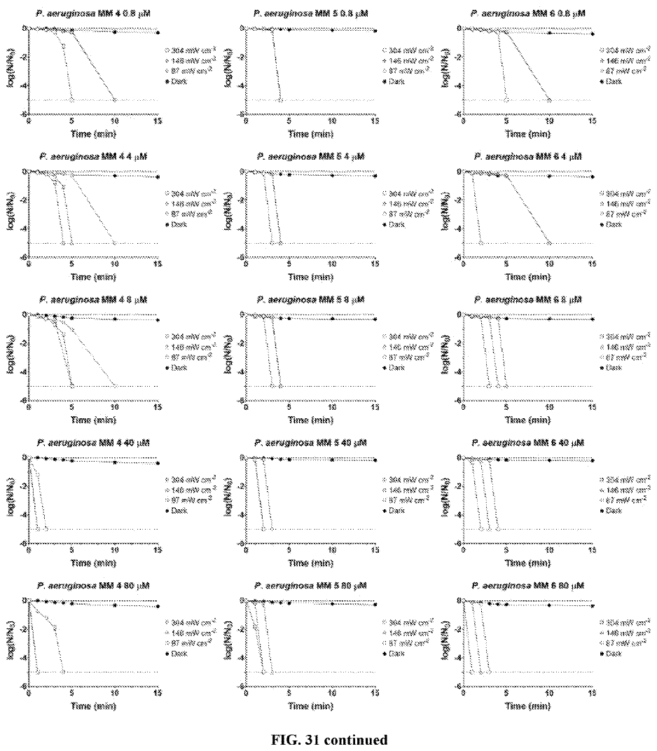

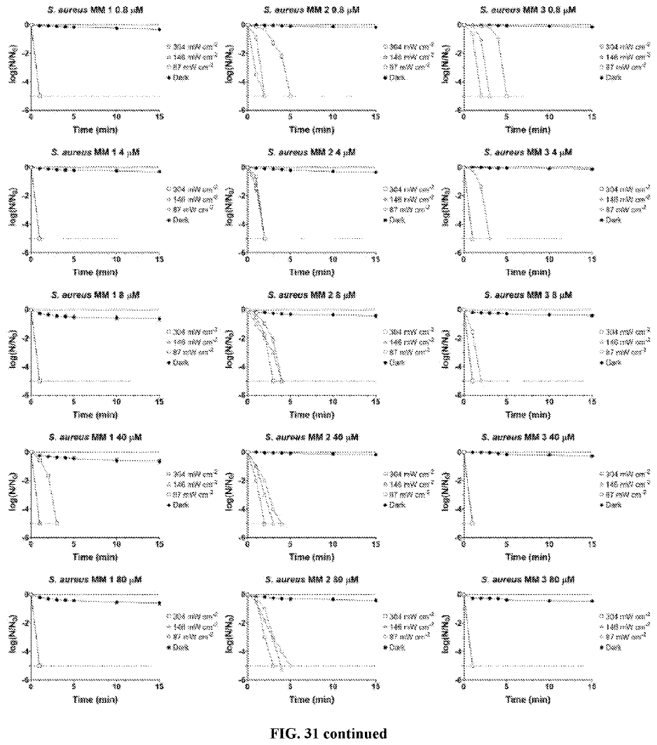

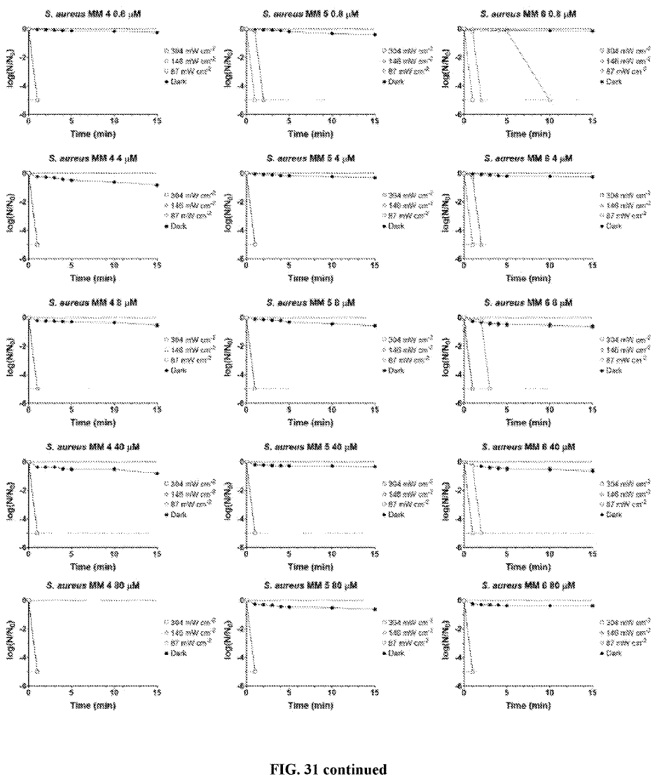

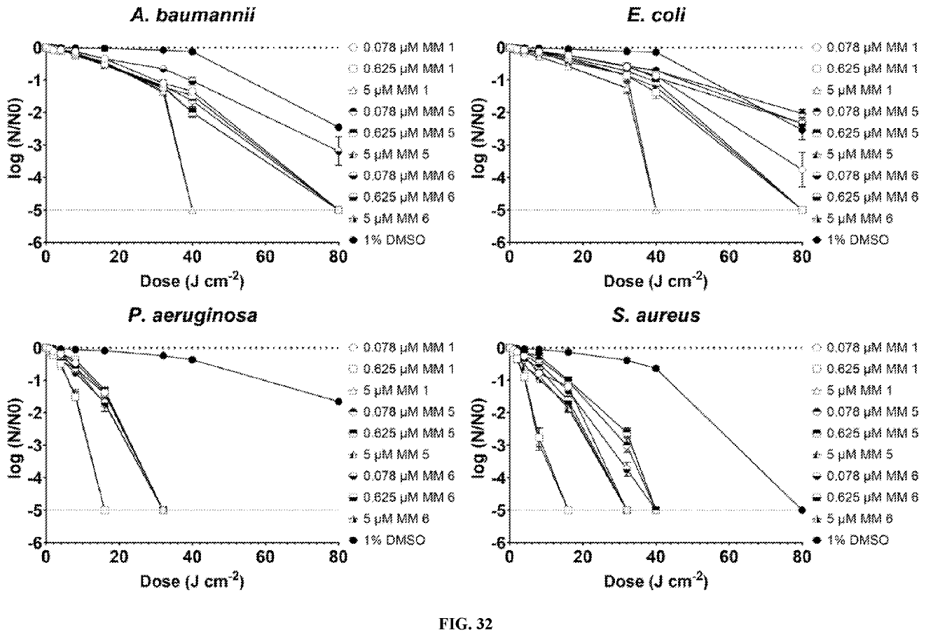

Killing by light-activated MMs varied in a concentration- and light-intensity-dependent manner, with enhanced MM concentration and light intensity resulting in higher MM-induced killing (FIG. 31). Some toxicity of the MM itself (in the absence of light) was detected, particularly for the TPP+containing MM 4 (FIG. 31), which was, therefore, excluded in subsequent “mode-of-action” experiments. Among the strains tested, S. aureus was particularly susceptible to killing by high MM concentrations even in the absence of light. S. aureus also exhibited substantial sensitivity to 405 nm light alone (FIG. 31). Light dose-dependent reduction of bacterial numbers by different concentrations of the most potent MMs (MM 1, MM 5, and MM 6) revealed that complete eradication of A. baumannii and E. coli required at least 40 J cm⁻² of 405 nm light in samples treated with the highest concentration of MMs tested (5 μM). Complete eradication of P. aeruginosa and S. aureus could be achieved with 16 J cm⁻² of 405 nm light and 0.625 to 5 μM of MM (FIG. 32).

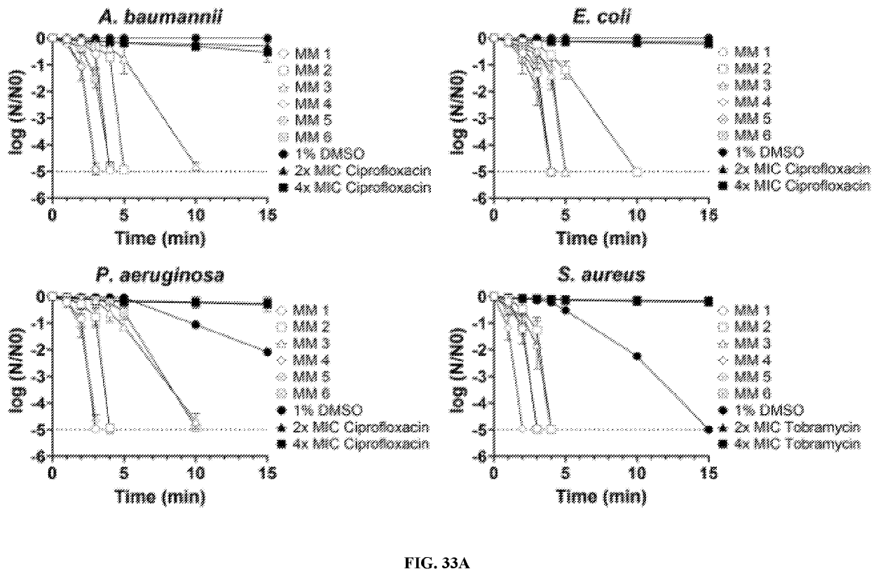

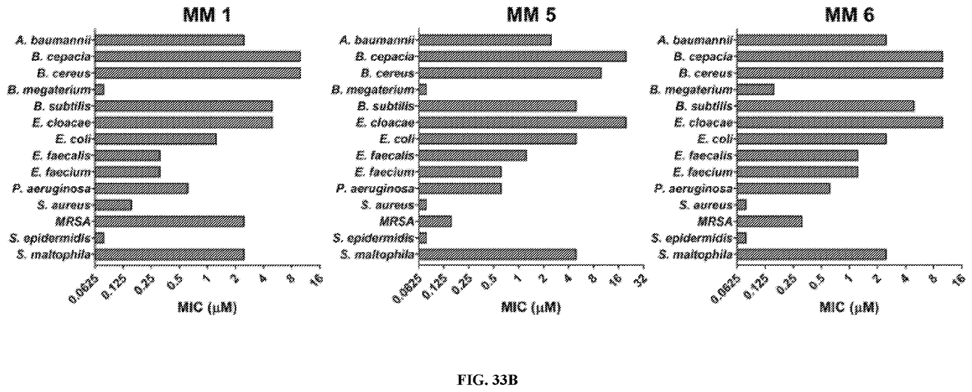

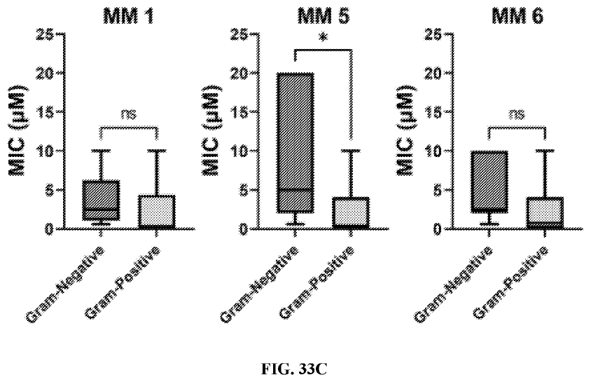

The bactericidal properties of MMs (2×MIC) were further examined at a fixed light intensity of 146 mW cm⁻² (Table 10, FIG. 33A). In A. baumannii, treatment with different MMs reduced cell number to the limit of detection in 3 min (MM 4) to 10 min (MM 3). In E. coli, bacterial numbers were reduced to the limit of detection in 4 min (MM 4, MM 5, MM 6) to 10 min (MM 2) of irradiation in the presence of 2×MIC of each MM. For P. aeruginosa, MM-induced reduction of cell numbers to the limit of detection was achieved in 3 min (MM 1, MM 4) to 10 min (MM 3, MM 6). Complete elimination of S. aureus was achieved in 2 min (MM 4) to 4 min (MM 2, MM 3) of irradiation.

TABLE 10
Antibiotic MIC (in μg per mL) of
different strains examined in Example 2.

| Antibiotic | Bacterial strain |
|---|---|
| | A. baumannii |
| Ciprofloxacin | 0.25 |
| Polymyxin B | 0.125 |
| Tetracycline | 8 |
| | E. coli |
| Ampicillin | 0.063 |
| Ciprofloxacin | 0.008 |
| Gentamicin | 1 |
| Novobiocin | 64 |
| Trimethoprim | 1 |
| | P. aeruginosa |
| Ciprofloxacin | 1 |
| Gentamicin | 1 |
| Trimethoprim | 128 |
| | S. aureus |
| Ciprofloxacin | 1 |
| Gentamicin | 2 |
| Tobramycin | 4 |

Results are the mean of at least 3 biological replicas determined using the broth microdilution method.

The antibacterial spectrum of action of the most efficient MMs (MM 1, MM 5, and MM 6) was assessed in additional strains, including methicillin-resistant S. aureus (MRSA) (FIG. 33B). The MIC of MM 1 ranged from 0.078 UM in B. megaterium and S. epidermidis to 10 μM in B. cepacia and B. cereus. The MIC of MM 5 ranged from 0.078 AM in B. megaterium, S. aureus, and S. epidermidis to 20 μM in B. cepacia and E. cloacae. The MIC of MM 6 ranged from 0.078 μM in S. aureus and S. epidermidis to 10 AM in B. cepacia, B. cereus, and E. cloacae. Overall, while the median MIC of MM 5 in Gram-positive bacteria was lower than that in Gram-negative bacteria (p<0.05), in the case of MM 1 and MM 6 significant differences in the MIC between the two bacterial groups were not observed (FIG. 33C).

TABLE 11
List of single gene efflux knockouts of E. coli analyzed in Example 2.

| Gene | Function |
|---|---|
| aaeA | P-hydroxybenzoic acid efflux pump subunit AaeA. Forms an efflux pump with AaeB. |
| acrA | Multidrug efflux pump subunit AcrA. AcrA-AcrB-AcrZ-TolC is a drug efflux protein |
| | complex with broad substrate specificity that uses the proton motive force to export |
| | substrates. |
| acrE | Multidrug export protein AcrE. Part of the tripartite efflux system AcreF-TolC. Involved |
| | in the efflux of indole and organic solvents. |
| bcr | Bicyclomycin resistance protein. Probable membrane translocase. Involved in sulfonamide |
| | (sulfathiazole) and bicyclomycin resistance. |
| emrA | Multidrug export protein EmrA. Part of the tripartite efflux system EmrAB-TolC, which confers |
| | resistance to antibiotics such as CCCP, FCCP, 2,4-dinitrophenol, and nalidixic acid. |
| emrD | Multidrug resistance protein D. Multidrug resistance pump that participates in a low |
| | energy shock adaptive response |
| emrE | Multidrug transporter EmrE. Expels positively charged hydrophobic drugs across the inner |
| | membrane, conferring resistance to a wide range of toxic compounds including methyl |
| | viologen, ethidium bromide, and acriflavine. |
| emrK | Probable multidrug resistance protein EmrK. Part of the tripartite efflux system EmrYK-TolC, |
| | which confers resistance to various drugs |
| fsr | Fosmidomycin resistance protein. Confers resistance against fosmidomycin |
| hsrA | Member of the major facilitator superfamily. Putative transport protein (MFS family). |
| macA | Macrolide export protein maca. Part of the tripartite efflux system MacAB-TolC. When |
| | overexpressed, the system confers resistance against macrolides composed of 14- and |
| | 15-membered lactones. |
| mdtA | Multidrug resistance protein MdtA. The MdtABC tripartite complex confers resistance |
| | against novobiocin and deoxycholate. |
| mdtE | Multidrug resistance protein MdtE. Part of the tripartite efflux system MdtEF-TolC, which |
| | confers resistance to compounds such as rhodamine 6G, erythromycin, doxorubicin, |
| | ethidium bromide, TPP, SDS, deoxycholate, crystal violet, and benzalkonium |
| mdtG | Multidrug resistance protein MdtG. Confers resistance to fosfomycin and deoxycholate |
| mdtI | Spermidine export protein MdtI. Can also confer resistance to deoxycholate and SDS. |
| mdtL | Multidrug resistance protein MdtL. Confers resistance to chloramphenicol |
| mdtM | Multidrug resistance protein MdtM. Confers resistance to acriflavine, chloramphenicol, |
| | norfloxacin, ethidium bromide, and TPP |
| mdtN | Multidrug resistance protein MdtN. Could be involved in resistance to puromycin, acriflavine, |
| | and tetraphenylarsonium chloride. |
| nimT | 2-nitroimidazole exporter. A ΔnimT mutant is more sensitive to the natural antibiotic, |
| | 2-nitroimidazole, than wild type. Overexpression of nimT from a plasmid confers resistance |
| | to the toxic chemical, bromoacetate |
| tolC | Outer membrane protein TolC; Outer membrane channel, which is required for the function of |
| | several efflux systems such as AcrAB-TolC, AcrEF-TolC, EmrAB-TolC and MacAB-TolC. These |
| | systems are involved in export of antibiotics and other toxic compounds from the cell. TolC is |
| | also involved in import of colicin E1 into the cells |
| ybhT | Multidrug efflux pump accessory protein AcrZ. Encodes a small membrane protein that associates |
| | with the AcraB-TolC multidrug efflux pump in an AcrB-dependent manner. |

All strains were obtained from CGSC, The Coli Genetic Stock Center. Information on function for each gene was obtained from https://string-db.org/ and https://ecocyc.org/.

The antibacterial properties of MMs were also assessed in a panel of E. coli single-gene knockouts, deleted for genes encoding different components of efflux pumps responsible for resistance to different antibiotics (Table 11). The MIC of the different single-gene knockouts was lower, equal, or superior to that of the wild-type (WT) parent strain depending on the gene deleted, with no particular trend towards resistance or sensitivity to MM treatment among the panel of strains tested (FIG. 34).

### MM Kill Persister Cells and Disrupt Established Biofilms

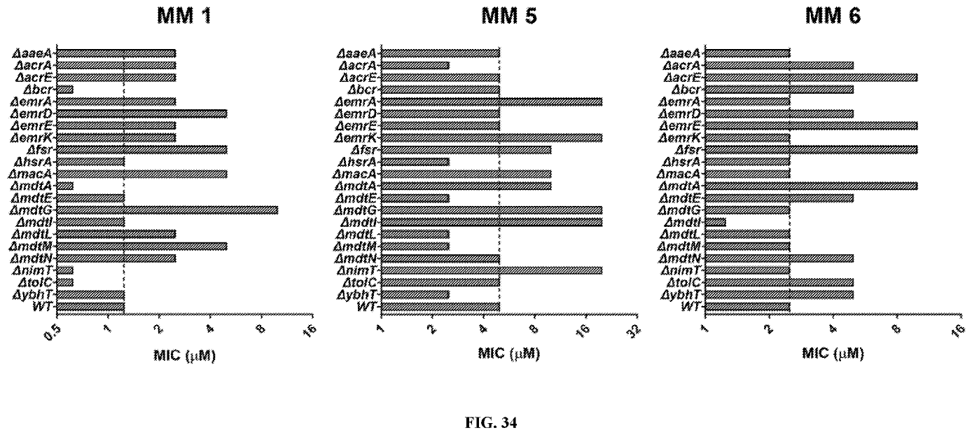

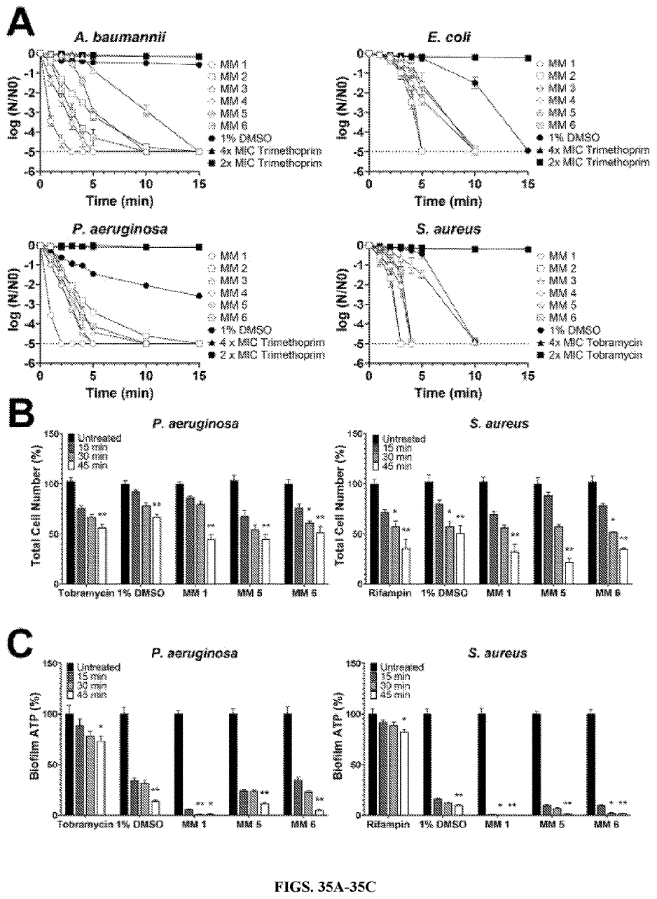

The ability of light-activated MMs (1×MIC) to kill antibiotic-tolerant persister cells (FIG. 3A) was investigated in the Gram-negative strains A. baumannii, E. coli, and P. aeruginosa. In S. aureus, stationary phase cells were used in persister eradication assays (Keren et al., 2004). In A. baumannii, treatment with MMs resulted in a reduction in the levels of persisters to the limit of detection in 3 min (MM 1) to 15 min (MM 3) of irradiation. The number of persister cells of E. coli was reduced to the limit of detection in 5 min (MM 2, MM 4, MM 6) to 10 min (MM 1, MM 5, MM 3) of irradiation. In the case of P. aeruginosa, persister levels were reduced to the limit of detection in 2 min (MM 1) to 15 min (MM 2) of irradiation. Reduction in the number of persister cells of S. aureus to the limit of detection was achieved in 3 min (MM 2, MM 4) to 10 min (MM 3) (FIG. 35A).

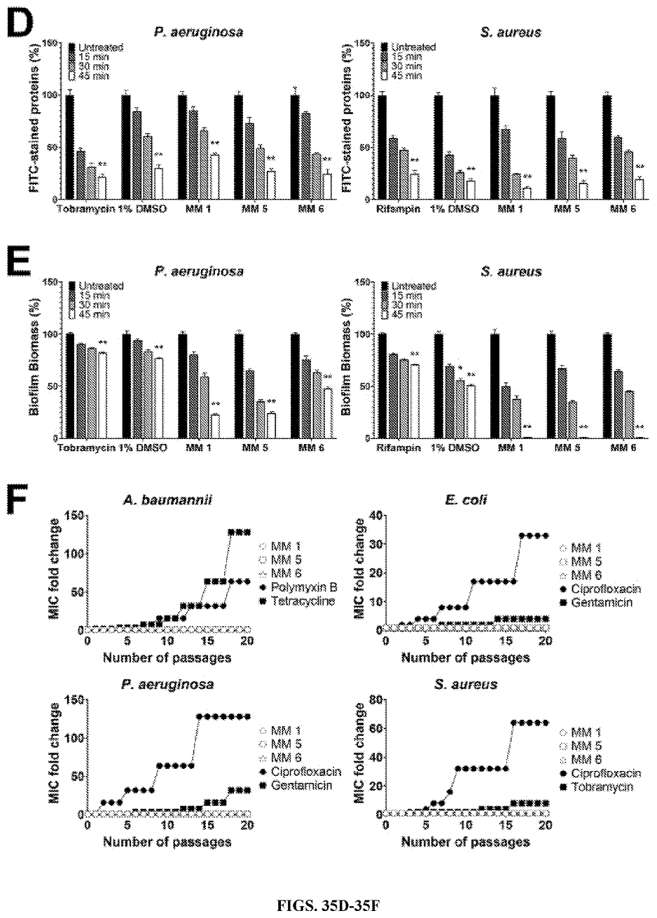

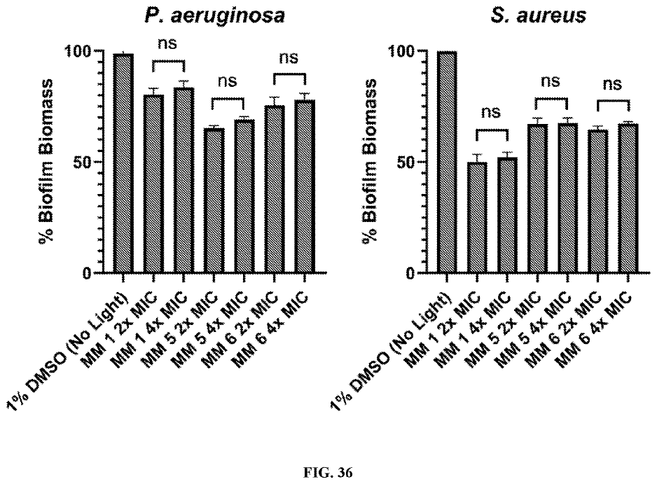

The antibiofilm potential of the most efficient MM was investigated in a 96-well plate format using a combination of methods targeting different components of the biofilm (Stiefel et al., 2016). Since preliminary experiments revealed that, for the same irradiation period, treatment with 1× or 2×MIC of MMs resulted in a similar reduction in biofilm biomass (FIG. 36), in subsequent experiments irradiation time, rather than MM concentrations, was varied. Mature biofilms of P. aeruginosa and S. aureus were established and then challenged with 1% DMSO or 2×MIC of MM 1, MM 5, and MM 6 followed by 405 nm irradiation at 146 mW cm⁻² for 15-, 30-and 45-min. Conventional antibiotics (rifampin and tobramycin) at 2× MIC were used as controls. The effect of visible light-activated MMs on total bacterial cell number within biofilms was assessed using the nucleic acid dye acridine orange (Stiefel et al., 2016). Compared to untreated controls, treatment with the antibiotics tobramycin and rifampin resulted in a reduction in total bacterial cell numbers of up to 43 and 64% (p<0.01), respectively. DMSO-treated samples showed a reduction in total cell numbers of up to 50% (p<0.01), while MM-treated cells showed up to 78% (p<0.01) reduction in total cell number, compared to the respective untreated controls (FIG. 35B).

ATP quantification was used as a proxy of the number of metabolically active cells within biofilms (Stiefel et al., 2016). The population of metabolically active cells was reduced by 18 to 27% (p<0.05) by rifampin and tobramycin, respectively, even after 45 min of treatment, while a 15-min treatment period with visible light-activated MM reduced the amount of metabolically active cells by as much as 94% (p<0.01), compared to a 66% reduction (p<0.01) in DMSO-treated samples, relatively to the respective untreated controls (FIG. 35C).

Treatment with control antibiotics resulted in a reduction in biofilm protein content of up to 78% (p<0.01). The same treatment time resulted in up to 82% reduction (p<0.01) in biofilm protein in DMSO-treated samples and up to 89% reduction (p<0.01) in MM-treated samples, compared to untreated controls (FIG. 35D).

Treatment with the antibiotics tobramycin and rifampin resulted in a reduction of biofilm biomass of up to 29% (FIG. 35E). Compared to untreated controls, treatment with visible light-activated MMs resulted in a biomass reduction of up to 99% (p<0.05), while DMSO-treated samples showed a reduction in biofilm biomass of up to 49% (FIG. 35E).

Repeated Exposure to MM does not Lead to the Development of Resistance

The ability of cells to develop resistance to successive MM exposure was assessed by serial passaging, whereby cells surviving treatment with 0.5×MIC of the different MMs followed by 5 min of irradiation with 405 nm light at 146 mW cm⁻² (43.8 J cm⁻²) were collected and subjected to 20 cycles of repeated exposure to MMs. Repeated exposure to MMs did not result in a change in the MIC, in contrast to the steep increase (32- to 128-fold) in the MIC through time that was observed in samples treated with conventional antibiotics (FIG. 35F). Importantly, mutants that evolved resistance to antibiotics did not exhibit cross-resistance to MM (Table 12).

TABLE 12
Susceptibility (assessed as the MIC) of antibiotic-resistant E. coli and S. aureus to MMs.

| | MIC (μM) | | | | | | | | | | | |
|---|---|---|---|---|---|---|---|---|---|---|---|---|
| | E. coli | | | | | | S. aureus | | | | | |
| Passage | Ciprofloxacin^{R} | | | Gentamycin^{R} | | | Ciprofloxacin^{R} | | | Gentamycin^{R} | | |
| number | MM 1 | MM 5 | MM 6 | MM 1 | MM 5 | MM 6 | MM 1 | MM 5 | MM 6 | MM 1 | MM 5 | MM 6 |
| 1 | 4 | 0.5 | 0.5 | 4 | 0.5 | 0.5 | 0.125 | 0.125 | 0.125 | 0.125 | 0.125 | 0.125 |
| 2 | 4 | 0.5 | 0.5 | 4 | 0.5 | 0.5 | 0.125 | 0.125 | 0.125 | 0.125 | 0.125 | 0.125 |
| 3 | 4 | 0.5 | 0.5 | 4 | 0.5 | 0.5 | 0.125 | 0.125 | 0.125 | 0.125 | 0.125 | 0.125 |
| 4 | 4 | 0.5 | 0.5 | 4 | 0.5 | 0.5 | 0.125 | 0.125 | 0.125 | 0.125 | 0.125 | 0.125 |
| 5 | 4 | 0.5 | 0.5 | 4 | 0.5 | 0.5 | 0.125 | 0.125 | 0.125 | 0.125 | 0.125 | 0.125 |
| 6 | 4 | 0.5 | 0.5 | 4 | 0.5 | 0.5 | 0.125 | 0.125 | 0.125 | 0.125 | 0.125 | 0.125 |
| 7 | 4 | 0.5 | 0.5 | 4 | 0.5 | 0.5 | 0.125 | 0.125 | 0.125 | 0.125 | 0.125 | 0.125 |
| 8 | 4 | 0.5 | 0.5 | 4 | 0.5 | 0.5 | 0.125 | 0.125 | 0.125 | 0.125 | 0.125 | 0.125 |
| 9 | 4 | 0.5 | 0.5 | 4 | 0.5 | 0.5 | 0.125 | 0.125 | 0.125 | 0.125 | 0.125 | 0.125 |
| 10 | 4 | 0.5 | 0.5 | 4 | 0.5 | 0.5 | 0.125 | 0.125 | 0.125 | 0.125 | 0.125 | 0.125 |
| 11 | 4 | 0.5 | 0.5 | 4 | 0.5 | 0.5 | 0.125 | 0.125 | 0.125 | 0.125 | 0.125 | 0.125 |
| 12 | 4 | 0.5 | 0.5 | 4 | 0.5 | 0.5 | 0.125 | 0.125 | 0.125 | 0.125 | 0.125 | 0.125 |
| 13 | 4 | 0.5 | 0.5 | 4 | 0.5 | 0.5 | 0.125 | 0.125 | 0.125 | 0.125 | 0.125 | 0.125 |
| 14 | 4 | 0.5 | 0.5 | 4 | 0.5 | 0.5 | 0.125 | 0.125 | 0.125 | 0.125 | 0.125 | 0.125 |
| 15 | 4 | 0.5 | 0.5 | 4 | 0.5 | 0.5 | 0.125 | 0.125 | 0.125 | 0.125 | 0.125 | 0.125 |
| 16 | 4 | 0.5 | 0.5 | 4 | 0.5 | 0.5 | 0.125 | 0.125 | 0.125 | 0.125 | 0.125 | 0.125 |
| 17 | 4 | 0.5 | 0.5 | 4 | 0.5 | 0.5 | 0.125 | 0.125 | 0.125 | 0.125 | 0.125 | 0.125 |
| 18 | 4 | 0.5 | 0.5 | 4 | 0.5 | 0.5 | 0.125 | 0.125 | 0.125 | 0.125 | 0.125 | 0.125 |
| 19 | 4 | 0.5 | 0.5 | 4 | 0.5 | 0.5 | 0.125 | 0.125 | 0.125 | 0.125 | 0.125 | 0.125 |
| 20 | 4 | 0.5 | 0.5 | 4 | 0.5 | 0.5 | 0.125 | 0.125 | 0.125 | 0.125 | 0.125 | 0.125 |

During serial passaging experiments used to track the evolution of antibiotic resistance, cells growing at half the MIC for each antibiotic were collected at each passage and stored at −80 °C for further testing of their MM MIC by irradiating cell suspensions amended with a range of MM concentrations with 405 nm light at 146 mW cm⁻² for 5 min, followed by inoculation into MHB and overnight growth, as described in sections that follow. Results are the mean of at least 3 biological replicas.

### MM Target the Cell Membrane

The mechanism of action of MM was investigated using RNAseq, an array of spectrophoto- and spectrofluorimetric methods, and electron microscopy (FIG. 25D). All mechanism of action studies were conducted under the same irradiation conditions: 5 min of irradiation with 405 nm light at 146 mW cm⁻² (light dose of 43.8 J cm⁻²).

RNAseq was conducted on E. coli treated with 0.5×MIC of the most potent MM (MM 1) or 1% DMSO and 43.8 J cm⁻² of 405 nm light (FIG. 37A). A total of 4311 transcripts were detected by RNAseq (FIG. 37B). Of these, 2694 showed significantly different levels (p <0.05) in MM-treated cells compared to DMSO controls. A total of 1362 transcripts were significantly more abundant in MM-treated samples, while 1332 transcripts were significantly more abundant in DMSO controls. MM 1-treated samples and DMSO controls exhibited distinct transcriptomic profiles (FIG. 37C), with some transcripts displaying as much as a 5-fold difference in abundance between treatments (FIG. 37D).

Gene ontology (GO) enrichment analysis revealed that transcripts more abundant in DMSO controls were significantly enriched (p<0.05) for membrane-associated biological processes and molecular functions, including respiration and transmembrane transport (Table 13, Table 14). Transcripts more abundant in DMSO controls were also significantly enriched (p<0.05) for membrane-related cellular components (Table 15).

TABLE 13
GO Enrichment Analysis for GO biological process of transcripts
significantly more abundant in DMSO-treated cells

| | E. coli | Transcripts significantly enriched in DMSO controls | | | | | |
|---|---|---|---|---|---|---|---|
| | (REF) | | | Fold | | raw P | |
| GO biological process | # | # | expected | Enrichment | +/− | value | FDR |
| aerobic respiration | 54 | 39 | 15.78 | 2.47 | + | 2.71E−05 | 6.13E−03 |
| cellular respiration | 114 | 77 | 33.32 | 2.31 | + | 2.82E−08 | 2.98E−05 |
| energy derivation by oxidation of organic | 129 | 83 | 37.70 | 2.20 | + | 3.45E−08 | 2.73E−05 |
| compounds | | | | | | | |
| generation of precursor metabolites and | 210 | 110 | 61.37 | 1.79 | + | 8.55E−07 | 3.38E−04 |
| energy | | | | | | | |
| cellular metabolic process | 2016 | 683 | 589.19 | 1.16 | + | 4.05E−06 | 1.28E−03 |
| metabolic process | 2184 | 740 | 638.29 | 1.16 | + | 6.06E−07 | 3.20E−04 |
| cellular process | 2971 | 961 | 868.29 | 1.11 | + | 5.96E−07 | 3.77E−04 |
| respiratory electron transport chain | 41 | 29 | 11.98 | 2.42 | + | 4.36E−04 | 4.31E−02 |
| electron transport chain | 107 | 58 | 31.27 | 1.85 | + | 2.05E−04 | 2.82E−02 |
| anaerobic respiration | 61 | 38 | 17.83 | 2.13 | + | 3.72E−04 | 3.80E−02 |
| cellular protein-containing complex assembly | 73 | 44 | 21.33 | 2.06 | + | 2.02E−04 | 2.91E−02 |
| protein-containing complex assembly | 136 | 72 | 39.75 | 1.81 | + | 6.32E−05 | 1.25E−02 |
| protein-containing complex subunit | 143 | 74 | 41.79 | 1.77 | + | 8.94E−05 | 1.49E−02 |
| organization | | | | | | | |
| translation | 118 | 64 | 34.49 | 1.86 | + | 9.42E−05 | 1.49E−02 |
| peptide biosynthetic process | 130 | 66 | 37.99 | 1.74 | + | 3.35E−04 | 3.66E−02 |
| peptide metabolic process | 148 | 73 | 43.25 | 1.69 | + | 2.88E−04 | 3.25E−02 |
| organonitrogen compound metabolic | 972 | 372 | 284.07 | 1.31 | + | 6.63E−07 | 3.00E−04 |
| process | | | | | | | |
| organonitrogen compound biosynthetic | 516 | 208 | 150.80 | 1.38 | + | 4.16E−05 | 8.77E−03 |
| process | | | | | | | |
| cation transport | 231 | 104 | 67.51 | 1.54 | + | 2.62E−04 | 3.07E−02 |
| ion transport | 614 | 232 | 179.44 | 1.29 | + | 3.62E−04 | 3.83E−02 |
| transport | 710 | 276 | 207.50 | 1.33 | + | 1.30E−05 | 3.75E−03 |
| establishment of localization | 719 | 277 | 210.13 | 1.32 | + | 2.45E−05 | 6.46E−03 |
| localization | 766 | 287 | 223.87 | 1.28 | + | 8.79E−05 | 1.55E−02 |
| small molecule catabolic process | 293 | 126 | 85.63 | 1.47 | + | 2.60E−04 | 3.16E−02 |
| small molecule metabolic process | 876 | 336 | 256.02 | 1.31 | + | 2.70E−06 | 9.51E−04 |
| ion transmembrane transport | 357 | 152 | 104.34 | 1.46 | + | 7.67E−05 | 1.43E−02 |
| transmembrane transport | 567 | 227 | 165.71 | 1.37 | + | 2.48E−05 | 6.03E−03 |
| carboxylic acid metabolic process | 505 | 198 | 147.59 | 1.34 | + | 2.46E−04 | 3.12E−02 |
| oxoacid metabolic process | 530 | 208 | 154.90 | 1.34 | + | 1.57E−04 | 2.36E−02 |
| organic acid metabolic process | 542 | 211 | 158.40 | 1.33 | + | 2.14E−04 | 2.83E−02 |
| Unclassified | 1004 | 187 | 293.42 | .64 | − | 4.03E−11 | 1.28E−07 |

GO Enrichment Analysis for GO biological process of transcripts significantly more abundant in DMSO-treated cells was conducted using Panther (http://pantherdb.org/). The Fisher's Exact test was used, and results were corrected for the false discovery rate (FDR). Only results for FDR P < 0.05 are shown.

TABLE 14
GO Enrichment Analysis for GO molecular function of transcripts significantly more abundant in DMSO-treated cells

| | E. coli | Transcripts significantly enriched in DMSO controls | | | | | |
|---|---|---|---|---|---|---|---|
| | (REF) | | | Fold | | raw P | |
| GO molecular function complete | # | # | expected | Enrichment | +/− | value | FDR |
| structural constituent of ribosome | 58 | 42 | 16.95 | 2.48 | + | 1.61E−05 | 3.59E−03 |
| structural molecule activity | 72 | 46 | 21.04 | 2.19 | + | 5.11E−05 | 9.62E−03 |
| electron transfer activity | 105 | 57 | 30.69 | 1.86 | + | 2.58E−04 | 3.52E−02 |
| oxidoreductase activity | 439 | 178 | 128.30 | 1.39 | + | 1.29E−04 | 1.97E−02 |
| catalytic activity | 2059 | 686 | 601.75 | 1.14 | + | 3.63E−05 | 7.41E−03 |
| inorganic cation transmembrane transporter activity | 123 | 64 | 35.95 | 1.78 | + | 2.42E−04 | 3.48E−02 |
| inorganic molecular entity transmembrane | 245 | 112 | 71.60 | 1.56 | + | 8.26E−05 | 1.44E−02 |
| transporter activity | | | | | | | |
| transmembrane transporter activity | 535 | 220 | 156.36 | 1.41 | + | 7.01E−06 | 2.15E−03 |
| transporter activity | 542 | 223 | 158.40 | 1.41 | + | 6.26E−06 | 2.19E−03 |
| cation transmembrane transporter activity | 168 | 83 | 49.10 | 1.69 | + | 1.05E−04 | 1.71E−02 |
| ion transmembrane transporter activity | 403 | 175 | 117.78 | 1.49 | + | 6.15E−06 | 2.516−03 |
| active transmembrane transporter activity | 234 | 104 | 68.39 | 1.52 | + | 3.63E−04 | 4.68E−02 |
| metal ion binding | 794 | 305 | 232.05 | 1.31 | + | 8.17E−06 | 2.22E−03 |
| cation binding | 806 | 308 | 235.56 | 1.31 | + | 1.09E−05 | 2.66E−03 |
| ion binding | 1329 | 477 | 388.41 | 1.23 | + | 4.15E−06 | 2.54E−03 |
| binding | 2432 | 814 | 710.76 | 1.15 | + | 2.81E−07 | 2.29E−04 |
| protein binding | 1307 | 470 | 381.98 | 1.23 | + | 4.44E−06 | 2.18E−03 |
| Unclassified | 1123 | 201 | 328.20 | .61 | − | 2.83E−14 | 3.46E−11 |

Enrichment Analysis for GO molecular function of transcripts significantly more abundant in DMSO-treated cells was conducted using Panther (http://pantherdb.org/). The Fisher's Exact test was used, and results were corrected for the FDR. Only results for FDR P < 0.05 are shown.

GO Enrichment Analysis for GO cellular component of transcripts
significantly more abundant in DMSO-treated cells.

| | E. coli | Transcripts significantly enriched in DMSO controls | | | | | |
|---|---|---|---|---|---|---|---|
| | (REF) | | | Fold | | raw P | |
| GO cellular component complete | # | # | expected | Enrichment | +/− | value | FDR |
| plasma membrane respiratory chain complex I | 16 | 14 | 4.68 | 2.99 | + | 3.50E−03 | 2.73E−02 |
| respiratory chain complex I | 16 | 14 | 4.68 | 2.99 | + | 3.50E−03 | 2.66E−02 |
| respiratory chain complex | 25 | 22 | 7.31 | 3.01 | + | 2.89E−04 | 4.39E−03 |
| membrane protein complex | 217 | 98 | 63.42 | 1.55 | + | 3.87E−04 | 4.80E−03 |
| membrane | 1384 | 470 | 404.48 | 1.16 | + | 7.12E−04 | 7.48E−03 |
| cellular anatomical entity | 2896 | 969 | 846.37 | 1.14 | + | 5.69E−11 | 1.55E−08 |
| protein-containing complex | 601 | 258 | 175.65 | 1.47 | + | 4.67E−08 | 3.19E−06 |
| respirasome | 27 | 24 | 7.89 | 3.04 | + | 1.28E−04 | 2.34E−03 |
| NADH dehydrogenase complex | 17 | 15 | 4.97 | 3.02 | + | 2.34E−03 | 2.06E−02 |
| oxidoreductase complex | 65 | 47 | 19.00 | 2.47 | + | 5.52E−06 | 1.51E−04 |
| catalytic complex | 292 | 129 | 85.34 | 1.51 | + | 7.84E−05 | 1.53E−03 |
| plasma membrane protein complex | 165 | 73 | 48.22 | 1.51 | + | 3.31E−03 | 2.66E−02 |
| plasma membrane | 1236 | 431 | 361.23 | 1.19 | + | 1.93E−04 | 3.09E−03 |
| cell periphery | 1346 | 454 | 393.38 | 1.15 | + | 1.52E−03 | 1.38E−02 |
| plasma membrane respirasome | 24 | 21 | 7.01 | 2.99 | + | 4.36E−04 | 5.17E−03 |
| cytosolic large ribosomal subunit | 32 | 25 | 9.35 | 2.67 | + | 3.46E−04 | 4.72E−03 |
| cytosolic ribosome | 57 | 44 | 16.66 | 2.64 | + | 2.91E−06 | 9.94E−05 |
| cytosol | 1051 | 394 | 307.16 | 1.28 | + | 1.48E−06 | 8.08E−05 |
| cytoplasm | 1418 | 487 | 414.42 | 1.18 | + | 1.90E−04 | 3.24E−03 |
| intracellular anatomical structure | 1457 | 509 | 425.82 | 1.20 | + | 2.25E−05 | 4.73E−04 |
| ribosome | 64 | 47 | 18.70 | 2.51 | + | 2.94E−06 | 8.91E−05 |
| intracellular non-membrane-bounded | 100 | 61 | 29.23 | 2.09 | + | 8.84E−06 | 2.19E−04 |
| organelle | | | | | | | |
| intracellular organelle | 107 | 63 | 31.27 | 2.01 | + | 1.52E−05 | 3.46E−04 |
| non-membrane-bounded organelle | 130 | 64 | 37.99 | 1.68 | + | 8.41E−04 | 8.51E−03 |
| organelle | 137 | 66 | 40.04 | 1.65 | + | 1.09E−03 | 1.07E−02 |
| large ribosomal subunit | 32 | 25 | 9.35 | 2.67 | + | 3.46E−04 | 4.50E−03 |
| ribosomal subunit | 57 | 44 | 16.66 | 2.64 | + | 2.91E−06 | 1.14E−04 |
| ribonucleoprotein complex | 59 | 45 | 17.24 | 2.61 | + | 2.37E−06 | 1.08E−04 |
| cytosolic small ribosomal subunit | 25 | 19 | 7.31 | 2.60 | + | 3.01E−03 | 2.57E−02 |
| small ribosomal subunit | 25 | 19 | 7.31 | 2.60 | + | 3.01E−03 | 2.49E−02 |
| integral component of plasma membrane | 580 | 216 | 169.51 | 1.27 | + | 1.36E−03 | 1.28E−02 |
| integral component of membrane | 1097 | 386 | 320.60 | 1.20 | + | 3.03E−04 | 4.35E−03 |
| intrinsic component of membrane | 1125 | 392 | 328.79 | 1.19 | + | 5.05E−04 | 5.74E−03 |
| intrinsic component of plasma membrane | 596 | 225 | 174.18 | 1.29 | + | 5.07E−04 | 5.54E−03 |
| Unclassified | 1438 | 300 | 420.26 | .71 | − | 8.82E−11 | 1.20E−08 |

GO Enrichment Analysis for GO cellular component of transcripts significantly more abundant in DMSO-treated cells was conducted using Panther (http://pantherdb.org/). The Fisher's Exact test was used, and results were corrected for the FDR. Only results for FDR P < 0.05 are shown.

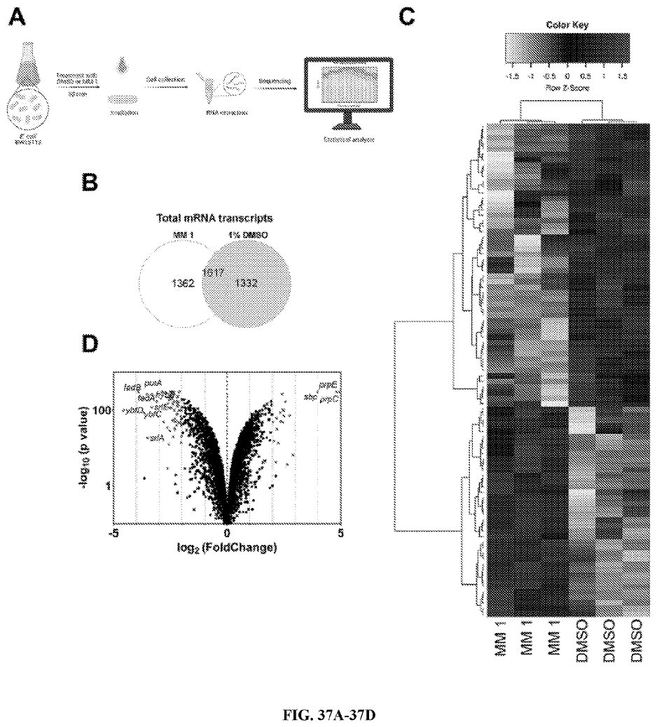

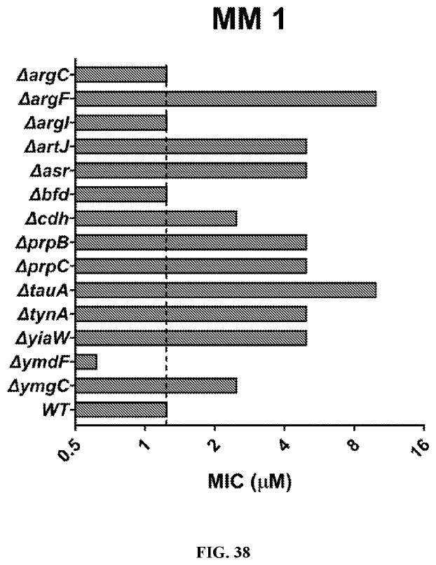

Interestingly, analysis of transcripts significantly more abundant in MM-treated cells did not reveal a significant enrichment for particular biological processes, molecular functions, or cellular components, denoting, without being bound by theory, the unspecific character of MM-induced cellular damage. To further understand whether the genes encoding the transcripts more abundant in MM-treated samples compared to DMSO controls play a role in susceptibility to MMs, the MIC for the corresponding single-gene knockouts (Table 16) was assessed. No consistent trend towards resistance or sensitivity to MM treatment was observed (FIG. 38), suggesting no particular relevance of these genes to the cell's response to MMs.

TABLE 16
List of gene transcripts significantly (p < 0.05) more abundant in MM 1-treated samples,
as determined by RNAseq, that were further examined for their susceptibility to MM treatment.

| | Transcript | | | |
|---|---|---|---|---|
| | Fold | | | |
| Gene | Change | Function | Pathway or Reaction | Location |
| argC | 2.0 | N-acetyl-gamma-glutamyl-phosphate reductase | L-ornithine biosynthesis I | Cytosol |
| argF | 2.2 | Ornithine carbamoyltransferase subunit F | L-arginine biosynthesis I (via L-ornithine) | Cytosol |
| argI | 2.8 | Ornithine carbamoyltransferase subunit I | L-arginine biosynthesis I (via L-ornithine) | Cytosol |
| artJ | 2.5 | ABC transporter arginine-binding protein 1 | Transport of L-arginine | Periplasmic space, membrane |
| asr | 2.2 | Acid shock protein | | Periplasmic space |
| bfd | 2.0 | Bacterioferritin-associated ferredoxin; | | Cytosol |
| cdh | 2.5 | CDP-diacylglycerol pyrophosphatase | Fatty acid and phosphatidic acid biosynthesis | Inner membrane |
| prpB | 5.5 | 2-methylisocitrate lyase | 2-methylcitrate cycle I | Cytosol |
| prpC | 5.0 | 2-methylcitrate synthase | 2-methylcitrate cycle I | Cytosol |
| tauA | 2.5 | Taurine-binding periplasmic protein | Transport of taurine | Inner membrane, periplasmic |
| | | | | space |
| tynA | 2.6 | Copper-containing amine oxidase | Phenylethylamine degradation I | Periplasmic space |
| | | | L-threonine degradation III (to methylglyoxal) | |
| yiaW | 2.2 | DUF3302 domain-containing protein YiaW | | Inner membrane |
| ymdF | 2.4 | Stress-induced bacterial acidophilic polypeptide | | Cytosol |
| ymgC | 2.3 | Protein involved in biofilm formation | | Cytosol |

List of gene transcripts significantly (p < 0.05) more abundant in MM 1-treated samples, as determined by RNAseq, that were further examined for their susceptibility to MM treatment by determining the MIC of the corresponding single-gene knockouts in E. coli. E. coli single-gene knockouts were obtained from CGSC, The Coli Genetic Stock Center. Information on function, pathway or reaction, and location of each gene was obtained from https://string-db.org/ and https://ecocyc.org/.

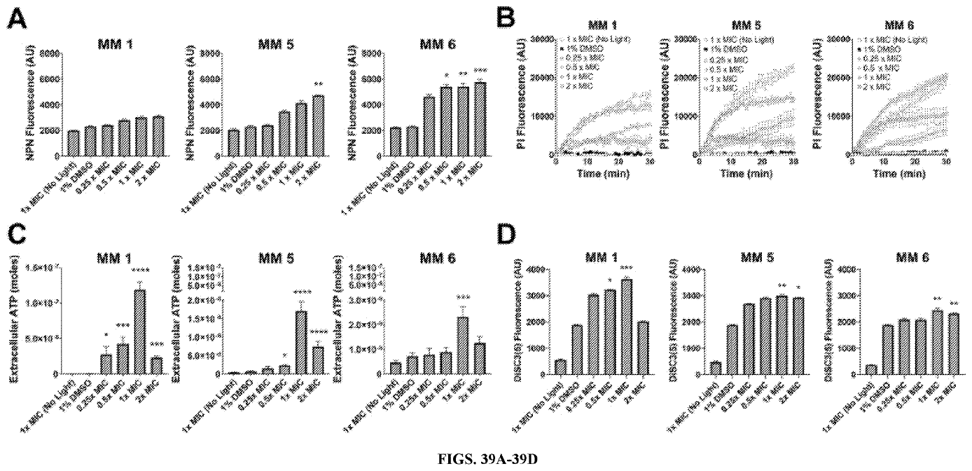

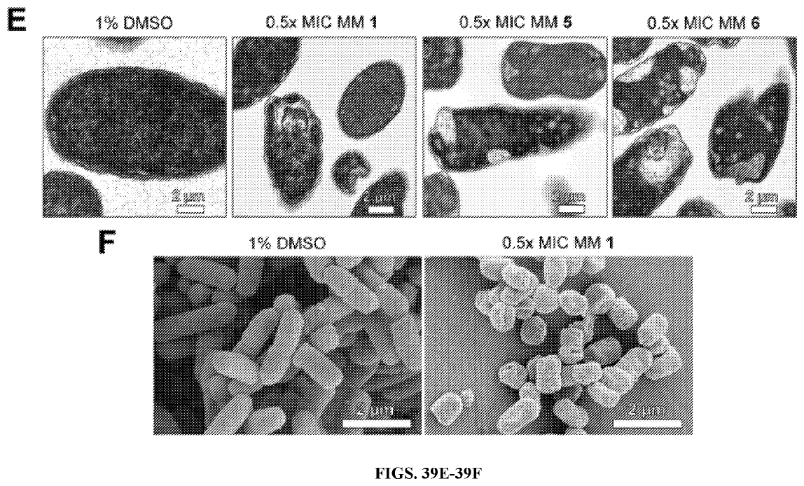

Based on the results obtained by RNAseq identifying the membrane as the major target of MMs, the investigation into the mode of action of MMs proceeded by examining their impact on inner and outer membrane integrity. The fluorescent probe N-phenyl-1-naphthylamine (NPN) was used to examine damage to the outer membrane (Helander and Matilla-Sandholm, 2000). Treatment of E. coli with MMs and 43.8 J cm⁻² of 405 nm light resulted in a concentration-dependent increase in NPN fluorescence by as much as 2.5-fold in the case of MM 6, compared to DMSO controls (FIG. 39A), denoting MM-induced damage to the outer membrane of the cell.

The effect of treatment with MMs in inner membrane permeability was examined by monitoring the fluorescence of propidium iodide (PI) in E. coli cells treated with different concentrations of each MM or DMSO in the presence and absence of 405 nm light (43.8 J cm⁻²). Treatment with MMs (0.5-1×MIC) resulted in an overall increase in PI fluorescence (FIG. 39B), denoting damage to the inner membrane of the cell in MM-treated samples.

Treatment with MMs also resulted in a significant increase in extracellular levels of ATP from 4.6×10⁻¹⁰ moles in dark DMSO controls to up to 1.2×10⁻⁷ moles in cells challenged with 1×MIC of MM 1 (FIG. 39C), denoting leakage of intracellular contents following MM treatment.

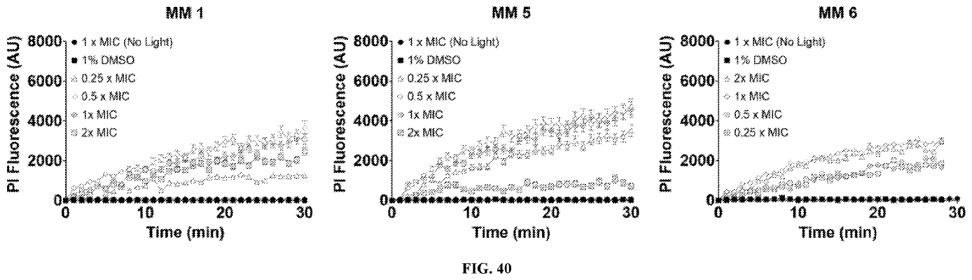

Membrane damage was accompanied by dissipation of the membrane potential, denoted by an increase in DiSC3 (5) fluorescence by as much as 1.9-, 1.6-and 1.3-fold following treatment with 1×MIC of MM 1, MM 5, and MM 6, respectively, compared to DMSO controls (FIG. 39D). Similar trends in terms of MM-induced membrane damage (FIG. 40) and loss of membrane potential (FIG. 41) were also observed in the Gram-positive S. aureus, demonstrating that the mechanism of action of MM is not species-specific.

Transmission electron microscopy (TEM) images revealed that treatment of E. coli with 0.5×MIC of MM 1, MM 5, and MM 6 and 43.8 J cm⁻² of 405 nm light resulted in substantial changes in cell morphology including the detachment of the inner membrane from the cell wall, damage to peptidoglycan, distortion of the cell surface, and formation of outer membrane vesicles denoting membrane and periplasmic stress (FIG. 39E). Scanning electron microscopy (SEM) showed that, compared to DMSO controls, samples treated with MM 1 displayed reduced cell size accompanied by deformation and wrinkling of the cell surface. MM-treated samples also exhibited multiple pore-like deformations throughout the cell surface, which were not detected in DMSO-treated cells (FIG. 39F).

### MM Potentiate Antibiotic Action

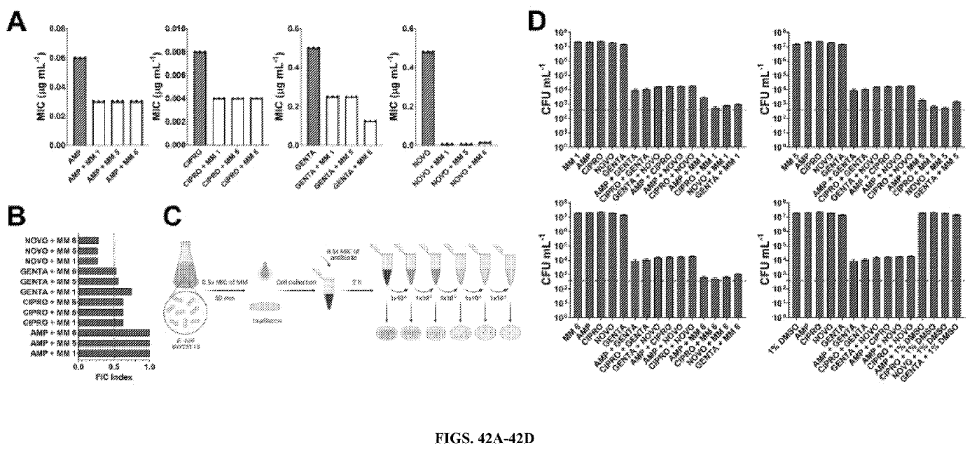

The interaction of MMs with conventional antibiotics was investigated by determining the MIC of different classes of antibiotics alone and following treatment of E. coli with sub-MIC concentrations of visible light-activated MM (FIG. 25D), using a modified checkerboard assay. In the case of the antibiotics gentamicin, ciprofloxacin, and ampicillin, pre-treatment of cells with light-activated MMs resulted in a decrease in the antibiotic MIC value by 2— to 4-fold. In the case of the antibiotic novobiocin, the MIC value was drastically reduced from 0.48 μg mL⁻¹ in untreated cells to 0.0075 μg mL⁻¹ when cells were pre-treated with MM 1 and MM 5, and to 0.015 μg mL⁻¹ in the case of pre-treatment with MM 6 (FIG. 6A). Calculation of the fractional inhibition concentration (FIC) index (Hall et al., 1983), revealed a synergistic interaction between MM and novobiocin (FIC index≤0.5), suggesting potentiation (up to 64-fold) of the action of novobiocin by pre-treatment with MM (FIG. 42B).

The ability of MMs to potentiate antibiotic killing was further examined by pre-treating cells with 0.5×MIC of MM and 43.8 J cm22 of 405 nm light prior to challenge with different antibiotics for 2 h (FIG. 42C). On average, treatment of E. coli with 4×MIC of different antibiotic combinations resulted in a 3-log reduction in bacterial numbers compared to treatment with individual antibiotics (FIG. 42D). However, cells that were pre-treated with sublethal concentrations of MMs exhibited a 4-log reduction in bacterial numbers following antibiotic challenge, compared to the treatment with individual antibiotics and antibiotic combinations. The observation that pre-treatment with MM 1, MM 5, and MM 6 resulted in a substantial increase in intracellular tetracycline fluorescence, compared to DMSO-treated samples (FIG. 42E) further indicates, without being bound by theory, that MMs potentiate antibiotic killing by permeabilizing the cell membrane and facilitating access of antibiotics to their intracellular targets.

The ability of pre-treatment with sublethal MM concentrations to potentiate antibiotic action was further investigated in P. aeruginosa treated with sub-MIC concentrations of the three most potent MM (MM 1, MM 5, and MM 6) and then challenged with increasing concentrations of the antibiotic vancomycin. Due to its large size, vancomycin (~1450 Da) usually cannot cross the outer membrane of Gram-negative bacteria (Rubenstein and Keynan, 2014). However, treatment with sub-MIC concentrations of MMs resulted in increased susceptibility of P. aeruginosa to vancomycin, denoted by inhibition of growth in checkerboard plates (FIG. 42F). Accordingly, P. aeruginosa cells pre-treated with 0.25×MIC of the different visible light-activated MMs were killed in 60 to 150 min of treatment with vancomycin (FIG. 42G).

### Therapeutic Doses of MM Mitigate Infection-Associated Mortality In Vivo

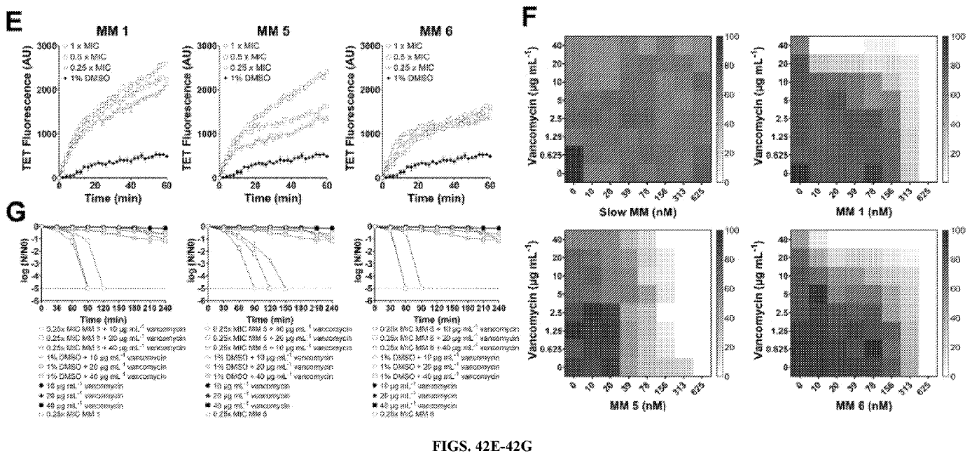

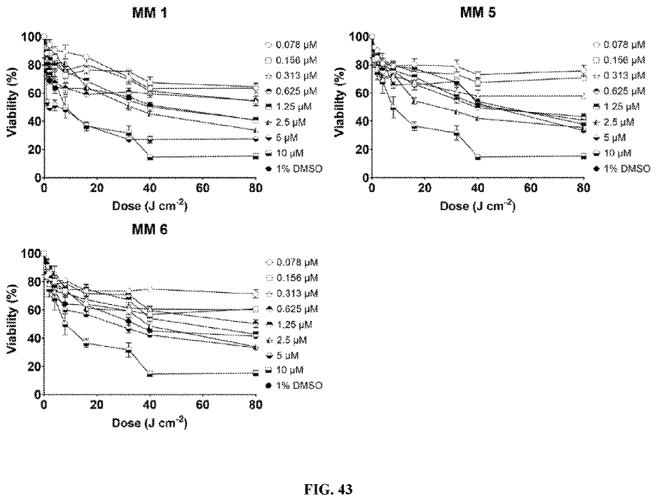

The toxicity of MM to mammalian cells was originally investigated by examining the light dose-dependent effects of different concentrations of the most potent MM (MM 1, MM 5, and MM 6) in human embryonic kidney cells (HEK). The results revealed a reduction in viability of HEK cells with increasing concentration of MM and increasing light dose (FIG. 43). The safety of MMs was further examined in both HEK cells and normal human dermal fibroblasts (NHDFs), by determining the MM concentration resulting in a 50% reduction in the viability of mammalian cells (IC50) following 5 min of irradiation at 146 mW cm⁻² (43.8 J cm-3), the same experimental conditions used to determine the bacterial MIC(Table 17). For NHDFs the IC50 ranged from 5 μM for MM 1 to 10 μM for MM 5 and MM 6, For HEK cells, the IC50 was 5 μM for the three MM tested. Based on these results, a concentration of 1× the MIC of each MM (Table 9) was used for subsequent in vivo experiments.

TABLE 17
IC₅₀ of MM 1, MM 5, and MM 6 in mammalian cell lines

| | IC50 (μM) | | MIC (μM) | |
|---|---|---|---|---|
| | NHDF | HEK | A. baumannii | S. aureus |
| MM 1 + light | 5 | 5 | 2.5 | 0.156 |
| MM 5 + light | 10 | 5 | 2.5 | 0.078 |
| MM 6 + light | 10 | 5 | 2.5 | 0.078 |

IC50 of MM 1, MM 5, and MM 6 in mammalian cell lines was determined using the CellTiter-Glo ® Luminescent Cell Viability Assay (Promega). Primary normal human dermal fibroblasts (NHDF). Human Embryonic Kidney cells (HEK). The corresponding MIC in the two bacterial strains used during in vivo assays is shown for comparison. The mean of at least 3 replicas is shown. IC50: MM concentration resulting in a 50% reduction in the viability of mammalian cells.

The in vivo antibacterial activity of MM was investigated in a burn wound infection model of the invertebrate Galleria mellonella (Maslova et al., 2020). Following the generation of a burn wound in the worm, wounds were infected with either the Gram-positive S. aureus or the Gram-negative A. baumannii. Infected wounds were then treated with 1% DMSO, 1×MIC of conventional antibiotics (polymyxin B in the case of A. baumannii infection and tobramycin in the case of S. aureus infection), 1% DMSO, or 1×MIC of MM 1, MM 5, or MM 6 for each bacterial strain and 43.8 J cm⁻² of 405 nm light (FIG. 44A). The survival of worms under different treatments was monitored for up to 7 days post-treatment.

All (100%) worms infected with A. baumannii and treated with 1% DMSO only (no light) died by day 6 post-treatment (FIG. 44B). Treatment with 1×MIC of polymyxin B attenuated mortality after 7 days to 69% (p<0.0001, Table 18), while treatment with MMs attenuated mortality after 7 days to 40-60% (p<0.0001). In the case of worms infected with S. aureus and treated with 1% DMSO (no light), 100% mortality was observed 7 days post-treatment. Treatment with 1×MIC of tobramycin mitigated mortality at 7 days to 33%, while treatment with MMs mitigated mortality after 7 days to 17-25% (p<0.0001).

TABLE 18
Statistical significance of the difference between survival curves of G. mellonella
infected with A. baumannii and S. aureus and treated with 1x MIC of different MM, 1x
MIC of the antibiotics polymyxin B (A. baumanni) or tobramycin (S. aureus).

| | | Control (1% DMSO, no light) versus | | | | |
|---|---|---|---|---|---|---|
| | | MM 1 | MM 5 | MM 6 | 1% DMSO | Antibiotic |
| A. baumannii | p-value | <0.0001 | <0.0001 | <0.0001 | 0.0003 | <0.0001 |
| | p-value | **** | **** | **** | *** | **** |
| | summary | | | | | |
| S. aureus | p-value | <0.0001 | <0.0001 | <0.0001 | 0.4051 | <0.0001 |
| | p-value | **** | **** | **** | ns | **** |
| | summary | | | | | |

Statistical significance of the difference between survival curves of G. mellonella infected with A. baumannii and S. aureus and treated with 1x MIC of different MM, 1x MIC of the antibiotics polymyxin B (A. baumannii) or tobramycin (S. aureus) was determined in GraphPad Prism 8 using the Log-rank (Mantel-Cox) test. Each survival curve was generated based on results obtained from 3 biological replicas each comprised of 16 individuals, totaling 48 data points.

### DISCUSSION

The present disclosure describes an antibacterial therapy based on the use of synthetic visible light-activated MMs that kill bacteria by mechanical damage. At therapeutic doses, synthetic MMs were activated by visible light to kill bacteria, including both Gram-positive and Gram-negative bacteria, such as methicillin-resistant S. aureus (MRSA). The presently disclose methods provide for the killing of bacteria within minutes, vastly outperforming conventional antibiotics (FIG. 33A, FIG. 33B).

Besides exponentially growing cells, MMs as disclosed herein also rapidly eliminated persister cells (FIG. 35A). Persister cells are defined as transiently antibiotic-tolerant fractions of bacterial populations that are metabolically inactive or dormant (Lewis, 2007). In this phenotypic state, the biosynthetic processes targeted by conventional antibiotics are inactive or significantly attenuated, making them highly tolerant to conventional antibiotics that typically affect growing bacteria with an active metabolism (Lewis, 2010; Allison et al., 2011; Conlon et al., 2013).

MMs were also able to significantly reduce the cell number and biomass of established biofilms of P. aeruginosa and S. aureus (FIGS. 35B-35F). Similar to persister cells, biofilms are considered resistant phenotypes, characterized by the presence of a heterogeneous dense extracellular polymeric matrix that includes extracellular DNA, proteins, and polysaccharides in which high densities of microbial cells are entrapped (Ch′ng et al., 2019). This complex milieu provides a barrier to antibiotic diffusion and penetration, making biofilm-associated infections frequently refractory to conventional antimicrobial therapy (Stewart, 2002; Vuotto et al., 2014; Donlan, 2000). However, the irradiation conditions necessary for complete elimination of biofilms are much higher than those safe for mammalian cells (Table 17), and at the extended irradiation times necessary to completely reduce biofilm biomass (up to 45 min), an effect of temperature cannot be excluded.

Contrary to conventional antibiotics, repeated exposure to MMs in serial passage experiments (Sommer et al., 2017) was not accompanied by a change in MIC value (FIG. 35F), suggesting a low propensity for the development of resistance to MM therapy across different bacteria.

Since MM-resistant mutants could not be isolated, the mechanism of action of these new molecules was investigated using gene expression analysis via RNAseq, and an array of spectrophoto- and spectrofluorimetric methods, and electron microscopy in E. coli. MM-and DMSO-treated cells displayed strikingly distinct transcriptomic profiles (FIG. 37C). Transcripts significantly more abundant in DMSO-treated cells compared to MM-treated cells were overwhelming enriched for membrane-associated processes (Tables 13-15), identifying the membrane as the major target of MM. Increased fluorescence of dyes used to monitor damage to the inner and outer bacterial membrane (FIG. 39A, FIG. 39B) further demonstrated that the mechanism of action of MMs involves unspecific, widespread damage to the cell envelope. Membrane damage was followed by leakage of intracellular components, denoted by increased levels of extracellular ATP (FIG. 39C), and loss of the ability to sustain the membrane potential, evidenced by increased fluorescence of the membrane potential dye DiSC3 (5) (FIG. 39D). Electron microscopy revealed extensive damage to the cell ultrastructure following MM treatment, particularly at the level of the membrane and cell wall, including the presence of physical deformities reminiscent of holes in the cell surface that were absent in DMSO controls (FIG. 39E, FIG. 39F).

These results suggest, without being bound by theory, that the mode of action of MM is distinct from that of membrane-targeting, pore-forming antibiotics such as nisin or daptomycin, which involve docking to specific binding sites in the membrane and the oligomerization of the antibiotic molecule to form a pore or ion channel (Kosmidis and Levine, 2010; Prince et al., 2016). Resistance to such antibiotics has been reported and attributed to altered cell wall and cell membrane composition and function in resistant mutants (Tran et al., 2015; Bayer et al., 2013). The fact that MMs were able to rapidly kill a range of Gram-positive and Gram-negative bacteria, including antibiotic-resistant strains and efflux knockouts, suggests that the antibacterial action of MMs in the presently disclosed methods does not involve binding to specific elements within the bacterial envelope. Rather, the mechano-bactericidal action of MMs via physical membrane disruption is unlike any other antibacterial modality known in the art. This molecular-level generalized, unspecific membrane damage can also possibly account, again without being bound by theory, for the ability of MMs to efficiently eradicate persisters, which are particularly susceptible to membrane-targeting agents (Hurdle et al., 2011).

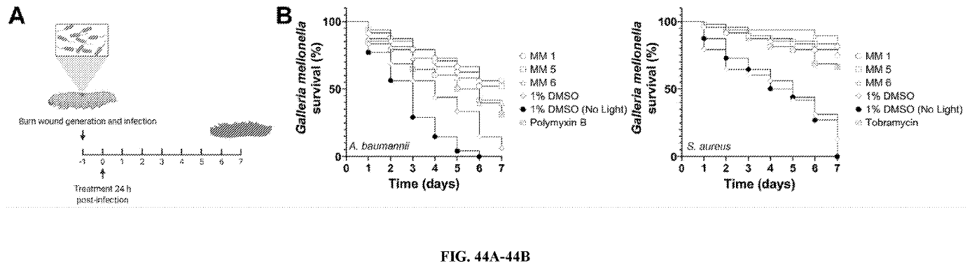

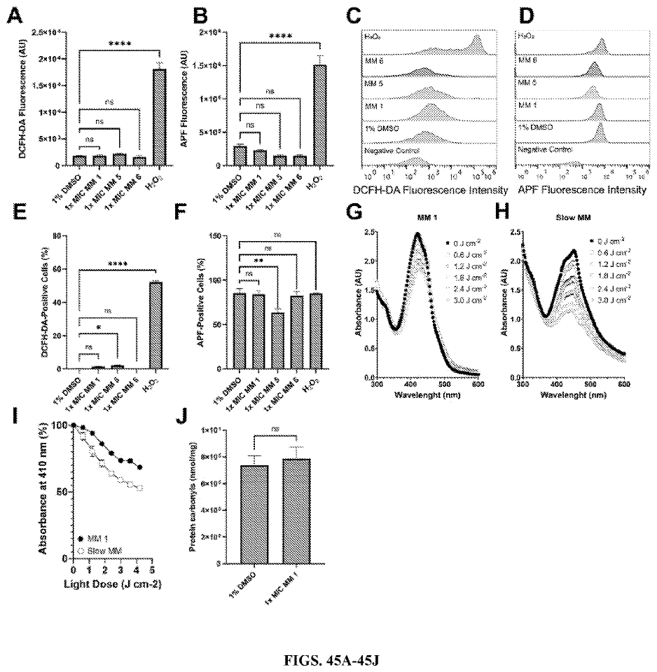

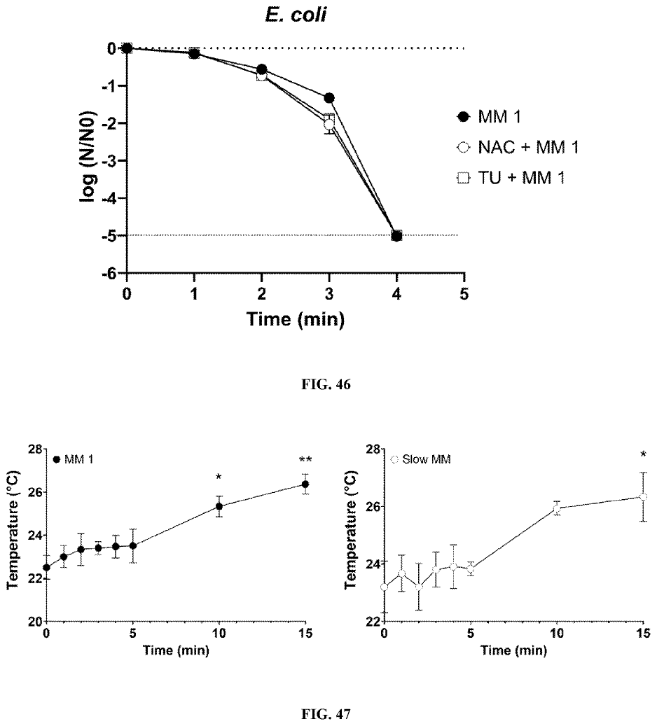

A mode of action that involves physical membrane disruption may also explain the undetectable levels of resistance after repeated exposure to MMs (FIG. 35F). A low propensity for resistance development could also be explained, without being bound by theory, by the involvement of two distinct modes of action in the antibacterial activity of MMs: (1) physical membrane damage resulting from fast rotation of MM following light activation, and (2) antibacterial effects of blue light. Blue light (400-490 nm) has well-known antibacterial properties (Hamblin et al., 2005; Dai et al., 2012; Wang et al., 2016; Amin et al, 2015) and also shows a low propensity for resistance development (Amin et al., 2016, Zhang et al., 2016). The antibacterial mechanism of action of blue light appears to involve, without being bound by theory, the generation of reactive oxygen species (ROS) and subsequent oxidative damage to biomolecules (Cieplik et al., 2014; Dai et al., 2013; O'Donoghue et al., 2016). However, a significant difference in ROS levels in MM-treated cells and DMSO-treated cells using the ROS-sensitive probes DCFH-DA and APF was not detected at either the population (microplate format) or single-cell levels (flow cytometry) in the case of MM 1 and 6, while treatment with MM 5 resulted in a small increase in DCFH-DA positive cells (FIGS. 45A-45F). Singlet oxygen generation by the irradiated antibacterial motor MM 1, detected using the singlet oxygen trap DPBF (García-López et al., 2017), was lower than that of its slow, inert counterpart (FIGS. 45G-451), suggesting, without being bound by theory, that singlet oxygen generation is not associated with the antibacterial potential of molecular motors. Likewise, the levels of protein carbonyls in cells treated with DMSO or the potent antibacterial motor MM 1 were not significantly changed (FIG. 45J). Additionally, the MM-induced inactivation profiles of E. coli treated with ROS scavengers prior to irradiation were similar to those of untreated cells (FIG. 46). Taken together, these results indicate, without being bound by theory, that ROS do not play a significant role in MM-induced bacterial killing. In fact, ROS quenching by the central twisted carbon-carbon double bond characteristic of MM was previously reported (Ayala Orozco et al., 2020) and, to a certain extent, was also observed in the present study (FIG. 45E).

Importantly, antibacterial effects were only observed in MM-treated cells that were irradiated, but not in those kept in the dark, denoting the importance of light for the antibacterial effects of MMs. Since the irradiation conditions used for most experiments (5 min of irradiation with 405 nm light at 146 mW cm⁻²), did not result in a significant increase in temperature, and the temperature variations in samples treated with fast antibacterial MM (23.9±1.3 ° C) and slow MM (24.4±1.3 ° C) without antibacterial activity were similar (FIG. 47), temperature alone cannot account for the antibacterial effects of MMs.

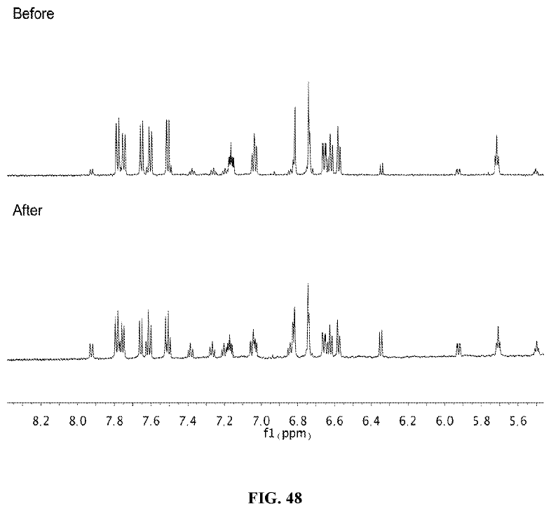

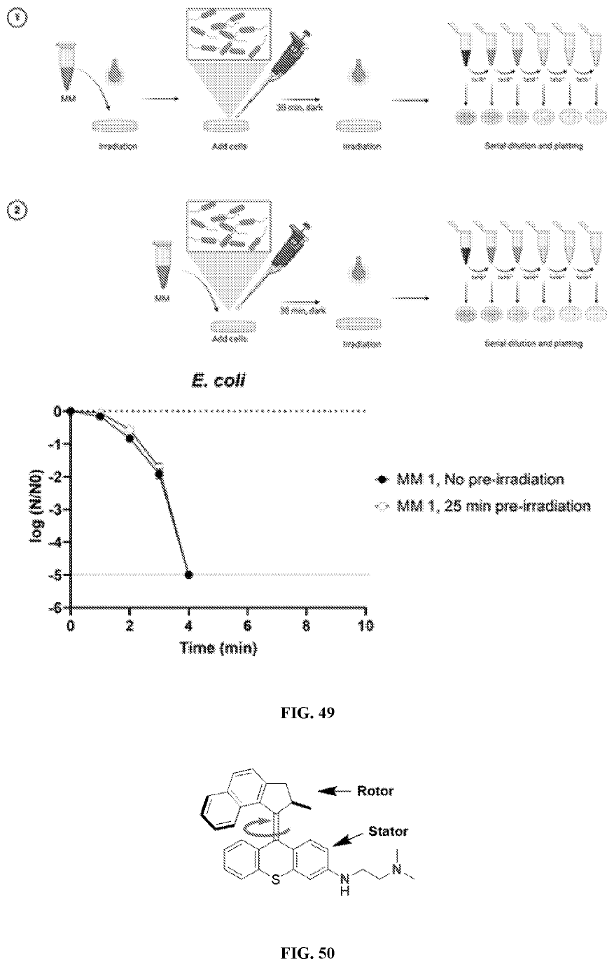

Also, irradiated MMs maintained their integrity and biological activity and did not undergo photodecomposition, as evidenced by NMR spectra before and after irradiation displaying only changes in isomer ratios but the absence of new aromatic signals (FIG. 48). Sustained antibacterial activity of extensively pre-irradiated MM (FIG. 49) provides further evidence that phototoxicity is not the mechanism of action of MM.

Finally, slow MM (10⁻³ Hz) chemically analogous to antibacterial MM (≈3 MHz) did not exhibit antibacterial activity (FIG. 27), demonstrating the importance of fast mechanical rotation for the antibacterial properties of MM.

Taken together, these results indicate that, without being bound by theory, under the experimental conditions examined, MM-induced antibacterial effects can be attributed to the rapid drilling-like unidirectional rotation of MM following light activation, whereby the rotor portion of the molecule spins around the central olefinic bond (FIG. 25B) propelling the molecule through the membrane (FIG. 25C). Subsequent leakage of cell contents and loss of membrane potential eventually culminate in bacterial cell death.

The reduced permeability of the Gram-negative membrane represents an important challenge for antibacterial therapy by posing a barrier that limits antibiotic entrance to the cell (Pagès et al., 2008; Niakido, 2003). The present disclosure provides, besides methods for killing bacteria, methods for potentiating the killing of E. coli by traditional antibiotics, as demonstrated by (1) a reduction of antibiotic MIC values when antibiotic treatment was preceded by exposure of cells to sublethal doses of MMs (FIG. 42A) and, (2) enhanced killing by antibiotics following pre-exposure of cells to sublethal MM (FIG. 42D). Increased intracellular tetracycline fluorescent signal in cells pre-treated with visible light-activated MMs (FIG. 42E) suggests, without being bound by theory, that the enhanced antibiotic killing of cells pre-treated with MMs is a result of MM-induced cell permeabilization and increased accessibility of antibiotics to their intracellular targets.

This effect was not only observed in E. coli but also in the opportunistic pathogen P. aeruginosa. Due to its hydrophilicity and large size, vancomycin (~1450 Da) usually cannot cross the outer membrane of Gram-negative bacteria (Rubenstein and Keynan, 2014). However, P. aeruginosa challenged with sublethal concentrations of fast light-activated MMs displayed substantial growth inhibition following subsequent treatment with vancomycin (FIG. 42F) and were completely killed in as little as 60 min by the otherwise ineffective vancomycin (FIG. 42G). These results demonstrate the ability of MMs to permeabilize the Gram-negative outer membrane to substances that would otherwise be excluded, including typical Gram-positive antibiotics, like vancomycin.

The presently disclosed MMs and methods of use thereof demonstrate that, by permeabilizing the Gram-negative outer membrane and improving the accessibility of antibiotics to intracellular targets, MMs exert an antibiotic co-adjuvant action. Future work should aim to identify other antibacterial molecules whose action can be potentiated by visible light active MM-induced membrane permeabilization.

The safety of MMs to mammalian cells was investigated in vitro in two mammalian cell lines subjected to the same irradiation conditions used to determine the bacterial MIC. The intensity (146 mW cm⁻²) and dose/fluence (43.8 J cm⁻²) of 405 nm light used throughout most of the presently described experiments are comparable, or lower, to those previously shown to be safe for mammalian cells in vitro and in vivo (40, 45-48). The proximity of the IC50 and MIC(Table 17), particularly in A. baumannii, demonstrates the broad destructive capabilities of MM, previously reported for UV-activated MMs (García-López et al., 2017; Gunasekera et al., 2020).

Given these safety concerns, the invertebrate infection model Galleria mellonella was used to investigate the in vivo anti-infective capabilities of MMs. G. mellonella is a well-established, inexpensive, and low maintenance model of fungal and bacterial infections (Ramarao et al., 2012; Harding et al., 2012; Mylonakis et al., 2005; Junior et al., 2013). While insects like G. mellonella do not have an adaptive immune response and cannot generate antibodies, their complex innate immune system shows some similarities to that of mammals (Wojda, 2017). Importantly, correlations between immune responses to pathogens in G. mellonella and mice demonstrate that results obtained using this invertebrate model can provide significant insights into the mammalian response (Jander et al., 2000; Borman, 2018; Brennan et al., 2002).

Due to their location, skin wounds, such as burns, are particularly amenable to light-mediated antimicrobial therapies. Systemic antimicrobials have limited efficiency in the treatment of such localized infections due to poor blood flow to these areas and the presence of dead tissue (Leaper, 2006; Halstead et al., 2015) while potentially contributing to the development of resistance in non-target organisms (Halstead et al., 2015). A G. mellonella burn wound infection model was recently described (Maslova et al., 2020).

In this work, burn wounds of G. mellonella were infected with two of the major bacterial pathogens typically associated with burn wounds, A. baumannii and S. aureus (Guggenheim et al., 2009; Fu et al., 2012). Treatment of infected worms with visible light-activated MMs mitigated the mortality associated with infection by both A. baumannii and S. aureus (FIG. 44B). Mortality mitigation by MMs (up to 83%) was similar or superior to that of conventional antibiotics. These results demonstrate the potential of MMs in the treatment of localized bacterial infections, despite the small therapeutic window.

### (i) Synthetic Chemistry

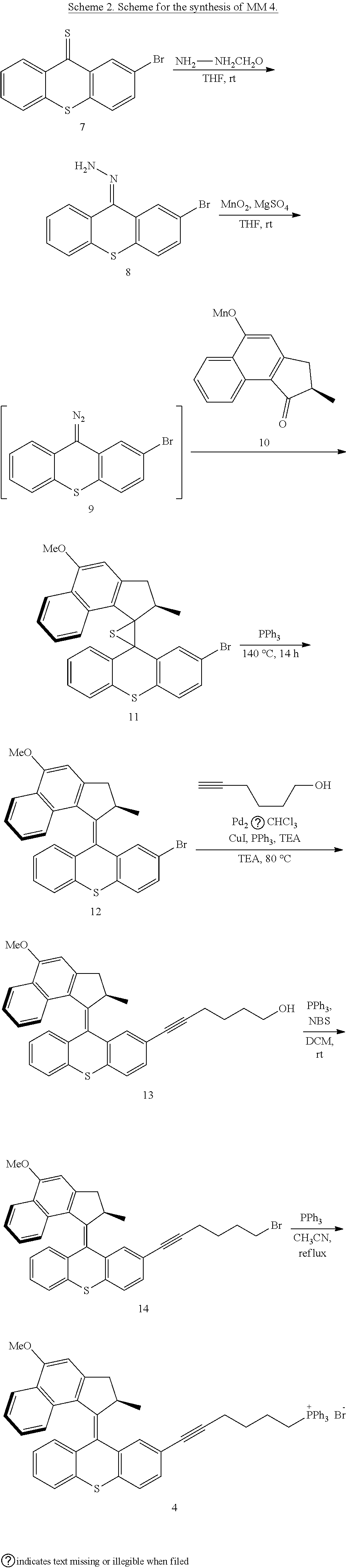

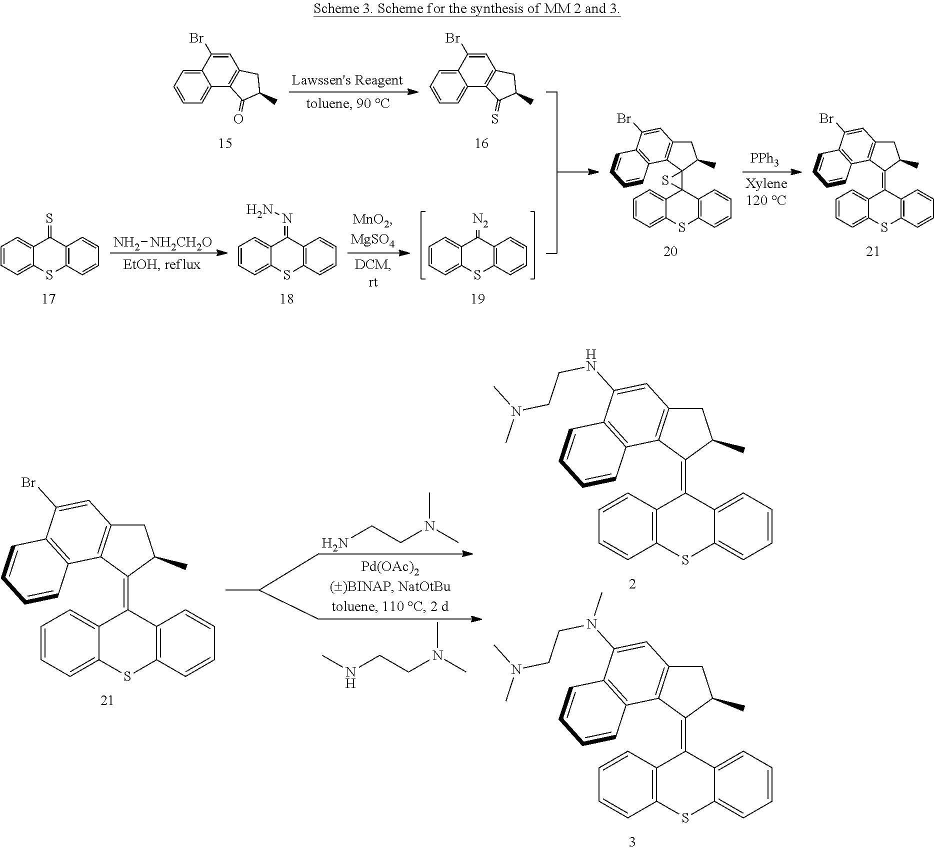

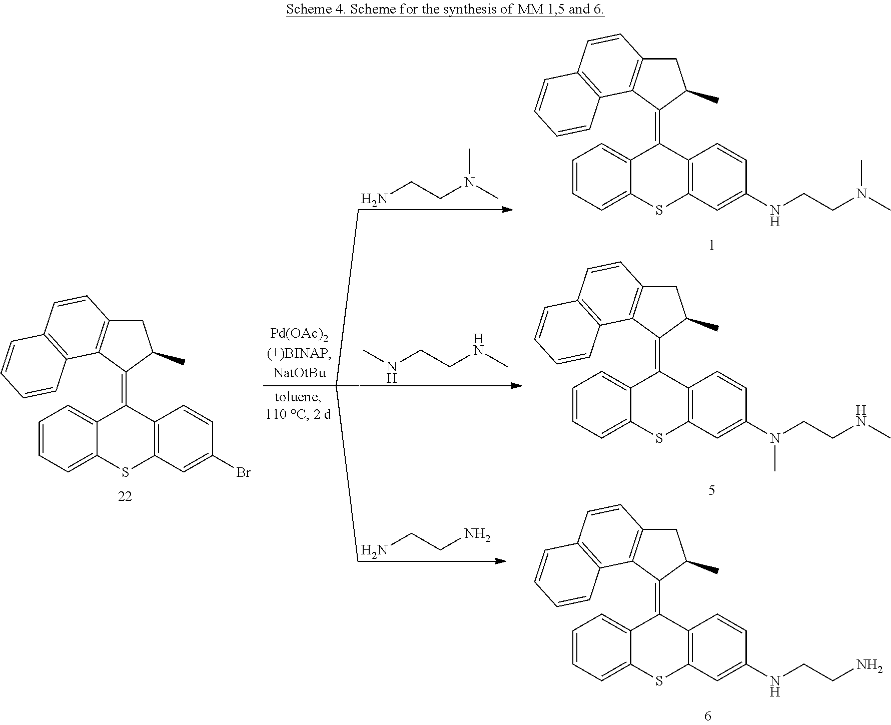

General Methods. All glassware was oven-dried overnight prior to use. Reagent grade dichloromethane (DCM, CH₂Cl₂) was distilled from calcium hydride (CaH₂) under N₂ atmosphere. All reactions were carried out under N₂ atmosphere unless otherwise noted. All other chemicals were purchased from commercial suppliers and used without further purification. Flash column chromatography was performed using 230-400 mesh silica gel from EM Science. Thin layer chromatography (TLC) was performed using glass plates pre-coated with silica gel 40 F254 0.25 mm layer thickness purchased from EM Science. ¹H NMR and ¹³C NMR spectra were recorded at 400/500 and 100/125 MHz, respectively. Chemical shifts (8) are reported in ppm from tetramethylsilane (TMS). Compound 7, 10, 15, and 17 was prepared by literature protocols (10, 11, 105, 106). The syntheses of motor 22 were reported previously (Saywell et al, 2016).

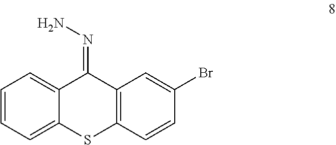 [SMILES: N/N=c1\c2ccccc2sc2ccc(Br)cc12]

(E)-(2-bromo-9H-thioxanthen-9-ylidene) hydrazine (8). An oven dried round-bottom flask equipped with a stir bar was charged with compound 7 (188 mg, 0.616 mmol) in THF (5.0 ml), hydrazine monohydrate (1.0 ml) was added and the mixture was stirred at rt for 1 h. The resulting mixture was concentrated in vacuo. The resulting concentrate was used for the next step without further purification.

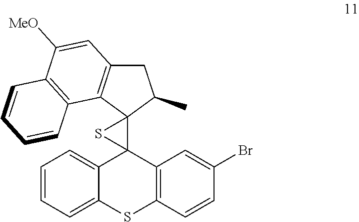

2″-bromo-5-methoxy-2-methyl-2,3-dihydrodispiro[cyclopenta[a]naphthalene-1,2′-thiirane-3′,9″-thioxanthene] (11). To an oven dried round-bottom flask charged with hydrazone 8 and MgSO₄ (200 mg, 100% w/w) was added THF (5.0 mL). To this suspension was quickly added MnO₂ (1.0 g, 11.4 mmol, Sigma-Aldrich >90%) at rt. The mixture was stirred for 1 h at the same temperature. The mixture was filtered, and the filtrate was concentrated in vacuo. To the resulting concentrate was added toluene (5.0 mL) and thioketone 10 (84.2 mg, 0.348 mmol). The mixture was heated to 100 ° C and stirred for 3 h. After the reaction mixture was cooled to room temperature. The organic phase was dried over anhydrous MgSO₄, filtered and the filtrate was concentrated in vacuo, followed by purification by column chromatography (SiO₂; 10% acetone in DCM) to afford 11 as a bright yellow solid (56.7 mg, 32% for 2 steps): ¹H NMR (500 MHz, Chloroform-d) δ 8.89 (ddd, J=8.5, 1.4, 0.6 Hz, 1H), 8.09-7.68 (m, 2H), 7.47-7.39 (m, 1H), 7.37-7.33 (m, 1H), 7.33-7.29 (m, 1H), 7.25-7.17 (m, 1H), 6.99 (ddd, J=7.7, 1.2, 0.5 Hz, 1H), 6.95 (td, J=7.6, 1.2 Hz, 1H), 6.86-6.80 (m, 1H), 6.80-6.76 (m, 1H), 6.57 (d, J=1.3 Hz, 1H), 3.92 (d, J=1.9 Hz, 3H), 3.46 (td, J=14.8, 6.5 Hz, 1H), 2.41 (dd, J=15.3, 7.4 Hz, 1H), 1.64-1.47 (m, 1H), 1.13 (t, J=7.1 Hz, 3H). ¹³C NMR (126 MHz, CDCl₃) δ 156.15, 156.08, 143.13, 143.08, 142.12, 138.98, 137.18, 136.10, 135.89, 135.37, 134.89, 134.67, 133.94, 131.68, 131.57, 131.53, 130.94, 129.76, 129.12, 128.95, 128.27, 127.86, 127.02, 126.83, 126.78, 126.70, 126.09, 124.83, 124.78, 124.58, 124.54, 124.49, 124.08, 123.82, 123.68, 122.72, 122.58, 121.57, 121.47, 120.62, 119.73, 101.96, 101.88, 72.68, 72.59, 61.26, 61,18, 55.35, 55.34, 40.71, 40.33, 38.90, 38.85, 22.17, 22.06.

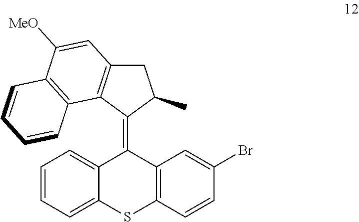

2-bromo-9-(5-methoxy-2-methyl-2,3-dihydro-1H-cyclopenta[a]naphthalen-1-ylidene)-9H-thioxanthene (12). To a 100 mL screw-capped tube charged with episulfide 11 (56 mg, 0.109 mmol) was added triphenylphosphine (57 mg, 0.218 mmol), and the mixture was stirred at 140 ° C for 14 h. After the reaction mixture was cooled to room temperature, the resulting mixture was partitioned between DCM (10 mL) and saturated NH₄Cl (aq) (10 mL). The organic layer was dried over anhydrous MgSO₄, filtered and the filtrate concentrated in vacuo. The resulting concentrate was purified by column chromatography (silica gel; 30% DCM in hexanes) to afford 12 as a pale-yellow solid (48.4 mg, 92%).

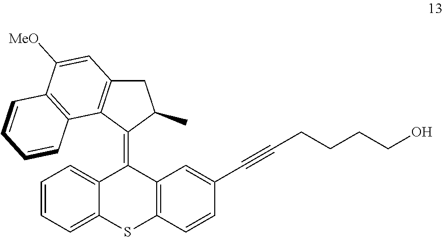

6-(9-(5-methoxy-2-methyl-2,3-dihydro-1H-cyclopenta[a]naphthalen-1-ylidene)-9H-thioxanthen-2-yl) hex-5-yn-1-ol (13). An oven dried round-bottom flask equipped with a stir bar was charged with motor 12 (48 mg, 0.1 mmol), tris(dibenzylideneacetone) dipalladium (0)-chloroform adduct (2.6 mg, 0.01 mmol), CuI (1.9 mg, 0.01 mmol), triphenylphosphine (5.3 mg, 0.02 mmol) and hex-5-yn-1-ol (0.08 mL, 1 mmol). NEt₃ (3 mL) was added and the mixture was stirred at 70 ° C overnight. The resulting mixture was filtered and the filtrate concentrated in vacuo. The resulting yellow solution was partitioned between DCM (20 mL) and water (20 mL). The organic phase was dried over anhydrous MgSO₄, filtered and the filtrate was concentrated in vacuo, followed by purification by column chromatography (SiO2; 10% acetone in DCM) to afford 13 as a bright yellow solid (31.4 mg, 63%).

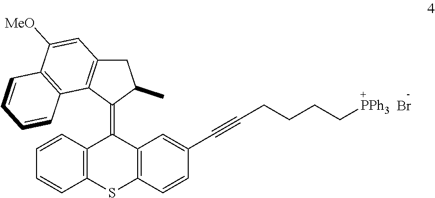

(6-(9-(5-methoxy-2-methyl-2,3-dihydro-1H-cyclopenta[a]naphthalen-1-ylidene)-9H-thioxanthen-2-yl) hex-5-yn-1-yl)triphenylphosphonium bromide (4). An oven dried round-bottom flask equipped with a stir bar was charged with motor 13 (25 mg, 0.05 mmol), NBS (18 mg, 0.1 mmol) and PPh₃ (26 mg, 0.1 mmol) at 0 ° C Saturated solutions of sodium thiosulfate (10 mL) and sodium bicarbonate (10 mL) were added to the reaction mixture. The organic phase was separated, and the aqueous phase was extracted with methylene chloride. The combined organic layers were washed with brine, dried over MgSO₄, and then concentrated in vacuo. The resulting concentrate was purified by flash column chromatography and the resulting bromide product 14 was used for the next step due to its instability.

To a stirred solution of compound 14 in dry acetonitrile (4 mL) at room temperature, PPh₃ (52 mg, 0.2 mmol) was added, and then the mixture was allowed to stir under reflux for 48 b. After TLC analysis indicated the consumption of the starting material, the solvent was subsequently removed under reduced pressure, and the residue was purified by flash chromatography to afford compound 1 as a bright yellow solid (17.7 mg, 43% for two steps).

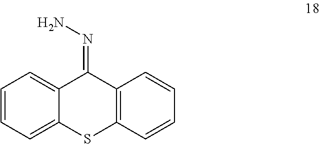 [SMILES: NN=c1c2ccccc2sc2ccccc12]

(9H-thioxanthen-9-ylidene) hydrazine (18). An oven dried round-bottom flask equipped with a stir bar was charged with compound 17 (50 mg, 0.221 mmol) in THF (5.0 ml), hydrazine monohydrate (1.0 ml) was added, and the mixture was stirred at rt for 1 h. The resulting mixture was concentrated in vacuo. The resulting concentrate was used for the next step without further purification.

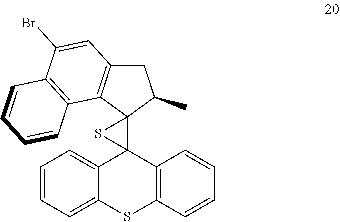

5-bromo-2-methyl-2,3-dihydrodispiro[cyclopenta[a]naphthalene-1,2′-thiirane-3′,9″-thioxanthene] (20). To an oven dried round-bottom flask charged with hydrazone 18 and MgSO₄ (100 mg, 200% w/w) was added THF (5.0 mL). To this suspension was quickly added MnO₂ (500.0 mg, 5.7 mmol, Sigma-Aldrich >90%) at rt. The mixture was stirred for 1 h at the same temperature. The mixture was filtered, and the filtrate was concentrated in vacuo. To the resulting concentrate was added toluene (5.0 mL) and thioketone 16 (34.6 mg, 0.119 mmol). The mixture was heated to 100 ° C and stirred for 3 h. After the reaction mixture was cooled to room temperature, the organic phase was dried over anhydrous MgSO₄, filtered and the filtrate was concentrated in vacuo, followed by purification by column chromatography (SiO2; 10% acetone in DCM) to afford 20 as a bright yellow solid (38.6 mg, 67% for 2 steps): ¹H NMR (500 MHz, Chloroform-d) δ 9.12-8.96 (m, 1H), 7.99-7.93 (m, 1H), 7.92-7.83 (m, 1H), 7.75-7.63 (m, 1H), 7.52 (s, 1H), 7.45 (ddd, J=7.6, 1.3, 0.5 Hz, 1H), 7.34-7.27 (m, 2H), 7.25-7.21 (m, 1H), 7.00 (ddd, J=7.7, 1.3, 0.4 Hz, 1H), 6.90 (td, J=7.6, 1.2 Hz, 1H), 6.77 (qd, J=7.6, 1.4 Hz, 1H), 3.48 (dd, J=15.3, 6.6 Hz, 1H), 2.38 (d, J=15.3 Hz, 1H), 1.54, (m, 1H), 1.07 (d. J=7.0 Hz, 3H). ¹³C NMR (126 MHz, Chloroform-d) ô 143.19, 139.17, 136.61, 135.77, 134.61, 132.20, 131.92, 130.68, 130.57, 128.77, 127.87, 126.99, 126.94, 126.82, 126.81, 126.65, 126.55, 125.83, 125.67, 125.25, 124.87, 123.64, 71.51, 62.39, 40.84, 37.94, 21.67.

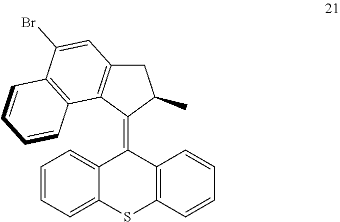

9-(5-bromo-2-methyl-2,3-dihydro-1H-cyclopenta[a]naphthalen-1-ylidene)-9H-thioxanthene (21). To a 100 mL screw-capped tube charged with episulfide 20 (101 mg, 0.208 mmol) was added triphenylphosphine (108 mg, 0.416 mmol), and the mixture was stirred at 140 ° C for 14 h. After the reaction mixture was cooled to room temperature, the resulting mixture was partitioned between DCM (10 mL) and saturated NH₄Cl (aq) (10 mL). The organic layer was dried over anhydrous MgSO₄, filtered and the filtrate concentrated in vacuo. The resulting concentrate was purified by column chromatography (silica gel; 20% DCM in hexanes) to afford 21 as a pale yellow solid (86.4 mg, 89%): ¹H NMR (500 MHz, Chloroform-d) δ 8.12 (ddd, J=8.5, 1.3, 0.7 Hz, 1H), 7.80 (dd, J=7.8, 1.4 Hz, 1H), 7.78 (s, 1H), 7.65-7.61 (m, 1H), 7.59 (ddd, J=7.8, 1.2, 0.5 Hz, 1H), 7.35 (td, J=7.6, 1.3 Hz, 1H), 7.30-7.27 (m, 1H), 7.26-7.22 (m, 1H), 7.01 (ddd, J=7.8, 7.3, 1.4 Hz, 1H), 6.94 (ddd, J=8.5, 1.3, 0.7 Hz, 1H), 6.84 (ddd, J=8.3, 6.7, 1.3 Hz, 1H), 6.71 (ddd, J=7.7, 1.5, 0.5 Hz, 1H), 6.62 (td, J=7.5, 1.2 Hz, 1H), 4.39-4.27 (m, 1H), 3.66 (dd, J=15.5, 6.2 Hz, 1H), 2.62 (d, J=15.5 Hz, 1H), 0.80 (d, J=6.9 Hz, 3H). ¹³C NMR (126 MHz, CDCl₃) δ 145.93, 145.08, 139.89, 137.60, 135.60, 135.58, 135.42, 130.93, 129.75, 129.22, 128.54, 127.98, 127.78, 127.56, 127.50, 126.86, 126.56, 126.42, 126.39, 126.26, 126.11, 125.54, 125.40, 124.21, 39.49, 37.86, 19.35.

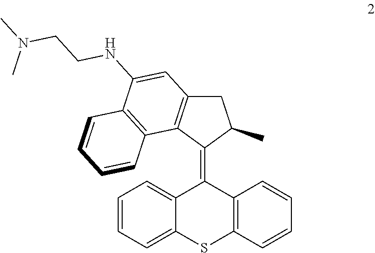

N¹,N¹-Dimethyl-N²-(2-methyl-1-(9H-thioxanthen-9-ylidene)-2,3-dihydro-1H-cyclopenta[a]naphthalen-5-yl) ethane-1,2-diamine (2). BINAP (2.5 mg, 0.0066 mmol) and palladium (II) acetate (0.5 mg, 0.0022 mmol) were dissolved in dry toluene (5 mL). This solution was stirred for 30 min at rt, where upon it turned from dark red to dark orange. After this period NaOtBu (25 mg, 0.22 mmol) was added, followed by bromo-substituted motor 20 (20 mg, 0.044 mmol) and N¹N¹-dimethylethane-1,2-diamine (20 mg, 0.22 mmol). The mixture was stirred at 90 ° C for 2 d. Subsequently the reaction mixture was poured into CH₂Cl₂ (10 mL). After filtration the solvents were evaporated. The crude product was dissolved in a small amount of CH₂Cl and purified using column chromatography (SiO₂; 10% MeOH in DCM) to afford 2 as a brown solid product (15.0 mg, 74%): ¹H NMR (500 MHz, CDCl₃) δ 7.80 (dd, J=7.8, 1.3 Hz, 1H), 7.75-7.70 (m, 1H), 7.60-7.58 (m, 1H), 7.55 (ddd, J=7.8, 1.2, 0.5 Hz, 1H), 7.36-7.29 (m, 1H), 7.19 (td, J=7.5, 1.3 Hz, 1H), 7.14 (ddd, J=8.3, 6.7, 1.3 Hz, 1H), 6.97 (ddd, J=7.8, 7.3, 1.4 Hz, 1H), 6.88 (ddd, J=8.5, 1.3, 0.6 Hz, 1H), 6.83-6.74 (m, 2H), 6.62 (td, J=7.5, 1.2 Hz, 1H), 6.53 (s, 1H), 4.24 (p, J=6.7 Hz, 1H), 3.59 (dd, J=15.4, 6.3 Hz, 1H), 3.40-3.29 (m, 2H), 2.74 (tt, J=9.4, 4.9 Hz, 2H), 2.56 (d, J=15.4 Hz, 1H), 2.34 (s, 6H), 0.79 (d, J=6.8 Hz, 3H). ¹³C NMR (125 MHz, CDCl₃) δ 148.00, 146.75, 145.69, 141.36, 138.77, 135.90, 135.52, 129.64, 128.80, 127.83, 127.67, 127.32, 127.05, 126.23, 126.10, 125.74, 125.49, 125.03, 124.65, 123.90, 122.97, 122.86, 119.85, 101.46, 57.71, 45.19, 40.92, 40.18, 37.51, 19.87.

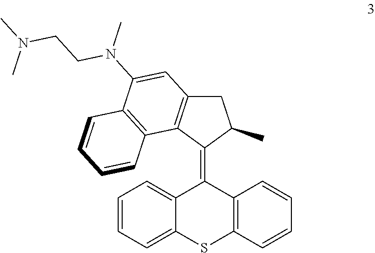

N¹,N¹,N²-trimethyl-N²-(2-methyl-1-(9H-thioxanthen-9-ylidene) ~2,3-dihydro-1H-cyclopenta[a]naphthalen-5-yl) ethane-1,2-diamine (3). BINAP (2.5 mg, 0.0066 mmol) and palladium (II) acetate (0.5 mg, 0.0011 mmol) were dissolved in dry toluene (5 mL). This solution was stirred for 30 min at rt, where upon it turned from dark red to dark orange. After this period NaOtBu (25 mg, 0.11 mmol) was added, followed by bromo-substituted motor 20 (10 mg, 0.022 mmol) and N₁,N¹,N²-trimethylethane-1,2-diamine (20 mg, 0.44 mmol). The mixture was stirred at 90 ° C for 2 d. Subsequently the reaction mixture was poured into CH₂Cl (10 mL). After filtration the solvents were evaporated. The crude product was dissolved in a small amount of CH₂Cl and purified using column chromatography (SiO₂; 10% MeOH in DCM) to afford 3 as a brown solid product (6.3 mg, 61%): ¹H NMR (500 MHz, Chloroform-d) δ 8.04 (dd, J=8.4, 1.2 Hz, 1H), 7.79 (dd, J=7.7, 1.4 Hz, 1H), 7.60 (dd, J=7.8, 1.3 Hz, 1H), 7.57 (ddd, J=7.8, 1.2, 0.5 Hz, 1H), 7.32 (td, J=7.5, 1.3 Hz, 1H), 7.22 (dd, J=7.6, 1.4 Hz, 1H), 7.17 (ddd, J=8.3, 6.7, 1.3 Hz, 1H), 7.05 (s, 1H), 7.00 (ddd, J=7.8, 7.3, 1.4 Hz, 1H), 6.87 (dt, J=8.4, 1.0 Hz, 1H), 6.80-6.72 (m, 2H), 6.64 (td, J=7.5, 1.2 Hz, 1H), 4.36-4.20 (m, 1H), 3.62 (dd, J=15.4, 6.4 Hz, 1H), 3.42 (t, J=7.0 Hz, 2H), 2.92 (s, 2H), 2.57 (d, J=15.5 Hz, 1H), 2.46 (s, 6H), 0.78 (d, J=6.9 Hz, 3H). ¹³C NMR (126 MHz, CDCl₃) δ 146.04, 145.97, 140.57, 138.23, 135.78, 135.46, 130.11, 128.64, 128.54, 128.46, 127.73, 127.70, 127.40, 127.33, 126.85, 126.30, 126.27, 126.17, 126.06, 125.78, 124.63, 123.73, 123.63, 112.90, 56.51, 44.99, 43.49, 39.93, 37.64, 29.70, 19.65.

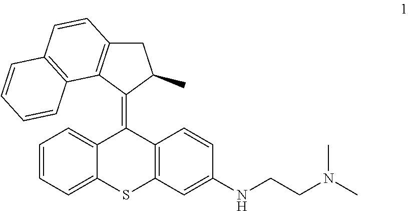 [SMILES: C[C@@H]1Cc2ccc3ccccc3c2/C1=C1\c2ccccc2Sc2cc(NCCN(C)C)ccc21]

N¹N¹-dimethyl-N²-(9-(2-methyl-2,3-dihydro-1H-cyclopenta[a]naphthalen-1-ylidene)-9H-thioxanthen-3-yl) ethane-1,2-diamine (1). BINAP (2.5 mg, 0.0066 mmol) and palladium (II) acetate (0.5 mg, 0.0011 mmol) were dissolved in dry toluene (5 mL). This solution was stirred for 30 min at rt, where upon it turned from dark red to dark orange. After this period NaOtBu (25 mg, 0.11 mmol) was added, followed by bromo-substituted motor 20 (10 mg, 0.022 mmol) and N¹,N¹,N²-trimethylethane-1,2-diamine (20 mg, 0.44 mmol). The mixture was stirred at 90 ° C for 2 d. Subsequently the reaction mixture was poured into CH₂Cl₂ (10 mL.). After filtration the solvents were evaporated. The crude product was dissolved in a small amount of CH₂Cl₂ and purified using column chromatography (SiO2; 10% MeOH in DCM) to afford 1 as a brown solid product (6.3 mg, 61%).

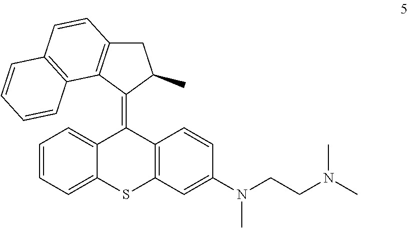 [SMILES: C[C@@H]1Cc2ccc3ccccc3c2/C1=C1\c2ccccc2Sc2cc(N(C)CCN(C)C)ccc21]

N¹,N¹-dimethyl-N²-(9-(2-methyl-2,3-dihydro-1H-cyclopenta[a]naphthalen-1-ylidene)-9H-thioxanthen-3-yl) ethane-1,2-diamine (5). BINAP (5.0 mg, 0.0132 mmol) and palladium (II) acetate (1.0 mg, 0.0022 mmol) were dissolved in dry toluene (3 mL). This solution was stirred for 30 min at rt, where upon it turned from dark red to dark orange. After this period NaOtBu (50 mg, 0.22 mmol) was added, followed by bromo-substituted motor 20 (20 mg, 0.044 mmol) and N¹,N²-dimethylethane-1,2-diamine (20 mg, 0.44 mmol). The mixture was stirred at 90 ° C for 2 d. Subsequently the reaction mixture was poured into CH₂Cl₂ (10 mL). After filtration the solvents were evaporated. The crude product was dissolved in a small amount of CH₂Ch and purified using column chromatography (SiO2; 10% MeOH in DCM) to afford 5 as a brown solid product (14.6 mg, 72%): ¹H NMR (500 MHz, Chloroform-d) δ 7.81-7.77 (m, 0.5H), 7.72-7.63 (m, 3H), 7.60-7.53 (m, 1H), 7.41 (dd, J=8.2, 2.9 Hz, 1H), 7.30 (td, J=7.6, 1.3 Hz, 0.5H), 7.19 (td, J=7.5, 1.3 Hz, 0.5H), 7.15 (ddd, J=8.0, 6.7, 1.2 Hz, 1H), 7.05 (d, J=8.5 Hz, 0.5H), 7.00-6.94 (m, 1H), 6.94 (d, J=2.6 Hz, 0.5H), 6.89 -6.85 (m, 0.5H), 6.84-6.78 (m, 1H), 6.76 (ddd, J=8.7, 4.2, 2.2 Hz, 1H), 6.69 (dd, J=7.8, 1.4 Hz, 0.5H), 6.62-6.52 (m, 1H), 6.02 (dd, J=8.6, 2.6 Hz, 0.5H), 4.28 (t, J=6.7 Hz, 1H), 3.73-3.56 (m, 2H), 3.49-3.34 (m, 1H), 3.29 (s, 1H), 3.00 (s, 1.5 H), 2.92 (t, J=6.6 Hz, 1H), 2.87 (s, 1.5 H), 2.74 (q, J=6.3 Hz, 1H), 2.60 (dd, J=15.4, 6.3 Hz, 1H), 2.52 (s, 1.5 H), 2.45 (s, 1.5 H), 0.79 (dd, J=15.7, 6.8 Hz, 3H).13C NMR (126 MHz, CDCl₃) δ 147.59, 147.29, 145.54, 145.45, 144.70, 144.46, 140.89, 138.61, 136.84, 136.57, 135.91, 135.65, 135.42, 135.18, 132.97, 132.91, 129.51, 129.35, 129.02, 128.95, 128.93, 128.52, 128.42, 128.36, 128.33, 127.71, 127.67, 127.64, 127.40, 127.36, 126.86, 126.72, 126.38, 126.14, 125.99, 125.95, 125.66, 124.53, 124.35, 124.00, 123.86, 123.80, 123.74, 111.31, 111.04, 110.97, 110.91, 51.32, 51.13, 48.03, 47.62, 39.83, 39.13, 38.80, 37.87, 37.74, 35.21, 35.14, 19.51, 19.47.

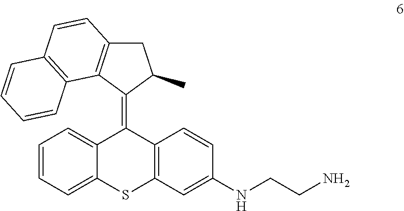 [SMILES: C[C@@H]1Cc2ccc3ccccc3c2/C1=C1\c2ccccc2Sc2cc(NCCN)ccc21]

N¹-(9-(2-methyl-2,3-dihydro-1H-cyclopenta[a]naphthalen-1-ylidene)-9H-thioxanthen-3-yl)ethane-1,2-diamine (6). BINAP (7.5 mg, 0.0198 mmol) and palladium (II) acetate (1.5 mg, 0.0033 mmol) were dissolved in dry toluene (3 mL). This solution was stirred for 30 min at rt, where upon it turned from dark red to dark orange. After this period NaOtBu (75 mg, 0.33 mmol) was added, followed by bromo-substituted motor 20 (30 mg, 0.066 mmol) and ethane-1,2-diamine (30 mg, 0.66 mmol). The mixture was stirred at 90 ° C for 2 d. Subsequently the reaction mixture was poured into CH₂Cl₂ (10 mL). After filtration the solvents were evaporated. The crude product was dissolved in a small amount of CH₂Cl and purified using column chromatography (SiO₂; 10% MeOH in DCM) to afford 6 as a brown solid product (20.9 mg, 73%): 1H NMR (500 MHz, Chloroform-d) δ 7.71-7.66 (m, 0.5H), 7.60 (ddd,)=8.1, 6.9, 3.0 Hz, 2H), 7.50-7.41 (m, 1H), 7.32 (d, J=8.2 Hz, 1H), 7.20 (td, J=7.6, 1.3 Hz, 0.5 H), 7.11-7.05 (m, 1.5 H), 7.05-6.99 (m, 1H), 6.87 (td, J=7.6, 1.4 Hz, 0.5H), 6.82-6.77 (m, 1H), 6.74 (dd, J=4.8, 2.3 Hz, 1H), 6.68 (ddd, J=8.4, 6.7, 1.4 Hz, 0.5H), 6.63-6.56 (m, 0.5H), 6.52-6.45 (m, 1H), 6.41 (d, J=8.4 Hz, 0.5H), 5.79 (dd, J=8.4, 2.3 Hz, 0.5 H), 4.17 (qd, J=6.7, 3.8 Hz, 1H), 3.53 (dt, J=15.3, 6.5 Hz, 1H), 3.14-3.05 (m, 1H), 2.99 (t, J=5.8 Hz, 1H), 2.85 (t, J=5.7 Hz, 1H), 2.74 (d, J=5.8 Hz, 1H), 2.58-2.45 (m, 1H), 0.69 (dd, J=12.3, 6.8 Hz, 3H). ¹³C NMR (126 MHz, CDC₃) δ 146.57, 146.19, 145.57, 145.49, 144.69, 144.39, 140.95, 138.68, 136.75, 136.49, 135.93, 135,67, 135.42, 135.17, 132.99, 132.95, 129.54, 129.45, 129.04, 128.98, 128.54, 128.52, 128.40, 127.73, 127.67, 127.41, 126.81, 126.38, 126.17, 126.04, 126.00, 125.68, 124.57, 124.51, 124.08, 124.03, 123.81, 123.78, 111.87, 111.84, 110.90, 45.43, 45.24, 40.54, 40.44, 39.85, 37.88, 37.84, 19.54, 19.45.

### (ii) Preparation of Cells for Irradiation Experiments

Cells from glycerol stocks maintained at −80 ° C were streaked onto LB agar plates to get single isolated colonies. One single colony was picked up from the plate and grown overnight in 5 mL of filter-sterilized LB media in a 50 mL falcon tube (220 rpm at 30 or 37 ° C depending on the strain). The following day, 1 mL of the overnight culture was added to 50 mL of filter-sterilized LB media in a 250 mL Erlenmeyer flask and incubated until an optical density at 600 nm (OD600) of approximately 1. A volume of 500 μL of this culture was diluted into 50 mL of fresh filter-sterilized LB media and centrifuged for 15 min at 5000 rpm, 25 °C. Afterward, the pellet was resuspended in phosphate-buffered saline (PBS) to a final OD₆₀₀≈0.05.

(iii) Irradiation Experiments

The appropriate volume of an MM stock at 8 mM necessary to achieve a concentration ranging from 0.3125 to 40 μM was pipetted into a 2 mL microcentrifuge tube to which 1 mL of cell suspension prepared as previously described was added. Corresponding negative controls (DMSO only) were prepared in the same way. The mixture was gently mixed by pipetting and incubated in the dark at 30 or 37 ° C, depending on the strain, for 30 min with agitation (220 rpm). Afterward, MM- or DMSO-treated cells were dispensed in one well of a 24-well plate positioned in the center of the light beam (405 nm LED Light, Prizmatix, UHP—F-5-405) placed at the appropriate distance necessary to achieve the desired light intensity of 304 mW cm-2, 146 mW cm2 or 87 mW cm2, as measured with a handheld digital power meter console coupled to an S415C thermal power sensor head (Thorlabs, Newton, MA, USA). The temperature during irradiation was monitored using a thermocouple probe (Model SC-TT-K-30-36-PP; Omega Engineering, Inc., Stanford, CT, USA). Samples were agitated during irradiation. Dark controls were prepared as previously described except that no irradiation was provided.

### (iv) Minimum Inhibitory Concentration (MIC)

For MIC determination, samples treated with a range of concentrations of the different MM, as described above and were irradiated one at a time for 5 min at 146 mW cm⁻² (43.8 J cm⁻²), after which irradiated aliquots were collected and inoculated into 1 mL MHB in a 2 mL microcentrifuge tube. Samples were incubated overnight at 30 or 37 ° C, depending on the strain, without agitation. Corresponding non-irradiated samples and negative controls (without bacteria) were also included. The following day, cultures were inspected for growth and the MIC was identified. The experimental procedure used to determine the MIC of visible light-activated MMs is schematically depicted in FIG. 26.

### (v) Time-Kill Experiments

For time-kill experiments, 50 AL aliquots in triplicate of samples treated with 2×MIC determined as described above were collected at different time points (0, 1, 2, 3, 4 and 5, 10 and 15 min) following 405 nm irradiation at different light intensities (87, 146 and 304 mW cm⁻², corresponding to a distance between sample and light source of 20 cm, 15 cm, and 10 cm, respectively). For light dose-dependency experiments, samples were exposed to different concentrations of the most potent MMs (MM 1, MM 5, and MM 6) and irradiated with 0, 1, 2, 4, 8, 16, 32, 40, and 80 J cm⁻². Serial dilutions of irradiated cell suspensions were prepared in PBS. A volume of 10 AL of the appropriate serial dilutions was spot-plated onto LB agar plates. Following overnight incubation at 30 or 37 ° C, depending on the strain, bacterial viability was assessed as colony forming units (CFU) per mL. Results were expressed as log (N/NO) whereby N is the CFU mL⁻¹ at each irradiation time point and NO is the initial CFU mL⁻¹ of the corresponding sample. Only dilutions that yielded 10-100 colonies were counted.

### (vi) Preparation and Eradication of Persister and Persister-Like Cells

Persisters of A. baumannii and P. aeruginosa were generated by growing cell cultures to late-stationary phase for 16 h at 37 ° C, followed by treatment with ciprofloxacin (10-fold MIC) for 4 h to kill non-persistent cells (Morones-Ramirez et al., 2013). Persister cells of E. coli were prepared by adding ampicillin (100 μg mL⁻¹) to exponential-phase cells (OD₆₀₀ of ≈0.8) followed by continuous agitation for another 3 b, as previously described (Keren et al., 2004). In the case of S. aureus, almost all stationary-phase are considered to be persistent (Keren et al., 2004). S. aureus cells were grown at 37 ° C and 220 rpm in LB broth to an OD600 of 0.3. Cells were then diluted 1:1000 in 25 mL LB and grown for 16 h at 37 ° C and 220 rpm in 250 mL flasks. Ampicillin-tolerant or stationary phase persister cells of E. coli and S. aureus, respectively, were collected and resuspended in PBS and then challenged with 1×MIC of MM or 1% DMSO followed by irradiation at 405 nm at a dose of 146 mW cm⁻², as described for exponential phase cells. Antibiotic controls (2× and 4×MIC) were processed in the same way, except that no light was provided. At specified time points (0, 1, 2, 3, 4 and 5, 10 and 15 min), 50-μL aliquots were removed, serially diluted and spot-plated onto LB agar plates to determine colony-forming units per mL (CFU mL⁻¹). Only dilutions that yielded 10-100 colonies were counted. Results were expressed as log (N/NO) whereby N is the CFU mL⁻¹ at each irradiation time point and NO is the initial CFU mL⁻¹ of the corresponding sample. Only dilutions that yielded 10-100 colonies were counted.

(vii) Antibiofilm Potential of Visible Light-Activated MM

The antibiofilm potential of the most potent MM (MM 1, MM 5, and MM 6) was assessed in a 96 well plate format using a combination of methods targeting different components of the biofilm (Stiefel et al., 2016): acridine orange was used to quantify the total bacteria cell number, ATP quantification was used to quantify metabolically active cells within the biofilm, crystal violet was used to quantify biofilm biomass, and fluorescein isothiocyanate was used to quantify total protein in the biofilm matrix. This combination of methods is effective at evaluating the antibiofilm potential of chemicals (Stiefel et al., 2016). P. aeruginosa and S. aureus were grown overnight in tryptic soy broth (TSB) medium. The overnight cultures were diluted in 1:100 in fresh media and 100 μL aliquots were distributed in a 96-well plate. After 24 h of static growth at 37 ° C, planktonic cells were removed by inverting the plate onto a stack of paper towels, and the biofilm was washed three times with PBS. After washing, MM 1, MM 5, or MM 6 were added at 2×MIC to the biofilm and incubated statically in the dark for 60 min. The biofilm was then irradiated for 15, 30, or 45 min at 146 mW cm2. For the determination of the total bacterial number, acridine orange solution (2% in H₂O) diluted 1:100 in Walpole's buffer (27.2 g L-1 sodium acetate trihydrate, adjusted to pH 4 with glacial acetic acid) was added to the wells. Following a 15 min incubation, the biofilm was washed three times with 0.9% NaCl, thoroughly resuspended in 100 AL 0.9% NaCl, and fluorescence intensity (excitation: 485 nm, emission: 528 nm) was measured in a microplate reader (BioTek Instruments Inc, Winooski, VT, USA) (Stiefel er al., 2016).

For quantification of viable cells in biofilms, following the addition of 100 μL hundred microliters of TSB to each well, bacteria were detached from the biofilm by thorough mixing, after which 100 AL of BacTiter-Glo™ reagent, prepared according to the manufacturer's instructions, was added to each well. After a 5 min incubation, the luminescence intensity was measured in a microplate reader (BioTek Instruments Inc, Winooski, VT, USA) (Stiefel et al., 2016).

For total biofilm protein quantification, 250 μL of FITC solution (20 μg mL⁻¹) was added to each well. Following a 30 min incubation, the biofilm was thoroughly washed with 0.9% NaCl and then resuspended in 100 μL ddH2O. Fluorescence intensity was measured (excitation: 485 nm, emission: 528 nm) in a microplate reader (BioTek Instruments Inc, Winooski, VT, USA) (Stiefel et al., 2016).

For total biofilm biomass quantification, 100 μL of TSB media was added to the irradiated biofilm, Following 24 b of recovery at 37 ° C, plates were again inverted onto a stack of paper towels and the biofilm was then washed with water by submersion of the plate. The washed biofilm was stained with a 0.1% solution of crystal violet in water. After 15 min of staining the plate was rinsed 3 times with water, and then blotted on a stack of paper towels. After overnight drying of the plate, 30% acetic acid in water was added to solubilize the crystal violet for 15 min. The solubilized crystal violet was transferred to a new flat-bottom microtiter plate and the absorbance at 550 nm was quantified in a microplate reader (BioTek Instruments Inc, Winooski, VT, USA), using 30% acetic acid in water as the blank (O'Toole, 2011). Unirradiated samples were used as controls. Control antibiotics rifampin (P. aeruginosa) and tobramycin (S. aureus) at 2×MIC were also included and processed as described for MMs.

(viii) Resistance Development

Isolation of MM-resistant mutants of E. coli, A. baumannii, P. aeruginosa, and S. aureus was attempted by irradiating cell suspensions treated with 4×MIC of the different MMs for 5 min at 146 mW cm⁻² (43.8 J cm⁻²). No visible colonies could be obtained for any of the strains tested.

For resistance development by sequential passaging (Ling et al., 2015), A. baumannii, E. coli, P. aeruginosa, and S. aureus cells in exponential phase were collected and processed as described for MIC determination. Cells were incubated at 37 ° C for 24 h after which they were inspected for growth. Cells able to grow at 0.5×MIC were collected and re-challenged with a range of MM concentrations and then irradiated. The antibiotics ciprofloxacin and gentamicin (for Gram-negatives) or ciprofloxacin and tobramycin (for S. aureus) were used as controls.

### (ix) RNAseq

Three independent, well-isolated colonies of E. coli were cultured to mid-log phase in MHB media. Cells were collected and resuspended in PBS (1×) to an OD600 of ≈0.05. Cells were treated with 0.5×MIC of MM 1, or 1% DMSO in the dark for 30 min. Cells were then irradiated for 5 min at 146 mW cm⁻² (43.8 J cm⁻²). Bacterial cells were collected onto a 0.2 μm PES filter by low vacuum filtration. Two volumes of RNAprotect (Qiagen, Valencia, CA) were added to the cells, and the samples were then centrifuged at 5000 g for 25 min at 4 ° C to pellet cells. RNA was isolated using the RNeasy Mini Kit (Qiagen, Valencia, CA) per the manufacturer's instructions. RNA sequencing (RNAseq) and data analysis were performed by DNA Link Inc. (Seoul, Republic of Korea). RNA-Seq libraries were constructed by using TruSeq Stranded Total RNA with Ribo-Zero Plus rRNA Depletion Kit and sequenced on the Illumina NovaSeq6000 platform (Illumina, San Diego, CA) in the 100 nt, paired-end configuration. From each sample, an average of 70 million reads was obtained. For gene expression analysis, reads were trimmed with cutadapt (Magoc et al., 2013) and aligned to the reference genome of Escherichia coli str. K-12 substr. MG1655 (NC_000913) using EDGE-pro pipeline with default setting. Differential expression analysis was performed with DESeq2 in Bioconductor (Love et al., 2014). Gene annotation was performed using an in-house script based on NCBI reference annotations.

Gene ontology (GO) enrichment analysis of the transcripts displaying significant differences (p<0.05) in abundance between MM- and DMSO-treated cells was performed using Panther (http://pantherdb.org/) using the Fisher's Exact test. Results were corrected for the false discovery rate (FDR).

### (x) Outer Membrane Permeability

The impact of treatment with MMs on the outer membrane permeability of E. coli was determined using the N-phenyl-1-naphthylamine (NPN) uptake assay as previously described (Helander and Mattila-Sandholm, 2000). The pore-forming antibiotic nisin was used as a positive control for membrane damage. Cells were washed and resuspended in buffer (5 mM HEPES, 5 mM glucose, pH 7.4) and treated with different concentrations of MM 1, MM 5, and MM 6, or 1% DMSO. Following irradiation as described for MIC determination, 100 μL of the bacteria suspension was mixed with NPN (final concentration: 2 μg mL⁻¹) in a 96-well black plate. NPN fluorescence was then monitored (Excitation: 350 nm, Emission: 420 nm) as a function of time using a microplate reader (BioTek Instruments Inc, Winooski, VT, USA). Results were expressed as relative fluorescent units (RFU) corrected for fluorescence in the absence of NPN and in the absence of cells.

### (xi) Inner Membrane Permeability

E. coli cells were prepared as previously described and treated with a range of concentrations of MM 1, MM 5, and MM 6 and irradiated with 405 nm light at an intensity of 146 mW cm⁻². Nisin was used as a positive control. Following irradiation, propidium iodide (PI) was added to the cells at a final concentration of 2 μg mL⁻¹ (69). After 30 min of incubation, 200 μL of bacterial suspension was added into a black 96-well plate and the time-dependent progression of fluorescence intensity (excitation: 535 nm, emission: 620 nm) was recorded in a microplate reader (BioTek Instruments Inc, Winooski, VT, USA). Results were expressed RFU corrected for fluorescence in the absence of cells and in the absence of PI.

(xii)Cytoplasmic Membrane Depolarization

The depolarization of the cytoplasmic membrane of E. coli by MM was assessed using the membrane potential-sensitive cyanine dye DiSC3 (5) (Friedrich et al., 2000). Briefly, exponential-phase bacteria were washed and resuspended in 5 mM HEPES-20 mM glucose buffer (pH 7.2) to an optical density of 0.05. This cell suspension was incubated with 100 mM KCl (to equilibrate cytoplasmic and external K⁺ concentration) and 0.4 AM DiSC₃(5) until stabilization of the fluorescent signal. Cells were then incubated with different concentrations of MMs and irradiated as described for MIC determination. CCCP was used as a positive control (71). Cells were then transferred to a black 96 well and the fluorescent signal was monitored (excitation: 622 nm, emission: 670 nm) using a microplate reader (BioTek Instruments Inc, Winooski, VT, USA). Results were expressed as RFU corrected for fluorescence in the absence of DiSC3 (5) and in the absence of cells.

(xiii) Extracellular ATP

Intracellular content leakage following treatment with MMs was determined by quantifying extracellular ATP (O'Neill et al, 2004). Following irradiation in the presence of different concentrations of MM 1, MM 5, and MM 6, or 1% DMSO, E. coli cells were centrifuged at 13,000 g for 5 min. Supernatants were recovered and stored at −20 ° C for ATP analysis. ATP analysis was conducted using the luminescence-based BacTiter-Glo™ assay (Promega) per the manufacturer's instructions. Nisin was used as a positive control. The levels of ATP in supernatants were derived via a standard curve of ATP standards from 1 nM to 1 μM. Luminescence measurements of ATP standards and culture supernatants were measured in triplicate on a microplate reader (BioTek Instruments Inc, Winooski, VT, USA). Results were expressed as relative light units (RLU) corrected for fluorescence in the absence of cells.

(xiv) Electron Microscopy

E. coli was cultured to mid-log phase in MHB media. Cells were collected and resuspended in PBS (1×) to an OD₆₀₀ of ≈0.05. Cells were treated with 0.5×MIC of MM 1, or 1% DMSO in the dark for 30 min. Cells were then irradiated for 5 min at 146 mW cm-2 (43.8 J cm⁻²) after which cells were fixed with Karnovsky's fixative (Carlson et al., 2003), and post-fixed with 1% osmium, and dehydrated with a series of ethanol washes. For TEM, samples were embedded in epoxy resin (PolyBed 812; Polyscienses, Inc., Warrington, PA, United States) after dehydration in a graduated 50-100% ethanol concentration series of washes. Ultrathin sections (65 nm) were cut using a Leica EM UC7 ultramicrotome (Leica Microsystems, Wetzlar, Germany) and post-stained with uranyl acetate and lead citrate. Specimens were observed using a JEOL JEM2100 TEM (Hitachi Corporation, Japan) operating at an accelerating voltage of 80 kV. For SEM, following ethanol dehydration samples were critical point dried using a Leica EM CPD300 (Leica Microsystems, Wetzlar, Germany), sputter-coated with 10 nm of gold, and imaged with an FEI Apreo SEM (FEI Apreo, ThermoFisher Scientific, Waltham, MA) using a secondary electron detector.

(xv) Interaction with Antibiotics

To evaluate synergy between conventional antibiotics and MM in E. coli, the fractional inhibitory concentration (FIC) index (Hall et al., 1983) was determined using a modified checkerboard microtiter test in an 8-by-8 well configuration. Briefly, E. coli cell suspensions were prepared as described for MIC determination with an increasing concentration (0.1-40 μM) of the different MMs, followed by irradiation for 5 min at 146 mW cm⁻². The irradiated cell suspensions were collected and distributed along the x-axis of a 96-well plate according to a gradient of increasing concentration, followed by the addition of a gradient of increasing concentration of antibiotic (0.00125-1 μg mL⁻¹) along the y-axis of the plate to the irradiated cells. MHB was then added to each well of the plate and the plate was incubated at 37 ° C with shaking at 220 rpm for 18 h under aerobic conditions. Bacterial growth was assessed by measuring the OD600 in a microplate reader (BioTek Instruments Inc, Winooski, VT, USA). The FIC was calculated after dividing the MIC of each antibiotic in combination with different MM by the MIC of the antibiotic alone. The FIC index, obtained by adding both FICs, was interpreted as indicating a synergistic effect if it was ≤0.5, as additive or indifferent if it was >0.5 and ≤2.0, and as antagonistic if it was >2.0 (Hall et al., 1983).

In order to evaluate the ability of pre-treatment with subinhibitory concentrations of MM to potentiate killing by antibiotics, E. coli were prepared as described for MIC determination and treated with 0.5×MIC of each MM (MM 1, MM 5, and MM 6) or 1% DMSO followed by 5-min irradiation at 146 mW cm⁻². Irradiated cell suspensions were then collected and challenged with 4×MIC of the antibiotics gentamicin, novobiocin, ciprofloxacin, and ampicillin. Following preparation of the appropriate serial dilutions, samples were spot plated onto LB agar plates and the number of CFU was determined. Non-irradiated, antibiotic-treated (4×MIC) cell suspensions were similarly processed.

To evaluate the ability of pre-treatment with MMs to potentiate killing by vancomycin, P. aeruginosa cell suspensions were prepared as described for the MIC assessment and treated with a range of concentrations (0 to 1×MIC) of the different antibacterial MMs (MM 1, MM 5 and MM 6) and irradiated for 5 min with 146 mW cm2 of 405 nm light. Following irradiation, cells were collected and distributed along the x-axis of a 96-well plate according to a gradient of increasing concentration, after which vancomycin was added according to a gradient of increasing concentration (0 to 40 μg mL⁻¹) along the y-axis of the plate to the irradiated cells. MHB was then added to each well of the plate and the plate was incubated at 37 ° C with shaking at 220 rpm for 18 h under aerobic conditions. Bacterial growth was assessed by measuring the OD600 in a microplate reader (BioTek Instruments Inc, Winooski, VT, USA). For time-kill experiments, P. aeruginosa cell suspensions prepared as previously described were treated with 0.25×MIC of the different MM (MM 1, MM 5, and MM 6) and irradiated for 5 min with 146 mW cm⁻² of 405 nm light. Vancomycin was then added (final concentration of 10, 20, and 40 μg mL⁻¹) and survival (CFU per mL) was monitored every 30 min for 4 h (240 min), as previously described. Controls treated with vancomycin only, MM only, and DMSO plus vancomycin were also included.

(xvi) Tetracycline Uptake

The ability of pre-treatment with subinhibitory concentrations of MMs to potentiate antibiotic killing was further evaluated by monitoring the fluorescence of tetracycline uptake. E. coli were prepared as described for MIC determination and treated with 0.5×MIC of each MM (MM 1, MM 5, and MM 6) or 1% DMSO followed by 5-min irradiation at 146 mW cm⁻². Irradiated cell suspensions were then collected, and tetracycline (128 μg mL⁻¹ final concentration) was added. A volume of 100 μL per well of tetracycline amended cell suspension was transferred to a black 96-well plate and fluorescence was read every 5 min for 60 min at room temperature in a microplate reader (Ex: 405 nm, Em: 535 nm) (BioTek Instruments Inc, Winooski, VT, USA). Results were expressed as RFU corrected for fluorescence in the absence of cells.

(xvii) Toxicity Profiling and Therapeutic Index Calculation

Biocompatibility of MM with primary NHDF and HEK293T cells was assessed using the CellTiter-Glo® Luminescent Cell Viability Assay (Promega), per the manufacturer's instructions. The MM concentrations that reduced cell viability by 50% (IC50) were identified and the therapeutic index was calculated as the ratio between the IC50 and the MIC at a light intensity of 146 mW cm⁻². For light dose-dependency experiments, HEK cells were treated with different concentrations of the most potent MM (MM 1, MM 5, and MM 6) and irradiated with 0, 1, 2, 4, 8, 16, 32, 40, and 80 J cm⁻².

(xviii) Animal Infection Model

Animal studies were conducted in the burn wound model Galleria mellonella recently described (Maslova et al., 2020). Larvae were purchased from a commercial source at a stage in their life cycle where they do not need to be fed. Larvae were sorted into Petri dishes lined with Whatman filter paper (Fisher Scientific, Pittsburgh, PA) and stored at 4 ° C until use. The larval bodies were sterilized with 70% ethanol. The burn was generated using a soldering iron (Weller WE1010 ESD-Safe Digital 70-Watt Soldering Station, 120V, Weller Company, Easton, PA, USA) to achieve a consistent burn area of ≈2 mm² in the middle section of the back of larvae. This location was chosen so the wound could be easily visualized without having to physically manipulate the larvae. Immediately post-burn, the wound was inoculated with 10 μL of 1:10 dilution of an overnight culture of A. baumannii or S. aureus. Any larva who showed distress or leakage of hemolymph after the burn process was immediately euthanized by incubating at −20 ° C for 20 min to minimize suffering. Following overnight incubation at 37 ° C for the establishment of infection, 10 μL of (1) different MMs at 1×MIC, (2) antibiotics polymixin B in the case of A. baumannii or tobramycin in the case of S. aureus, or (3) 1% DMSO were applied to the wound. Following a 30 min incubation period in the dark, larvae were physically restrained, covered with an opaque material, leaving only the wound exposed, and then irradiated for 5 min with 146 mW cm⁻² of 405 nm light (43.8 J cm⁻²). In order to minimize any potential temperature effects, a constant flow of 0.2 μm pore-size-filtered air was provided to the surface of the worm. Dark 1% DMSO controls were also included. The mortality of larvae post-treatment was monitored for up to 7 days. Mortality was recorded by complete melanization of the larval body and complete loss of motility. A brief overview of the protocol is schematically depicted in FIG. 44A. Work in Galleria mellonella was reviewed and approved by the Office of Sponsored Projects and Research Compliance (SPARC) of Rice University.

(xix) Statistical Analysis

Unless otherwise mentioned, the arithmetic mean and the standard error of the mean across multiple biological and technical replicas were used as the measures of center and spread. The number of replicates for each experiment type are included in the respective figure legends, where appropriate. Unless otherwise noted, all statistical analyses were performed using GraphPad Prism 8.0 (San Diego, CA, USA). When appropriate, data were min-max normalized. Depending on the sample size, data normality was assessed using an Anderson-Darling normality test, D'Agostino-Pearson omnibus normality test, Shapiro-Wilk normality test, or Kolmogorov-Smirnov normality test with Dallal-Wilkinson-Lilliefor's test for p-value. Comparisons between two groups were conducted using a t-test for parametric data, or a Mann-Whitney U test for non-parametric data. Multiple group comparisons were performed using ANOVA or a Kruskal-Wallis test with Dunn's multiple comparisons test. A Mantel-Cox test was used to determine the statistical significance in Galleria mellonella survival assays. A value of p<0.05 was considered statistically significant. Where appropriate, asterisks are used to denote the significance of differences. * p<0.05, ** p<0.01, *** p<0.001, **** p<0.0001. Unless otherwise noted, all figures were generated in GraphPad Prism 8.0 (San Diego, CA, USA).

### (x) DFT Calculations

### Software

All computations were run on the Rice Night Owls Time-Sharing High-Performance Computing Cluster. The calculations were set up in GaussView 6 and performed using Parallel Gaussian 16 (G16) Rev. A.03 with Linda (76, 87). The free-energy plots were generated using mechaSVG (Feringa, 2007).

### Geometry Optimization of the Core

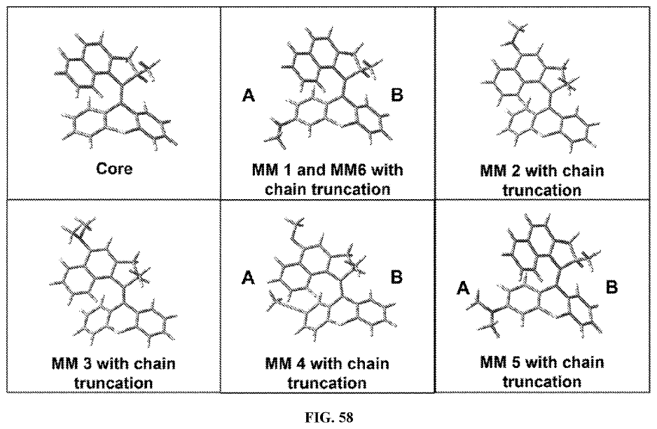

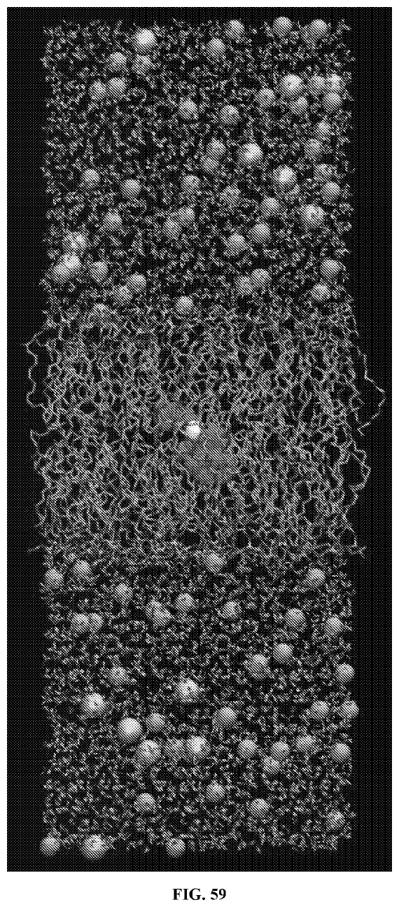

Since the motors shared a common core, the geometry of the core was first optimized, and the functional groups were subsequently added (FIG. 58). The geometry of the core was optimized using the non-empirical pure meta-GGA exchange-correlation functional TPSS with Ahlrich's def2-SVP basis set on the pruned (99,590) G16 Ultrafine integration grid (Weigend, 2005; Frisch, 2016; Dennington, 2019; Stratmann, 1996; Tao, 2003). Dispersion was accounted for using Grimme's Becke-Johnson damped D3 dispersion model (GD3BJ) (Grimme, 2011). The density fitting approximation was used to accelerate the calculations (Dunlap, 2000).

Optimization at the TPSSTPSS (GD3BJ)/def2-SVP level occurred in two stages. During the first (calcfc) stage, the force constants were calculated analytically during the initial step, after which the Hessian was updated for subsequent steps using the G16 Berny Optimization Algorithm. Subsequently, during the second stage, the calcall keyword was specified, to request further optimization of the geometry with calculation of the analytic Hessian at every step. Frequency analysis is automatically performed after the last step of a calcall optimization. The minima had zero negative Hessian eigenvalues (no imaginary frequencies), and the transition state had only one negative eigenvalue (first-order saddle point).

An intrinsic reaction coordinate (IRC) calculation was performed on the transition state to ensure that the transition state connects the correct reactants and products. The calculation was performed at the same level of theory using the local quadratic approximation and a step size of 0.5 bohr. Calcall optimization of the IRC endpoints led to the correct minima for the molecular motor.

### Geometry Optimization of Functionalized Motors

From the TPSSTPSS (GD3BJ)/def2-SVP geometries of the core, the remaining types of motors in this study were constructed. Since in quantum chemistry the aim is to achieve a high level of accuracy while remaining within a reasonable computational cost, the side chains of the molecular motors were truncated and terminated with hydrogen. However, the sterically important moieties along with all atoms connected to electronically relevant conjugated portions of the motor were retained.

The geometry was subsequently refined by first increasing the basis set to def2-TZVP, which is of triple-ζ quality, and then, by increasing the integration grid size to the G16 SuperFineGrid (Weigend, 2005; Frisch, 2016). Previous studies have recommended the (99,590) grid for convergence of meta-GGA functionals, but we chose to further increase the grid to enhance accuracy (Weigend, 2005). The SuperFineGrid keyword requests a pruned (175,974) grid for atoms of the first row, and a (250,974) grid for heavier atom types (Frisch, 2016). Energies and forces were converged to within 1*10⁻⁷ hartrees and 2*10⁻⁵ hartrees/bohr. Frequency analysis confirmed that the optimized structures had the correct number of negative eigenvalues. The TPSSTPSS (GD3BJ)/def2-SVP coordinates can be found in Table S13.

TABLE 19
Geometries optimized at the TPSSTPSS(GD3BJ)/def2-
TZVP level on the Gaussian 16 Superfine grid.

| Metastable Conformation of the Core | | | | | | | |
|---|---|---|---|---|---|---|---|
| 0 1 | | | | | | | |
| C | 1.992790 | 3.382726 | −0.917765 | H | −0.913749 | −2.135890 | −2.900933 |
| C | 1.980291 | 2.024493 | −0.565984 | C | 1.319551 | −2.720650 | −1.623473 |
| C | 0.763329 | 1.310093 | −0.402862 | H | 1.246280 | −3.657494 | −2.188144 |
| C | −0.410666 | 2.032757 | −0.709982 | H | 1.564244 | −1.915459 | −2.324985 |
| C | −0.397279 | 3.371455 | −1.074362 | H | 2.146158 | −2.820203 | −0.918070 |
| C | 0.510400 | 4.066577 | −1.159407 | C | −3.673301 | −1.806740 | −1.450375 |
| H | 2.949791 | 3.889764 | −1.008765 | C | −4.489529 | −1.001982 | −0.679003 |
| H | −1.362008 | 1.525306 | −0.681842 | H | −5.569932 | −1.040861 | −0.795180 |
| H | −1.336919 | 3.870624 | −1.292349 | C | −3.946220 | −0.146118 | 0.315235 |
| H | 0.833042 | 5.119468 | −1.424154 | C | −2.523104 | −0.064941 | 0.490509 |
| C | 0.780540 | −0.130548 | −0.084531 | C | −4.787491 | 0.608724 | 1.173855 |
| C | 1.991915 | −0.690055 | 0.541877 | C | −4.260222 | 1.380007 | 2.185423 |
| C | 3.290353 | −0.161743 | 0.401059 | H | −4.916448 | 1.948808 | 2.838120 |
| C | 1.846271 | −1.785160 | 1.420379 | C | −2.015628 | 0.712374 | 1.560493 |
| C | 4.395408 | −0.779041 | 1.004133 | H | −0.942405 | 0.745526 | 1.720180 |
| C | 2.934369 | −2.402752 | 2.019968 | C | −2.862562 | 1.415973 | 2.389955 |
| H | 0.841541 | −2.142492 | 1.623028 | H | −5.863495 | 0.553399 | 1.025892 |
| C | 4.223971 | −1.911933 | 1.789189 | H | −2.453049 | 2.001867 | 3.208033 |
| H | 5.384193 | −0.350957 | 0.863321 | H | −1.510610 | −3.601814 | −2.103148 |
| H | 2.780201 | −3.251284 | 2.679961 | H | −4.103216 | −2.496397 | −2.1772450 |
| H | 5.085552 | −2.383854 | 2.252387 | | | | |
| S | 3.578347 | 1.288269 | −0.530463 | | | | |
| C | −0.247907 | −1.020201 | −0.372032 | | | | |
| C | −1.693531 | −0.828709 | −0.394717 | | | | |
| C | −0.015762 | −2.440031 | −0.934322 | | | | |
| C | −2.281985 | −1.731463 | −1.284979 | | | | |
| H | −0.189132 | −3.193360 | −0.151471 | | | | |
| C | −1.211423 | −2.562609 | −1.931952 | | | | |
| Transition State for Thermal Helix Inversion of the Core | | | | | | | |
| 0 1 | | | | | | | |
| C | −0.768147 | 3.177858 | 1.359564 | H | 0.355422 | −3.973964 | 0.943358 |
| C | −0.986072 | 1.857723 | 0.955451 | C | −1.764187 | −2.859284 | 0.416801 |
| C | −0.525797 | 1.391032 | −0.293736 | H | −1.852017 | −3.943182 | 0.553769 |
| C | −0.070371 | 2.350544 | −1.207136 | H | −1.607806 | −2.404641 | 1.401046 |
| C | 0.124297 | 3.674640 | −0.824430 | H | −2.711369 | −2.502741 | 0.016651 |
| C | −0.173537 | 4.077508 | 0.479077 | C | 2.996632 | −2.858103 | 0.804351 |
| H | −1.116468 | 3.504947 | 2.334748 | C | 3.971462 | −1.897956 | 0.929896 |
| H | 0.130385 | 2.053368 | −2.232045 | H | 4.911987 | −2.117775 | 1.429223 |
| H | 0.502649 | 4.393451 | −1.545377 | C | 3.794706 | −0.619835 | 0.339506 |
| H | −0.008976 | 5.105935 | 0.787174 | C | 2.558483 | −0.296928 | −0.323619 |
| C | −0.802847 | −0.047148 | −0.591393 | C | 4.858134 | 0.318171 | 0.325354 |
| C | −2.281270 | −0.210468 | −0.613692 | C | 4.762423 | 1.494794 | −0.384090 |
| C | −2.969372 | 0.139013 | 0.564626 | H | 5.588763 | 2.199881 | −0.394152 |
| C | −3.009340 | −0.676205 | −4.711717 | C | 1.539703 | 0.873266 | −1.110542 |
| C | −4.345123 | −0.068324 | 0.673274 | H | 1.682043 | 1.050106 | −1.736611 |
| C | −4.386195 | −0.873831 | −1.610796 | C | 3.603056 | 1.750629 | −1.143973 |
| H | −2.482520 | −0.917183 | −2.630778 | H | 3.770727 | 0.069596 | 0.862086 |
| C | −5.047893 | −0.590180 | −0.413318 | H | 3.548619 | 2.637941 | −1.768764 |
| H | −4.861129 | 0.188098 | 1.593826 | H | 0.891079 | 4.252268 | −0.708234 |
| H | −4.942820 | −1.254826 | −2.462156 | H | 3.154404 | −3.864447 | 1.184334 |
| H | −6.119401 | −0.748727 | −0.332008 | | | | |
| S | −2.030378 | 0.794381 | 1.925552 | | | | |
| C | 0.018499 | −1.135697 | −0.525804 | | | | |
| C | 1.473126 | −1.238777 | −0.246886 | | | | |
| C | −0.549268 | −2.285981 | −0.494464 | | | | |
| C | 1.765660 | −2.523914 | 0.207659 | | | | |
| H | −0.809825 | −2.875086 | −1.519906 | | | | |
| C | 0.623811 | −3.465643 | 0.009201 | | | | |
| Stable Conformation of the Core | | | | | | | |
| 0 1 | | | | | | | |
| C | 0.879529 | 3.471945 | 0.322081 | H | −1.163337 | −4.402869 | 0.156293 |
| C | 1.219066 | 2.146492 | 0.033087 | C | 0.305035 | −3.094768 | −1.640475 |
| C | 0.589593 | 1.078911 | 0.699360 | H | 0.457475 | −4.169126 | −1.793236 |
| C | −0.334791 | 1.376453 | 1.709715 | H | −0.646738 | −2.809675 | −2.101736 |
| C | −0.673379 | 2.693425 | 2.002929 | H | 1.108712 | −2.554137 | −2.149805 |
| C | −0.076836 | 3.741721 | 1.297932 | C | −3.373168 | −2.594143 | 0.634908 |
| H | 1.373925 | 4.281380 | −0.207226 | C | −4.229283 | −1.538805 | 0.404235 |
| H | −0.804442 | 0.555786 | 2.243551 | H | −5.299589 | −1.652567 | 0.559463 |
| H | −1.406087 | 2.903505 | 2.776332 | C | −3.747002 | −0.301895 | −0.102534 |
| H | −0.339537 | 4.771513 | 1.522284 | C | −2.338960 | −0.117886 | −0.321921 |
| C | 0.935085 | 0.312244 | 0.335607 | C | −4.641266 | 0.742177 | −0.453868 |
| C | 2.400797 | −0.536318 | 0.244571 | C | −4.183975 | 1.896070 | −1.050339 |
| C | 3.170317 | 0.373971 | −0.509191 | H | −4.880007 | 2.685647 | −1.319594 |
| C | 3.080745 | −1.568992 | 0.908309 | C | −1.912018 | 1.054827 | −0.991860 |
| C | 4.543999 | 0.180565 | 0.689048 | H | −0.865047 | 1.164833 | −1.245163 |
| C | 4.449088 | −1.761318 | 0.737996 | C | −2.810307 | 2.037395 | −1.346904 |
| H | 2.527142 | −2.217957 | 1.579120 | H | −5.702847 | 0.602160 | −0.263408 |
| C | 5.178477 | −0.901485 | −0.083012 | H | −2.457919 | 2.926425 | −1.862211 |
| H | 5.111017 | 0.894510 | −1.279416 | H | −0.645307 | −3.606302 | 1.649956 |
| H | 4.947706 | −2.575045 | 1.256576 | H | −3.758177 | −3.557631 | 0.959622 |
| H | 6.245673 | −1.047449 | −0.225319 | | | | |
| S | 2.409741 | 1.802296 | −1.231802 | | | | |
| C | 0.010926 | −1.277329 | 0.081115 | | | | |
| C | −1.462492 | −1.176018 | 0.071684 | | | | |
| C | 0.300830 | −2.760814 | −0.138490 | | | | |
| C | −1.990537 | −2.410264 | 0.448354 | | | | |
| H | 1.252694 | −3.070310 | 0.288685 | | | | |
| C | −0.892225 | −3.434709 | 0.592667 | | | | |
| Metastable Conformation on the Functionalized | | | | | | | |
| Side of the Stator, for MM 1 and 6 with Chain | | | | | | | |
| Truncation | | | | | | | |
| 0 1 | | | | | | | |
| C | 2.772538 | 2.206073 | −0.296040 | H | −1.820549 | −1.716705 | −3.114675 |
| C | 2.296621 | 0.895535 | −0.150016 | C | 0.069190 | −3.173687 | −2.003645 |
| C | 0.916962 | 0.575685 | −0.200250 | H | −0.305829 | −3.691092 | −2.668152 |
| C | 0.074589 | 1.666382 | −0.529691 | H | 0.563207 | −2.412586 | −2.617766 |
| C | 0.527660 | 2.958551 | −0.685998 | H | 0.818612 | −3.619512 | −1.347281 |
| C | 1.895838 | 3.267885 | −0.535595 | C | −4.297698 | −0.62690 | −1.531289 |
| H | 3.842641 | 2.377942 | −0.236430 | C | −4.786507 | 0.328321 | −0.679195 |
| H | −0.977712 | 1.483767 | −0.688386 | H | −5.80794 | 0.689392 | −0.771760 |
| H | −0.180347 | 3.748529 | −0.926512 | C | −3.983308 | 0.824909 | 0.383096 |
| C | 0.434510 | −0.808115 | −0.085651 | C | −2.630432 | 0.372275 | 0.532592 |
| C | 1.349588 | −1.826139 | 0.462838 | C | −4.510322 | 1.741701 | 1.331030 |
| C | 2.755768 | −1.722987 | 0.482704 | C | −3.753776 | 2.171509 | 2.398631 |
| C | 0.796871 | −2.948322 | 1.118737 | H | −4.171096 | 2.870281 | 3.118419 |
| C | 3.555137 | −2.753638 | 0.999155 | C | −1.890858 | 0.809947 | 1.658009 |
| C | 1.579342 | −3.971574 | 1.632313 | H | −0.880394 | 0.440596 | 1.795702 |
| H | −0.283087 | −2.993470 | 1.221210 | C | −2.438370 | 1.686182 | 2.571140 |
| C | 2.973281 | −3.889026 | 1.546190 | H | −5.533219 | 2.089205 | 1.204599 |
| H | 4.4636145 | −2.642245 | 0.983820 | H | −1.858835 | 2.004758 | 3.430437 |
| H | 1.108465 | −4.821603 | 2.117335 | H | −2.894435 | −2.974446 | −2.477836 |
| H | 3.600795 | −4.681561 | 1.943336 | H | −4.933189 | −1.033247 | −2.333837 |
| S | 3.583877 | −0.301811 | −0.106674 | N | 2.326141 | 4.580362 | −0.611946 |
| C | −0.832703 | 1.235343 | −0.478625 | H | 1.679201 | 5.210257 | −1.067955 |
| C | −2.114405 | −0.544050 | −0.443800 | C | 3.733394 | 4.915283 | −0.750227 |
| C | −1.099198 | −2.557431 | −1.233736 | H | 4.283730 | 4.610297 | 0.146604 |
| C | −2.973739 | −1.068644 | −1.414827 | H | 4.202322 | 4.435674 | −1.623680 |
| H | −1.525694 | −3.311402 | −0.554797 | H | 3.824620 | 5.998765 | −0.850394 |
| C | −2.254209 | −2.132918 | −2.193119 | | | | |
| Transition State for Thermal Helix Inversion on | | | | | | | |
| the Functionalized Side of the Stator, for MM 1 | | | | | | | |
| and 6 with Chain Truncation | | | | | | | |
| 0 1 | | | | | | | |
| C | 1.061933 | 2.795516 | −0.879022 | H | −0.7877480 | −4.202283 | −1.402749 |
| C | 1.124761 | 1.421767 | −0.637844 | C | 1.421601 | −3.387640 | −0.717734 |
| C | 0.585049 | 0.837321 | 0.523443 | H | 1.405326 | −4.447065 | −0.998720 |
| C | 0.210691 | 1.726608 | 1.542686 | H | 1.329917 | −2.792336 | −1.632738 |
| C | 0.153969 | 3.094833 | 1.335533 | H | 2.391228 | −3.183481 | −0.267614 |
| C | 0.513981 | 3.649989 | 0.090375 | C | −3.313444 | −2.862058 | −1.119417 |
| H | 1.501787 | 3.194373 | −1.786394 | C | −4.187406 | −1.801297 | −1.118215 |
| H | −0.033509 | 1.335383 | 2.525381 | H | −5.138010 | −1.859200 | −1.642948 |
| H | −0.168925 | 3.750757 | 2.140919 | C | −3.893399 | −0.635555 | −0.364857 |
| C | 0.720132 | −0.646076 | 0.637359 | C | −2.640685 | −0.527928 | 0.336180 |
| C | 2.174138 | −0.959335 | 0.633255 | C | −4.857442 | 0.395787 | −0.228818 |
| C | 2.924335 | −0.524210 | −0.45698 | C | −4.653976 | 1.454034 | 0.628837 |
| C | 2.826169 | −1.638150 | 1.686325 | H | −5.405351 | 2.232174 | 0.729953 |
| C | 4.276307 | −0.848754 | −9.574087 | C | −2.515709 | 0.521213 | 1.270312 |
| C | 4.180658 | −1.952965 | 1.579238 | H | −1.652632 | 0.526208 | 1.913624 |
| H | 2.254179 | −1.948999 | 2.556318 | C | −3.485823 | 1.489949 | 1.417420 |
| C | 4.298211 | −1.577487 | 0.440934 | H | −5.782602 | 0.313445 | −0.794627 |
| H | 4.838230 | 0.523185 | −1.444573 | H | −3.351357 | 2.278495 | 2.152806 |
| H | 4.675447 | −2.497059 | 2.378726 | H | −1.376087 | −4.648195 | 0.193175 |
| H | 5.951707 | −1.827403 | 0.353169 | H | −3.564633 | −3.789047 | −1.629132 |
| S | 2.089182 | 0.397451 | 1.728460 | N | 0.358548 | 5.010462 | −0.133398 |
| C | −0.200315 | −1.633563 | 0.432354 | C | 0.993755 | 5.666644 | −1.264387 |
| C | −1.652920 | −1.554919 | 0.137767 | H | 0.765331 | 6.733664 | −1.219190 |
| C | 0.222116 | −3.116959 | 0.214507 | H | 2.087713 | 5.536921 | −1.280221 |
| C | −2.064236 | −2.733800 | −0.482617 | H | 0.587060 | 5.272305 | −2.201720 |
| H | 0.432899 | −3.564028 | 1.193739 | H | 0.304615 | 5.5670787 | 0.710184 |
| C | −1.022138 | −3.801364 | −0.408995 | | | | |
| Stable Conformation on the Functionalized Side | | | | | | | |
| of the Stator, for MM 1 and 6 with Chain | | | | | | | |
| Truncation | | | | | | | |
| 0 1 | | | | | | | |
| C | −0.293546 | 3.152622 | −0.017974 | H | 0.262850 | −4.942997 | −0.481493 |
| C | −0.898823 | 1.903804 | 0.143300 | C | −1.002317 | −3.557324 | 1.408242 |
| C | −0.482489 | 0.778634 | −0.585118 | H | −1.338859 | −4.598865 | 1.464797 |
| C | 0.519282 | 0.970180 | −1.550960 | H | −0.028006 | −3.479736 | 1.903017 |
| C | 1.122210 | 2.200746 | −1.734865 | H | −1.715023 | −2.931863 | 1.954611 |
| C | 0.740297 | 3.312533 | −0.954143 | C | 2.765064 | −3.513919 | −0.769658 |
| H | −0.642146 | 3.992656 | 0.513111 | C | 3.784508 | −2.647272 | −0.438126 |
| H | 0.840026 | 0.118925 | −2.144017 | H | 4.823032 | −2.933163 | −0.587733 |
| H | 1.903048 | 2.317158 | −2.482728 | C | 3.508101 | −1.391214 | 0.166668 |
| C | −1.087384 | −0.541597 | −0.322559 | C | 2.147267 | −0.983608 | 0.381806 |
| C | −2.568842 | −0.495918 | −0.240548 | C | 4.558334 | −0.551129 | 0.619467 |
| C | −3.162257 | 0.499296 | 0.564774 | C | 4.289950 | 0.610882 | 1.308270 |
| C | −3.426071 | −1.344339 | −0.958151 | H | 5.104208 | 1.240775 | 1.656204 |
| C | −4.549421 | 0.555965 | 0.736373 | C | 1.909541 | 0.185807 | 1.145542 |
| C | −4.807830 | −1.286323 | −0.796732 | H | 0.889766 | 0.455331 | 1.389779 |
| H | −2.999704 | −2.046387 | −1.666997 | C | 2.953034 | 0.963996 | 1.597072 |
| C | −5.370850 | −0.352722 | 0.075528 | H | 5.585057 | −0.857778 | 0.432750 |
| C | −4.977634 | 1.329698 | 1.367193 | H | 2.743837 | 1.853794 | 2.184500 |
| C | −5.444952 | −1.962243 | −1.359828 | H | −0.070320 | −3.943423 | −1.904366 |
| H | −6.447631 | −0.303759 | 0.209283 | H | 2.986748 | −4.500129 | −1.170274 |
| S | −2.162403 | 1.720060 | 1.370796 | N | 1.346389 | 4.541648 | −1.166612 |
| C | −0.356648 | −1.677065 | −0.143748 | H | 2.245850 | 4.486671 | −1.626241 |
| C | 1.111598 | −1.835450 | −0.114074 | C | 1.202352 | 5.630129 | −0.214178 |
| C | −0.902377 | −3.101606 | −0.057869 | H | 0.167402 | 5.989477 | −0.201040 |
| C | 1.430109 | −3.107561 | −0.587470 | H | 1.479786 | 5.339037 | 0.811271 |
| H | −1.881547 | −3.206064 | −0.521122 | H | 1.841171 | 6.456567 | −0.532508 |
| C | 0.175012 | −3.908577 | −0.833306 | | | | |
| Metastable Conformation on the Non- | | | | | | | |
| Functionalized Side of the Stator, for MM 1 and | | | | | | | |
| 6 with Chain Truncation | | | | | | | |
| 0 1 | | | | | | | |
| C | 0.781035 | 3.854918 | −0.805163 | H | −1.493637 | −1.910184 | −3.110130 |
| C | 1.039531 | 2.487993 | −0.620261 | C | 0.975404 | −2.171316 | −2.225891 |
| C | −0.012421 | 1.546830 | −0.465552 | H | 0.987165 | −3.059510 | −2.868738 |
| C | −1.317707 | 2.066598 | −0.615506 | H | 0.956152 | −1.283219 | −2.867234 |
| C | −1.577720 | 3.415273 | −0.813540 | H | 1.905911 | −2.158441 | −1.655544 |
| C | −0.519959 | 4.328892 | −0.886086 | C | −4.012619 | −2.182828 | −1.276187 |
| H | 1.621631 | 4.537972 | −0.896754 | C | −4.827428 | −1.588389 | −0.331335 |
| H | −2.156329 | 1.387692 | −0.594931 | H | −5.893552 | −1.800973 | −0.309832 |
| H | −2.601969 | 3.751089 | −0.912670 | C | −4.288054 | −0.726189 | 0.659485 |
| H | −0.707077 | 5.389968 | −1.021819 | C | −2.884738 | −0.419559 | 0.656691 |
| C | 0.277351 | 0.109479 | −0.310570 | C | −5.106469 | −0.182796 | 1.684172 |
| C | 1.626673 | −0.291743 | 0.105704 | C | −4.560404 | 0.597580 | 2.683662 |
| C | 2.798500 | 0.469220 | −0.069386 | H | −5.208069 | 1.003503 | 3.463272 |
| C | 1.786751 | −1.500078 | 0.824226 | C | −2.360059 | 0.359356 | 1.716785 |
| C | 4.058025 | 0.002073 | 0.323130 | H | −1.294249 | 0.560559 | 1.739510 |
| C | 3.017668 | −1.984370 | 1.213612 | C | −3.180290 | 0.854544 | 2.708206 |
| H | 0.892585 | −2.060163 | 1.078461 | H | −6.170519 | −0.407805 | 1.671708 |
| C | 4.193633 | −1.250728 | 0.935007 | H | −2.755940 | 1.445773 | 3.514916 |
| H | 4.924388 | 0.637047 | 0.168514 | H | −1.710846 | −3.518366 | −2.397441 |
| H | 3.087176 | −2.923655 | 1.757641 | H | −4.427656 | −2.878682 | −2.001311 |
| S | 2.739788 | 2.064649 | −0.780374 | N | 5.427523 | −1.769528 | 1.277935 |
| C | −0.634232 | −0.967013 | −0.582073 | H | 5.400887 | −2.512287 | 1.963849 |
| C | −2.077820 | −0.967013 | −0.395511 | C | 6.632070 | −0.956547 | 1.273433 |
| C | −0.263911 | −2.207499 | −1.331563 | H | 6.859794 | −0.624676 | 0.254619 |
| C | −2.642065 | −1.884921 | −1.287353 | H | 6.547730 | −0.067241 | 1.917030 |
| H | −0.178962 | −3.048832 | −0.626846 | H | 7.465292 | −1.568896 | 1.623981 |
| C | −1.562293 | −2.459837 | −2.159587 | | | | |
| Transition State for Thermal Helix Inversion on | | | | | | | |
| the Non-Functionalized Side of the Stator, for | | | | | | | |
| MM 1 and 6 with Chain Truncation | | | | | | | |
| 0 1 | | | | | | | |
| C | 0.154591 | 3.244145 | −1.327501 | H | −0.699669 | −3.955174 | −0.962749 |
| C | 0.416682 | 1.932612 | −0.921820 | C | 1.359598 | −2.740131 | −0.411079 |
| C | −0.064504 | 1.437639 | 0.309328 | H | 1.493754 | −3.817115 | −0.565645 |
| C | −0.591465 | 2.373256 | 1.209118 | H | 1.194867 | −2.274608 | −1.388898 |
| C | −0.831608 | 3.689820 | 0.825371 | H | 2.286667 | −2.351054 | 0.005867 |
| C | −0.509270 | 4.112788 | −0.465417 | C | −3.386112 | −2.948404 | −0.865405 |
| H | 0.521528 | 3.590964 | −2.289053 | C | −4.400977 | −2.031083 | −0.997523 |
| H | −0.809320 | 2.064839 | 2.226963 | H | −5.322735 | −2.286794 | −1.514499 |
| H | −1.265071 | 4.386982 | 1.536830 | C | −4.290497 | −0.753774 | −0.389794 |
| H | −0.709648 | 5.134264 | −0.775595 | C | −3.079799 | −0.384568 | 0.296028 |
| C | 0.267681 | 0.011627 | 0.610326 | C | −5.394566 | 0.136316 | −0.379953 |
| C | 1.748561 | −0.093185 | 0.668791 | C | −5.362010 | 1.306040 | 0.346453 |
| C | 2.460868 | 0.292305 | −0.478902 | H | −6.219173 | 1.973468 | 0.352932 |
| C | 2.478776 | −0.558980 | 1.788337 | C | −3.124901 | 0.774231 | 1.098641 |
| C | 3.844000 | 0.139119 | −0.570061 | H | −2.284748 | 0.978794 | 1.739930 |
| C | 3.855076 | −0.718620 | 1.697085 | C | −4.226757 | 1.602765 | 1.127892 |
| H | 1.949320 | −0.833228 | 2.676778 | H | −6.287248 | −0.144943 | −0.933894 |
| C | 4.358336 | −0.393922 | 0.517485 | H | −4.221627 | 2.482743 | 1.765344 |
| H | 4.357867 | 0.450658 | −1.472774 | H | −1.247533 | −4.259528 | 0.680284 |
| H | 4.403127 | −1.105090 | 2.553567 | H | −3.493188 | −3.956679 | −1.258035 |
| S | 1.539985 | 0.926052 | −1.863195 | N | 5.927965 | −0.613255 | 0.451674 |
| C | −0.507884 | −1.109875 | 0.527818 | H | 6.384900 | −0.660234 | 1.353174 |
| C | −1.951793 | −1.275132 | 0.225852 | C | 6.733632 | −0.047409 | −0.618018 |
| C | 0.124578 | −2.533240 | 0.491510 | H | 6.450836 | −0.491719 | −1.578535 |
| C | −2.181131 | −2.567554 | −0.244502 | H | 6.632333 | 1.046382 | −0.699886 |
| H | 0.389818 | −2.821591 | 1.515922 | H | 7.780977 | −0.292226 | −0.429037 |
| C | −1.002895 | −3.460643 | −0.031858 | | | | |
| Stable Conformation on the Non-Functionalized | | | | | | | |
| Side of the Stator, for MM 1 and 6 with Chain | | | | | | | |
| Truncation | | | | | | | |
| 0 1 | | | | | | | |
| C | 0.155517 | 3.540660 | 0.399038 | H | −1.485532 | −4.429431 | 0.172688 |
| C | 0.576559 | 2.235398 | 0.125611 | C | −0.013955 | −3.055097 | −1.573355 |
| C | −0.033036 | 1.133537 | 0.753904 | H | 0.197411 | −4.121198 | −1.715295 |
| C | −1.022200 | 1.381196 | 1.713428 | H | −0.959246 | −2.818822 | −2.073973 |
| C | −1.441207 | 2.678031 | 1.994307 | H | 0.781732 | −2.476001 | −2.052145 |
| C | −0.863189 | 3.758334 | 1.323517 | C | −3.801905 | −2.733091 | 0.538658 |
| H | 0.636510 | 4.376247 | −0.101353 | C | −4.700198 | −1.724003 | 0.264263 |
| H | −1.477224 | 0.535739 | 2.222521 | H | −5.769253 | −1.891956 | 0.371768 |
| H | −2.222348 | 2.846910 | 2.729758 | C | −4.258624 | −0.466059 | −0.227172 |
| H | −1.188813 | 4.772700 | 1.535611 | C | −2.853033 | −0.211142 | −0.383632 |
| C | 0.402099 | −0.237960 | 0.408900 | C | −5.187780 | 0.530041 | −0.624859 |
| C | 1.877196 | −0.385384 | 0.385280 | C | −4.763365 | 1.703284 | −1.207601 |
| C | 2.643193 | 0.5655404 | −0.314488 | H | −5.485853 | 3.455372 | −1.512682 |
| C | 2.590739 | −1.392005 | 1.059381 | C | −2.456757 | 0.979368 | −1.041337 |
| C | 4.030062 | 0.452510 | −0.443383 | H | −1.406119 | 1.141988 | −1.246641 |
| C | 3.966489 | −1.513496 | 0.955310 | C | −3.386570 | 1.913271 | −1.443243 |
| H | 2.051335 | −2.083125 | 1.698985 | H | −6.248458 | 0.336876 | −0.481187 |
| C | 4.711983 | −0.607260 | 0.173063 | H | −3.056699 | 2.816986 | −1.947872 |
| H | 4.571955 | 1.212758 | −0.995634 | H | −1.070627 | 3.598168 | 1.679985 |
| H | 4.483157 | −2.302107 | 1.497683 | H | −4.151363 | −3.714297 | 0.850660 |
| S | 1.853494 | 1.962014 | −1.069970 | N | 6.091710 | −0.729211 | 0.098993 |
| C | −0.466202 | −1.247417 | 0.124377 | H | 6.442287 | −1.651264 | 0.321932 |
| C | −1.941222 | −1.220696 | 0.055961 | C | 6.864294 | 0.007933 | −0.887434 |
| C | −0.095464 | −2.716266 | −0.074801 | H | 6.499588 | −0.144386 | −1.915327 |
| C | −2.423614 | −2.479173 | 0.414336 | H | 6.835993 | 1.081108 | −0.668278 |
| H | 0.852248 | 2.977697 | 0.391735 | H | 7.904309 | −0.319654 | −0.828600 |
| C | −1.281946 | 3.445955 | 0.611907 | | | | |
| Metastable Conformation, for MM 2 with Chain | | | | | | | |
| Truncation | | | | | | | |
| 0 1 | | | | | | | |
| C | 2.782011 | 3.145969 | −1.170242 | H | −0.911947 | −2.135476 | −2.628071 |
| C | 2.639971 | 1.816673 | −0.748010 | C | 1.361681 | −2.854561 | −1.510974 |
| C | 1.364045 | 1.253139 | −0.475905 | H | 1.153172 | −3.815211 | −1.996716 |
| C | 0.261336 | 2.089133 | −0.760050 | H | 1.610191 | −2.123248 | −2.287956 |
| C | 0.400995 | 3.398063 | −1.201743 | H | 2.238562 | −2.984911 | −0.874512 |
| C | 1.669767 | 3.948299 | −1.386826 | C | −3.462631 | −1.402365 | −1.001399 |
| H | 3.782380 | 3.535231 | −1.340981 | C | −4.152804 | −0.450838 | −0.246317 |
| H | −0.736702 | 1.693237 | −0.655445 | C | −3.418226 | 0.422126 | 0.640749 |
| H | −0.489622 | 3.988040 | −1.398683 | C | −1.987741 | 0.329867 | 0.703854 |
| H | 1.793134 | 4.978017 | −1.708952 | C | −4.070847 | 1.350976 | 1.487364 |
| C | 1.242470 | −0.159602 | −0.072311 | C | −3.370029 | 2.114765 | 2.398548 |
| C | 2.420760 | −0.822136 | 0.514299 | H | −3.897130 | 2.820489 | 3.034035 |
| C | 3.758697 | −0.455430 | 0.266527 | C | −1.302673 | 1.100781 | 1.674741 |
| C | 2.213834 | −1.846380 | 1.463213 | H | −0.225423 | 0.994948 | 1.748184 |
| C | 4.825140 | −1.160174 | 0.840710 | C | −1.973834 | 1.967774 | 2.510186 |
| C | 3.264025 | −2.555445 | 2.030117 | H | −5.146721 | 1.491245 | 1.421835 |
| H | 1.191550 | −2.074233 | 1.748481 | H | −1.424120 | 2.544795 | 3.248298 |
| C | 4.582078 | −2.226881 | 1.697900 | H | −1.588104 | −3.461889 | −1.666737 |
| H | 5.843741 | −0.854279 | 0.617378 | H | −4.006994 | −2.108798 | −1.619844 |
| H | 3.059454 | −3.347474 | 2.744693 | N | −5.523423 | −0.336871 | −0.329784 |
| H | 5.414670 | −2.769894 | 2.135682 | H | −5.977550 | 0.103059 | 0.457743 |
| S | 4.141591 | 0.895352 | −0.777422 | C | −6.333344 | −1.296884 | −1.059496 |
| C | 0.097983 | −0.935643 | −0.238929 | H | −6.204769 | −2.326688 | −0.692297 |
| C | −1.308189 | −0.577577 | −0.174312 | H | −6.076475 | −1.275616 | −2.124033 |
| C | 0.129388 | −2.400739 | −0.728052 | H | −7.382391 | −1.012941 | −0.955164 |
| C | −2.070208 | −1.460622 | −0.943795 | | | | |
| H | −0.040262 | −3.086779 | 0.114926 | | | | |
| C | −1.160345 | −2.455488 | −1.605621 | | | | |
| Transition Sate for Thermal Helix Inversion, | | | | | | | |
| for MM 2 with Chain Truncation | | | | | | | |
| 0 1 | | | | | | | |
| C | −1.773379 | 2.934721 | 1.641016 | H | 0.850706 | −3.705808 | 0.477027 |
| C | −1.738952 | 1.638538 | 1.119033 | C | −1.502294 | −3.063499 | 0.207919 |
| C | −1.292834 | 1.389168 | −0.196373 | H | −1.338766 | −4.146298 | 0.258549 |
| C | −1.122164 | 2.501678 | −1.031086 | H | −1.361998 | −2.651492 | 1.213204 |
| C | −1.178189 | 3.798779 | −0.528618 | H | −2.536532 | −2.902933 | −0.090779 |
| C | −1.450997 | 4.014643 | 0.823718 | C | 3.157556 | −2.027137 | 0.219079 |
| H | −2.105237 | 3.092716 | 2.662977 | C | 3.932174 | −0.884216 | 0.374723 |
| H | −0.948771 | 2.346455 | −2.081431 | C | 3.405908 | 0.375944 | −0.092464 |
| H | −1.016453 | 4.641556 | −1.194536 | C | 2.074831 | 0.435996 | −0.632466 |
| H | −1.481782 | 5.023890 | 1.224126 | C | 4.199431 | 1.548052 | −0.107606 |
| C | −1.279209 | −0.044698 | −0.619984 | C | 3.770253 | 2.707538 | −0.721284 |
| C | −2.681144 | −0.536327 | −0.560225 | H | 4.401459 | 3.591452 | −0.719555 |
| C | −3.333124 | −0.441338 | 0.685120 | C | 1.717195 | 1.605645 | −1.333950 |
| C | −3.376518 | −1.076208 | −1.646246 | H | 0.788218 | 1.594021 | −1.878827 |
| C | −4.616652 | −0.960062 | 0.860758 | C | 2.531955 | 2.716749 | −1.386551 |
| C | −4.661924 | −1.589933 | −1.477141 | H | 5.174891 | 1.551485 | 0.371700 |
| H | −2.885570 | −1.123536 | −2.614423 | H | 2.215253 | 3.595572 | −1.941294 |
| C | −5.272495 | −1.548911 | −0.221082 | H | 1.280110 | −3.742909 | −1.228346 |
| H | −5.099681 | −0.894921 | 1.831361 | H | 3.560521 | −3.003921 | 0.467483 |
| H | −5.186524 | −2.026755 | −2.322162 | N | 5.189876 | −0.935903 | 0.939000 |
| H | −6.272416 | −1.951728 | −0.087234 | H | 5.813312 | −0.182406 | 0.685468 |
| S | −2.450216 | 0.295280 | 2.041607 | C | 5.839238 | −2.201998 | 1.231920 |
| C | −0.233406 | −0.921901 | −0.707383 | H | 5.948804 | −2.841246 | 0.341973 |
| C | 1.221481 | −0.719522 | −0.551946 | H | 5.264082 | −2.753685 | 1.983200 |
| C | −0.464763 | −2.462102 | −0.762614 | H | 6.829615 | −1.995565 | 1.642879 |
| C | 1.835693 | −1.927947 | −0.232885 | | | | |
| H | −0.749170 | −2.730432 | −1.787468 | | | | |
| C | 0.914037 | −3.087760 | −0.427148 | | | | |
| Stable Conformation, for MM 2 with Chain Truncation | | | | | | | |
| 0 1 | | | | | | | |
| C | 1.957267 | 3.335487 | 0.471968 | H | −1.484435 | −4.019050 | −0.190702 |
| C | 2.059235 | 1.981357 | 0.138380 | C | 0.355048 | −2.945539 | −1.782520 |
| C | 1.212311 | 1.024451 | 0.727997 | H | 0.323307 | −4.023389 | −1.978942 |
| C | 0.305691 | 1.455043 | 1.706702 | H | −0.484631 | −2.471745 | −2.302572 |
| C | 0.199867 | 2.801054 | 2.042062 | H | 1.285550 | −2.544590 | −2.196143 |
| C | 1.016853 | 3.744832 | 1.414446 | C | −3.345980 | −1.868716 | 0.302903 |
| H | 2.619626 | 4.056418 | 0.001330 | C | −4.009222 | −0.658781 | 0.111955 |
| H | −0.335394 | 0.717643 | 2.180476 | C | −3.264395 | 0.491278 | −0.348786 |
| H | −0.522277 | 3.116403 | 2.789484 | C | −1.836798 | 0.411563 | −0.476116 |
| H | 0.937136 | 4.796910 | 1.672791 | C | −3.915821 | 1.684039 | −0.744900 |
| C | 1.318338 | −0.394232 | 0.324696 | C | −3.212635 | 2.749306 | −1.270485 |
| C | 2.720543 | −0.879390 | 0.283369 | H | −3.738674 | 3.649811 | −1.574482 |
| C | 3.679956 | −0.100344 | −0.396726 | C | −1.156582 | 1.499637 | −1.074383 |
| C | 3.171936 | −2.041000 | 0.929647 | H | −0.093170 | 1.409777 | −1.255833 |
| C | 5.004116 | −0.531771 | −0.524353 | C | −1.821230 | 2.645338 | −1.455437 |
| C | 4.489708 | −2.474186 | 0.807724 | H | −4.998757 | 1.756402 | −0.691307 |
| H | 2.479399 | −2.601165 | 1.549398 | H | −1.270080 | 3.461859 | −1.913065 |
| C | 5.403125 | −1.732786 | 0.055606 | H | −0.952576 | −3.414206 | 1.385500 |
| H | 5.718618 | 0.088459 | −1.057951 | H | −3.899464 | −2.763557 | 0.569385 |
| H | 4.806408 | −3.383713 | 1.310176 | N | −5.376398 | −0.555447 | 0.276630 |
| H | 6.432117 | −2.065705 | −0.044869 | H | −5.721529 | 0.378019 | 0.449474 |
| S | 3.222545 | 1.464582 | −1.093584 | C | −6.151864 | −1.625994 | 0.878788 |
| C | 0.242766 | −1.165855 | −0.002473 | H | −5.767756 | −1.921741 | 1.867332 |
| C | −1.178372 | −0.799655 | −0.093560 | H | −6.148054 | −2.509144 | 0.230355 |
| C | 0.276706 | −2.670399 | −0.270828 | H | −7.184368 | −1.287460 | 0.985772 |
| C | −1.952277 | −1.925548 | 0.177128 | | | | |
| H | 1.115502 | −3.165228 | 0.215290 | | | | |
| C | −1.075359 | −3.143553 | 0.327200 | | | | |
| Metastable Conformation, | | | | | | | |
| for MM 3 with Chain Truncation | | | | | | | |
| 0 1 | | | | | | | |
| C | 3.064310 | 3.142423 | −1.068443 | H | −0.665009 | −2.030117 | −2.723134 |
| C | 2.902873 | 1.807333 | −0.669559 | C | 1.568958 | −2.836275 | −1.579165 |
| C | 1.615836 | 1.243138 | −0.461053 | H | 1.352750 | −3.779420 | −2.094726 |
| C | 0.527272 | 2.082950 | −0.787223 | H | 1.848132 | −2.089959 | −2.331067 |
| C | 0.686875 | 3.397313 | −1.202590 | H | 2.429202 | −3.000805 | −0.928046 |
| C | 1.963700 | 3.948671 | −1.321879 | C | −3.243086 | −1.325116 | −1.092987 |
| H | 4.071428 | 3.532611 | −1.190440 | C | −3.931369 | −0.411754 | −0.297499 |
| H | −0.474375 | 1.685305 | −0.735540 | C | −3.131692 | 0.474313 | 0.563688 |
| H | −0.193392 | 3.990666 | −1.432516 | C | −1.763323 | 0.360907 | 0.651232 |
| H | 2.101880 | 4.982496 | −1.624133 | C | −3.834454 | 1.486618 | 1.320686 |
| C | 1.467956 | −0.173232 | −0.080094 | C | −3.131106 | 2.270419 | 2.211027 |
| C | 2.623773 | −0.864695 | 0.516695 | H | −3.642859 | 3.046871 | 2.772819 |
| C | 3.971682 | −0.499569 | 0.324877 | C | −1.080459 | 1.141569 | 1.616023 |
| C | 2.380776 | −1.919364 | 1.423490 | H | −0.008907 | 1.009542 | 1.723075 |
| C | 5.014597 | −1.235165 | 0.904982 | C | −1.748201 | 2.063388 | 2.393396 |
| C | 3.406959 | −2.656055 | 1.997563 | H | −4.894989 | 1.655127 | 1.167229 |
| H | 1.348841 | −2.147041 | 1.670978 | H | −1.202508 | 2.651424 | 3.125913 |
| C | 4.737329 | −2.320547 | 1.715863 | H | −1.383937 | −3.377417 | −1.823289 |
| H | 6.041914 | −0.930234 | 0.724323 | H | −3.790410 | −2.021193 | −1.721275 |
| H | 3.174446 | −3.469061 | 2.159216 | N | −5.338865 | −0.328147 | −0.315209 |
| H | 5.551936 | −2.893763 | 2.159216 | C | −6.016786 | −0.882214 | −1.480770 |
| S | 4.404976 | 0.889864 | −0.643471 | H | −6.028964 | −1.986895 | −1.491167 |
| C | 0.314532 | −0.927674 | −0.282385 | H | −7.055107 | −0.535663 | −1.470713 |
| C | −1.088122 | −0.548436 | −0.227993 | H | −5.532930 | −0.524561 | −2.392665 |
| C | 0.328602 | −2.379468 | −0.811189 | C | −6.030838 | −0.696744 | 0.929009 |
| C | −1.848543 | −1.394023 | −1.039099 | H | −7.020101 | −0.227236 | 0.950643 |
| H | 0.124943 | −3.086138 | 0.007004 | H | −6.159243 | −1.790508 | 0.997223 |
| C | −0.941865 | −2.381055 | −1.718223 | H | −5.457405 | −0.362758 | 1.792863 |
| Transition State for Thermal Helix Inversion, | | | | | | | |
| for MM 3 with Chain Truncation | | | | | | | |
| 0 1 | | | | | | | |
| C | −2.162435 | 2.933377 | 1.559703 | H | 0.708576 | −3.590222 | 0.623396 |
| C | −2.052304 | 1.626264 | 1.077001 | C | −1.656287 | −3.102383 | 0.207183 |
| C | −1.582902 | 1.365657 | −0.227552 | H | −1.445804 | −4.176272 | 0.273711 |
| C | −1.472542 | 2.459440 | −1.096740 | H | −1.577172 | −2.679521 | 1.214556 |
| C | −1.607544 | 3.765266 | −0.634090 | H | −2.683082 | −2.991553 | −0.135903 |
| C | −1.898967 | 1.004787 | 0.710667 | C | 3.024994 | −1.930156 | 0.195749 |
| H | −2.508152 | 3.103031 | 2.575116 | C | 3.794749 | −0.783094 | 0.281635 |
| H | −1.277838 | 2.280666 | −2.150281 | C | 3.202056 | 0.472679 | −0.089239 |
| H | −1.491755 | 4.595919 | −1.324293 | C | 1.855388 | 0.512939 | −0.595414 |
| H | −1.990533 | 5.022055 | 1.080161 | C | 3.925258 | 1.686411 | 0.024028 |
| C | −1.494372 | −0.076490 | −0.610972 | C | 3.430505 | 2.868970 | −0.483587 |
| C | −2.880140 | −0.616422 | −0.558850 | H | 3.997764 | 3.789546 | −0.378442 |
| C | −3.544699 | −0.528054 | 0.679846 | C | 1.440120 | 1.715379 | −1.205382 |
| C | −3.550199 | −1.181910 | −1.647374 | H | 0.505693 | 1.710045 | −1.768545 |
| C | −4.811783 | −1.087050 | 0.850612 | C | 2.199934 | 2.864743 | −1.167144 |
| C | −4.821350 | −1.731571 | −1.484169 | H | 4.881928 | 1.667410 | 0.535016 |
| H | −3.050481 | −1.221622 | −2.611368 | H | 1.835926 | 3.768962 | −1.647234 |
| C | −5.440732 | −1.703179 | −0.232002 | H | 1.160902 | −3.755482 | −1.068298 |
| H | −5.305111 | −1.028017 | 1.816353 | H | 3.457861 | −2.900690 | 0.419508 |
| H | −5.327188 | −2.187924 | −2.330236 | N | 5.139184 | −0.810191 | 0.713944 |
| H | −6.428750 | −2.135655 | −0.102610 | C | 5.546706 | −1.972340 | 1.494863 |
| S | −2.702179 | 0.270682 | 2.027968 | C | 6.148502 | −0.476163 | −0.302514 |
| C | −0.418399 | −0.916527 | −0.675363 | H | 5.656988 | −2.888086 | 0.886334 |
| C | 1.037252 | −0.669089 | −0.523149 | H | 6.517355 | −1.755456 | 1.952371 |
| C | −0.602237 | −2.464268 | −0.721034 | H | 4.815648 | −2.158482 | 2.285185 |
| C | 1.680199 | −1.860820 | −0.197005 | H | 7.074253 | −0.163493 | 0.192209 |
| H | −0.830510 | −2.746564 | −1.756583 | H | 6.368712 | −1.348943 | −0.941241 |
| C | 0.778575 | −3.043517 | −0.325853 | H | 5.791276 | 0.334103 | −0.937108 |
| Stable Conformation, for MM 3 with Chain Truncation | | | | | | | |
| 0 1 | | | | | | | |
| C | 2.229076 | 3.324597 | 0.382908 | H | −1.307230 | −4.000350 | 0.039068 |
| C | 2.307987 | 1.963991 | 0.070256 | C | 0.444703 | −2.996368 | −1.698217 |
| C | 1.468705 | 1.027565 | 0.701491 | H | 0.388827 | −4.079193 | −1.857987 |
| C | 0.598137 | 1.482770 | 1.701177 | H | −0.418007 | −2.527926 | −2.184323 |
| C | 0.517108 | 2.835022 | 2.017331 | H | 1.354216 | −2.621324 | −2.177562 |
| C | 1.322854 | 3.758995 | 1.347425 | C | −3.133452 | −1.815847 | 0.442247 |
| H | 2.883516 | 4.030815 | −0.119929 | C | −3.796076 | −0.621791 | 0.187851 |
| H | −0.035819 | 0.760510 | 2.206656 | C | −3.039035 | 0.536192 | −0.211780 |
| H | −0.178552 | 3.170473 | 2.780743 | C | −1.619311 | 0.432087 | −0.402579 |
| H | 1.261529 | 4.816092 | 1.589729 | C | −3.658450 | 1.793774 | −0.424951 |
| C | 1.546774 | −0.398794 | 0.318209 | C | −2.950611 | 2.861042 | −0.935991 |
| C | 2.942374 | −0.900534 | 0.248974 | H | −3.441952 | 3.818969 | −1.082806 |
| C | 3.989271 | −0.144764 | −0.473396 | C | −0.944042 | 1.519241 | −1.010886 |
| C | 3.398606 | −2.054209 | 0.905402 | H | 0.101911 | 1.404699 | −1.266572 |
| C | 5.204077 | −0.594890 | −0.631343 | C | −1.593774 | 2.702273 | −1.286013 |
| C | 4.707784 | −2.503975 | 0.755365 | H | −4.704234 | 1.907530 | −0.159865 |
| H | 2.717766 | −2.594337 | 1.555186 | H | −1.050547 | 3.521404 | −1.748485 |
| C | 5.606433 | −1.788217 | −0.038353 | H | −0.689291 | −3.328291 | 1.556216 |
| H | 5.909810 | 0.006198 | −1.197457 | H | −3.695578 | −2.710075 | 0.694100 |
| H | 5.029496 | −3.406341 | 1.267320 | N | −5.200850 | −0.510711 | 0.300167 |
| H | 6.528496 | −2.134596 | −0.161414 | C | −5.929321 | −0.287075 | −0.957859 |
| S | 3.431369 | 1.413610 | −1.183782 | C | −5.869992 | −1.511944 | 1.121966 |
| C | 0.455730 | −1.162681 | 0.032802 | H | −6.076601 | −1.235483 | −1.502786 |
| C | −0.966699 | −0.778774 | −0.016164 | H | −6.911341 | 0.145381 | −0.738301 |
| C | 0.457839 | −2.674120 | −0.193945 | H | −5.373460 | 0.396204 | −1.599086 |
| C | −1.737399 | −1.895187 | 0.330881 | H | −6.894116 | −1.173614 | 1.309731 |
| H | 1.316521 | −3.165978 | 0.258983 | H | −5.925536 | −2.504622 | 0.639528 |
| C | −0.865491 | −3.106255 | 0.494126 | H | −5.348695 | −1.611675 | 2.077043 |
| Metastable Conformation on the Functionalized | | | | | | | |
| Side of the Stator, for MM 4 with Chain Truncation | | | | | | | |
| 0 1 | | | | | | | |
| C | 2.209198 | 3.422883 | −0.650601 | H | −0.256686 | −2.295505 | −2.822738 |
| C | 2.405049 | 2.048949 | −0.442189 | C | 2.164612 | −2.657469 | −1.858395 |
| C | 1.307584 | 1.172365 | −0.224695 | H | 2.135245 | −3.556950 | −2.484663 |
| C | 0.032604 | 1.755535 | −0.335579 | H | 2.219793 | −1.782956 | −2.515990 |
| C | −0.176244 | 3.123423 | −0.554608 | H | 3.078949 | −2.694030 | −1.264018 |
| C | 0.938963 | 3.970808 | −0.691192 | C | −2.837159 | −2.366664 | −1.088351 |
| H | 3.079565 | 4.058773 | −0.789235 | C | −3.656110 | −1.697415 | −0.190277 |
| H | −0.844525 | 1.133646 | −0.271326 | C | −3.102443 | −0.846357 | 0.821316 |
| H | 0.799183 | 5.036416 | −0.839527 | C | −1.686159 | −0.634846 | 0.862880 |
| C | 1.529329 | −0.275471 | −0.044946 | C | −3.925652 | −0.240783 | 1.801271 |
| C | 2.862695 | −0.723479 | 0.394833 | C | −3.374082 | 0.520887 | 2.809841 |
| C | 4.064567 | −0.024947 | 0.164247 | H | −4.015488 | 0.980582 | 3.556407 |
| C | 2.953838 | −1.892200 | 1.180844 | C | −1.153613 | 0.122356 | 1.934200 |
| C | 5.298017 | −0.539179 | 0.586393 | H | −0.078301 | 0.254332 | 1.991718 |
| C | 4.171312 | −2.411435 | 1.598562 | C | −1.975868 | 0.687158 | 2.886040 |
| H | 2.029879 | −2.389040 | 1.459651 | H | −4.997270 | −0.398683 | 1.749955 |
| C | 5.357270 | −1.743492 | 1.277045 | H | −1.543518 | 1.264578 | 3.698314 |
| H | 6.205100 | 0.022651 | 0.380351 | H | −0.587126 | −3.861934 | −2.062657 |
| H | 4.197490 | −3.321989 | 2.189998 | H | −3.247566 | −3.057548 | −1.816791 |
| H | 6.317535 | −2.135705 | 1.598976 | O | −5.014758 | −1.816631 | −0.164935 |
| S | 4.067066 | 1.518836 | −0.659205 | C | −5.621305 | −2.664126 | −1.144684 |
| C | 0.579181 | −1.260168 | −0.294881 | H | −6.694158 | −2.598665 | −0.962085 |
| C | −0.869794 | −1.239297 | −0.150594 | H | −5.391372 | −2.314455 | −2.158433 |
| C | 0.902692 | −2.597159 | −0.997197 | H | −5.285072 | −3.701403 | −1.025839 |
| C | −1.454823 | −2.141390 | −1.039585 | C | −1.506503 | 3.625107 | −0.622475 |
| H | 0.920637 | −3.417070 | −0.264060 | C | −2.651989 | 4.022311 | −0.660303 |
| C | −0.384271 | −2.805515 | −1.856717 | C | −4.031657 | 4.485830 | −0.697027 |
| | | | | H | −4.411458 | 4.516523 | −1.725615 |
| | | | | H | −4.680836 | 3.818394 | −0.118081 |
| | | | | H | −4.120432 | 5.494410 | −0.276591 |
| Transition State for Thermal Helix Inversion on | | | | | | | |
| the Functionalized Side of the Stator, | | | | | | | |
| for MM 4 with Chain Truncation | | | | | | | |
| 0 1 | | | | | | | |
| C | −1.826810 | 1.779196 | 2.327651 | H | 1.328758 | −4.184170 | −0.314989 |
| C | −1.726449 | 0.626723 | 1.541492 | C | −1.082293 | −3.725010 | −0.351523 |
| C | −1.314623 | 0.702871 | 0.193477 | H | −0.819727 | −4.772676 | −0.538352 |
| C | −1.254997 | 1.969051 | −0.389146 | H | −0.928313 | −3.520439 | 0.713632 |
| C | −1.366496 | 3.141235 | 0.377515 | H | −2.140714 | −3.608157 | −0.576624 |
| C | −1.603618 | 3.026565 | 1.761626 | C | 3.430835 | −2.256253 | −0.159330 |
| H | −2.125151 | 1.695059 | 3.368449 | C | 4.066071 | −1.095616 | 0.234948 |
| H | −1.115840 | 2.062232 | −1.460406 | C | 3.432031 | 0.173828 | 0.041078 |
| H | −1.677611 | 3.924731 | 2.366082 | C | 2.092528 | 0.221286 | −0.473873 |
| C | −1.192443 | −0.599575 | −0.528328 | C | 4.141649 | 1.376782 | 0.268713 |
| C | −2.539013 | −1.229125 | −0.548600 | C | 3.595351 | 2.596100 | −0.074554 |
| C | −3.151844 | −1.454644 | 0.699537 | H | 4.151346 | 3.512458 | 0.102260 |
| C | −3.219660 | −1.599000 | −1.712039 | C | 1.621781 | 1.478753 | −0.903814 |
| C | −4.375031 | −2.121239 | 0.783707 | H | 0.691277 | 1.509355 | −1.444469 |
| C | −4.445391 | −2.258997 | −1.632995 | C | 2.339844 | 2.640605 | −0.708964 |
| H | −2.761819 | −1.397325 | −2.676504 | H | 5.139104 | 1.315742 | 0.689667 |
| C | −5.011893 | −2.536373 | −0.386298 | H | 1.932953 | 3.587026 | −1.053705 |
| H | −4.827370 | −2.303902 | 1.754061 | H | 1.691441 | −3.769624 | −1.985678 |
| H | −4.957560 | −2.561454 | −2.541911 | H | 3.926712 | −3.219656 | −0.108657 |
| H | −5.965121 | −3.053370 | −0.323181 | O | 5.323880 | −1.047262 | 0.760195 |
| S | −2.296867 | −0.931784 | 2.168788 | C | 6.007426 | −2.290907 | 0.937034 |
| C | −0.072454 | −1.324335 | −0.825173 | H | 6.967039 | −2.035207 | 1.387047 |
| C | 1.362078 | −1.008680 | −0.644747 | H | 5.443947 | −2.953392 | 1.605331 |
| C | −0.155611 | −2.832882 | −1.203644 | H | 6.168173 | −2.788740 | −0.027183 |
| C | 2.096034 | −2.189926 | −0.598051 | C | −1.229077 | 4.414998 | −0.241478 |
| H | −0.464052 | −2.910838 | −2.253122 | C | −1.096307 | 5.496278 | −0.775852 |
| C | 1.291812 | −3.366896 | −1.045657 | C | −0.944521 | 6.796659 | −1.413806 |
| | | | | H | −0.383983 | 7.486014 | −0.771528 |
| | | | | H | −0.406783 | 6.707807 | −2.364853 |
| | | | | H | −1.921341 | 7.250344 | −1.620984 |
| Stable Conformation on the Functionalized Side | | | | | | | |
| of the Stator, for MM 4 with Chain Truncation | | | | | | | |
| 0 1 | | | | | | | |
| C | 1.565316 | 3.238424 | −0.802648 | C | 1.263104 | −3.604357 | −0.923645 |
| C | 1.944113 | 1.900755 | −0.642793 | H | 1.429970 | −4.674726 | −0.757444 |
| C | 1.199607 | 1.040739 | 0.186578 | H | 0.408725 | −3.485651 | −1.598834 |
| C | 0.119360 | 1.566901 | 0.898529 | H | 2.148237 | −3.187074 | −1.413545 |
| C | −0.278252 | 2.904354 | 0.743406 | C | −2.746390 | −2.654204 | 0.538462 |
| C | 0.456731 | 3.732760 | −0.128921 | C | −3.556395 | −1.713147 | −0.072940 |
| H | 2.147145 | 3.888599 | −1.449620 | C | −2.990804 | −0.627622 | −0.820502 |
| H | −0.447156 | 0.915798 | 1.555759 | C | −1.566013 | −0.477287 | −0.872768 |
| H | 0.158106 | 4.768374 | −0.255427 | C | −3.814281 | 0.259653 | −1.554088 |
| C | 1.580717 | −0.384542 | 0.289910 | C | −3.258361 | 1.231798 | −2.358694 |
| C | 3.037232 | −0.579399 | 0.498478 | H | −3.899782 | 1.907521 | −2.917389 |
| C | 3.934896 | 0.113543 | −0.340107 | C | −1.034682 | 0.489238 | −1.760387 |
| C | 3.585404 | −1.368655 | 1.521702 | H | 0.039315 | 0.553586 | −1.880788 |
| C | 5.316589 | −0.075806 | −0.233718 | C | −1.857687 | 1.325009 | −2.484379 |
| C | 4.960233 | −1.557347 | 1.634838 | H | −4.890505 | 0.146049 | −1.486107 |
| H | 2.920090 | −1.825616 | 2.247057 | H | −1.420883 | 2.058285 | −3.156423 |
| C | 5.828115 | −0.931872 | 0.737355 | H | −0.241950 | −3.281455 | 2.175464 |
| H | 5.982905 | 0.468168 | −0.897031 | H | −3.165706 | −3.517947 | 1.042981 |
| H | 5.354636 | −2.182510 | 2.430740 | O | −4.920992 | −1.759997 | −0.065057 |
| H | 6.901542 | −1.076124 | 0.818701 | C | −5.536624 | −2.840279 | 0.640156 |
| S | 3.325480 | 1.263731 | −1.543548 | H | −6.610469 | −2.679179 | 0.541276 |
| C | 0.691073 | −1.409520 | 0.172858 | H | −5.261604 | −3.804762 | 0.195565 |
| C | −0.758014 | −1.366067 | −0.094224 | H | −5.251654 | −2.827721 | 1.699427 |
| C | 0.999961 | −2.886753 | 0.411717 | C | −1.415834 | 3.401856 | 1.437803 |
| C | −1.352656 | −2.471563 | 0.505647 | C | −2.396263 | 3.814111 | 2.020987 |
| H | 1.860391 | −3.036606 | 1.061112 | C | −3.579783 | 4.300904 | 2.715369 |
| C | −0.305446 | −3.390638 | 1.083458 | H | −3.511887 | 4.107700 | 3.792639 |
| H | −0.511537 | −4.446036 | 0.869614 | H | −3.703517 | 5.380582 | 2.573100 |
| | | | | H | −4.483951 | 3.805100 | 2.341764 |
| Metastable Conformation on the Non-Functionalized | | | | | | | |
| Side of the Stator for MM 4 with Chain Truncation | | | | | | | |
| 0 1 | | | | | | | |
| C | 1.288680 | 4.443328 | 0.049776 | H | −1.110440 | −0.646946 | −3.367802 |
| C | 1.557669 | 3.078235 | −0.125105 | C | 1.411188 | −1.085000 | −2.736489 |
| C | 0.527591 | 2.101556 | −0.062996 | H | 1.361981 | −1.781248 | −3.581938 |
| C | −0.781044 | 2.611737 | 0.085659 | H | 1.367483 | −0.063293 | −3.129617 |
| C | −1.050055 | 3.965001 | 0.236301 | H | 2.377907 | −1.221209 | −2.249071 |
| C | −0.009136 | 4.894401 | 0.245978 | C | −3.498321 | −1.398702 | −1.509923 |
| H | 2.116198 | 5.147192 | 0.015873 | C | −4.253083 | −1.075563 | −0.391402 |
| H | −1.616002 | 1.929440 | 0.059440 | C | −3.644233 | −0.487484 | 0.765414 |
| H | −2.080028 | 4.290205 | 0.353329 | C | −2.248291 | −0.165033 | 0.737181 |
| H | −0.204012 | 5.953374 | 0.386641 | C | −4.387544 | −0.259367 | 1.948771 |
| C | 0.833077 | 0.677827 | −0.297750 | C | −3.774070 | 0.236281 | 3.080080 |
| C | 2.221906 | 0.222116 | −0.102693 | H | −4.353970 | 0.403854 | 3.983241 |
| C | 3.363498 | 1.038416 | −0.232527 | C | −1.647329 | 0.312376 | 1.927140 |
| C | 2.431969 | −1.101835 | 0.322470 | H | −0.583046 | 0.522681 | 1.922479 |
| C | 4.651483 | 0.507009 | −0.072183 | C | −2.389888 | 0.506362 | 3.072778 |
| C | 3.709924 | −1.653531 | 0.480532 | H | −5.445730 | −0.496215 | 1.952033 |
| H | 1.565780 | −1.719929 | 0.531490 | H | −1.904877 | 0.868684 | 3.974884 |
| C | 4.830852 | −0.827780 | 0.250988 | H | −1.284945 | −2.381782 | −3.048875 |
| H | 5.512828 | 1.158400 | −0.192866 | H | −3.942928 | −1.900714 | −2.362394 |
| H | 5.829727 | −1.234445 | 0.370806 | O | −5.590330 | −1.319872 | −0.279071 |
| S | 3.219867 | 2.738795 | −0.599302 | C | −6.247489 | −1.923010 | −1.397382 |
| C | −0.085572 | −0.271179 | −0.733153 | H | −7.295324 | −2.007313 | −1.108389 |
| C | −1.511956 | −0.400132 | −0.471569 | H | −6.154319 | −1.293137 | −2.290360 |
| C | 0.234785 | −1.345726 | −1.795522 | H | −5.835280 | −2.918635 | −1.602105 |
| C | −2.134510 | −1.076079 | −1.521276 | C | 3.868929 | −3.010792 | 0.878136 |
| H | 0.361101 | −2.327664 | −1.318515 | C | 4.003282 | −4.160967 | 1.213668 |
| C | −1.117330 | −1.408902 | −2.574705 | C | 4.162724 | −5.558925 | 1.617884 |
| | | | | H | 3.363363 | −5.861803 | 2.304220 |
| | | | | H | 4.131705 | −6.228524 | 0.749683 |
| | | | | H | 5.121368 | −5.712537 | 2.126721 |
| Transition State for Thermal Helix Inversion on | | | | | | | |
| the Non-Functionalized Side of the Stator, | | | | | | | |
| for MM 4 with Chain Truncation | | | | | | | |
| 0 1 | | | | | | | |
| C | −0.197106 | 3.708220 | 1.323192 | H | 0.807260 | −3.453773 | 1.379031 |
| C | −0.510817 | 2.360442 | 1.127172 | C | −1.390045 | −2.367991 | 1.303066 |
| C | −0.394958 | 1.758587 | −0.143506 | H | −1.429975 | −3.429337 | 1.574038 |
| C | −0.194848 | 2.611561 | −1.236867 | H | −0.987217 | −1.814273 | 2.158115 |
| C | 0.093502 | 3.961417 | −1.056373 | H | −2.412095 | −2.038350 | 1.126900 |
| C | 0.146446 | 4.500095 | 0.230861 | C | 3.302580 | −2.464699 | 0.390148 |
| H | −0.278760 | 4.139466 | 2.316540 | C | 4.281056 | −1.523045 | 0.144662 |
| H | −0.271352 | 2.209830 | −2.242692 | C | 3.950049 | −0.298117 | −0.519076 |
| H | 0.269703 | 4.594608 | −1.921080 | C | 2.578521 | −0.016166 | −0.839004 |
| H | 0.385406 | 5.549356 | 0.378040 | C | 4.968468 | 0.592164 | −0.934073 |
| C | −0.737518 | 0.305178 | −0.210130 | C | 4.673244 | 1.691701 | −1.712678 |
| C | −2.167733 | 0.175243 | 0.178002 | H | 5.464649 | 2.364605 | −2.030343 |
| C | −2.521290 | 0.659544 | 1.453479 | C | 2.339768 | 1.065711 | −1.710547 |
| C | −3.150298 | −0.385176 | −0.634823 | H | 1.338504 | 1.200996 | −2.081647 |
| C | −3.820075 | 0.490830 | 1.937584 | C | 3.348633 | 1.903830 | −2.137611 |
| C | −4.466242 | −0.553678 | −0.164707 | H | 5.992203 | 0.372151 | −0.652448 |
| H | −2.888120 | −0.733887 | −1.628821 | H | 3.117494 | 2.723320 | −2.812764 |
| C | −4.780052 | −0.124549 | 1.141325 | H | 0.873984 | −3.940280 | −0.310067 |
| H | −4.080531 | 0.849186 | 2.929152 | H | 3.541921 | −3.435162 | 0.811523 |
| H | −5.791991 | −0.254413 | 1.511301 | O | 5.601989 | −1.690252 | 0.440458 |
| S | −1.262338 | 1.427253 | 2.442119 | C | 5.980638 | −2.915703 | 1.073141 |
| C | 0.076943 | −0.791082 | −0.249928 | H | 7.054651 | −2.836651 | 1.243348 |
| C | 1.548695 | −0.907258 | −0.369020 | H | 5.457072 | −3.038398 | 2.029078 |
| C | −0.457549 | −2.216288 | 0.083314 | H | 5.767819 | −3.772981 | 0.422654 |
| C | 1.959915 | −2.142654 | 0.12486 | C | −5.457541 | −1.156240 | −0.989314 |
| H | −0.975875 | −2.603191 | −0.802524 | C | −6.300648 | −1.671164 | −1.693515 |
| C | −0.810098 | −3.067112 | 0.352185 | C | −7.313761 | −2.285851 | −2.539902 |
| | | | | H | −7.789524 | −1.540323 | −3.188576 |
| | | | | H | −6.873741 | −3.057669 | −3.182093 |
| | | | | H | −8.098448 | −2.755370 | −1.935511 |
| Stable Conformation on the Non-Functionalized | | | | | | | |
| Side of the Stator, for MM 4 with chain Truncation | | | | | | | |
| 0 1 | | | | | | | |
| C | 0.136510 | 3.998456 | 0.744921 | H | −0.616507 | −3.971779 | −0.753384 |
| C | 0.659426 | 2.816745 | 0.210813 | C | 0.521652 | −2.138552 | −2.308783 |
| C | 0.264194 | 1.561055 | 0.707989 | H | 0.838926 | −3.126962 | −2.660040 |
| C | −0.616498 | 1.525810 | 1.797820 | H | −0.487851 | −1.941825 | −2.685915 |
| C | −1.138555 | 2.698489 | 2.333962 | H | 1.197636 | −1.387748 | −2.729400 |
| C | −0.773097 | 3.935742 | 1.797912 | C | −3.039815 | −2.704263 | 0.154866 |
| H | 0.452619 | 4.956669 | 0.342706 | C | −4.078645 | −1.789813 | 0.173039 |
| H | −0.906648 | 0.559918 | 2.200211 | C | −3.846853 | −0.405010 | −0.120099 |
| H | −1.834891 | 2.648905 | 3.165786 | C | −2.507949 | 0.048290 | −0.360889 |
| H | −1.179496 | 4.853825 | 2.212402 | C | −4.924371 | 0.505602 | −0.236926 |
| C | 0.801620 | 0.329236 | 0.091790 | C | −4.704009 | 1.808608 | −0.631476 |
| C | 2.271113 | 0.381262 | −0.114735 | H | −5.538912 | 2.497967 | −0.721103 |
| C | 2.819880 | 1.522050 | −0.738190 | C | −2.331182 | 1.374686 | −0.823377 |
| C | 3.153066 | −0.613015 | 0.320388 | H | −1.336988 | 1.704536 | −1.096581 |
| C | 4.188071 | 1.592400 | −1.024161 | C | −3.400031 | 2.235114 | −0.955046 |
| C | 4.529978 | −0.555994 | 0.044800 | H | −5.928573 | 0.151609 | −0.031728 |
| H | 2.774180 | −1.448526 | 0.897998 | H | −3.233511 | 3.246924 | −1.313835 |
| C | 5.033870 | 0.553794 | −0.662252 | H | −0.138054 | −3.390584 | 0.848858 |
| H | 4.587211 | 2.476830 | −1.512260 | H | −3.218041 | −3.764120 | 0.301054 |
| H | 6.094839 | 0.608563 | −0.883321 | O | −5.382165 | −2.119670 | 0.410019 |
| S | 1.786334 | 2.896401 | −1.154068 | C | −5.674109 | −3.493884 | 0.674765 |
| C | 0.028868 | −0.729526 | −0.278568 | H | −6.748737 | −3.534554 | 0.854566 |
| C | −1.434774 | −0.886896 | −0.211431 | H | −5.415902 | −4.121500 | −0.187146 |
| C | 0.545205 | −2.080833 | −0.771505 | H | −5.132138 | −3.844198 | 1.561852 |
| C | −1.730123 | −2.237317 | −0.053754 | C | 5.395511 | −1.597683 | 0.482594 |
| H | 1.556925 | −2.294665 | −0.432279 | C | 6.131702 | −2.487317 | 0.854874 |
| C | −0.475639 | −3.069511 | −0.146748 | C | 7.016040 | −3.554029 | 1.303071 |
| | | | | H | 6.834353 | −4.478657 | 0.741937 |
| | | | | H | 8.067124 | −3.276522 | 1.163850 |
| | | | | H | 6.862079 | −3.771335 | 2.366716 |
| Metastable Conformation on the Functionalized | | | | | | | |
| Side of the Stator, for MM 5 with Chain Truncation | | | | | | | |
| 0 1 | | | | | | | |
| C | −3.255096 | −0.886877 | −0.168919 | H | 2.415889 | 1.021956 | −3.144056 |
| C | −2.340217 | 0.167238 | −0.082240 | C | 1.194358 | 3.097187 | −2.083659 |
| C | −0.937776 | −0.030520 | −0.155977 | H | 1.823409 | 3.674120 | −2.771902 |
| C | −0.557668 | −1.357192 | −0.462508 | H | 0.452228 | 2.548882 | −2.674486 |
| C | −1.446090 | −2.408961 | −0.567352 | H | 0.663735 | 3.804316 | −1.443646 |
| C | −2.832230 | −2.211009 | −0.369045 | C | 4.335277 | −0.853653 | −1.544817 |
| H | −4.310904 | −0.649744 | −0.099202 | C | 4.448826 | −1.896373 | −0.645261 |
| H | 0.484091 | −1.570401 | −0.651613 | H | 5.265625 | −2.609461 | −0.726601 |
| H | −1.056358 | −3.390324 | −0.810596 | C | 3.531412 | −2.035610 | 0.430038 |
| C | 0.012940 | 1.087742 | −0.088958 | C | 2.438681 | −1.113509 | 0.565547 |
| C | −0.465419 | 2.384239 | 0.425530 | C | 3.692407 | −3.055798 | 1.403638 |
| C | −1.814343 | 2.795099 | 0.452701 | C | 2.840816 | −3.149914 | 2.482048 |
| C | 0.463688 | 3.252175 | 1.040737 | H | 2.979565 | −3.933811 | 3.221535 |
| C | −2.180964 | 4.060463 | 0.934906 | C | 1.600540 | −1.219648 | 1.701774 |
| C | 0.109923 | 4.504406 | 1.520104 | H | 0.797572 | −0.501717 | 1.828312 |
| H | 1.487899 | 2.908784 | 1.139430 | C | 1.796915 | −2.211387 | 2.639557 |
| C | −1.221402 | 4.926916 | 1.440270 | H | 4.515384 | −3.757519 | 1.287976 |
| H | −3.229768 | 4.345390 | 0.926584 | H | 1.145217 | −2.270751 | 3.506887 |
| H | 0.862443 | 5.142558 | 1.973994 | H | 3.879693 | 1.819201 | −2.542357 |
| H | −1.515649 | 5.904468 | 1.811017 | H | 5.067975 | −0.729146 | −2.338545 |
| S | −3.108075 | 1.750625 | −0.085049 | N | −3.728276 | −3.265140 | −0.383278 |
| C | 1.344533 | 1.012580 | −0.494623 | C | −3.286422 | −4.570867 | −0.851871 |
| C | 2.285177 | −0.098245 | −0.437031 | C | −5.155319 | −2.983199 | −0.415555 |
| C | 2.066385 | 2.122492 | −1.291585 | H | −2.429260 | −4.917176 | −0.265786 |
| C | 3.267176 | 0.047530 | −1.422400 | H | −3.001057 | −4.565825 | −1.915923 |
| H | 2.744149 | 2.690535 | −0.536360 | H | −4.101699 | −5.283262 | −0.712206 |
| C | 2.978983 | 1.278012 | −2.234147 | H | −5.442749 | −2.370848 | 0.445768 |
| | | | | H | −5.462002 | −2.456198 | −1.333446 |
| | | | | H | −5.698843 | −3.927932 | −0.356463 |
| Transition State for Thermal Helix Inversion on | | | | | | | |
| the Functionalized Side of the Stator, | | | | | | | |
| for MM 5 with Chain Truncation | | | | | | | |
| 0 1 | | | | | | | |
| C | 2.392935 | −1.377129 | −0.991232 | H | −4.329844 | 1.352293 | −1.295756 |
| C | 1.033496 | −1.274607 | −0.706424 | C | −3.770431 | −0.948545 | −0.654140 |
| C | 0.554140 | −0.664652 | 0.470072 | H | −4.826763 | −0.798596 | −0.905617 |
| C | 1.515796 | −0.411236 | 1.455711 | H | −3.192611 | −0.922335 | −1.584460 |
| C | 2.876924 | −0.519214 | 1.208110 | H | −3.675947 | −1.940363 | −0.216075 |
| C | 3.350877 | −0.922092 | −0.061368 | C | −2.658847 | 3.677518 | −1.059777 |
| H | 2.696154 | −1.875616 | −1.904073 | C | −1.494147 | 4.406846 | −1.090553 |
| H | 1.192221 | −0.128913 | 2.453379 | H | −1.443745 | 5.358215 | −1.514684 |
| H | 3.571455 | −0.298144 | 2.009758 | C | −0.355168 | 3.962995 | −0.370353 |
| C | −0.929581 | −0.612825 | 0.629254 | C | −0.390401 | 2.705055 | 0.328719 |
| C | −1.424939 | −2.014913 | 0.657287 | C | 0.795388 | 4.785597 | −0.264122 |
| C | −1.117731 | −2.816385 | −0.460073 | C | 1.841430 | 4.445571 | 0.564789 |
| C | −2.155276 | −2.573327 | 1.709951 | H | 2.712140 | 5.090533 | 0.643940 |
| C | −1.614484 | −4.116361 | −0.561495 | C | 0.658564 | 2.443958 | 1.234675 |
| C | −2.643000 | −3.876703 | 1.617695 | H | 0.569622 | 1.586114 | 1.879003 |
| H | −2.368046 | −1.964902 | 2.584735 | C | 1.747718 | 3.281078 | 1.354175 |
| C | −2.390607 | −4.638545 | 0.474297 | H | 0.817563 | 5.715046 | −0.828471 |
| H | −1.385498 | −4.716716 | −1.437164 | H | 2.531206 | 3.045225 | 2.069209 |
| H | −3.225370 | −4.296426 | 2.433001 | H | −4.636955 | 1.982516 | 0.316935 |
| H | −2.774810 | −5.651762 | 0.398080 | H | −3.559901 | 4.048129 | −1.542452 |
| S | −0.134423 | −2.107261 | −1.761150 | N | 4.704597 | −0.930897 | −0.360019 |
| C | −1.797229 | 0.427306 | 0.455609 | C | 5.669583 | −0.771735 | 0.717089 |
| C | −1.541209 | 1.858428 | 0.159620 | C | 5.158266 | −1.592955 | −1.573148 |
| C | −3.327624 | 0.198214 | 0.278625 | H | 5.629983 | −1.593847 | 1.450207 |
| C | −2.674967 | 2.420519 | −0.425708 | H | 5.498023 | 0.171405 | 1.246337 |
| H | −3.771243 | 0.034563 | 1.268292 | H | 6.671069 | −0.738764 | 0.283689 |
| C | −3.866023 | 1.525346 | −0.316916 | H | 4.988508 | −2.682141 | −1.555034 |
| | | | | H | 4.642766 | −1.178002 | −2.445301 |
| | | | | H | 6.227882 | −1.408302 | −1.691176 |
| Stable Conformation on the Functionalized Side | | | | | | | |
| of the Stator, for MM 5 with Chain Truncation | | | | | | | |
| 0 1 | | | | | | | |
| C | −1.352622 | −2.675036 | 0.187975 | C | 3.074334 | 2.494457 | 1.265822 |
| C | −0.142420 | −1.987983 | 0.266051 | H | 3.939044 | 3.167933 | 1.263670 |
| C | 0.113220 | −0.842293 | −0.505377 | H | 2.249973 | 2.989250 | 1.790920 |
| C | −0.872792 | −0.460845 | −1.425147 | H | 3.340226 | 1.589508 | 1.820838 |
| C | −2.081189 | −1.130466 | −1.525284 | C | −0.174644 | 4.523211 | −0.805691 |
| C | −2.358340 | −2.248564 | −0.704200 | C | −1.487753 | 4.385446 | −0.409506 |
| H | −1.499269 | −3.545834 | 0.816029 | H | −2.194626 | 5.200319 | −0.547249 |
| H | −0.692150 | 0.408111 | −2.051105 | C | −1.933347 | 3.205599 | 0.245934 |
| H | −2.818520 | −0.775435 | −2.235013 | C | −1.024400 | 2.111510 | 0.447523 |
| C | 1.367396 | −0.087169 | −0.322825 | C | −3.252266 | 3.108723 | 0.760595 |
| C | 2.568749 | −0.957818 | −0.279116 | C | −3.648151 | 2.012795 | 1.494839 |
| C | 2.537765 | −2.096409 | 0.554284 | H | −4.659118 | 1.954990 | 1.889131 |
| C | 3.718700 | −0.756536 | −1.057961 | C | −1.445749 | 1.029437 | 1.259273 |
| C | 3.658989 | −2.922058 | 0.688669 | H | −0.740393 | 0.241764 | 1.492258 |
| C | 4.834279 | −1.580340 | −0.932948 | C | −2.724563 | 0.979875 | 1.769533 |
| H | 3.727391 | 0.048206 | −1.785593 | H | −3.940048 | 3.932559 | 0.583614 |
| C | 4.815934 | −2.649884 | −0.035683 | H | −3.021980 | 0.140141 | 2.391573 |
| H | 3.607921 | −3.788360 | 1.342044 | H | −3.021980 | 0.140141 | 2.391573 |
| H | 5.713015 | −1.393815 | −1.543625 | H | 0.174692 | 5.453860 | −1.246294 |
| H | 5.683453 | −3.294908 | 0.069822 | N | −3.566665 | −2.921842 | −0.793006 |
| S | 1.066878 | −2.519765 | 1.447065 | C | −4.664478 | −2.306693 | −1.526048 |
| C | 1.409437 | 1.266508 | −0.176875 | C | −3.898510 | −3.911682 | 0.220497 |
| C | 0.285634 | 2.223085 | −0.114185 | H | −4.394077 | −2.162775 | −2.577779 |
| C | 2.663143 | 2.139717 | −0.173700 | H | −4.955589 | −1.330788 | −1.106561 |
| C | 0.711431 | 3.442609 | −0.638574 | H | −5.525263 | −2.976559 | −1.488805 |
| H | 3.508032 | 1.665064 | −0.668932 | H | −3.165238 | −4.725752 | 0.219862 |
| C | 2.186103 | 3.394508 | −0.955680 | H | −3.935249 | −3.481059 | 1.234288 |
| H | 2.710149 | 4.307464 | −0.649984 | H | −4.875230 | −4.337966 | −0.014703 |
| Metastable Conformation on the Non-Functionalized | | | | | | | |
| Side of the Stator, for MM 5 with Chain Truncation | | | | | | | |
| 0 1 | | | | | | | |
| C | 0.198094 | 4.060391 | −0.533150 | H | −1.677072 | −1.675672 | −3.265536 |
| C | 0.581936 | 2.711369 | −0.482952 | C | 0.842967 | −1.785947 | −2.501259 |
| C | −0.377793 | 1.669300 | −0.384675 | H | 0.902170 | −2.613291 | −3.218453 |
| C | −1.728087 | 2.080392 | −0.449128 | H | 0.718195 | −0.853186 | −3.062328 |
| C | −2.108191 | 3.412620 | −0.514325 | H | 1.793548 | −1.736583 | −1.967117 |
| C | −1.141391 | 4.420336 | −0.532159 | C | −4.077642 | −2.320884 | −1.368297 |
| H | 0.971820 | 4.822065 | −0.585915 | C | −4.897352 | −1.880315 | −0.346524 |
| H | −2.502680 | 1.329072 | −0.467561 | H | −5.939505 | −2.187507 | −0.303671 |
| H | −3.164660 | 3.661804 | −0.552614 | C | −4.389909 | −1.057383 | 0.693364 |
| H | −1.425180 | 5.468108 | −0.563698 | C | −3.019287 | −0.628589 | 0.664742 |
| C | 0.040290 | 0.255550 | −0.368352 | C | −5.205539 | −0.673571 | 1.789940 |
| C | 1.435034 | −0.058686 | −0.039206 | C | −4.693288 | 0.067757 | 2.831320 |
| C | 2.530904 | 0.816039 | −0.181370 | H | −5.329167 | 0.351005 | 3.665528 |
| C | 1.732172 | −1.300349 | 0.563139 | C | −2.516576 | 0.105250 | 1.766741 |
| C | 3.838438 | 0.429060 | 0.118644 | H | −1.471805 | 0.397333 | 1.767169 |
| C | 3.016300 | −1.710830 | 0.864552 | C | −3.331425 | 0.443918 | 2.826129 |
| H | 0.902069 | −1.956070 | 0.806548 | H | −6.246069 | −0.990173 | 1.797887 |
| C | 4.121059 | −0.863776 | 0.598081 | H | −2.923743 | 1.003140 | 3.663670 |
| H | 4.630997 | 1.158918 | −0.001920 | H | −1.722777 | −3.349891 | −2.685709 |
| H | 3.162574 | −2.675497 | 1.335711 | H | −4.463772 | −2.988956 | −2.134381 |
| S | 2.305126 | 2.459743 | −0.733861 | N | 5.418505 | −1.274910 | 0.835491 |
| C | −0.792545 | −0.813235 | −0.694150 | C | 5.657856 | −2.512570 | 1.563886 |
| C | −2.215961 | −1.014896 | −0.459336 | C | 6.500172 | −0.303708 | 0.766733 |
| C | −0.346783 | −2.007038 | −1.567079 | H | 5.175400 | −3.355062 | 1.057516 |
| C | −2.739312 | −1.902800 | −1.405146 | H | 5.287764 | −2.471278 | 2.600338 |
| H | −0.157454 | −2.892668 | −0.941439 | H | 6.732720 | −2.700624 | 1.584434 |
| C | −1.654850 | −2.305443 | −2.363693 | H | 6.522484 | 0.174396 | −0.218481 |
| | | | | H | 6.409456 | 0.483112 | 1.532209 |
| | | | | H | 7.447398 | −0.825793 | 0.912320 |
| Transition State for Thermal Helix Inversion on the | | | | | | | |
| Non-Functionalized Side of the Stator, | | | | | | | |
| for MM 5 with Chain Truncation | | | | | | | |
| 0 1 | | | | | | | |
| C | 0.221264 | 3.298382 | 1.320580 | H | 0.874894 | −3.927889 | 1.053822 |
| C | −0.087218 | 1.986462 | 0.950487 | C | −1.171226 | −2.670883 | 0.547907 |
| C | 0.336482 | 1.456978 | −0.287540 | H | −1.327062 | −3.741499 | 0.725155 |
| C | 0.852875 | 2.362293 | −1.223714 | H | −0.963053 | −2.193874 | 1.511801 |
| C | 1.138949 | 3.679348 | −0.874783 | H | −2.101186 | −2.264648 | 0.154394 |
| C | 0.874185 | 4.134119 | 0.418440 | C | 3.583127 | −3.000233 | 0.840804 |
| H | −0.101687 | 3.671994 | 2.287862 | C | 4.626418 | −2.109312 | 0.921997 |
| H | 1.025726 | 2.029594 | −2.242603 | H | 5.559303 | −2.382670 | 1.409133 |
| H | 1.562988 | 4.352112 | −1.614656 | C | 4.527829 | −0.838147 | 0.299576 |
| H | 1.110533 | 5.155849 | 0.701297 | C | 3.303236 | −0.445800 | −0.347752 |
| C | −0.042596 | 0.035132 | −0.550793 | C | 5.654023 | 0.21593 | 0.237343 |
| C | −1.526583 | −0.030754 | −0.553870 | C | 5.626122 | 1.182148 | −0.503790 |
| C | −2.187421 | 0.393914 | 0.612273 | H | 6.499847 | 1.826100 | −0.550449 |
| C | 2.313009 | −0.484143 | −1.614873 | C | 3.349852 | 0.700975 | −1.167386 |
| C | −3.566587 | 0.282161 | 0.756311 | H | 2.492582 | 0.920285 | −1.780427 |
| C | −3.693190 | −0.600537 | −1.496069 | C | 4.471358 | 1.499204 | −1.247736 |
| H | −1.829926 | −0.781338 | −2.541941 | H | 6.558413 | −0.276316 | 0.762870 |
| C | −4.348926 | −0.254167 | −0.290993 | H | 4.466058 | 2.370853 | −1.896531 |
| H | −4.027542 | 0.630363 | 1.672768 | H | 1.355042 | −4.277526 | −0.601359 |
| H | −4.262905 | −0.963293 | −2.343316 | H | 3.677272 | −4.005257 | 1.244949 |
| S | −1.198258 | 1.024375 | 1.951365 | N | −5.718802 | −0.416338 | −0.146030 |
| C | 0.706165 | −1.105023 | 0.477885 | C | −6.382345 | 0.162009 | 1.012551 |
| C | 2.155221 | −1.304571 | −0.224374 | C | −6.528924 | −0.688366 | −1.324096 |
| C | 0.038369 | −2.510291 | −0.397493 | H | −6.301328 | 1.260661 | 1.043938 |
| C | 2.366797 | −2.595626 | 0.257890 | H | −5.957174 | −0.240798 | 1.938186 |
| H | −0.267862 | −2.806973 | −1.408007 | H | −7.438909 | −0.109955 | 0.980230 |
| C | 1.158075 | −3.459166 | 0.103404 | H | −6.472071 | 0.118382 | −2.072413 |
| | | | | H | −6.214763 | −1.623224 | −1.800992 |
| | | | | H | −7.568473 | −0.802756 | −1.011841 |
| Stable Conformation on the Non-Functionalized | | | | | | | |
| Side of the Stator, for MM 5 with Chain Truncation | | | | | | | |
| 0 1 | | | | | | | |
| C | −0.235445 | 3.582336 | 0.427615 | H | −1.536692 | −4.450536 | 0.101365 |
| C | 0.232484 | 2.301808 | 0.115936 | C | −0.170104 | −2.986869 | −1.656875 |
| C | −0.314594 | 1.162666 | 0.735764 | H | 0.085275 | −4.040245 | −1.819829 |
| C | −1.288309 | 1.349500 | 1.726530 | H | −1.136773 | −2.786339 | −2.131716 |
| C | −1.753808 | 2.621595 | 2.043185 | H | 0.587389 | −2.365990 | −2.145099 |
| C | −1.238502 | 3.738555 | 1.381188 | C | −3.916502 | −2.865843 | 0.551761 |
| H | 0.197347 | 4.447276 | −0.066926 | C | −4.865586 | −1.893955 | 0.316818 |
| H | −1.694408 | 0.475679 | 2.227043 | H | −5.923020 | −2.11506 | 0.447823 |
| H | −2.522343 | 2.742506 | 2.800852 | C | −4.493162 | −0.609682 | −0.163695 |
| H | −1.600628 | 4.733868 | 1.622446 | C | −3.104777 | −0.289371 | −0.350589 |
| C | 0.168646 | −0.182679 | 0.354058 | C | −5.475606 | 0.349997 | −0.520790 |
| C | 1.646916 | −0.266931 | 0.286606 | C | −5.118885 | 1.550560 | −1.093185 |
| C | 2.354896 | 0.728783 | −0.414065 | H | −5.881719 | 2.274181 | −1.367085 |
| C | 2.424341 | −1.241641 | 0.929412 | C | −2.778812 | 0.928502 | −0.996715 |
| C | 3.739277 | 0.682850 | −0.573211 | H | −1.742024 | 1.141686 | −1.224974 |
| C | 3.803281 | −1.306668 | 0.787682 | C | −3.759271 | 1.826153 | −1.358994 |
| H | 1.936846 | −1.958750 | 1.582374 | H | −6.522621 | 0.107003 | −0.354298 |
| C | 4.492825 | −0.369831 | −0.015971 | H | −3.482953 | 2.751994 | −1.855627 |
| H | 4.224758 | 1.491502 | −1.106507 | H | −1.121705 | −3.624416 | 1.611420 |
| H | 4.345723 | −2.080146 | 1.318281 | H | −4.213873 | −3.866571 | 0.855677 |
| S | 1.487213 | 2.106516 | −1.117859 | N | 5.861200 | −0.461964 | −0.222439 |
| C | −0.662205 | −1.225599 | 0.078293 | C | 6.646512 | −1.365094 | 0.608888 |
| C | −2.137915 | −1.264007 | 0.048500 | C | 6.559928 | 0.655827 | −0.838831 |
| C | −0.230795 | −2.673175 | −0.151926 | H | 6.282177 | −2.393331 | 0.503186 |
| C | −2.554494 | −2.548361 | 0.397705 | H | 6.612731 | −1.094061 | 1.676635 |
| H | 0.738787 | −2.897214 | 0.288572 | H | 7.681102 | −1.336286 | 0.271001 |
| C | −1.366203 | −3.465652 | 0.551396 | H | 6.132294 | 0.876598 | −1.822439 |
| | | | | H | 6.517822 | 1.572809 | −0.228725 |
| | | | | H | 7.605929 | 0.377123 | −0.979684 |

Geometries optimized at the TPSSTPSS(GD3BJ)/def2-TZVP level on the Gaussian 16 Superfine grid.
The motor goes from Metastable Functionalized −> Transition State (TS) Functionalized −> Stable Functionalized −> Metastable Nonfunctionalized −> TS Nonfunctionalized −> Stable Nonfunctionalized.
Further details are provided in elsewhere in Example 2.

### Refinement of the Electronic Energy

Geometries, frequencies, and thermochemical corrections to the electronic energy are fairly converged at the TPSSTPSS (GD3BJ)/def2-TZVP G16 SuperFineGrid level of theory. However, the electronic energy can usually be improved. The basis set was further increased to def2-TZVPPD (retrieved from the Basis Set Exchange for H, C, N, O, and S), which is a large triple-(quality basis set augmented with diffuse functions, and we performed single-point energy calculations on the SuperFineGrid to further refine the electronic energy (Weigend 2005; Frisch, 2016; Feller, 1996; Pritchard, 2019; Rappoport, 2010; Schuchardt, 2007). Both the TPSSTPSS mGGA and Truhlar's empirical Minnesota 2006 double-exchange (M06-2X) hybrid mGGA (Weigend 2005) were used. Although the local noncovalent interactions were mostly parameterized into the M06-2X functional, Grimme's D3 dispersion was applied to describe noncovalent interactions at a longer range (Grimme, 2010). Since both TPSS and M06-2X are widely benchmarked well-tested functionals, the average of the two electronic energies was used to form the best guess of the electronic energy. Then, the electronic energies from the single-point electronic energy calculations were combined with the free energy thermochemical corrections from the TPSSTPSS (GD3BJ)/def2-TZVP SuperFineGrid level of theory, to provide accurate approximations for the free energy differences between the metastable, transition, and ground states of the motors (Weigend 2005). Thus, a composite method comprised of seven TPSSTPSS (GD3BJ) steps and one M06-2X (D3) single-point energy (TMiX71) was used. The overall computational workflow is shown in Scheme 5. This composite method was able to calculate the free energy barrier of the Thermal Helix Inversion of the core to within ~1.5 kJ/mol of the experimental value.

### Equilibrium Molecular Dynamics Simulations

To study interactions between membrane bilayer lipid and single MM molecules MM 1 and MM 2, all-atom molecular dynamics (MD) simulations were performed using GROMACS 2020.6 package and CHARMM36 force field (Páll, 2020; Hess, 2008; Abraham, 2021). CHARMM-GUI was used to generate the different topological conformations and parameters for the simulated systems (Jo, 2008). To generate topological conformations for MM 1 and MM 2 embedded within the bilayer membranes, geometry optimization of the molecules was conducted with Gaussian09 package using B3LYP/6-31G* level of theory, followed by parametrization using CHARMM-GUI Ligand Modeller and CHARMM36 force field parameters (Kim, 2017; Frisch, 2009). Both MM molecules were considered in the protonated states. Next, the bilayer membranes were built around the single MM molecules. CHARMM-GUI Membrane Builder was used to build IPOPE/3POPG lipid bilayers containing 11 POPE and 33 POPG molecules (1:3 POPE: POPG ratio) in each layer with the desired MM in the middle (Jo, 2007; Jo, 2009; Wu, 2014; Lee, 2016; Klauda, 2010; Venable, 2014). The resulting membranes were then neutralized by adding counter-ions of sodium and then solvated by adding 5 nm of TIP3P water on each side with 150 mM of NaCl buffer to simulate the ionic strength of the buffer used in experiments. The resulting system dimensions were about 5×5×14.0 nm (FIG. 59). In all simulations the membrane is located parallel to the XY plane. Prior to all MD simulations, the energy minimization (steepest descent until maximum force in the system was less than 1000 kJ/mol/nm) and six steps of equilibration with decreasing harmonic restraining forces were performed using the default CHARMM-GUI protocol (Jo, 2008). After equilibration, both systems were simulated using NPT ensemble with periodic boundary conditions in all directions, leap-frog integrator and 2 fs integration step. Constant pressure of 1 atm was maintained using semi-isotropic Parrinello-Rahman barostat. Constant temperature of 298 K was maintained using Nose-Hoover thermostat. The Particle-mesh Ewald (PME) algorithm was used to calculate the long-range electrostatic interactions. In all simulations, the TIP3P model of water was utilized. For equilibrium simulations, 5 production runs with a duration of 100 ns each were performed for each MM (not including equilibration). During the analysis, the first 10 ns of each trajectory were discarded. GridMAT-MD was used to calculate the thickness of the lipid bilayer membrane. Grid size was 20×20 and thickness was measured using phosphorus atoms on POPE and POPG molecules by time-averaging all simulations (500 ns in total for each MM) (Allen, 2009).

### Umbrella Sampling Simulations

A combination of steered MD (SMD), umbrella sampling, and weighted histogram analysis (WHAM) methods was used to describe the binding profiles for both MMs. First, each MM was slowly pulled out of the membrane. For this, a steering force was applied to the Center of Mass (COM) of the MM using a spring constant of 1000 kJ/mol/nm² along the Z-axis only. This force was then used to pull the MM out of the membrane at a rate of 1 nm/ns. From the resulting trajectory a subset of snapshots was selected with increasing distance between MM and the center of the membrane along Z axis; the distance varied between 0 nm and 4 nm with 0.2 nm step, which resulted in 21 snapshots for each MM. These snapshots were used as starting points for umbrella sampling simulation windows. During umbrella sampling simulations, a force with a spring constant of 1000 kJ/mol/nm² was used to hold the MM molecule at its initial position along the Z-axis. For each window at least two 100 ns simulations were performed, resulting in a total simulation time of 4.2 μs for each MM. Final trajectories were analyzed using WHAM to yield Potential of Mean Force (PMF) curves (Hub, 2010).

### Example 3—Molecular Machine Therapy in Cancer

Described below is the treatment of melanoma and oral cancer cells using wide field 405 nm LED light and stimulus activated molecular machines according to the presently disclosed methods, showing that various types of cancer cell lines treated with stimulus activated molecular machines exhibited almost 100% loss in viability. Likewise, stimulus activated molecular machines were evaluated in subcutaneous tumor model of B16-F10 melanoma in C57BL/6J mice and their therapeutic efficacy is discussed in the sections that follow. The exemplary stimulus activated molecular machine used in the assays described below is shown in FIG. 50 and is also referred to herein as M96.

### Molecular Machine M96 is Effective to Kill Mouse Melanoma Cancer Cells B16-F10 In Vitro.

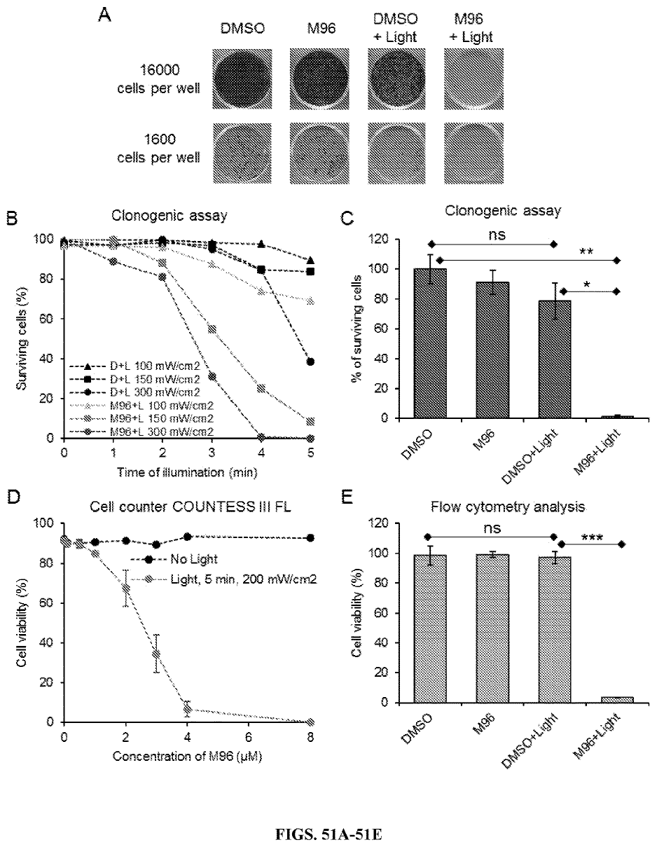

Mouse melanoma B16-F10 cells (1 mL of cell suspensions in media containing 200,000 cells/mL) were treated with molecular machine M96, which upon 405 nm light actuation cause necrotic cell death by cell membrane permeabilization. Cell death was assessed by cell culture of the surviving cells for a period of 9 days and counting the number of colony-forming cancer cells, also known as the clonogenic assay (FIG. 51A). The number of surviving cells upon treatment with blue-light-activated M96 (8 AM M96 and illumination with 405 nm light at 300 mW/cm² for 5 min) were nearly zero, as shown in FIG. 51A. By seeding a high number of cells (16000 cells per well) the results stood out and showed that almost none of the cells survived the treatment. The light dose, power intensity in m W/cm2 and illumination time were each varied to optimize the therapeutic effect of light-activated-M96 at 8 AM (FIG. 51B). Illumination for 4-5 min with a power intensity of 300 mW/cm² (72-90 J/cm²) at 8 μM M96 is sufficient to kill nearly 100% of the cells. In contrast, a 5 min illumination with a power intensity of 150 mW/cm² (45 J/cm²) and 8 μM M96 is slightly suboptimal, killing nearly 90% of the cells. FIG. 51C supports, by multiple repetitions of the clonogenic assay and statistical analysis, that 5 min of illumination at 300 m W/cm2 and 8 μM was enough to kill nearly 100% of the cells. FIG. 51D shows that by propidium iodide (PI) staining and counting the PI positive (death) cells in an automatic cell counter, illumination at 200 mW/cm² for 5 min (60 J/cm²) and 8 μM M96 was sufficient to kill nearly 100% of the cells. In this case the analysis was performed 2 h after the light treatment in contrast to the clonogenic assay which is read at 9 days after the treatment. Similarly, conducting the analysis by flow cytometry to count the PI positive cells (indicated dead cells) at 2 h after the treatment and analyzing a larger cell population of 10,000 cells, an illumination of 300 mW/cm2 for 5 min (90 J/cm²) was sufficient to kill nearly 100% of the cells (FIG. 51E). In summary, by utilizing different methods of analysis and optimizing concentration and light dose, we confirm that using 8 μM M96 and 5 min illumination at 200-300 mW/cm² (60-90 J/cm⁻²) is sufficient to kill nearly 100% of the B16-F10 cells in vitro.

### The Half Maximal Inhibitory Concentration (IC₅₀) of M96 Measures its Potency in Killing Cancer Cells.

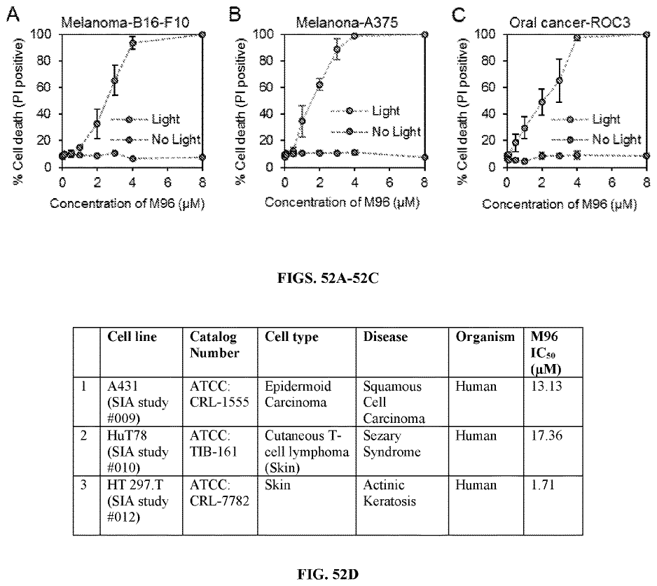

The IC₅₀ was measured in 1 mL cell suspensions of cancer cells containing 200,000 cells/mL. The in vitro IC₅₀ of molecular machine M96 upon illumination with 405 nm light at 200 mW/cm2 for 5 min was ~3 AM in mouse melanoma B16-F10 cells (FIG. 52A). In the human melanoma A375 cells, the in vitro IC₅₀ of stimulus activated molecular machine M96 upon illumination with 405 nm light at 150 mW/cm² for 5 min was ≈3 AM (FIG. 52B). In the mouse oral cancer ROC3 cells, the in vitro IC₅₀ of stimulus activated molecular machine M96 upon illumination with 405 nm light at 200 mW/cm² for 5 min was ~2 μM. Under these illumination conditions, the concentration of stimulus activated molecular machine M96 at 4 μM is demonstrated to be sufficient to kill nearly 95-99% of the cancer cells in cell lines. A concentration of M96 of 8 μM is shown to be sufficient to kill nearly 100% of the cells in vitro in cell lines B16-F10, A375 and ROC3. Human cells from various skin conditions were tested for the IC₅₀ of M96 upon illumination with 405 nm light at 200 mW/cm² for 5 minutes (FIG. 52D).

Time-Course Flow Cytometry Analysis Shows that the Cellular Membrane Permeabilization to Propidium Iodide (PI) is Immediate Upon Light-Activated-Molecular Machine M96 Treatment.

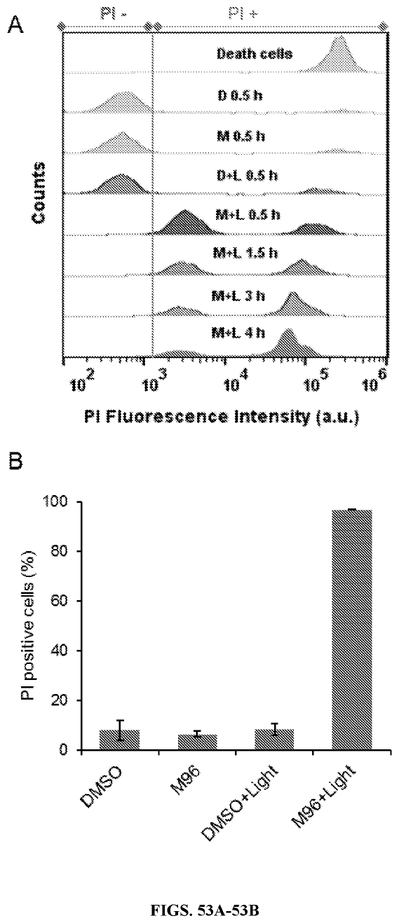

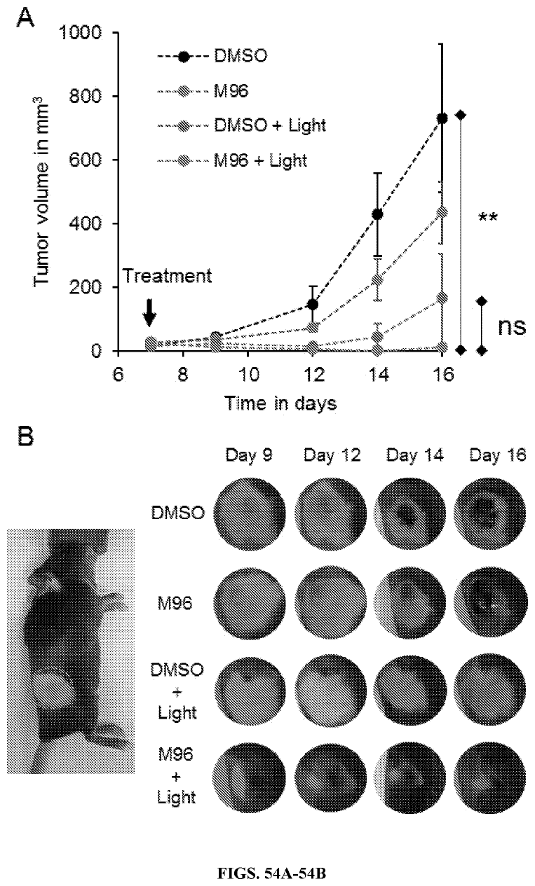

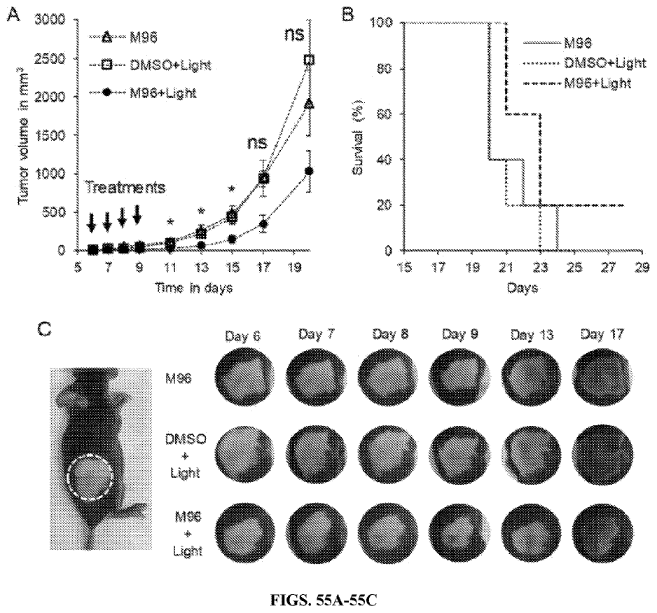

B16-F10 cells (1 mL cell suspension containing 200,000 cells/mL) were treated with 8 μM stimulus activated molecular machine M96 and illumination with 405 nm light at 300 mW/cm2 for 5 min, PI added for staining, and the cells were analyzed by flow cytometry. The PI enters the cells when the cell membrane is disrupted upon treatment with light-activated-M96 and stains the cellular DNA. Flow cytometry analysis detects and quantifies PI positive (that is, dead) cells (FIG. 53A). Time course flow cytometry was conducted to show that PI staining is immediate upon treatment, and that two PI positive subpopulations are detected. The first is a low intensity PI positive population (~10³ fluorescence intensity) and the second is a high intensity PI positive population (~10⁵) as shown in FIG. 53A. The high intensity PI positive population correspond to cells that are fully PI stained and death. The low intensity PI positive population corresponds to cells wherein their membrane was partially compromised and are in the process of dying. Over time, here from 0.5 hours to 4 hours, the dying subpopulation (low intensity PI) converted into the death subpopulation (high intensity PI). Ultimately, the total number of PI positive cells after light-activated M96 treatment is ~97% as shown in FIG. 53B.

In Vivo Therapeutic Efficacy of Light-Activated M96 in Subcutaneous Tumors of B16-F10 at Illumination of 300 mW/cm².

Subcutaneous tumor models of mouse melanoma B16-F10 were obtained in C57BL/6J mice. The tumors were treated by intratumoral injection of 50 μL of 8 μM M96, followed by irradiation with a 405 nm light at 300 mW/cm2 30 minutes later for a period of 5 min. The tumor sizes were measured after the treatment (FIG. 54A). The combination of M96 and light treatment caused a delay of the tumor growth. However, the treatment with 0.1% DMSO solution and the same light irradiation conditions caused a tumor growth delay which was non-statistically significant relative to the M96 and light combination. A light dose of 300 mW/cm2 for 5 min was sufficient to cause tissue damage in vivo. FIG. 54B shows that the intratumoral injection of 50 μL solution, either with 0.1% DMSO or 8 μM M96, followed by 405 nm light irradiation at 300 mW/cm² for 5 min was sufficient to cause necrotic skin damage including the tumor. Therefore, further work was undertaken to optimize the light dose to minimize generalized skin damage due to exposure to the light irradiation.

In Vivo Therapeutic Efficacy of Light-Activated M96 in Subcutaneous Tumors of B16-F10 at Illumination of 200 mW/cm².

Subcutaneous tumor models of mouse melanoma B16-F10 were obtained in C57BL/6J mice. The tumors were treated once a day for 4 days by intratumoral injection of 20 μL of 8 μM M96, followed 30 minutes later by irradiation with a 405 nm light at 200 mW/cm² for 5 min. The tumor sizes were measured after the treatment (FIG. 55A). In contrast to the illumination at 300 mW/cm², the irradiation at 200 mW/cm² for 5 min did not cause necrotic skin damage (FIG. 55B). The growth of tumors was delayed by the treatment with M96 and light combination. However, these tumors were able to continue growing and the mice were sacrificed when the tumors reached 2000 mm³, which is reflected in the minor survival rate as shown in FIG. 55B.

Comparison of In Vivo Therapeutic Efficacy Experiments with Different Concentrations of M96.

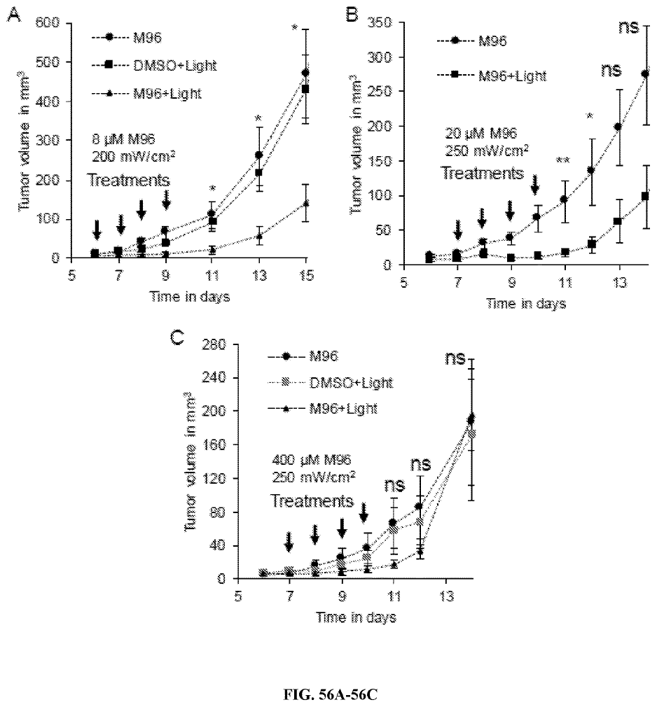

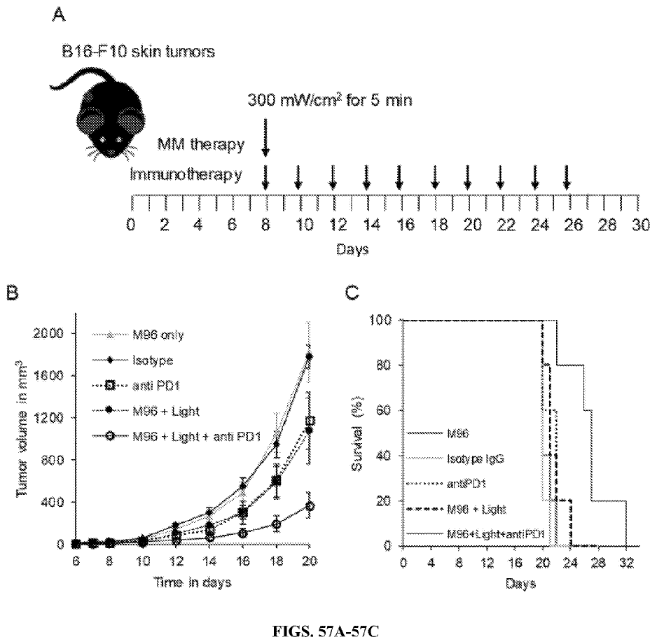

Subcutaneous tumor model of mouse melanoma B16-F10 were obtained in C57BL/6J mice and treated as described before with M96 and 405 nm light irradiation. When comparing various in vivo experiments that were conducted using different concentrations of M96 (8 μM, 20 μM, and 400 μM), it was observed that increasing the concentration does not have a better outcome in the reduction of the tumor sizes (FIG. 56). Counterintuitively, the higher concentration (400 μM) has a worst outcome in the reduction of the tumor sizes (FIG. 56C). This may be due, without being bound by theory, to the high absorbance of the more concentrated solution, corresponding to reduced transmittance of the light in the tissue, which in turn may cause less effective illumination of the tumor and result in suboptimum activation of M96.

Combination of Molecular Machine Therapy with Immunotherapy.

The immune check point blockade therapy with antibodies that targets the programmed cell death protein 1 pathway (PD-1/PD-L1 have demonstrated promised in a variety of malignancies (Postow et al., 2015). Pembrolizumab (anti-PD-1) is approved by the FDA for treatment of advanced melanoma. PD-1 is primarily believed to inhibit effector T-cell activity in the effector phase within tissue and tumors and by blocking PD-1 the immune system is activated against the tumors (Dong et al., 2002). The rationale in combining anti-PD-1 therapy with molecular machine therapy is that the mechanical action of molecular machines may destroy cancer cells and then release immunogenic molecules that may prime the immune system (Jiang et al., 2016; Krombach et al., 2019; Cushman et al., 2018; Bhalla et al., 2018; Vatner et al., 2014). Priming of the immune system in combination with the methods involving molecular machines as described herein may synergize with anti-PD-1 immunotherapy. FIG. 57 shows the results of the combination of molecular machine therapy with anti-PD-1 immunotherapy. An improvement in the tumor growth delay is observed with the combination (FIG. 57B), which is reflected in a slight improvement in the survival (FIG. 57C).

### Materials and Methods

### (i) Cancer Cell Lines

Mouse melanoma B16-F10 and ROC3 were obtained from the laboratory of Dr. Roberto Rangel at The University of Texas MD Anderson Cancer Center. ROC3 cell line was developed by Dr. Roberto Rangel. The A375 cell line was purchased from ATCC.

(ii)Chemicals

Molecular machine M96 was originally synthesized in the laboratory of Dr. James M. Tour (see Example 2). Molecular machine M96 was also obtained from Taros Chemicals GmbH and Co. KG (Germany).

(iii) Light Source

High power blue light 405 nm LED (Product: UHP—F-405 nm, Prizmatix, Israel) with 5 mm fiber optic (Product: LLG-5=liquid light guide, 5 mm) and light collimator to spread the beam to 1 inch output (Product: LLG-C5).

(iv)Culture of Mouse Melanoma B16-F10 Cells

Mouse melanoma B16-F10 cells were cultured in 10 cm polystyrene tissue culture treated dish (Corning) containing DMEM with 4.5 g/L glucose (Gibco, 11960-044) and supplemented with 10% FBS (SAFC Industries-Sigma-Aldrich, 12303C), 2X (10 mL) MEM vitamin solution (Corning, 25-020-Cl), 1X (5 mL) non-essential amino acid (NEAA) mixture (Lonza, 13-114E), 1X (5 mL) of L-glutamine (Lonza, 17-605E), and 1X (5 mL) of penicillin/streptomycin (Hyclone, SV30010). Typically, 0.5-1 million cells were inoculated per dish, cultured for 2-3 days in incubator at 37 ° C and 5% CO₂, then transferred to a new dish when confluency reached nearly 95-100%. For the passage to a new culture dish, cells were detached with 0.05% trypsin-EDTA (Gibco, 25-300-054). Then, the trypsinized cells were collected and mixed with 3 volumes of media to stop the action of the trypsin. Then e the cells were centrifuged at 1200 rpm for 5 min, resuspended in media, and inoculated to a new culture dish. A375 and ROC3 were cultured under the same conditions as described for B16-F10 cell line.

(v) Treatment of Cancer Cells with Molecular Machine and Light In Vitro.

The cells were harvested by trypsinizing them with 0.05% trypsin-EDTA. The trypsinized cells were collected and mixed with 3 volumes of media to stop the action of the trypsin. Then, the cells were centrifuged at 1200 rpm for 5 min. After the centrifugation, then the cells were resuspended using RPMI 1640 media without L-glutamine and without phenol red (SIGMA-ALDRICH, R7509) and supplemented with 10% FBS and penicillin/streptomycin. The final cell concentration was adjusted to 200,000 cells per mL. To 1 AL of molecular machine M96 at 8 mM in DMSO was added 1 mL of cells (200,000 cells per mL) on top of the volume of the M96 molecular machine. The mixture at the final concentration was 200000 cells, 8 μM M96, 0.1% DMSO. In a separated sample, DMSO without molecular machine was added as control containing 0.1% DMSO (1 μL of DMSO is added into 1 mL of cells at 200,000 cells per mL). Typically, 4 experimental groups were conducted: 1. M96+Light, 2. M96 only, 3. DMSO+Light, 4. DMSO only. The mixture was incubated at 37 ° C and 5% CO₂ for 30 min. After incubation 1 μL of PI was added (PI stock solution is at 1 mg/ml). Then, the mixture containing the cells and M96/DMSO was transferred into a 35 mm plastic culture dish. The mixture was then treated under the 405 nm LED light (PRIZMATIX, Israel, UHP—F-5-405) for 5 min at optical power of 300 mW/cm² (different optical powers were used, typically around the range of 150-300 mW/cm⁻²). The sample was placed sitting on top of an aluminum-block painted in black color while is treated under the light (Thermo-block, Thermo Scientific™ Dry Baths/Block Heaters, cat. #88-870-103). The light intensity was measured and adjusted using an Optical Power Meter from Thorlabs, sensor model S302C and console model PM100D. After the light treatment, the PI positive cells (death) in each sample were analyzed by flow cytometry or using an Automatic Cell Counter (Countess III FL Automatic Cell Counter, Invitrogen).

### (vi) Flow Cytometry

Immediately after the light treatments, the samples were transferred to a new FACS tube. The samples were incubated at 37 ° C and 5% CO₂ until all the samples were collected to be analyzed. The samples were analyzed typically at about 2 h after the light treatment.

(vii) Automatic Cell Counter.

Countes III fluorescence cell counter (Invitrogen) with fluorescence filters for detection of PI positive cells was used. After the light treatment, the sample was transferred into a clean 1.5 mL Eppendorf tube. The samples were centrifuged at 1200 rpm for 3.5 min. The supernatant was removed. The cells were dispersed in 150 μL of media. To obtain a cell suspensions at ~1−1.5×10⁶ cells per mL. The sample is loaded into the cell counter to obtain automatically the percentage of death cells. The results are saved for each sample and the images were analyzed semi-manually in ImageJ (free online software) to count the percentage of death cells.

(viii) Clonogenic Assay (Cell Death Quantification by Crystal Violet).

The cancer cells were treated the same way as described in section (v) above except that PI was not added. After the light treatment, the sample was transferred to a clean Eppendorf tube. Then, the cells were serially diluted 1:20 (to get 10,000 cells per mL) and 1:200 (to get 1000 cells per mL) in the media with phenol red (DMEM with 4.5 g/L glucose (Gibco, 11960-044) and supplemented with 10% FBS (SAFC Industries-Sigma-Aldrich, 12303C), 2X (10 mL) MEM vitamin solution (Corning, 25-020-C1), 1X (5 mL) non-essential amino acid (NEAA) mixture (Lonza, 13-114E), 1X (5 mL) of L-glutamine (Lonza, 17-605E), 1X (5 mL) of sodium pyruvate (Lonza, 13-115E) and 1X (5 mL) of penicillin/streptomycin (Hyclone, SV30010). 1000 cells (1 mL of suspension of 1000 cells per mL) were plated in each well of a 6-well cell culture plate containing appropriate total volume of about 1.5 mL. The cells were incubated at 37 ° C and 5% CO₂ for 7-12 days until the colonies were formed. The principle of the test is that death cells will not grow and viable cells will form colonies. Once the colonies were visible, colonies were stained. First, the media was removed and the cells were washed with ~1.5 mL of PBS buffer. Then, about 2 mL of 0.05% w/v crystal violet solution in methanol was added to the cells and allowed to stain for 5 min. Then, the crystal violet was removed and the excess of crystal violet was washed with water several times (~4-5 times). The cells contained in the 6-well plate were dried at room temperature. Then, the colonies in the 6-well plate were scanned and digitalized. Then, the colonies were counted and the percentage of survival was calculated relative to the control without treatment.

### (ix) In Vivo Studies.

All animal studies were approved by the Institutional Animal Care and Use Committee (IACUC) of the University of Texas MD Anderson Cancer Center (Houston, TX). 7-8 week old female C57BL/6J mice were used in the experiments described below.

### (x) Injection of B16-F10 Cells Subcutaneously in C57BL/6J Mice to Generate the Melanoma Tumors.

The B16-F10 cells were cultured as described above. Cells were harvested from confluent plates ~100%, fresh media was added to the cells the day before harvesting. The cells were harvested using 0.05% trypsin-EDTA (Gibco, 25-300-054). In detail, the cells were washed with PBS solution first before the trypsinization. Then, upon addition of ~1.5 mL of trypsin solution, the cells were incubated for about 1-2 min in the incubator at 37 ° C and 5% CO₂. To stop the trypsinization, 6 mL of DMEM media with supplements was added (the same used for the culture of the cells as described before). The cells were centrifuged at 300 r.c.f. (relative centrifugal force) for 3 min. The media was removed and discharged. Then, the cells were re-dispersed in ≈5 mL of DMEM media without supplements. The cell suspension was kept in ice. The cells were injected (100 μL of 1×10⁶ cells/mL suspension per mouse, corresponding to 100,000 cells per mouse) subcutaneously in the right flank of 7-8 weeks old female mouse (C57BL/6J), in which the hair in the right flank was previously depilated using an electric barber machine. The tumors were allowed to grow for 6 days counting from the day of cell injection. At day 6 the hair of the mouse was removed using on-the-shelf hair remover cream (Nair Hair Remover Lotion). For this purpose, a drop of the cream was placed on the skin, on top of the area where the tumor was injected. The mice were anesthetized using isoflurane while the hair remover cream was applied. Starting at day 7 the tumors were measured using a caliper. The tumors can be observed as a black spot (due to the melanin present in the B16-F10 cells) under the skin after the cream depilation. The typical volume of the tumors at about 7 days was approximately 15 mm³. The volume of the tumor was calculated as: (½)×length×width×height. When the height was not possible to measure in the case of the tumors which were too small (usually <100 mm³), then the tumor volume was calculated as: (½)×length×width².

### (xi) Preparation of Fresh Solution of 8 μM M96 and 0.1% DMSO the Day of Treatment

On the day of treatment of B16-F10 tumors with the M96, fresh solution 8 μM (or 20 μM) of M96 was prepared by diluting the 8 mM stock in PBS buffer. As control 0.1% DMSO in PBS was prepared by diluting 1 AL of DMSO in 1 mL PBS buffer. For the 20 μM M96, 2.5 AL of the stock 8 mM were used in 1 mL of PBS. And for this second case 0.25% DMSO was prepared as control.

(xii) Treatment of B16-F10 Tumors with M96 and Blue 405 nm Light

The tumors were treated at day 7 from the day of cell injection. At day 7 the tumors size typically 15 mm³. Typically, mice were divided in 4 groups (5-10 mice per group): 1) M96 only, 2) 0.1% DMSO only, 3) M96+light, and 4) 0.1% DMSO+light. The day of treatment, fresh solutions (8 μM or 20 μM of M96 in PBS and controls 0.1% DMSO or 0.25% DMSO in PBS) were prepared as described before. The mice were anesthetized with isoflurane using a vaporizer. Then, each mouse was injected with 30 AL of 8 μM M96 solution in PBS or 0.1% DMSO intratumorally. The tumors were small and many times is not possible deliver the whole 30 AL intratumorally, but the 30 μL are delivery as adjacent as possible to the tumor. Then mice were kept for 30 min in the cages to let the M96 solution or DMSO solution interact with the tumors. Then, after the 30 min of incubation, the mice were treated (under anesthesia, using isoflurane) with 405 nm light source from Prizmatix applying a power intensity of 250 mW/cm² for 5 min (Other powers were also investigated such as 200 mW/cm² or 300 mW/cm⁻²). The light intensity was measured using an Optical Power Meter from Thorlabs, sensor model S302C and console model PM100D. When the treatment was finished the mice were put back into the cages and housed in the animal facility. The treatment was repeated once a day for 4 days. The tumor sizes were measured every day starting the day of hair removal with cream. The tumors were measured using a caliper. Then after the 4 treatments, the tumors were measured every other day.

(xiii) Combination of Immunotherapy and MM Therapy

The mice were prepared, tumors generated, and treated as described before for the B16-F10 tumor model in C57BL/6J mice. The therapeutic regime of molecular machine therapy in combination with immunotherapy is described in FIG. 57. The immunotherapy was conducted by intraperitoneal injection of 100 AL of anti-PD1 solution in PBS at 2 μg/μL (200 μg per mouse). Mouse-IgG1 (isotype) was injected in the same way and concentration as a control. Anti-PD1 was purchased from BioXcell (anti-mouse-PD1 (CD279), In VivoPlus™, cat #BP0146). Mouse-IgGI isotype control was purchased from BioXcell (InVivoMab, clone MOPC-21, cat #BE0083).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references to the extent that they provide exemplary procedural or other details supplementary to those set forth herein, are specifically incorporated herein by reference.
CDC, Antibiotic resistance threats in the United States. US Dep. Heal. Hum. Serv. Centres Dis. Control Prev. (2019), pp. 1-113.
J. O'Neill, Review on antimicrobial resistance: tackling drug-resistant infections globally—final report and recommendations (Wellcome Trust, UK Government, 2016) (2016).
WHO, “No Time to Wait: Securing the future from drug-resistant infections” (2019)
M. S. Butler, D. L. Paterson, Antibiotics in the clinical pipeline in October 2019. J. Antibiot. (Tokyo). 73, 329-364 (2020).
Pew Research Center, Antibiotics Currently in Global Clinical Development (2020), (available at https://www.pewtrusts.org/-/media/assets/2017/05/antibiotics-currently-in-clinical-development-03-2017.pdf).
J. M. V Makabenta, A. Nabawy, C.-H. Li, S. Schmidt-Malan, R. Patel, V. M. Rotello, Nanomaterial-based therapeutics for antibiotic-resistant bacterial infections. Nat. Rev. Microbiol., 1-14 (2020).
D. P. Linklater, V. A. Baulin, S. Juodkazis, R. J. Crawford, P. Stoodley, E. P. Ivanova, Mechano-bactericidal actions of nanostructured surfaces. Nat. Rev. Microbiol., 1-15 (2020).
S. Cheeseman, A. J. Christofferson, R. Kariuki, D. Cozzolino, T. Daeneke, R. J. Crawford, V. K. Truong, J. Chapman, A. Elbourne, Antimicrobial Metal Nanomaterials: From Passive to Stimuli-Activated Applications. Adv. Sci. 7, 1902913 (2020).
B. L. Feringa, The art of building small: from molecular switches to molecular motors. J. Org. Chem. 72, 6635-6652 (2007).
M. Klok, N. Boyle, M. T. Pryce, A. Meetsma, W. R. Browne, B. L. Feringa, MHz unidirectional rotation of molecular rotary motors. J. Am. Chem. Soc. 130, 10484-10485 (2008).
V. García-López, F. Chen, L. G. Nilewski, G. Duret, A. Aliyan, A. B. Kolomeisky, J. T. Robinson, G. Wang, R. Pal, J. M. Tour, Molecular machines open cell membranes. Nature. 548, 567 (2017).
T. Galbadage, D. Liu, L. B. Alemany, R. Pal, J. M. Tour, R. S. Gunasekera, J. D. Cirillo, Molecular Nanomachines Disrupt Bacterial Cell Wall, Increasing Sensitivity of Extensively Drug-Resistant Klebsiella pneumoniae to Meropenem. ACS Nano. 13, 14377-14387 (2019).
S. Roy, in Ultraviolet Light in Human Health, Diseases and Environment, S. Ahmad, Ed. (Springer, 2017), pp. 207-219.
I. Keren, N. Kaldalu, A. Spoering, Y. Wang, K. Lewis, Persister cells and tolerance to antimicrobials. FEMS Microbiol. Lett. 230, 13-18 (2004).
P. Stiefel, U. Rosenberg, J. Schneider, S. Mauerhofer, K. Maniura-Weber, Q. Ren, Is biofilm removal properly assessed? Comparison of different quantification methods in a 96-well plate system. Appl. Microbiol. Biotechnol. 100, 4135-4145 (2016).
I. M. Helander, T. Mattila-Sandholm, Fluorometric assessment of Gram-negative bacterial permeabilization. J. Appl. Microbiol. 88, 213-219 (2000).
M. J. Hall, R. F. Middleton, D. Westmacott, The fractional inhibitory concentration (FIC) index as a measure of synergy. J. Antimicrob. Chemother. 11, 427-433 (1983).
E. Rubinstein, Y. Keynan, Vancomycin revisited-60 years later. Front. public Heal. 2, 217 (2014).
E. Maslova, Y. Shi, F. Sjöberg, H. S. Azevedo, D. W. Wareham, R. R. McCarthy, An Invertebrate Burn Wound Model That Recapitulates the Hallmarks of Burn Trauma and Infection Seen in Mammalian Models. Front. Microbiol. 11, 998 (2020).
K. Lewis, Persister cells, dormancy and infectious disease. Nat. Rev. Microbiol. 5, 48-56 (2007).
K. Lewis, Persister cells. Annu. Rev. Microbiol. 64, 357-372 (2010).
K. R. Allison, M. P. Brynildsen, J. J. Collins, Metabolite-enabled eradication of bacterial persisters by aminoglycosides, Nature. 473, 216-220 (2011).
B. P. Conlon, E. S. Nakayasu, L. E. Fleck, M. D. LaFleur, V. M. Isabella, K. Coleman, S. N. Leonard, R. D. Smith, J. N. Adkins, K. Lewis, Activated ClpP kills persisters and cradicates a chronic biofilm infection. Nature. 503, 365-370 (2013).
J.-H. Ch'ng, K. K. L. Chong, L. N. Lam, J. J. Wong, K. A. Kline, Biofilm-associated infection by enterococci. Nat. Rev. Microbiol. 17, 82-94 (2019).
P. S. Stewart, Mechanisms of antibiotic resistance in bacterial biofilms. Int. J. Med. Microbiol. 292, 107-113 (2002).
C. Vuotto, F. Longo, M. P. Balice, G. Donelli, P. E. Varaldo, Antibiotic resistance related to biofilm formation in Klebsiella pneumoniae. Pathogens. 3, 743-758 (2014).
R. M. Donlan, Role of biofilms in antimicrobial resistance. ASAIO J. 46, S47-S52 (2000).
M. O. A. Sommer, C. Munck, R. V. Toft-Kehler, D. I. Andersson, Prediction of antibiotic resistance: time for a new preclinical paradigm? Nat. Rev. Microbiol. 15, 689-696 (2017).
C. Kosmidis, D. P. Levine, Daptomycin: pharmacology and clinical use. Expert Opin. Pharmacother. 11, 615-625 (2010).
A. Prince, P. Sandhu, P. Ror, E. Dash, S. Sharma, M. Arakha, S. Jha, Y. Akhter, M. Saleem, Lipid-II independent antimicrobial mechanism of nisin depends on its crowding and degree of oligomerization. Sci. Rep. 6, 1-15 (2016).
T. T. Tran, J. M. Munita, C. A. Arias, Mechanisms of drug resistance: daptomycin resistance. Ann. N. Y. Acad. Sci. 1354, 32 (2015).
A. S. Bayer, T. Schneider, H.-G. Sahl, Mechanisms of daptomycin resistance in Staphylococcus aureus: role of the cell membrane and cell wall. Ann. N. Y. Acad. Sci. 1277, 139 (2013).
J. G. Hurdle, A. J. O'Neill, I. Chopra, R. E. Lee, Targeting bacterial membrane function: an underexploited mechanism for treating persistent infections. Nat. Rev. Microbiol. 9, 62-75 (2011).
M. R. Hamblin, J. Viveiros, C. Yang, A. Ahmadi, R. A. Ganz, M. J. Tolkoff, Helicobacter pylori accumulates photoactive porphyrins and is killed by visible light. Antimicrob. Agents Chemother. 49, 2822-2827 (2005).
T. Dai, A. Gupta, C. K. Murray, M. S. Vrahas, G. P. Tegos, M. R. Hamblin, Blue light for infectious diseases: Propionibacterium acnes, Helicobacter pylori, and beyond? Drug Resist. Updat. 15, 223-236 (2012).
Y. Wang, X. Wu, J. Chen, R. Amin, M. Lu, B. Bhayana, J. Zhao, C. K. Murray, M. R. Hamblin, D. C. Hooper, Antimicrobial blue light inactivation of gram-negative pathogens in biofilms: in vitro and in vivo studies. J. Infect. Dis. 213, 1380-1387 (2016).
R. M. Amin, B. Bhayana, M. R. Hamblin, T. Dai, Antimicrobial blue light inactivation of Pseudomonas aeruginosa by photo-excitation of endogenous porphyrins: In vitro and in vivo studies. Lasers Surg. Med. 48, 562-568 (2016).
Y. Zhang, Y. Zhu, J. Chen, Y. Wang, M. E. Sherwood, C. K. Murray, M. S. Vrahas, D. C. Hooper, M. R. Hamblin, T. Dai, Antimicrobial blue light inactivation of Candida albicans: In vitro and in vivo studies. Virulence. 7, 536-545 (2016).
F. Cieplik, A. Späth, C. Leibl, A. Gollmer, J. Regensburger, L. Tabenski, K.-A. Hiller, T. Maisch, G. Schmalz, Blue light kills Aggregatibacter actinomycetemcomitans due to its endogenous photosensitizers. Clin. Oral Investig. 18, 1763-1769 (2014).
T. Dai, A. Gupta, Y.-Y. Huang, R. Yin, C. K. Murray, M. S. Vrahas, M. E. Sherwood, G. P. Tegos, M. R. Hamblin, Blue light rescues mice from potentially fatal Pseudomonas aeruginosa burn infection: efficacy, safety, and mechanism of action. Antimicrob. Agents Chemother. 57, 1238-1245 (2013).
B. O'Donoghue, K. NicAogáin, C. Bennett, A. Conneely, T. Tiensuu, J. Johansson, C. O'Byrne, Blue-light inhibition of Listeria monocytogenes growth is mediated by reactive oxygen species and is influenced by σB and the blue-light sensor Lmo0799. Appl. Environ. Microbiol. 82, 4017-4027 (2016).
C. Ayala Orozco, D. Liu, Y. Li, L. B. Alemany, R. Pal, S. Krishnan, J. M. Tour, Visible-Light-Activated Molecular Nanomachines Kill Pancreatic Cancer Cells. ACS Appl. Mater. Interfaces. 12, 410-417 (2019).
J.-M. Pagès, C. E. James, M. Winterhalter, The porin and the permeating antibiotic: a selective diffusion barrier in Gram-negative bacteria. Nat. Rev. Microbiol. 6, 893-903 (2008).
H. Nikaido, Molecular basis of bacterial outer membrane permeability revisited. Microbiol. Mol. Biol. Rev. 67, 593-656 (2003).
M. Lu, S. Wang, T. Wang, S. Hu, B. Bhayana, M. Ishii, Y. Kong, Y. Cai, T. Dai, W. Cui, Bacteria-specific phototoxic reactions triggered by blue light and phytochemical carvacrol. Sci. Transl. Med. 13 (2021)
P. Ramakrishnan, M. Maclean, S. J. MacGregor, J. G. Anderson, M. H. Grant, Cytotoxic responses to 405 nm light exposure in mammalian and bacterial cells: involvement of reactive oxygen species. Toxicol. Vitr. 33, 54-62 (2016).
C. E. Thurman, A. Muthuswamy, M. M. Klinger, G. S. Roble, Safety Evaluation of a 405-nm LED Device for Direct Antimicrobial Treatment of the Murine Brain. Comp. Med. 69, 283-290 (2019).
X. Liu, Q. Chang, R. Ferrer-Espada, L. G. Leanse, X. S. Goh, X. Wang, J. A. Gelfand, T. Dai, Photoinactivation of Moraxella catarrhalis using 405-nm blue light: Implications for the treatment of otitis media. Photochem. Photobiol. 96, 611-617 (2020).
R. S. Gunasckera, T. Galbadage, C. Ayala-Orozco, D. Liu, V. García-López, B. E. Troutman, J. J. Tour, R. Pal, S. Krishnan, J. D. Cirillo, Molecular Nanomachines Can Destroy Tissue or Kill Multicellular Eukaryotes. ACS Appl. Mater. Interfaces. 12, 13657-13670 (2020).
N. Ramarao, C. Nielsen-Leroux, D. Lereclus, The insect Galleria mellonella as a powerful infection model to investigate bacterial pathogenesis. J. Vis. Exp. JoVE. 70, e4392 (2012).
C. R. Harding, G. N. Schroeder, S. Reynolds, A. Kosta, J. W. Collins, A. Mousnier, G. Frankel, Legionella pneumophila pathogenesis in the Galleria mellonella infection model. Infect. Immun. 80, 2780-2790 (2012).
E. Mylonakis, R. Moreno, J. B. El Khoury, A. Idnurm, J. Heitman, S. B. Calderwood, F. M. Ausubel, A. Diener, Galleria mellonella as a model system to study Cryptococcus neoformans pathogenesis. Infect. Immun. 73, 3842-3850 (2005).
J. C. Junior, B. B. Fuchs, C. P. Sabino, J. C. Junqueira, A. O. C. Jorge, M. S. Ribeiro, M. S. Gilmore, L. B. Rice, G. P. Tegos, M. R. Hamblin, Photodynamic and antibiotic therapy impair the pathogenesis of Enterococcus faecium in a whole animal insect model. PLOS One. 8, e55926 (2013).
I. Wojda, Immunity of the greater wax moth Galleria mellonella. Insect Sci. 24, 342-357 (2017).
G. Jander, L. G. Rahme, F. M. Ausubel, Positive correlation between virulence of Pseudomonas aeruginosa mutants in mice and insects. J. Bacteriol. 182. 3843-3845 (2000).
A. M. Borman, Of mice and men and larvae: Galleria mellonella to model the early host-pathogen interactions after fungal infection. Virulence. 9, 9-12 (2018).
M. Brennan, D. Y. Thomas, M. Whiteway, K. Kavanagh, Correlation between virulence of Candida albicans mutants in mice and Galleria mellonella larvae. FEMS Immunol. Med. Microbiol. 34, 153-157 (2002).
D. J. Leaper, Silver dressings: their role in wound management. Int. Wound J. 3, 282-294 (2006).
F. D. Halstead, M. Rauf, A. Bamford, C. M. Wearn, J. R. B. Bishop, R. Burt, A. P. Fraise, N. S. Moiemen, B. A. Oppenheim, M. A. Webber, Antimicrobial dressings: Comparison of the ability of a panel of dressings to prevent biofilm formation by key burn wound pathogens. Burns. 41, 1683-1694 (2015).
M. Guggenheim, R. Zbinden, A. E. Handschin, A. Gohritz, M. A. Altintas, P. Giovanoli, Changes in bacterial isolates from burn wounds and their antibiograms: a 20-year study (1986-2005). Burns. 35, 553-560 (2009).
Y. Fu, B. Xie, D. Ben, K. Lv, S. Zhu, W. Lu, H. Tang, D. Cheng, B. Ma, G. Wang, Pathogenic alteration in severe burn wounds. Burns. 38, 90-94 (2012).
E. Glukhov, M. Stark, L. L. Burrows, C. M. Deber, Basis for selectivity of cationic antimicrobial peptides for bacterial versus mammalian membranes. J. Biol. Chem. 280, 33960-33967 (2005).
G. K. Rajarao, N. Nekhotiaeva, L. Good, Peptide-mediated delivery of green fluorescent protein into yeasts and bacteria. FEMS Microbiol. Lett. 215, 267-272 (2002).
J. R. Morones-Ramirez, J. A. Winkler, C. S. Spina, J. J. Collins, Silver enhances antibiotic activity against gram-negative bacteria. Sci. Transl. Med. 5, 190ra81-190ra81 (2013).
G. A. O'Toole, Microtiter dish biofilm formation assay. JoVE (Journal Vis. Exp., e2437 (2011).
L. L. Ling, T. Schneider, A. J. Peoples, A. L. Spoering, I. Engels, B. P. Conlon, A. Mueller, T. F. Schäberle, D. E. Hughes, S. Epstein, A new antibiotic kills pathogens without detectable resistance. Nature. 517, 455 (2015).
T. Magoc, D. Wood, S. L. Salzberg, EDGE-pro: estimated degree of gene expression in prokaryotic genomes. Evol. Bioinform. 9, 127-136 (2013).
M. I. Love, W. Huber, S. Anders, Moderated estimation of fold change and dispersion for RNA-seq data with DESeq2. Genome Biol. 15, 1-21 (2014).
X. Ding, C. Yang, W. Moreira, P. Yuan, B. Periaswamy, P. F. de Sessions, H. Zhao, J. Tan, A. Lee, K. X. Ong, A Macromolecule Reversing Antibiotic Resistance Phenotype and Repurposing Drugs as Potent Antibiotics. Adv. Sci. 7, 2001374 (2020).
C. L. Friedrich, D. Moyles, T. J. Beveridge, R. E. W. Hancock, Antibacterial action of structurally diverse cationic peptides on gram-positive bacteria. Antimicrob. Agents Chemother. 44, 2086-2092 (2000).
D. Novo, N. G. Perlmutter, R. H. Hunt, H. M. Shapiro, Accurate flow cytometric membrane potential measurement in bacteria using diethyloxacarbocyanine and a ratiometric technique. Cytom. J. Int. Soc. Anal. Cytol. 35, 55-63 (1999).
A. J. O'Neill, K. Miller, B. Oliva, 1. Chopra, Comparison of assays for detection of agents causing membrane damage in Staphylococcus aureus. J. Antimicrob. Chemother. 54, 1127-1129 (2004).
E. C. Carlson, J. L. Audette, N. J. Veitenheimer, J. A. Risan, D. I. Latumus, P. N. Epstein, Ultrastructural morphometry of capillary basement membrane thickness in normal and transgenic diabetic mice. Anat. Rec. Part A Discov. Mol. Cell. Evol. Biol. An Off. Publ. Am. Assoc. Anat. 271, 332-341 (2003).
H. Eyring, The activated complex in chemical reactions. J. Chem. Phys. 3, 107-115 (1935).
F. Weigend, R. Ahlrichs, Balanced basis sets of split valence, triple zeta valence and quadruple zeta valence quality for H to Rn: Design and assessment of accuracy. Phys. Chem. Chem. Phys. 7, 3297-3305 (2005).
M. J. Frisch, G. W. Trucks, H. B. Schlegel, G. E. Scuseria, M. A. Robb, J. R. Cheeseman, G. Scalmani, V. Barone, G. A. Petersson, H. Nakatsuji, Gaussian 16 revision a. 03. Wallingford CT. 2 (2016).
A. L. Santos, V. Oliveira, I. Baptista, I. Henriques, N. C. M. Gomes, A. Almeida, A. Correia, Â. Cunha, Wavelength dependence of biological damage induced by UV radiation on bacteria. Arch. Microbiol. 195, 63-74 (2013),
K. Brudzynski, R. Lannigan, Mechanism of honey bacteriostatic action against MRSA and VRE involves hydroxyl radicals generated from honey's hydrogen peroxide. Front. Microbiol. 3, 36 (2012).
P. Belenky, D. Y. Jonathan, C. B. M. Porter, N. R. Cohen, M. A. Lobritz, T. Ferrante, S. Jain, B. J. Korry, E. G. Schwarz, G. C. Walker, Bactericidal antibiotics induce toxic metabolic perturbations that lead to cellular damage. Cell Rep. 13, 968-980 (2015).
D. A. Rowe-Magnus, A. Y. Kao, A. C. Prieto, M. Pu, C. Kao, Cathelicidin peptides restrict bacterial growth via membrane perturbation and induction of reactive oxygen species. MBio. 10 (2019).
M. M. Roy, D., Todd, A.K. John, Gauss View 6 (2019).
R. E. Stratmann, G. E. Scuseria, M. J. Frisch, Achieving linear scaling in exchange-correlation density functional quadratures. Chem. Phys. Lett. 257, 213-223 (1996).
J. Tao, J. P. Perdew, V. N. Staroverov, G. E. Scuseria, Climbing the density functional ladder: Nonempirical meta-generalized gradient approximation designed for molecules and solids. Phys. Rev. Lett. 91, 146401 (2003).
S. Grimme, S. Ehrlich, L. Goerigk, Effect of the damping function in dispersion corrected density functional theory. J. Comput. Chem. 32, 1456-1465 (2011).
B. I. Dunlap, Robust and variational fitting: Removing the four-center integrals from center stage in quantum chemistry. J. Mol. Struct. THEOCHEM. 529, 37-40 (2000).
D. Feller, The role of databases in support of computational chemistry calculations. J. Comput. Chem. 17, 1571-1586 (1996).
B. P. Pritchard, D. Altarawy, B. Didier, T. D. Gibson, T. L. Windus, New basis set exchange: An open, up-to-date resource for the molecular sciences community. J. Chem. Inf. Model. 59, 4814-4820 (2019).
D. Rappoport, F. Furche, Property-optimized Gaussian basis sets for molecular response calculations. J. Chem. Phys. 133, 134105 (2010).
K. L. Schuchardt, B. T. Didier, T. Elsethagen, L. Sun, V. Gurumoorthi, J. Chase, J. Li, T. L. Windus, Basis set exchange: a community database for computational sciences. J. Chem. Inf. Model. 47, 1045-1052 (2007).
S. Grimme, J. Antony, S. Ehrlich, H. Krieg, A consistent and accurate ab initio parametrization of density functional dispersion correction (DFT-D) for the 94 elements H—Pu. J. Chem. Phys. 132, 154104 (2010).
S. Páll, A. Zhmurov, P. Bauer, M. Abraham, M. Lundborg, A. Gray, B. Hess, E. Lindahl, Heterogeneous parallelization and acceleration of molecular dynamics simulations in GROMACS. J. Chem. Phys. 153, 134110 (2020).
B. Hess, C. Kutzner, D. Van Der Spoel, E. Lindahl, GROMACS 4: algorithms for highly efficient, load-balanced, and scalable molecular simulation. J. Chem. Theory Comput. 4, 435-447 (2008).
Lindahl, Abraham, Hess, van der Spoel, GROMACS 2020.6 Source code (2021), doi: 10.5281/ZENODO.4576055.
S. Kim, J. Lee, S. Jo, C. L. Brooks III, H. S. Lee, W. Im, CHARMM-GUI ligand reader and modeler for CHARMM force field generation of small molecules (2017).
M. J. Frisch, G. W. Trucks, H. B. Schlegel, G. E. Scuseria, M. A. Robb, J. R. Cheeseman, J. A. Gonzalez, J. A. Pople, Gaussian 09, Revision E. 01, Gaussian, Inc, Wallingford, CT, 2004. Inc., Wallingford CT. 121, 150-166 (2009).
S. Jo, T. Kim, W. Im, Automated builder and database of protein/membrane complexes for molecular dynamics simulations. PLOS One. 2, e880 (2007).
S. Jo, J. B. Lim, J. B. Klauda, W. Im, CHARMM-GUI Membrane Builder for mixed bilayers and its application to yeast membranes. Biophys. J. 97, 50-58 (2009).
E. L. Wu, X. Cheng, S. Jo, H. Rui, K. C. Song, E. M. Dávila-Contreras, Y. Qi, J. Lee, V. Monje-Galvan, R. M. Venable, CHARMM-GUI membrane builder toward realistic biological membrane simulations (2014).
J. Lec, X. Cheng, J. M. Swails, M. S. Ycom, P. K. Eastman, J. A. Lemkul, S. Wei, J. Buckner, J. C. Jeong, Y. Qi, CHARMM-GUI input generator for NAMD, GROMACS, AMBER, OpenMM, and CHARMM/OpenMM simulations using the CHARMM36 additive force field. J. Chem. Theory Comput. 12, 405-413 (2016).
J. B. Klauda, R. M. Venable, J. A. Freites, J. W. O'Connor, D. J. Tobias, C. Mondragon-Ramirez, I. Vorobyov, A. D. Mackerell Jr, R. W. Pastor, Update of the CHARMM all-atom additive force field for lipids: validation on six lipid types. J. Phys. Chem. B. 114, 7830-7843 (2010).
R. M. Venable, A. J. Sodt, B. Rogaski, H. Rui, E. Hatcher, A. D. Mackerell Jr, R. W. Pastor, J. B. Klauda, CHARMM all-atom additive force field for sphingomyelin: elucidation of hydrogen bonding and of positive curvature. Biophys. J. 107, 134-145 (2014).
W. J. Allen, J. A. Lemkul, D. R. Bevan, GridMAT-MD: a grid-based membrane analysis tool for use with molecular dynamics. J. Comput. Chem. 30, 1952-1958 (2009).
J. S. Hub, B. L. De Groot, D. Van Der Spoel, g_wham—A Free Weighted Histogram Analysis Implementation Including Robust Error and Autocorrelation Estimates. J. Chem. Theory Comput. 6, 3713-3720 (2010)
M. M. Pollard, P. V Wesenhagen, D. Pijper, B. L. Feringa, On the effect of donor and acceptor substituents on the behaviour of light-driven rotary molecular motors. Org. Biomol. Chem. 6, 1605-1612 (2008).
J. Conyard, A. Cnossen, W. R. Browne, B. L. Feringa, S. R. Meech, Chemically optimizing operational efficiency of molecular rotary motors. J. Am. Chem. Soc. 136, 9692-9700 (2014).
A. Saywell, A. Bakker, J. Mielke, T. Kumagai, M. Wolf, V. García-López, P.-T. Chiang, J. M. Tour, L. Grill, Light-induced translation of motorized molecules on a surface. ACS Nano. 10, 10945-10952 (2016).
P.-T. Chiang, J. Mielke, J. Godoy, J. M. Guerrero, L. B. Alemany, C. J. Villagomez, A. Saywell, L. Grill, J. M. Tour, Toward a light-driven motorized nanocar: Synthesis and initial imaging of single molecules. ACS Nano. 6, 592-597 (2012).
V. García-López, L. B. Alemany, P.-T. Chiang, J. Sun, P.-L. Chu, A. A. Marti, J. M. Tour, Synthesis of light-driven motorized nanocars for linear trajectories and their detailed NMR structural determination. Tetrahedron. 73, 4864-4873 (2017).
D. Liu, V. García-López, R. S. Gunasekera, L. Greer Nilewski, L. B. Alemany, A. Aliyan, T. Jin, G. Wang, J. M. Tour, R. Pal, Near-infrared light activates molecular nanomachines to drill into and kill cells. ACS Nano. 13, 6813-6823 (2019).
G. A. Gray, Carbon-13 nuclear magnetic resonance of organophosphorus compounds. VIII. Triphenylphosphoranes and triphenylphosphonium salts. J. Am. Chem. Soc. 95, 7736-7742 (1973).
R. S. Reid, The proton NMR spectrum of ascorbic acid: A relevant example of deceptively simple second-order behavior. J. Chem. Educ. 66, 344 (1989).
F. Balkau, M. W. Fuller, M. L. Heffernan, Deceptive simplicity in ABMX NMR spectra. I. Dibenzothiophen and 9, 9′-dicarbazyl. Aust. J. Chem. 24, 2293-2303 (1971).
J. B. Stothers, C. T. Tan, N. K. Wilson, 13C nmr studies of some phenanthrene and fluorene derivatives. Org. Magn. Reson. 9, 408-413 (1977).
J. A. G. Drake, D. W. Jones, High-resolution NMR spectra of fluorene and its derivatives-V 1H and 13C solvent and lanthanide-shift-reagent studies of fluorene-9-one. Spectrochim. Acta Part A Mol. Spectrosc. 36, 23-28 (1980).
J. Gmach, K. Huben, K. M. Błażewska, a-Fluoro phosphonocarboxylates: the NMR analysis of the heteronuclear ABMX spin system. Phosphorus. Sulfur. Silicon Relat. Elem. 189, 1216-1225 (2014).
R. A. van Delden, N. Koumura, A. Schoevaars, A. Meetsma, B. L. Feringa, A donor-acceptor substituted molecular motor: unidirectional rotation driven by visible light. Org. Biomol. Chem. 1, 33-35 (2003).
V. Diemer, F. R. Leroux, F. Colobert, Efficient and complementary methods offering access to synthetically valuable 1,2-dibromobenzenes. Euro. J. Org. Chem. 2011, 327-340 (2011).
T. N. Murakami, N. Koumura, E. Yoshida, T. Funaki, S. Takano, M. Kimura, S. Mori, An Alkyloxyphenyl Group as a Sterically Hindered Substituent on a Triphenylamine Donor Dye for Effective Recombination Inhibition in Dye-Sensitized Solar Cells. Langmuir. 32, 1178-1183 (2016).
R. J. Abraham, H. J. Bemstein, The analysis of nuclear magnetic resonance spectra: V. The analysis of deceptively simple spectra. Can. J. Chem. 39, 216-230 (1961).
P. Diehl, R. J. Chuck, The analysis of an ABXY NMR spectrum using sub-spectral and direct methods. Mol. Phys. 13, 417-423 (1967).
A. Agarwal, J. A. Barnes, J. L. Fletcher, M. J. McGlinchey, B. G. Sayer, Ring currents, local anisotropy, and the problem of the out-of-plane protons: a reinvestigation of the nuclear magnetic resonance spectrum of [10]-paracyclophane. Can. J. Chem. 55, 2575-2581 (1977).
T. Kaneda, T. Otsubo, H. Horita, S. Misumi, Layered compounds. LXI. Carbon-13 NMR study of [n]-[n. n]-, and multilayered [2.2] paracyclophanes. Bull. Chem. Soc. Jpn. 53, 1015-1018 (1980).
J. F. Waters, J. K. Sutter, M. A. B. Meador, L. J. Baldwin, M. A. Meador, Addition curing thermosets endcapped with 4-amino [2.2] paracyclophane. J. Polym. Sci. Part A Polym. Chem. 29, 1917-1924 (1991).
J. Lahann, H. Höcker, R. Langer, Synthesis of amino [2.2] paracyclophanes-beneficial monomers for bioactive coating of medical implant materials. Angew. Chemie Int. Ed. 40, 726-728 (2001).
G. Ricci, R. Ruzziconi, E. Giorgio, Atropisomeric (R. R)-2, 2′—Bi ([2] paracyclo[2] (5, 8) quinolinophane) and (R, R)-1, 1′—Bi ([2] paracyclo[2] (5, 8) isoquinolinophane): Synthesis, Structural Analysis, and Chiroptical Properties. J. Org. Chem. 70, 1011-1018 (2005).
L. Erst, V. Boekelheide, H. Hopf, ¹H and ¹³C NMR spectra of multibridged [2n] cyclophanes. Magn. Reson. Chem. 31, 669-676 (1993).
L. Ernst, The conformational equilibrium of [2.2] paracyclophanes in solution. Liebigs Ann. 1995, 13-17 (1995).
J. F. Schneider, R. Fröhlich, J. Paradies, Synthesis of enantiopure planar-chiral thiourea derivatives. Synthesis (Stuttg). 2010, 3486-3492 (2010).
M. Dracínsky, P. Jansa, P. Bour, Computational and experimental evidence of through-space NMR spectroscopic J coupling of hydrogen atoms. Chem. Eur. J. 18, 981-986 (2012).
M. K. J. ter Wiel, R. A. van Delden, A. Meetsma, B. L. Feringa, Increased speed of rotation for the smallest light-driven molecular motor. J. Am. Chem. Soc. 125, 15076-15086 (2003).
A. L. Santos, D. Liu, A. K. Reed, A. M. Wyderka, A. van Venrooy, J. T. Li, V. D. Li, M. Misiura, O. Samoylova, J. L. Beckham, C. Ayala-Orozco, A. B. Kolomeisky, L. B. Alemany, A. Oliver, G. P. Tegos, J. M. Tour, Sci. Adv. 2022, 8, eabm2055.
CLSI, CLSI Doc. M27, 4th Ed. 2017.
CLSI, CLSI Doc. M38-A2 2008.
L. B. Rice, R. A. Bonomo, in Antibiot. Lab. Med. 4th Ed. Williams Wilkins, Balt. Md, 1996, pp. 453-501.
D. J. Frost. K. D. Brandt, D. Cugier, R. Goldman, J. Antibiot. (Tokyo). 1995, 48, 306.
E. J. Helmerhorst, R. F. Troxler, F. G. Oppenheim, Proc. Natl. Acad. Sci. 2001, 98, 14637.
E. Rayens, K. A. Norris, J. F. Cordero, Clin. Infect. Dis. 2022, 74, 309.
A. G. Stewart, D. L. Paterson, Expert Opin. Pharmacother. 2021, 22, 1857.
M. C. Fisher, N. J. Hawkins, D. Sanglard, S. J. Gurr, Science 2018, 360, 739.
J. R. Perfect, Nat. Rev. Drug Discov. 2017, 16, 603.
D. Farmakiotis, D. P. Kontoyiannis, Int. J. Antimicrob. Agents 2017, 50, 318.
B. M. Kuehn, JAMA 2020, 323, 702.
A. Casadevall, D. P. Kontoyiannis, V. Robert, MBio 2019, 10, DOI 10.1128/mBio.01397-19.
S. B. Ozdemir, N. Demirbas, A. Demirbas, F. A. Ayaz, N. Colak, J. Heterocycl. Chem. 2018, 55,2744,
J. Guinea, T. Peláez, S. Recio, M. Torres-Narbona, E. Bouza, Antimicrob. Agents Chemother. 2008, 52, 1396.
J. E. Nett, M. T. Cain, K. Crawford, D. R. Andes, J. Clin. Microbiol. 2011, 49, 1426.
M. Martins, P. Uppuluri, D. P. Thomas, I. A. Cleary, M. Henriques, J. L. Lopez-Ribot, R. Oliveira, Mycopathologia 2010, 169, 323.
K.-J. Kim, W. S. Sung, B. K. Suh, S.-K. Moon, J.-S. Choi, J. G. Kim, D. G. Lec, Biometals 2009, 22, 235.
T. Shibata, T. Takahashi, E. Yamada, A. Kimura, H. Nishikawa, H. Hayakawa, N. Nomura, J. Mitsuyama, Antimicrob. Agents Chemother. 2012, 56, 5892.
C. A. Lanteri, B. L. Trumpower, R. R. Tidwell, S. R. Meshnick, Antimicrob. Agents Chemother. 2004, 48, 3968.
H. M. McBride, M. Neuspiel, S. Wasiak, Curr. Biol. 2006, 16, R551.
H. Van Genderen, H. Kenis, P. Lux, L. Ungeth, C. Maassen, N. Deckers, J. Narula, L. Hofstra, C. Reutelingsperger, Nat. Protoc. 2006, 1, 363.
F. C. Odds, J. Antimicrob. Chemother. 2003, 52, 1.
C. Tsui, E. F. Kong, M. A. Jabra-Rizk, Pathog. Dis. 2016, 74, ftw018.
F. L. Mayer, D. Wilson, B. Hube, Virulence 2013, 4, 119.
L. Sagan, J. Theor. Biol. 1967, 14, 255.
A. S. Joshi, J. Zhou, V. M. Gohil, S. Chen, M. L. Greenberg, Biochim. Biophys. Acta 2009, 1793, 212.
C. Giorgi, S. Marchi, P. Pinton, Nat. Rev. Mol. cell Biol. 2018, 19, 713.
J. K. Pittman, Cell Calcium 2011, 50, 139.
M. Carraro, P. Bemardi, Cell Calcium 2016, 60, 102.
P. C. Bradshaw, D. W. Jung, D. R. Pfeiffer, J. Biol. Chem. 2001, 276, 40502.
T. Eisenberg, D. Carmona-Gutierrez, S. Büttner, N. Tavernarakis, F. Madeo, Apoptosis 2010, 15,257.
V. M. Gohil, M. N. Thompson, M. L. Greenberg, J. Biol. Chem. 2005, 280, 35410.
S. Liu, Y. Hou, W. Liu, C. Lu, W. Wang, S. Sun, Eukaryot. Cell 2015, 14, 324.
Y. Li, Y. Zhang, C. Zhang, H. Wang, X. Wei, P. Chen, L. Lu, Proc. Natl. Acad. Sci. 2020, 117, 1711.
P. R. Juvvadi, S. C. Lee, J. Heitman, W. J. Steinbach, Virulence 2017, 8, 186.
J. Jia, F. Zhu, X. Ma, Z. W. Cao, Y. X. Li, Y. Z. Chen, Nat. Rev. Drug Discov. 2009, 8, 111.
P. Dong, Y. Zhan, S. Jusuf, J. Hui, Z. Dagher, M. K. Mansour, J. Cheng, Adv. Sci. 2022, 9, 2104384.
T. Parkinson, D. J. Falconer, C. A. Hitchcock, Antimicrob. Agents Chemother. 1995, 39, 1696.
F. S. Clark, T. Parkinson, C. A. Hitchcock, N. A. Gow, Antimicrob. Agents Chemother. 1996, 40,419.
R. D. Cannon, E. Lamping, A. R. Holmes, K. Niimi, P. V Baret, M. V Keniya, K. Tanabe, M. Niimi, A. Goffeau, B. C. Monk, Clin. Microbiol. Rev. 2009, 22, 291.
K. R. Iyer, K, Camara, M. Daniel-Ivad, R. Trilles, S. M. Pimentel-Elardo, J. L. Fossen, K. Marchillo, Z. Liu, S. Singh, J. F. Muñoz, S. H. Kim, J. A. J. Porco, C. A. Cuomo, N. S. Williams, A. S. Ibrahim, J. E. J. Edwards, D. R. Andes, J. R. Nodwell, L. E. Brown, L. Whitesell, N. Robbins, L. E. Cowen, Nat. Commun. 2020, 11, 6429.
N. Sun, W. Fonzi, H. Chen, X. She, L. Zhang, L. Zhang, R. Calderone, Antimicrob. Agents Chemother. 2013, 57, 532.
D.-D. Li, L. Deng, G.-H. Hu, L.-X. Zhao, D.-D. Hu, Y.-Y. Jiang, Y. Wang, Biol. Pharm. Bull. 2013, 36, 1482.
J. L. Slater, L. Gregson, D. W. Denning, P. A. Wam, Med. Mycol. 2011, 49, S107.
D. F. Q. Smith, A. Casadevall, Pathog. Dis. 2021, 79, DOI 10.1093/femspd/ftab013.
A. K. Gupta, R. R. Mays, S. G. Versteeg, N. H. Shear, V. Piguet, Expert Rev. Anti. Infect. Ther. 2018, 16, 929.
M. Schlame, S. Brody, K. Y. Hostetler, Eur. J. Biochem. 1993, 212, 727.
A. M. P. Melo, T. M. Bandeiras, M. Teixeira, Microbiol. Mol. Biol. Rev. 2004, 68, 603.
D. A. Santos, J. S. Hamdan, J. Clin. Microbiol. 2005, 43, 1917.
C. G. Pierce, P. Uppuluri, S. Tummala, J. L. Lopez-Ribot, J. Vis. Exp. 2010, DOI 10.3791/2287,
H. T. Taff, J. E. Nett, D. R. Andes, Med. Mycol. 2012, 50, 214.
M. Kapoor, M. Moloney, Q. A. Soltow, C. M. Pillar, K. J. Shaw, Antimicrob. Agents Chemother. 2019, 64, e01387.
H. Ma, X. Zhao, L. Yang, P. Su, P. Fu, J. Peng, N. Yang, G. Guo, Infect. Drug Resist. 2020, 13, 2509.
M. Edgerton, S. E. Koshlukova, T. E. Lo, B. G. Chrzan, R. M. Straubinger, P. A. Raj, J. Biol. Chem. 1998, 273, 20438.
S. E. Koshlukova, T. L. Lloyd, M. W. B. Araujo, M. Edgerton, J. Biol. Chem. 1999, 274, 18872.
C. de C. Spadari, T. Vila, S. Rozental, K. Ishida, Antimicrob. Agents Chemother. 2018, 62, e00312.
R. I. Benhamou, M. Bibi, J. Berman, M. Fridman, Angew. Chemie Int. Ed. 2018, 57, 6230.
T. A. Vida, S. D. Emr, J. Cell Biol. 1995, 128, 779.
X.-Z. Wu, A.-X. Cheng, L.-M. Sun, S.-J. Sun, H.-X. Lou, Biochim. Biophys. Acta (BBA)-General Subj. 2009, 1790, 770.
C. Pina-Vaz, F. Sansonetty, A. G. Rodrigues, S. Costa-Oliveira, C. Tavares, J. Martinez-de-Oliveira, Clin. Microbiol. Infect. 2001, 7, 609.
J. Lee, D. G. Lee, FEMS Microbiol. Lett. 2014, 355, 36.
H. Tian, S. Qu, Y. Wang, Z. Lu, M. Zhang, Y. Gan, P. Zhang, J. Tian, Appl. Microbiol. Biotechnol. 2017, 101, 3335.
D. Puleston, Cold Spring Harb. Protoc. 2015, DOI 10.1101/pdb.prot086298.
J. Yun, D. G. Lee, FEMS Yeast Res. 2016, 16, DOI 10.1093/femsyr/fow089.
E. Cantón, J. Pemán, M. Gobernado, A. Viudes, A. Espinel-Ingroff, Antimicrob. Agents Chemother. 2005, 49, 1593.
S. Maesaki, P. Marichal, H. Vanden Bossche, D. Sanglard, S. Kohno, J. Antimicrob. Chemother. 1999, 44, 27.
D. C. Sheppard, J. R. Graybill, L. K. Najvar, L. Y. Chiang, T. Doedt, W. R. Kirkpatrick, R. Bocanegra, A. C. Vallor, T. F. Patterson, S. G. Filler, Antimicrob. Agents Chemother. 2006, 50, 3501.
W. Ma, M. Zhang, Z. Cui, X. Wang, X. Niu, Y. Zhu, Z. Yao, F. Ye, S. Geng, C. Liu, Microb. Biotechnol. 2022, 15, 499.
P. M. Quatrin, T. F. A. Kaminski, S. J. Berlitz, I. C. K. Guerreiro, R. F. S. Canto, A. M. Fuentefria, J. Mycol. Med. 2020, 30, 100938.
Postow. M. A.; Callahan, M. K.; Wolchok, J. D. Immune Checkpoint Blockade in Cancer Therapy. J. Clin. Oncol. 2015, 33 (17), 1974.
Dong, H.; Strome, S. E.; Salomao, D. R.; Tamura, H.; Hirano, F.; Flies, D. B.; Roche, P. C.; Lu, J.; Zhu, G.; Tamada, K.; et al. Tumor-Associated B7-HI Promotes T-Cell Apoptosis: A Potential Mechanism of Immune Evasion. Nat. Med. 2002, 8 (8), 793-800.
Jiang, W.; Chan, C. K.; Weissman, I. L.; Kim, B. Y. S.; Hahn, S. M. Immune Priming of the Tumor Microenvironment by Radiation. Trends in Cancer 2016, 2 (11), 638-645.
Krombach, J.; Hennel, R.; Brix, N.; Orth, M.; Schoetz, U.; Ernst, A.; Schuster, J.; Zuchtriegel, G.; Reichel, C. A.; Bierschenk, S.; et al. Priming Anti-Tumor Immunity by Radiotherapy: Dying Tumor Cell-Derived DAMPs Trigger Endothelial Cell Activation and Recruitment of Myeloid Cells. Oncoimmunology 2019, 8 (1).
Cushman, T. R.; Gomez, D.; Kumar, R.; Likacheva, A.; Chang, J. Y.; Cadena, A. P.; Paris, S.; Welsh, J. W. Combining Radiation plus Immunotherapy to Improve Systemic Immune Response. J. Thorac. Dis. 2018, 10 (3), S468-S479.
Bhalla, N.; Brooker, R.; Brada, M. Combining Immunotherapy and Radiotherapy in Lung Cancer. J. Thorac. Dis. 2018, 10 (Suppl 13), S1447.
Vatner, R. E.; Cooper, B. T.; Vanpouille-Box, C.; Demaria, S.; Formenti, S. C. Combinations of Immunotherapy and Radiation in Cancer Therapy. Front. Oncol. 2014, 4 (NOV), 325.

What is claimed:

1. A method of treating a fungal infection in a patient comprising:
(A) administering to the patient in need thereof a stimulus activated molecular machine, wherein the stimulus activated molecule machine is configured to be activated by an appropriate stimulus; and
(B) exposing the patient to a stimulus sufficient to activate the molecular machine.

2. The method of claim 1, wherein the stimulus activated molecular machine comprises a rotor of the formula: 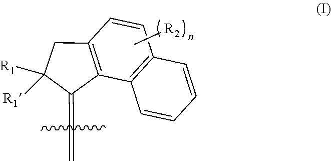
wherein:
R₁ and R₁′ are each independently hydrogen, a C1-C12 alkyl or a C1-C12 substituted alkyl;
R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₂ is a group of the formula: —Y₁—X₁—R₂′, wherein:
Y₁ is —O—, —S—, or —NRₐ—, wherein: R₃ is hydrogen, C1-C6 alkyl, or substituted C1-C6 alkyl;
X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
R₂′ is —NR_{b}R_{b}′R_{b}″, wherein R_{b} and R_{f}′ are each independently hydrogen, C1-C6 alkyl, or C1-C6 substituted alkyl, and R_{b}″ is absent, hydrogen, C1-C6 alkyl, or C1-C6 substituted alkyl; and
n is 0, 1, 2, 3, or 4.

3. The method of claim 1, wherein the stimulus activated molecular machine comprises a stator of the formula: 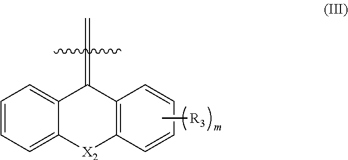
wherein:
X₂ is a covalent bond, O, S, NR_{c}, or CR_{d}R_{d}′, wherein R_{c}, R_{d}, and R_{d}′ are each independently hydrogen, C1-C6 alkyl, or substituted C1-C6 alkyl;
R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₃ is a group of the formula: —Y₂—X₃—R₃′, wherein:
Y₂ is a covalent bond, —O—, —S—, or —NRₑ—, wherein: Rₑ is hydrogen, C1-C6 alkyl, or substituted C1-C6 alkyl;
X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
R₃′ is —NR_{f}R_{f}′R_{f}″, wherein R_{f} and R_{f}′ are each independently hydrogen, C1-C6 alkyl, or C1-C6 substituted alkyl, and R_{f}′ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
m is 0, 1, 2, 3, or 4.

4. The method of claim 1, wherein the stimulus activated molecular motor comprises a rotor of the formula: 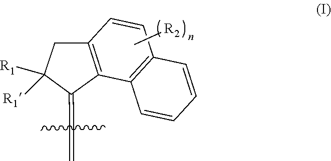
wherein:
R₁ and R₁′ are each C1-C12 alkyl or C1-C12 substituted alkyl;
R₁′ is hydrogen;
R₂ is hydrogen, C1-C12 alkoxy, or substituted C1-C12 alkoxy; or
R₂ is a group of the formula: —Y₁ —X₁—R₂′, wherein:
Y₁ is —NRₐ—, wherein: Rₐ is hydrogen, C1-C6 alkyl, or substituted C1-C6 alkyl;
X₁ is C1-C12 alkanediyl or C1-C12 substituted alkanediyl; and
R_{f}′ is —NR_{b}R_{b}′R_{b}″, wherein R_{b} and R_{f}′ are each independently hydrogen, C1-C6 alkyl, or C1-C6 substituted alkyl, and R_{b}″ is absent, hydrogen, C1-C6 alkyl, or C1-C6 substituted alkyl; and
n is 0 or 1,
and a stator of the formula: 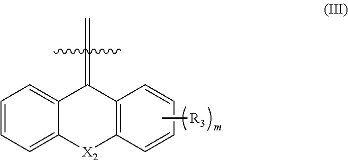
wherein:
X₂ is a covalent bond or S;
R₃ is hydrogen or halo; or
R₃ is a group of the formula: —Y₂—X₃—R₃′, wherein:
Y₂ is a covalent bond or —NRₑ—, wherein: Rₑ is hydrogen, C1-C6 alkyl, or substituted C1-C6 alkyl;
X₃ is C1-C12 alkanediyl, C1-C12 substituted alkanediyl, C2-C12 alkynediyl, or C2-C12 substituted alkynediyl; and
R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C1-C12 heterocycloalkyl, C1-C12 substituted heterocycloalkyl, C18-C24 triarylphosphine or C18-C24 substituted triarylphosphine; and
m is 1 or 2.

5. The method of claim 1, wherein the stimulus activated molecular motor is further defined as:

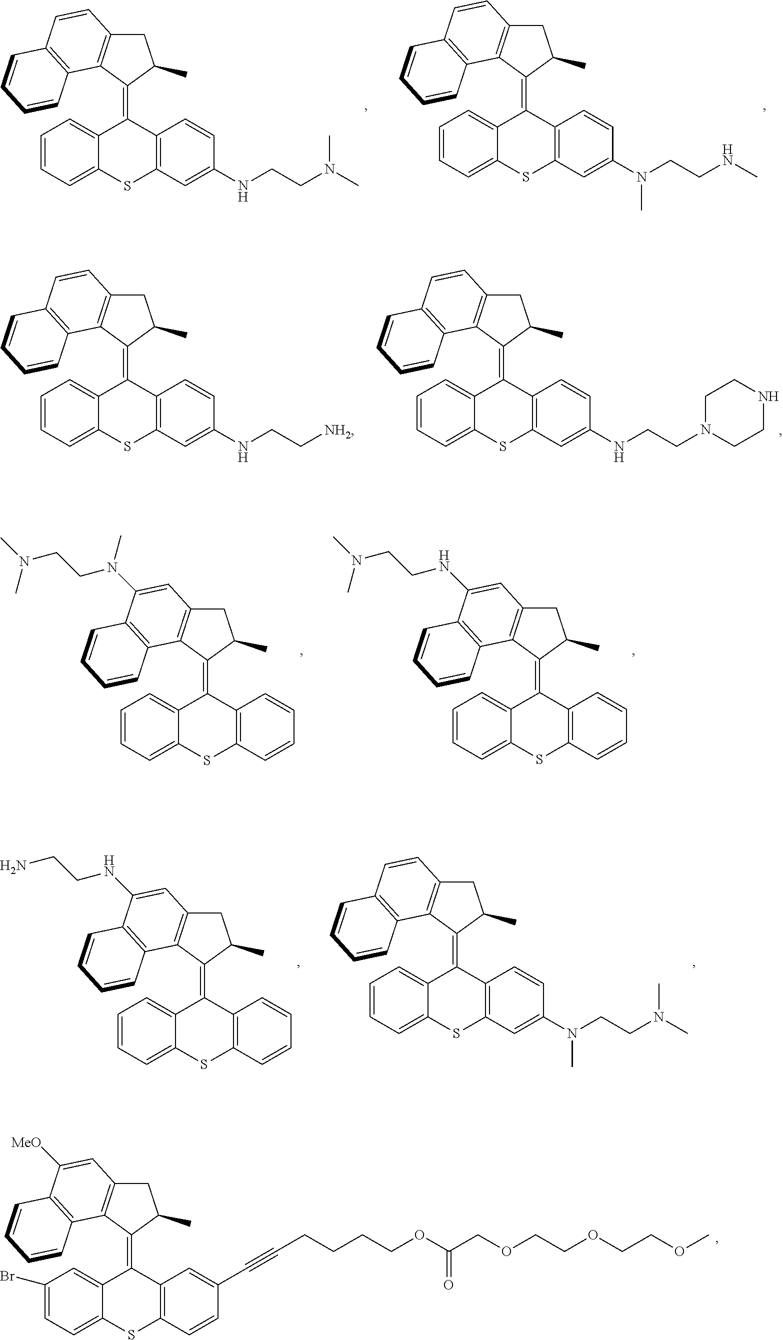

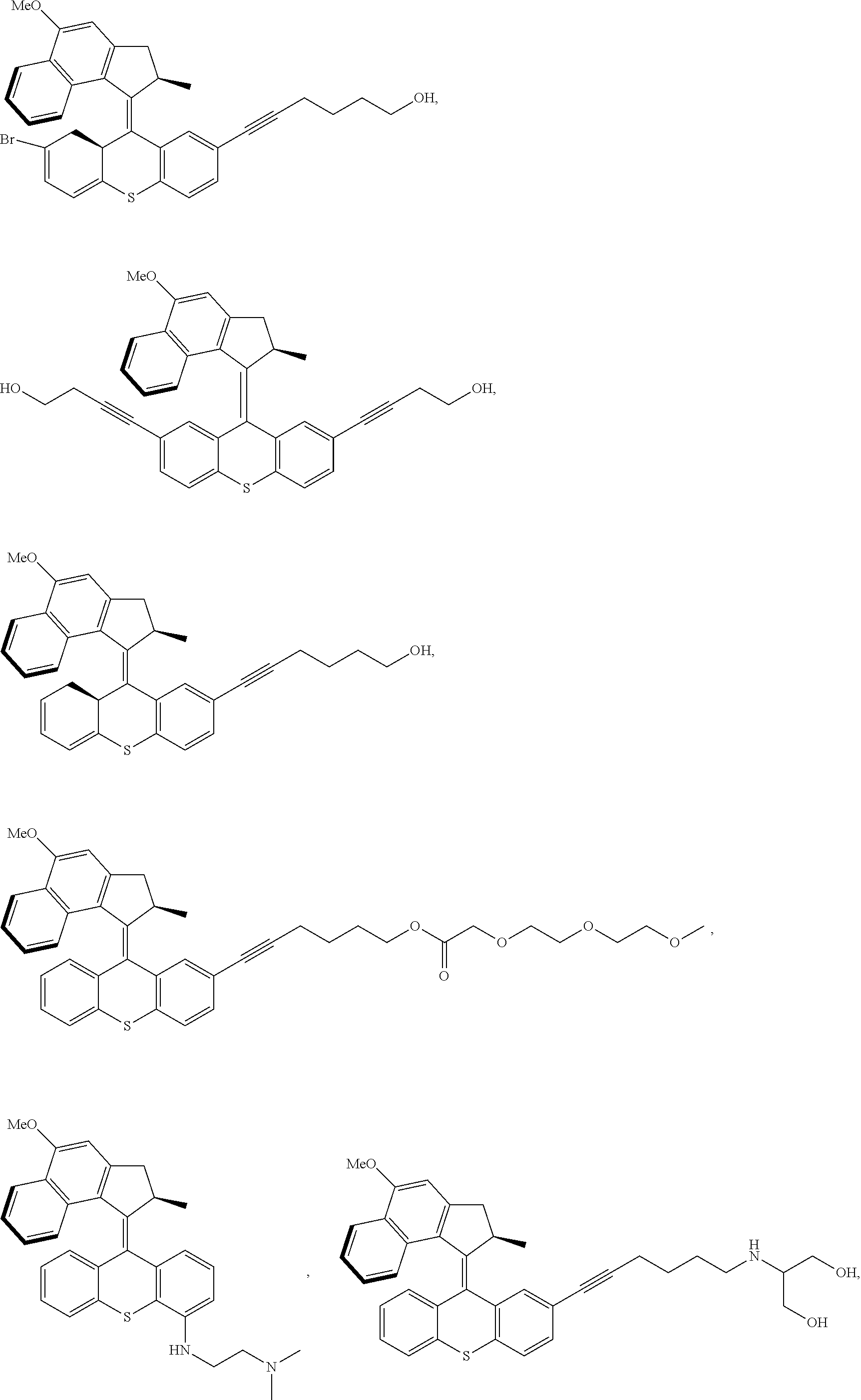

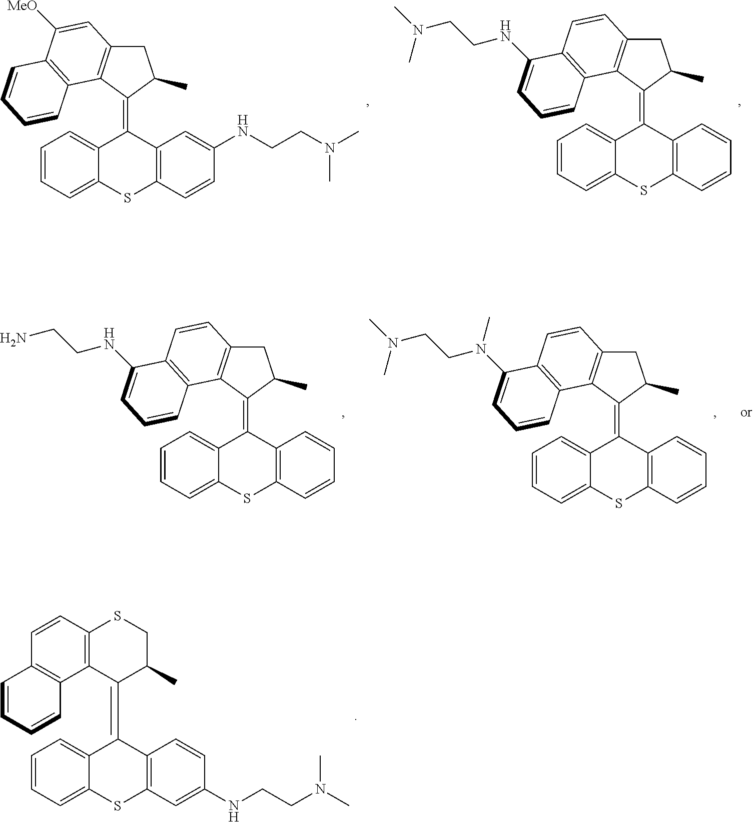

6. A method of treating a disease or disorder in a patient caused by an infection of a microorganism comprising:
(A) administering to the patient in need thereof a stimulus activated molecular machine, wherein the stimulus activated molecule machine is configured to be activated by an appropriate stimulus; and
(B) exposing the patient to a stimulus sufficient to activate the molecular machine.

7. The method of claim 6, wherein the microorganism is a bacterium.

8. The method of claim 7, wherein the bacterium is sensitive to one or more antibiotics.

9. The method of claim 6, wherein the microorganism is a fungus.

10. The method of claim 6, wherein the method further comprises administering a second anti-fungal therapy.

11. The method of claim 6, wherein the stimulus activated molecular machine comprises a rotor of the formula: 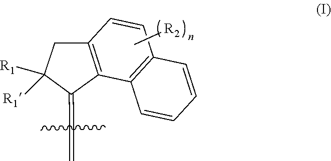
wherein:
R₁ and R₁′ are each independently hydrogen, a C1-C12 alkyl or a C1-C12 substituted alkyl;
R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₂ is a group of the formula: —Y₁ —X₁—R₂′, wherein:
Y₁ is —O—, —S—, or —NRₐ —, wherein: R₃ is hydrogen, C1-C6 alkyl, or substituted C1-C6 alkyl;
X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
R_{f}′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
n is 0, 1, 2, 3, or 4.

12. The method of claim 6, wherein the stimulus activated molecular machine comprises a stator of the formula: 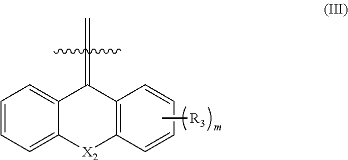
wherein:
X₂ is a covalent bond, O, S, NR_{c}, or CRₐR_{d}′, wherein R_{c}, R_{d}, and R_{d}′ are each independently hydrogen, C1-C6 alkyl, or substituted C1-C6 alkyl;
R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₃ is a group of the formula: —Y₂—X₃—R₃′, wherein:
Y₂ is a covalent bond, —O—, —S—, or —NRₑ—, wherein: Rₑ is hydrogen, C1-C6 alkyl, or substituted C1-C6 alkyl;
X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
m is 0, 1, 2, 3, or 4.

13. The method of claim 6, wherein the stimulus activated molecular motor comprises a rotor of the formula: 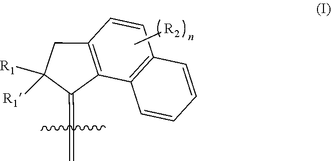
wherein:
R₁ is C1-C12 alkyl or C1-C12 substituted alkyl;
R₁′ is hydrogen;
R₂ is hydrogen, C1-C12 alkoxy, or substituted C1-C12 alkoxy; or
R₂ is a group of the formula: —Y₁—X₁—R₂′, wherein:
Y₁ is —NRₐ—, wherein: R₃ is hydrogen, C1-C6 alkyl, or substituted C1-C6 alkyl;
X₁ is C1-C12 alkanediyl or C1-C12 substituted alkanediyl; and
R₂′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
n is 0 or 1,
and a stator of the formula: 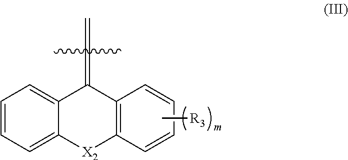
wherein:
X₂ is a covalent bond or S;
R₃ is hydrogen or halo; or
R₃ is a group of the formula: —Y₂—X₃—Rₑ′, wherein:
Y₂ is a covalent bond, —O—, —S—, or —NRₑ—, wherein: Rₑ is hydrogen, C1-C6 alkyl, or substituted C1-C6 alkyl;
X₃ is C1-C12 alkanediyl, C1-C12 substituted alkanediyl, C2-C12 alkynediyl, or C2-C12 substituted alkynediyl; and
R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C1-C12 heterocycloalkyl, C1-C12 substituted heterocycloalkyl, C18-C24 triarylphosphine or C18-C24 substituted triarylphosphine; and
m is 1 or 2.

14. The method of claim 6, wherein the stimulus activated molecular motor is further defined as:

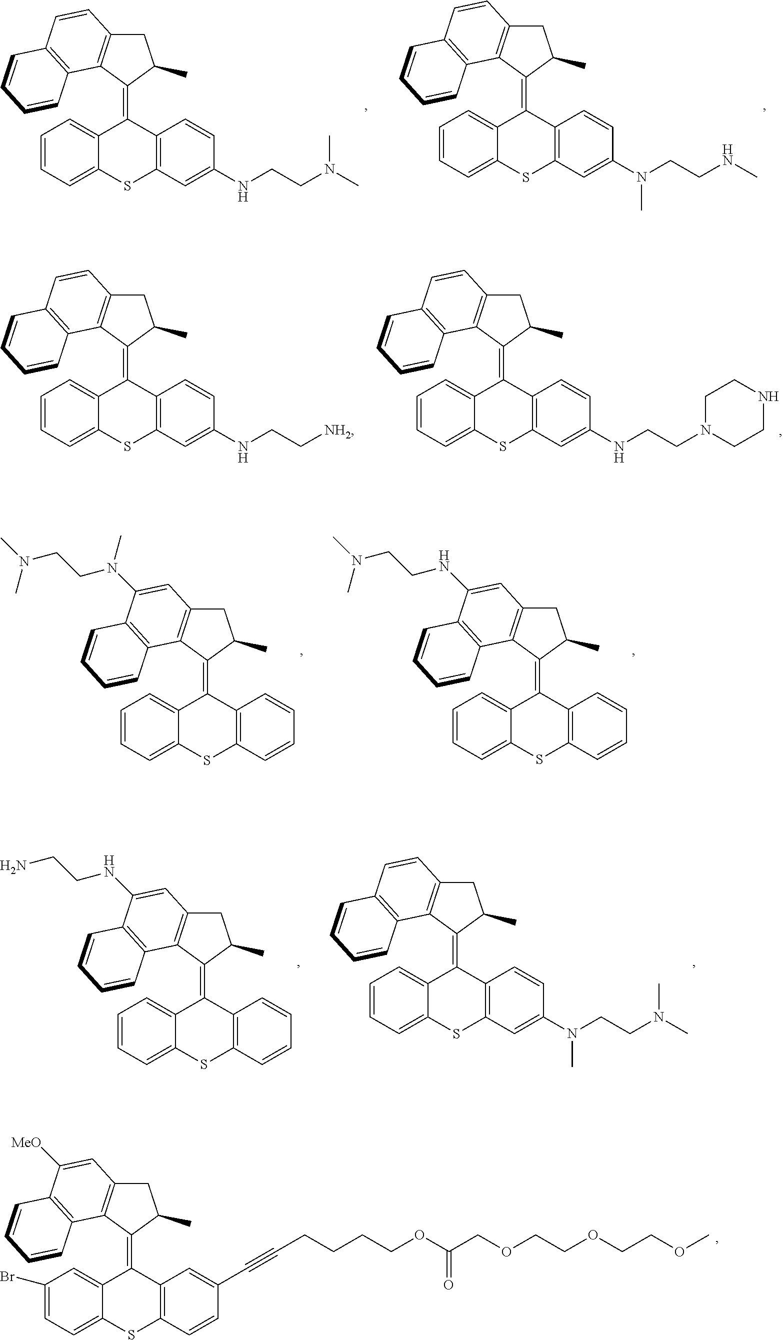

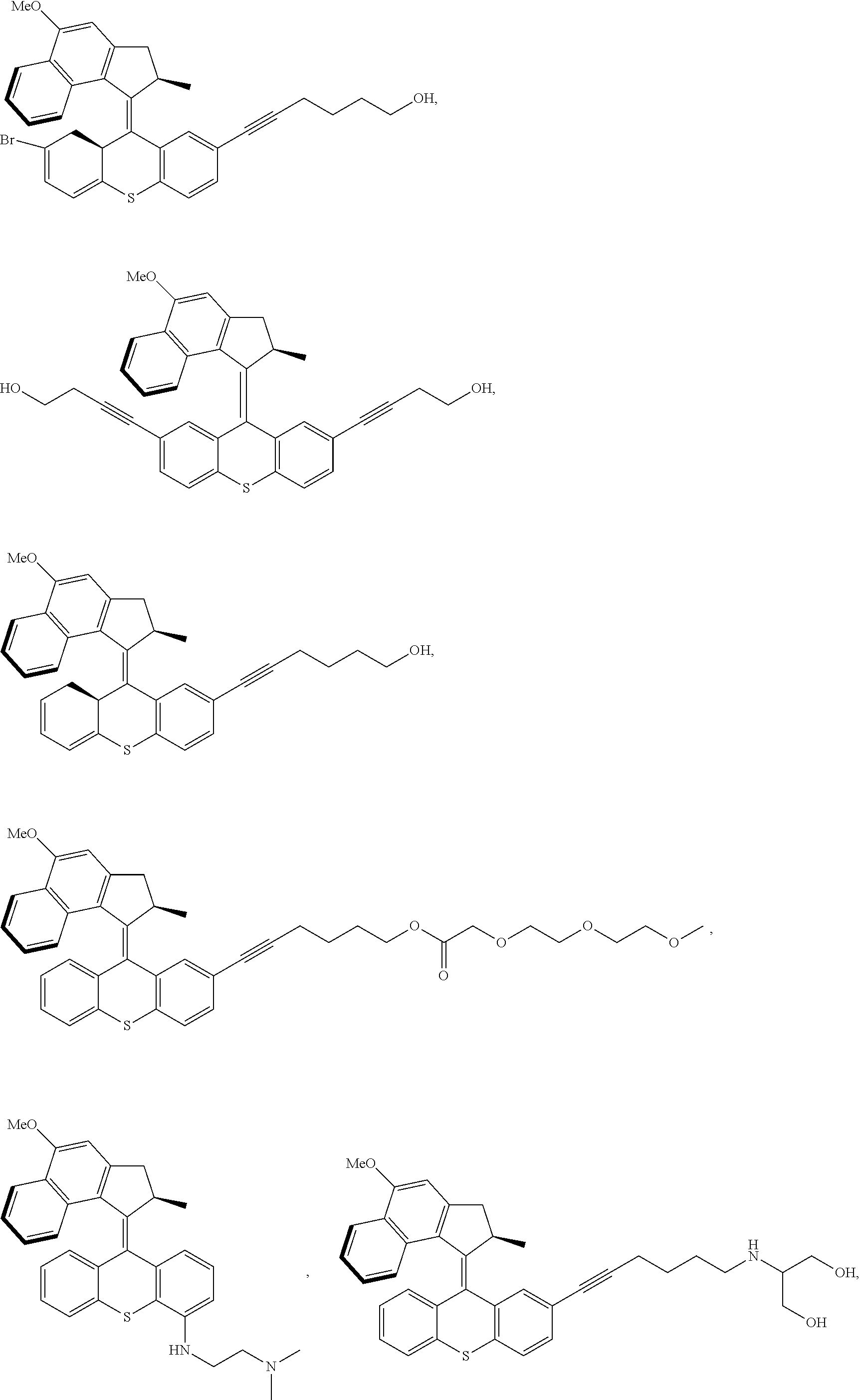

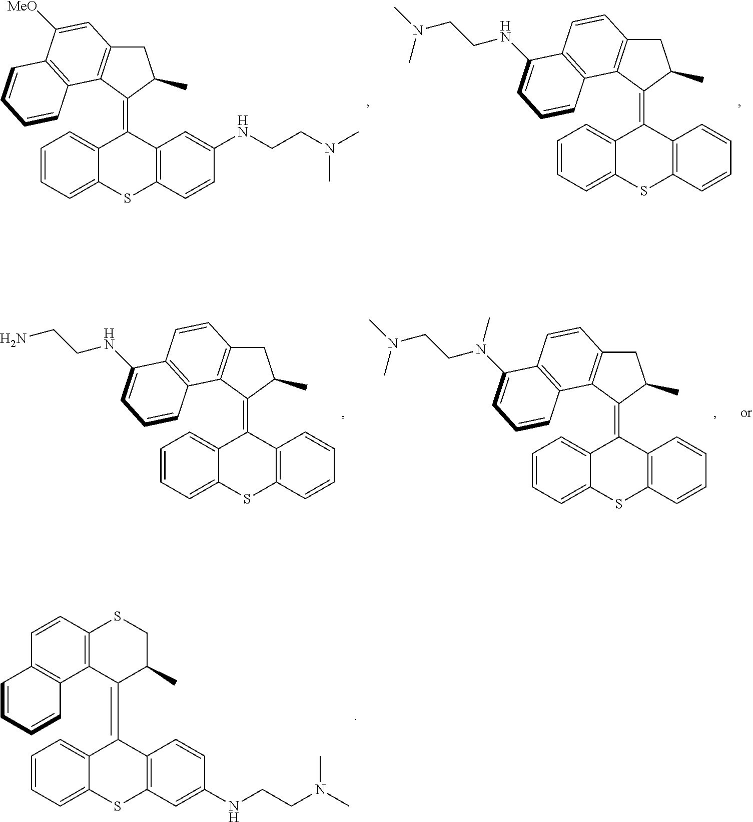

15. The method of claim 6, wherein the stimulus activated molecular motor is further defined as:

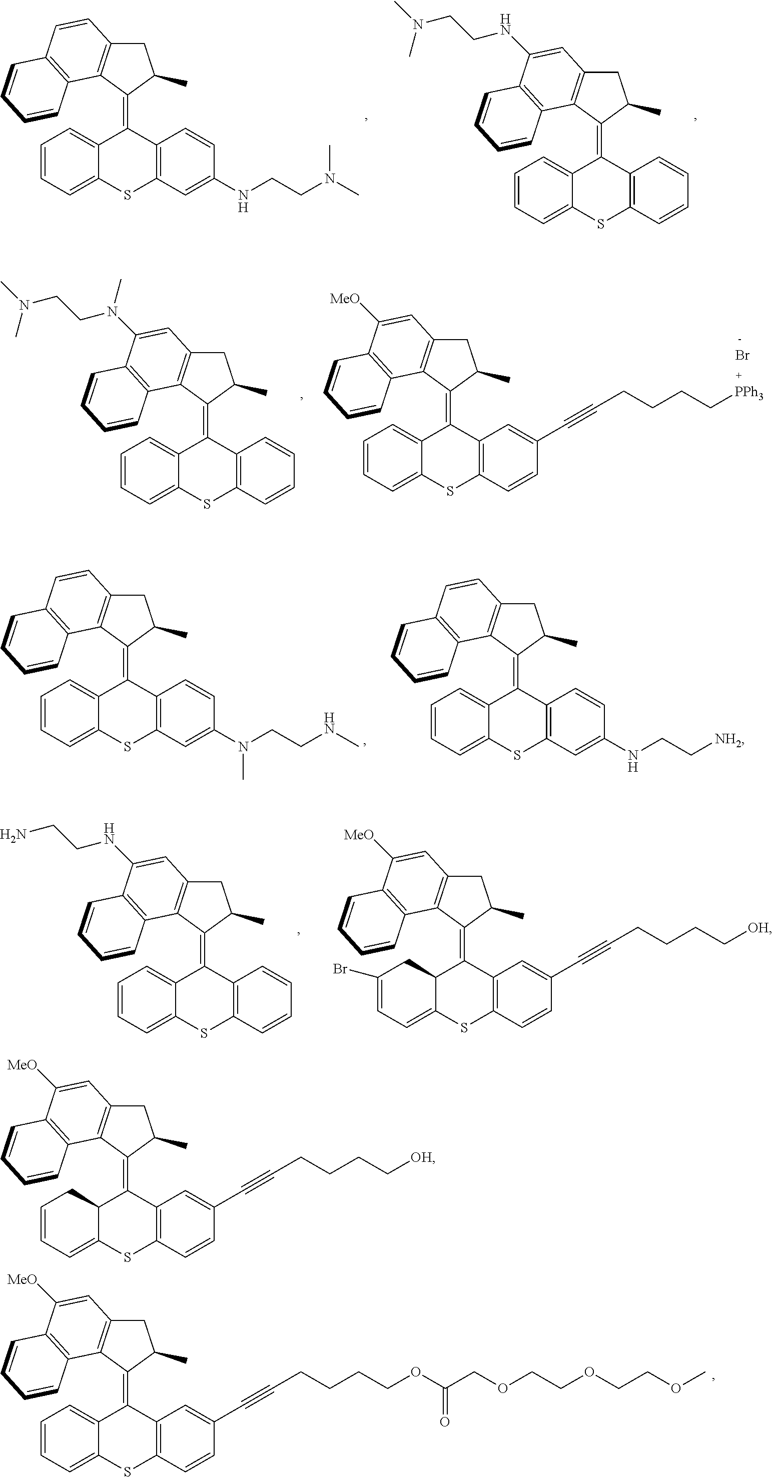

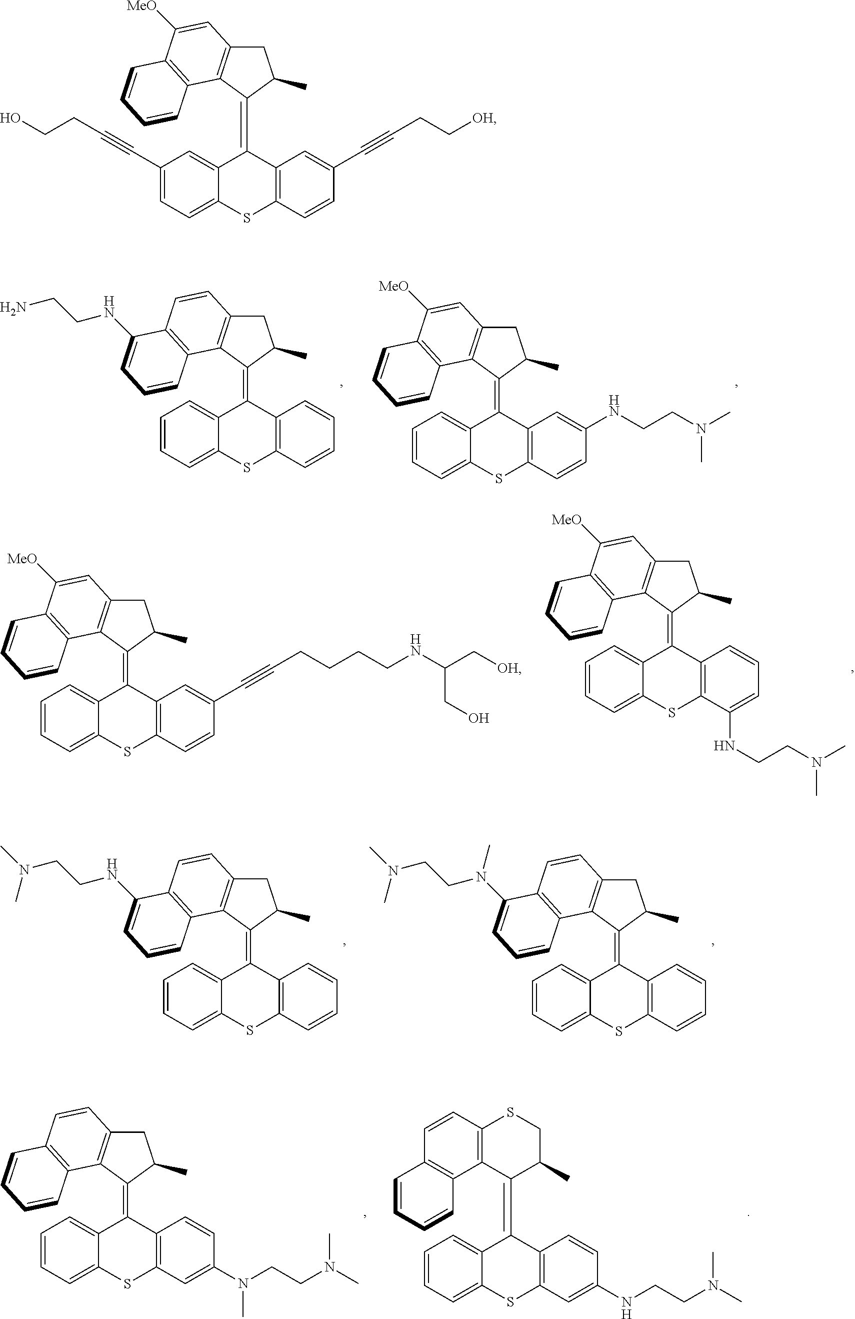

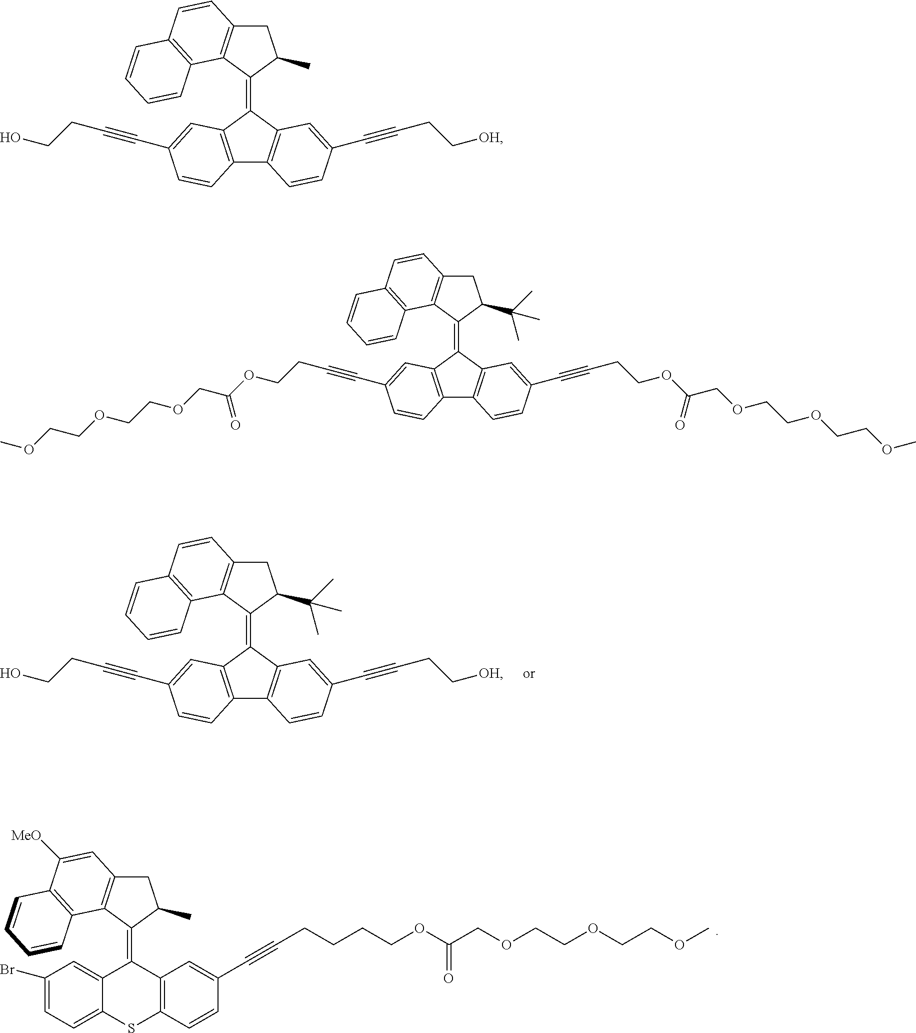

16. A method of treating cancer in a patient in need thereof comprising administering to the patient a stimulus activated molecular machine, wherein the stimulus activated molecule machine is configured to be activated by an appropriate stimulus, and exposing the patient to an appropriate stimulus wherein the stimulus activated molecular machine comprises a rotor of the formula: 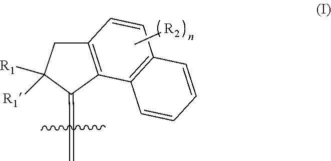
wherein:
R₁ and R₁′ are each independently hydrogen, a C1-C12 alkyl or a C1-C12 substituted alkyl;
R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₂ is a group of the formula: —Y₁—X₁—R₂′, wherein:
Y₁ is —O—, —S—, or —NRₐ—, wherein: R₃ is hydrogen, C1-C6 alkyl, or substituted C1-C6 alkyl;
X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
R_{f}′ is —NR_{b}R_{b}′R_{b}, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
n is 0, 1, 2, 3, or 4; and
a stator of the formula: 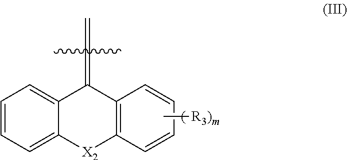
wherein:
X₂ is a covalent bond, O, S, NR_{c}, or CRₐR_{d}′, wherein R_{c}, R_{d}, and R_{d}′ are each independently hydrogen, C1-C6 alkyl, or substituted C1-C6 alkyl;
R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₃ is a group of the formula: —Y₂—X₃—R₃′, wherein:
Y₂ is a covalent bond, —O—, —S—, or —NRₑ—, wherein: Rₑ is hydrogen, C1-C6 alkyl, or substituted C1-C6 alkyl;
X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
R₃′ is —NR_{f}R_{f}′R_{f}″,wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
m is 0, 1, 2, 3, or 4;
provided that the compound is not:

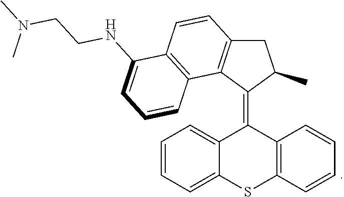

17. The method of claim 16, wherein the stimulus activated molecular motor comprises a rotor of the formula: 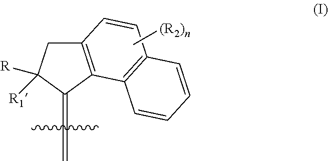
wherein:
R₁ and R₁′ are each C1-C12 alkyl or C1-C12 substituted alkyl;
R₁′ is hydrogen; and
n is 0,
and a stator of the formula: 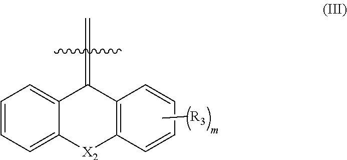
wherein:
X₂ is S;
R₃ is hydrogen; or
R₃ is a group of the formula: —Y₂ X₃—R₃′, wherein:
Y₂ is —NRₑ—, wherein: Rₑ is hydrogen, C1-C6 alkyl, or substituted C1-C6 alkyl;
X₃ is C1-C12 alkanediyl or C1-C12 substituted alkanediyl; and
R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl; and
m is 1.

18. The method of claim 16, wherein the stimulus activated molecular machine is further defined as:

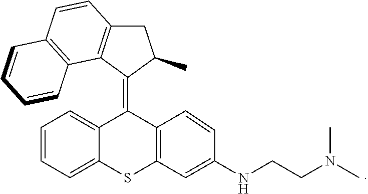

19. The method of claim 16, wherein the cancer is a carcinoma, sarcoma, lymphoma, leukemia, melanoma, mesothelioma, multiple myeloma, or seminoma.

20. The method of claim 16, wherein the cancer is of the bladder, blood, bone, brain, breast, central nervous system, cervix, colon, endometrium, esophagus, gall bladder, genitalia, genitourinary tract, bead, kidney, larynx, liver, lung, muscle tissue, neck, oral or nasal mucosa, ovary, pancreas, prostate, skin, spleen, small intestine, large intestine, stomach, testicle, or thyroid.

21. The method of claim 16, wherein the method further comprises administering a second therapeutic agent.

22. The method of claim 1, wherein the stimulus activated molecular machine comprises a Feringa-type molecular machine.

23. The method of claim 1, wherein the stimulus activated molecular machine comprises a rotor that is connected to a stator through an alkenyl or alkynyl group.

24. The method of claim 23, wherein the stimulus activated molecular machine comprises a rotor that is connected to a stator through an atropisomeric alkene.

25. The method of claim 1, wherein the stimulus activated molecular motor further comprises a rotor is further defined as: 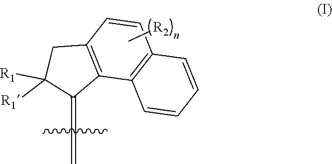
wherein:
R₁ and R₁′ are each independently hydrogen, a C1-C12 alkyl or a C1-C12 substituted alkyl;
R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₂ is a group of the formula: —Y₁—X₁—R₂′, wherein:
Y₁ is —O—, —S—, or —NRₐ—, wherein: R₃ is hydrogen, C1-C6 alkyl, or substituted C1-C6 alkyl;
X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
R₃′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
n is 0, 1, 2, 3, or 4.

26. The method of claim 25, wherein the rotor is further defined as: 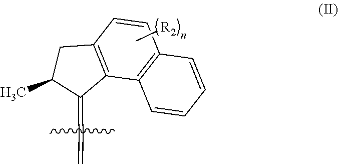
wherein:
R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₂ is a group of the formula: —Y₁ —X₁—R₂′, wherein:
Y₁ is —O—, —S—, or —NRₐ—, wherein: R₃ is hydrogen, C1-C6 alkyl, or substituted C1-C6 alkyl;
X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
R₂′ is —NR_{b}R_{b}′R_{b}″, wherein R_{b} and R₁′ are each independently hydrogen, C1-C6 alkyl, or C1-C6 substituted alkyl, and R_{b}″ is absent, hydrogen, C1-C6 alkyl, or C1-C6 substituted alkyl; and
n is 0, 1, 2, 3, or 4.

27. The method of claim 25, wherein R₁ is C1-C12 alkyl or substituted C1-C12 alkyl.

28. The method of claim 25, wherein R₂ is —Y₁ —X₁—R₂.

29. The method of claim 25, wherein R₂ is —NHCH₂CH₂N(Me)₂.

30. The method of claim 25, wherein n is 0 or 1.

31. The method of claim 1, wherein the stimulus activated molecular motor further comprises a stator further defined as: 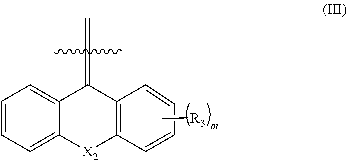
wherein:
X₂ is a covalent bond, O, S, NR_{c}, or CR_{d}R_{d}′, wherein R_{c}, R_{d}, and R_{d}′ are each independently hydrogen, C1-C6 alkyl, or substituted C1-C6 alkyl;
R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₃ is a group of the formula: —Y₂—X₃—R₃′, wherein:
Y₂ is a covalent bond, —O—, —S—, or —NRₑ—, wherein: Rₑ is hydrogen, C1-C6 alkyl, or substituted C1-C6 alkyl;
X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
m is 0, 1, 2, 3, or 4.

32. The method of claim 31, wherein the stator is further defined as: 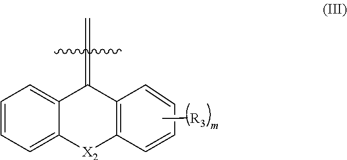
wherein:
X₂ is a covalent bond, O, S, NR_{c}, or CR_{d}R_{d}′, wherein R_{c}, R_{d}, and R_{d}′ are each independently hydrogen, C1-C6 alkyl, or substituted C1-C6 alkyl;
R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₃ is a group of the formula: —Y₂X₃—R₃′, wherein:
Y₂ is a covalent bond, —O—, —S—, or —NRₑ—, wherein: Rₑ is hydrogen, C1-C6 alkyl, or substituted C1-C6 alkyl;
X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is —OC(O)CH₂(OCH₂CH₂)ₐR₃″, wherein R₃″ is hydroxy, C1-C6 alkoxy, or C1-C6 substituted alkoxy, and a is 1, 2, 3, or 4 or R₃′ is hydroxy, C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
m is 0 or 1.

33. The method of claim 31, wherein R₃ is —Y₂—X₃—R₃′.

34. The method of claim 31, wherein R₃′ is —NR_{f}R_{f}′R_{f}″.

35. The method of claim 31, wherein R_{f} and R_{f}′ are each independently C1-C6 alkyl or C1-C6 substituted alkyl.

36. The method of claim 31, wherein R₃ is —NHCH₂CHEN(Me)₂ or —NHCH₂CH₂(CH₂CH₂)₂NH.

37. The method of claim 31, wherein m is 0 or 1.

38. The method of claim 31, wherein X₂ is S.

39. The method of claim 1, wherein the stimulus activated molecular machine is further defined as:

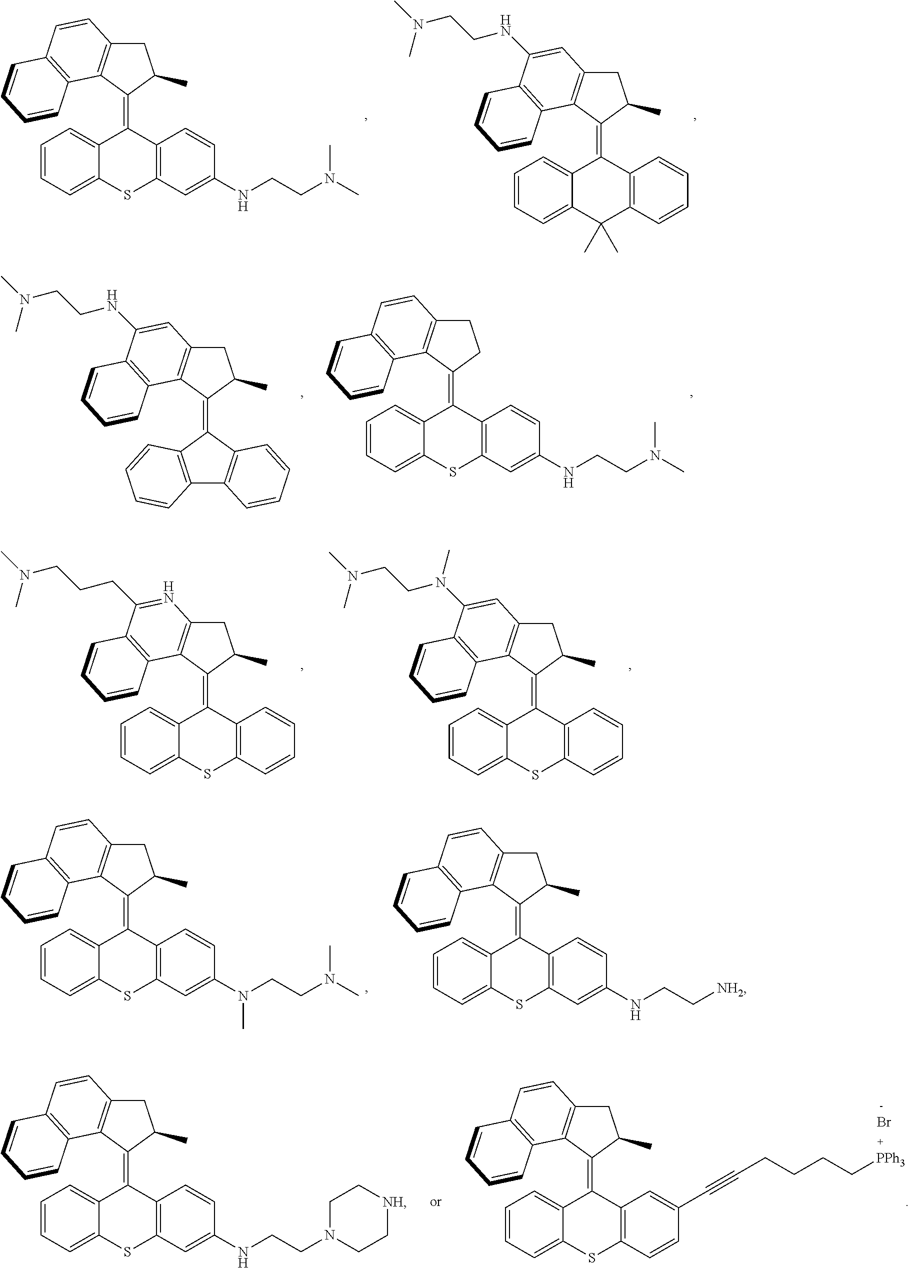

40. The method of claim 1, wherein the rotational component of the stimulus activated molecular machine rotates at a speed greater than 1 Hz.

41. The method of claim 1, wherein the stimulus activated molecular machine is activated by a stimulus.

42. The method of claim 41, wherein the stimulus is electromagnetic radiation.

43. The method of claim 42, wherein the electromagnetic radiation comprises UV light, visible light, or near infrared light.

44. The method of claim 1, wherein the stimulus activated molecular machine is activated for a controlled time period.

45. The method of claim 1, wherein the energy source is a laser.

46. The method of claim 1, wherein the intensity of the energy source is controlled.

47. A molecular machine comprising:
(A) a rotor of the formula: 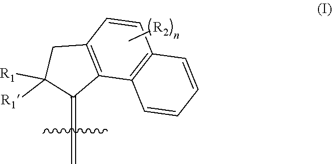
wherein:
R₁ and R₁′ are each independently hydrogen, a C1-C12 alkyl or a C1-C12 substituted alkyl;
R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₂ is a group of the formula: —Y₁—X₁—R₂′, wherein:
Y₁ is —O—, —S—, or —NRₐ—, wherein: R₃ is hydrogen, C1-C6 alkyl, or substituted C1-C6 alkyl;
X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
R₂′ is —NR_{b}R_{b}′R_{b}″, wherein R_{b} and R₁′ are each independently hydrogen, C1-C6 alkyl, or C1-C6 substituted alkyl, and R_{b}″ is absent, hydrogen, C1-C6 alkyl, or C1-C6 substituted alkyl; and
n is 0, 1, 2, 3, or 4; and
(B) a stator of the formula: 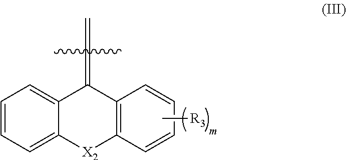
wherein:
X₂ is a covalent bond, O, S, NR_{c}, or CRₐR_{d}′, wherein R_{c}, R_{d}, and R_{d}′ are each independently hydrogen, C1-C6 alkyl, or substituted C1-C6 alkyl;
R₃ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₃ is a group of the formula: —Y₂—X₃—R₃′, wherein:
Y₂ is a covalent bond, —O—, —S—, or —NRₑ—, wherein: Rₑ is hydrogen, C1-C6 alkyl, or substituted C1-C6 alkyl;
X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
R₃′ is —NR_{f}R_{f}′R_{f}″, wherein R_{f} and R′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
m is 0, 1, 2, 3, or 4;
provided at least one of R₂ is a group of the formula: —Y₁—X₁—R₂′ or at least one of R₃ is a group of the formula: —Y₂—X₃—R₃′, and provided that the molecular machine is not a compound of the formula:

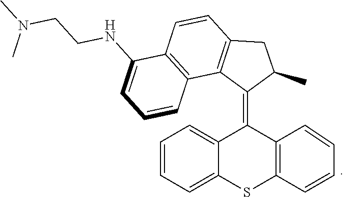

48. The molecular machine of claim 47, wherein the rotor is further defined as: 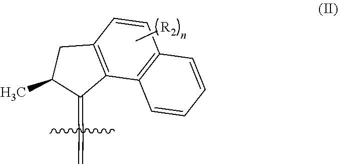
wherein:
R₂ is hydrogen, amino, cyano, halo, hydroxy, C1-C12 alkyl, C3-C12 cycloalkyl, C1-C12 alkenyl, C1-C12 alkynyl, C1-C12 aryl, C1-C12 aralkyl, C1-C12 heteroaryl, C1-C12 heteroaralkyl, C1-C12 heterocycloalkyl, C1-C12 alkoxy, C1-C12 alkylamino, C1-C12 dialkylamino, C1-C12 acyl, C1-C12 amido, C1-C12 acyloxy, or a substituted version of any of these groups; or
R₂ is a group of the formula: —Y₁ —X₁—R₂′, wherein:
Y₁ is —O—, —S—, or —NRₐ—, wherein: R₃ is hydrogen, C1-C6 alkyl, or substituted C1-C6 alkyl;
X₁ is C1-C12 alkanediyl, C1-C12 substituted alkanediyl, C3-C12 cycloalkanediyl, C3-C12 substituted cycloalkanediyl, C6-C12 arenediyl, and C6-C12 substituted arenediyl; and
R₂′ is —NR_{b}R_{b}′R_{b}″, wherein R_{b} and R_{b}′ are each independently hydrogen, C1-C6 alkyl, or C1-C6 substituted alkyl, and R_{b}″ is absent, hydrogen, C1-C6 alkyl, or C1-C6 substituted alkyl; and
n is 0, 1, 2, 3, or 4.

49. The molecular machine of claim 47, wherein R₁ is C1-C12 alkyl or substituted C1-C12 alkyl.

50. The molecular machine according to any one of claims 47-49, wherein R₂ is —Y₁ —X₁—R₂′.

51. The molecular machine of claim 47, wherein Rₑ is —NHCH₂CH₂N(Me)₂.

52. The molecular machine of claim 47, wherein n is 0.

53. The molecular machine of claim 47, wherein the stator is further defined as: 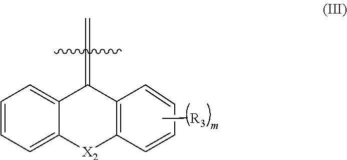
wherein:
Y₂ is a covalent bond, —O—, —S—, or —NRₑ—, wherein: Rₑ is hydrogen, C1-C6 alkyl, or substituted C1-C6 alkyl;
X₃ is C1-C12 alkanediyl, C1-C12 substituted alkanediyl, C3-C12 cycloalkanediyl, C3-C12 substituted cycloalkanediyl, C2-C12 alkenediyl, C2-C12 substituted alkenediyl, C2-C12 alkynediyl, C2-C12 substituted alkynediyl, C6-C12 arenediyl, and C6-C12 substituted arenediyl; and
R₃′ is —NR_{f}R_{f}′R_{f}″, wherein R_{f} and R_{f}′ are each independently hydrogen, C1-C6 alkyl, or C1-C6 substituted alkyl, and R_{f}″ is absent, hydrogen, C1-C6 alkyl, or C1-C6 substituted alkyl, or R₃′ is C3-C12 cycloalkyl, C3-C12 substituted cycloalkyl, C1-C12 heterocycloalkyl, C1-C12 substituted heterocycloalkyl, C18-C24 triarylphosphine or C18-C24 substituted triarylphosphine; and
m is 0 or 1.

54. The molecular machine of claim 47, wherein R₃ is —Y₂—X₃—R₃′.

55. The molecular machine of claim 47, wherein R₃′ is —NR_{f}R_{f}′R_{f}″.

56. The molecular machine of claim 47, wherein R_{f} and R_{f}′ are each independently C1-C6 alkyl or C1-C6 substituted alkyl.

57. The molecular machine of claim 47, wherein R₃′ is C1-C12 heterocycloalkyl or C1-C12 heterocycloalkyl.

58. The molecular machine of claim 47, wherein R₃ is —NHCH₂CH₂N(Me)₂ or —NHCH₂CH₂N(CH₂CH₂)₂NH.

59. The molecular machine of claim 47, wherein n is 0.

60. The molecular machine of claim 47, wherein X₂ is S.

61. The molecular machine of claim 47, wherein the stimulus activated molecular machine is further defined as: [chemical structure]
